(19) United States
(12) Patent Application Publication
(10) Pub. No.: US 2004/0131625 A1
(43) Pub. Date: Jul. 8, 2004

(54) Vaccine composition

(76) Inventors: Francois-Xavier Jacques Berthet, Rixensart (BE); Yves Lobet, Rixensart (BE); Jan Poolman, Rixensart (BE); Vincent Georges Christian Louis Verlant, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/467,534

(22) PCT Filed: Feb. 8, 2002
(86) PCT No.: PCT/EP02/01356

(51) Int. Cl. A61K 39/00; A61K 39/38; A61K 39/118

(52) U.S. Cl. 424/184.1

(57) ABSTRACT

The present invention relates to the field of Gram-negative bacterial vaccine compositions, their manufacture, and the use of such compositions in medicine. More particularly it relates to the field of useful Gram-negative bacterial outer membrane vesicle (or bleb) compositions comprising heterologously expressed Chlamydia antigens, and advantageous methods of rendering these compositions more effective and safer as a vaccine.

### CROSS REFERENCE TO RELATED APPLICATIONS

This application is a National Stage Application filed under 35 U.S.C. §371 of International Application No: PCT/EP02/01356, filed 08 Feb. 2002.

### FIELD OF THE INVENTION

The present invention relates to the field of Gram-negative bacterial vaccine compositions, their manufacture, and the use of such compositions in medicine. More particularly it relates to the field of useful Gram-negative bacterial outer membrane vesicle (or bleb) compositions comprising heterologously expressed Chlamydia antigens, and advantageous methods of rendering these compositions more effective and safer as a vaccine.

### BACKGROUND OF THE INVENTION

Chlamydiae are obligate intracellular Gram negative bacteria which replicate only in cytoplasmic inclusions of eukaryotic cells. They have a unique developmental cycle which is represented by two major forms, the spore-like elementary body (EB) which is the infectious form transmitted from cell to cell, and the non infectious, metabolically active reticulate body (RB) which replicates within the host-cell.

Of the four known chlamydial species, Chlamydia trachomatis and C. pneumoniae are the important human pathogens. The recently defined species C. pneumoniae (Grayston 1989) is now recognized as a major cause of respiratory tract infections (Grayston 1993) and data are now growing for an association with atherosclerosis. The association is supported by seroepidemiological studies, studies demonstrating the presence of the bacterium in the atherosclerotic lesions, studies showing C. pneumoniae capability to replicate in the different cell types present in the atheroclerotic lesions, interventional trials with antibiotics in patients with coronary artery disease and experimental respiratory tract infection in rabbits or apolipoprotein-E deficient mice which leads to inflammatory changes in the aorta (Danesh 1997, Fong 1997, Laitinen 1997, Moazed 1997). Overall, those data implicate C. pneumoniae as a causative and/or aggravating factor of atherosclerosis.

C. trachomatis is a major human pathogen; transmitted from human to human (there is no known animal reservoir), it causes ocular and genital infections which can result in long term sequelae. Trachoma, a chlamydial ocular infection, is endemic in several developing countries and is the world's leading cause of preventable blindness with millions people affected by the disease. Genital chlamydial infections constitute the most common bacterial sexually transmitted disease (STD) worldwide. In 1996, WHO generated a new set of global estimates for four major STDs drawing an extensive review of the published and unpublished prevalence data (Gerbase 1998). It has been estimated that in 1995, 4 and 5.2 million new cases of C. trachomatis infection occured in individuals aged 15-49 for North America and Western Europe, respectively; worlwide, C. trachomatis totalized an estimate of 89.1 million new cases. Collectively, data show higher infection rates in women as compared to men (Washington 1987, Peeling 1995, Cates 1991); higher incidence is found in adolescent and young adults, approximately 70% of the chlamydial infections being reported in the 15-24 years of age group (Peeling 1995).

There is a clear need for effective vaccines against Chlamydia trachomatis and Chlamydia pneumoniae.

Outer Membrane Vesicles (blebs)

Gram-negative bacteria are separated from the external medium by two successive layers of membrane structures. These structures, referred to as the cytoplasmic membrane and the outer membrane (OM), differ both structurally and functionally. The outer membrane plays an important role in the interaction of pathogenic bacteria with their respective hosts. Consequently, the surface exposed bacterial molecules represent important targets for the host immune response, making outer-membrane components attractive candidates in providing vaccine, diagnostic and therapeutics reagents.

Whole cell bacterial vaccines (killed or attenuated) have the advantage of supplying multiple antigens in their natural micro-environment. Drawbacks around this approach are the side effects induced by bacterial components such as endotoxin and peptidoglycan fragments. On the other hand, acellular subunit vaccines containing purified components from the outer membrane may supply only limited protection and may not present the antigens properly to the immune system of the host.

Proteins, phospholipids and lipopolysaccharides are the three major constituents found in the outer-membrane of all Gram-negative bacteria. These molecules are distributed asymmetrically: membrane phospholipids (mostly in the inner leaflet), lipooligosaccharides (exclusively in the outer leaflet) and proteins (inner and outer leaflet lipoproteins, integral or polytopic membrane proteins). For many bacterial pathogens which impact on human health, lipopolysaccharide and outer-membrane proteins have been shown to be immunogenic and amenable to confer protection against the corresponding disease by way of immunization.

The OM of Gram-negative bacteria is dynamic and, depending on the environmental conditions, can undergo drastic morphological transformations. Among these manifestations, the formation of outer-membrane vesicles or “blebs” has been studied and documented in many Gram-negative bacteria (Zhou, L et al. 1998. FEMS Microbiol. Lett. 163: 223-228). Among these, a non-exhaustive list of bacterial pathogens reported to produce blebs include: Bordetella pertussis, Borrelia burgdorferi, Brucella melitensis, Brucella ovis, Chlamydia psittaci, Chlamydia trachomatis, Esherichia coli, Haemophilus influenzae, Legionella pneumophila, Neisseria gonorrhoeae, Neisseria meningitidis, Pseudomonas aeruginosa and Yersinia enterocolitica. Although the biochemical mechanism responsible for the production of OM blebs is not fully understood, these outer membrane vesicles have been extensively studied as they represent a powerful methodology in order to isolate outer-membrane protein preparations in their native conformation.

Examples of bacterial species from which bleb vaccines can be made have been reviewed in WO 01/09350 (incorporated by reference herein). For example, N. meningitidis serogroup B (menB) excretes outer membrane blebs in sufficient quantities to allow their manufacture on an industrial scale. Such multicomponent outer-membrane protein vaccines from naturally-occurring menB strains have been found to be efficacious in protecting teenagers from menB disease and have become registered in Latin America. An alternative method of preparing outer-membrane vesicles is via the process of detergent extraction of the bacterial cells (EP 11243).

### SUMMARY OF THE INVENTION

The present inventors have found that Gram-negative bacterial blebs are an ideal context to present Chlamydia outer membrane proteins. In particular gonococcal blebs are useful in the case of presenting C. trachomatis OMPs and meningococcal blebs are useful in the case of presenting C. pneumoniae OMPs. This is because a) these outer-membrane proteins can integrate into such blebs in a native (or near-native) conformation thus retaining a useful immunological effect; b) blebs (particularly from Neisseria strains) can be produced in industrial quantities, c) blebs may be mucosally administered, and d) the combination of Chlamydia antigens with native bleb antigens can have important interactions for certain conditions such as salpingitis.

The present invention thus provides advantageous Gram-negative bacterial bleb preparations (derived from bleb-producing bacterial strains listed above, and preferably not derived from Chlamydia) presenting on its surface one or more recombinant (and preferably heterologous) protein antigens from Chlamydia trachomatis or Chlamydia pneumoniae. Advantagous vaccine formulations and methods of administration are also provided.

### DESCRIPTION OF THE INVENTION

The present invention provides a Gram-negative bacterial bleb presenting on its surface one or more outer membrane protein from Chlamydia.

In the context of this application the term “presenting on its surface” indicates that the Chlamydia protein should be exposed to the outer surface of the bleb and tethered to the outer membrane (preferably by being integrated into the outer membrane). Most preferably it should take up its native fold within the heterologous bleb context.

An efficient strategy to modulate the composition of a Bleb preparation in this way is to deliver one or more copies of a DNA segment containing an expression cassette comprising a gene encoding said Chlamydia outer membrane protein into the genome of a Gram-negative bacterium.

A non exhaustive list of preferred bacterial species that could be used as a recipient for such a cassette includes: Bordetella pertussis, Borrelia burgdorferi, Brucella melitensis, Brucella ovis, Chlamydia psittaci, Chlamydia trachomatis, Esherichia coli, Haemophilus influenzae, Legionella pneumophila, Neisseria gonorrhoeae, Neisseria meningitidis, Pseudomonas aeruginosa and Yersinia enterocolitica. Neisseria meningitidis, Neisseiria gonorrhoeae, Moraxella catarrhalis, Haemophilus influenzae, Pseudomonas aeruginosa, Chlamydia trachomatis, Chlamydia pneumoniae are more preferred for this purpose, and Neisseria gonorrhoeae and Neisseria meningitidis are most preferred for making the blebs of this invention. Preferably the Chlamydia OMPs are expressed heterologously, and in such situations Chlamydia strains should not be used to make the blebs of the invention.

The gene(s) contained in the expression cassette may be homologous (or endogenous) (i.e. exist naturally in the genome of the manipulated bacterium) or, preferably, heterologous (i.e. do not exist naturally in the genome of the manipulated bacterium). The introduced expression cassette may consist of unmodified, “natural” promoter/gene/operon sequences or engineered expression cassettes in which the promoter region and/or the coding region or both have been altered. A non-exhaustive list of preferred promoters (preferably strong) that could be used for expression includes the promoters porA, porB, lbpB, tbpB, p110, lst, hpuAB from N. meningitidis or N. gonorroheae, the promoters p2, p5, p4, ompF, p1, ompH, p6, hin47 from H. influenzae, the promoters ompH, ompG, ompCD, ompE, ompB1, ompB2, ompA of M. catarrhalis, the promoter •pL, lac, tac, araB of Escherichia coli or promoters recognized specifically by bacteriophage RNA polymerase such as the E. coli bacteriophage T7.

In a preferred embodiment of the invention the expression cassette is delivered and integrated in the bacterial chromosome by means of homologous and/or site specific recombination (as discussed in WO 01/09350 incorporated by reference herein). Integrative vectors used to deliver such genes and/or operons can be conditionally replicative or suicide plasmids, bacteriophages, transposons or linear DNA fragments obtained by restriction hydrolysis or PCR amplification. Integration is preferably targeted to chromosomal regions dispensable for growth in vitro. A non exhaustive list of preferred loci that can be used to target DNA integration includes the porA, porB, opa, opc, rmp, omp26, lecA, cps, lgtB genes of Neisseiria meningitidis and Neisseria gonorrhoeae, the P1, P5, hmw1/2, IgA-protease, fimE genes of NTHi; the lecA1, lecA2, omp106, uspA1, uspA2 genes of Moraxella catarrhalis. Alternatively, the expression cassette used to modulate the expression of bleb component(s) can be delivered into a bacterium of choice by means of episomal vectors such as circular/linear replicative plasmids, cosmids, phasmids, lysogenic bacteriophages or bacterial artificial chromosomes. Selection of the recombination event can be selected by means of selectable genetic marker such as genes conferring resistance to antibiotics (for instance kanamycin, erythromycin, chloramphenicol, or gentamycin), genes conferring resistance to heavy metals and/or toxic compounds or genes complementing auxotrophic mutations (for instance pur, leu, met, aro). Blebs may be made from the resulting modified strain.

The expression of some heterologous proteins in bacterial blebs may require the addition of outer-membrane targeting signal(s). The preferred method to solve this problem is by creating a genetic fusion between a heterologous gene and a gene coding for a resident OMP as a specific approach to target recombinant proteins to blebs. Most preferably, the heterologous gene is fused to the signal peptides sequences of such an OMP.

A particularly preferred application of this invention is the introduction of Chlamydia (trachomatis or pneumoniae) protective antigens (preferably outer membrane proteins) into Gram-negative bacterial blebs (preferably not from Chlamydia strains). This has several advantages including the fact that such blebs (and vaccines comprising them) are extremely suitable for mucosal administration, which is beneficial as a mucosal (IgA) immune response against the Chlamydia antigens present in the bleb will be more protective against Chlamydia infections which manifest themselves in the mucosa. Recombinant bacteria capable of producing blebs of the invention, processes of making such bacteria, and processes of making bleb preparations are further aspects of this invention.

Chlamydia trachomatis Antigens Integrated into a Gram Negative Bacterial bleb

A particularly preferred embodiment is in the field of the prophylaxis or treatment of sexually-transmitted diseaseses (STDs). It is often difficult for practitioners to determine whether the principal cause of a STD is due to gonococcus or Chlamydia trachomatis infection. These two organisms are major causes of salpingitis—a disease which can lead to sterility in the host. It would be useful if a STD could be vaccinated against or treated with a combined vaccine effective against disease caused by both organisms. The Major Outer Membrane Protein (MOMP or OMP1 or OMPI) of C. trachomatis has been shown to be the target of protective antibodies. However, the structural integrity of this integral membrane protein is important for inducing such antibodies. In addition, the epitopes recognised by these antibodies are variable and define more than 10 serovars. The bleb context of the invention allows the proper folding of one or more MOMP or other Chlamydia membrane proteins for vaccine purposes. The engineering of (preferably) a gonococcal strain expressing one or more C. trachomatis MOMP serovars and/or one or more other protective Chlamydia OMPs in the outer membrane, and the production of blebs therefrom, produces a single solution to the multiple problems of correctly folded membrane proteins, the presentation of sufficient MOMP serovars and/or other Chlamydia OMPs to protect against a wide spectrum of serovars, and the simultaneous prophylaxis/treatment of gonococcal infection (and consequently the non-requirement of practitioners to initially decide which organism is causing particular clinical symptoms—both organisms can be vaccinated against simultaneously thus allowing the treatment of the STD at a very early stage). Preferred loci for gene insertion in the gonoccocal chromosome are give above. Other preferred, protective C. trachomatis genes that could be incorporated are HMWP, PmpG and those OMPs disclosed in WO 99/28475 (incorporated by reference herein).

A particularly preferrred embodiment of the invention provides a Gram-negative bacterial bleb (preferably gonococcal) presenting on its surface the PorB outer membrane protein (see below) from Chlamydia trachomatis. A bacterial strain capable of producing such a bleb is a further aspect of the invention.

PorB Chlamydia trachomatis serovar D (D/UW-3/Cx) DNA sequence [SEQ ID NO:1]
ATGAGTAGCAAGCTAGTGAACTATCTCCGTTTGACTTTCCTATCTTTTTTAGGGATCGCATCTACTTCATT
AGACGCTATGCCTGCGGGGAATCCGGCGTTTCCAGTCATCCCGGGGATTAATATTGAACAGAAAAATGCCT
GTTCTTTCATTTATGTAATTCTTATGATGTACTATCCGCACTGTCCGGTAACCTGAAGCTCTGCTTCTGCG
GAGATTATATCTTTTCGAAGAAGCTCAGGTAAAAGATGTCCCTGTCGTTACCTCTGTGACAACAGCTGGGG
TTGGTCCTTCTCCTGATATTACTTCGACAACCAAAACGCGAAATTTCGATCTCGTGAACTGTAATCTCAAT
ACAAACTGTGTAGCTGTAGCTTTTTCCCTTCCTATCGTTCGCTGAGCGCGATTCCTCTGTTTGATGTGAGT
TTCGAAGTGAAAGTAGGAGGACTGAAACAATACTACCGCCTTCCCATGAATGCCTATCGAGACTTCACCTC
GGAACCTCTCAATTCTGAATCAGAAGTTACGGACGGGATGATTGAAGTACGTCCAATTACGGATTTGTTTG
GGATGTTAGCTTGAAAAAAGTCATATGGAAAGATGGCGTTTCCTTTGTAGGCGTCGGTCAGACTATCGCCA
TGCTTCTTGCCCTATTGACTACATCATTGCAAACAGTCAAGCTAATCCAGAAGTATTCATCGCTGATCGGA
TGGGAAACTGAACTTCAAGGAGTGGAGTGTCTGCGTAGGTCTTACTACCTATGTGAATGACTACGTTCTTC
CTTCTTAGCGTTTTCTATAGGGAGTGTTTCTCGCCAAGCTCCGGACGACAGCTTCAAAAAATTAGAAGATC
GCTTCACTAACTCAAATTTAAAGTTCGTAAAATTACCAGCTCTCATCGTGGAAACATCTGCATCGGAGCGA
CAAACTATGTCGCCGATAATTCTTCTACAACGTAGAAGGAAGATGGGGAAGCCAGCGCGCTGTGAACGTCT
CCGGAGGATTCCAATTCTAA
Translated amino acid sequence [SEQ ID NO:2]
MSSKLVNYLR LTFLSFLGIA STSLDAMPAG NPAFPVIPGI
NIEQKNACSF DLCNSYDVLS ALSGNLKLCF CGDYIFSEEA QVKDVPVVTS
VTTAGVGPSP DITSTTKTRN FDLVNCNLNT NCVAVAFSLP DRSLSAIPLF
DVSFEVKVGG LKQYYRLPMN AYRDFTSEPL NSESEVTDGM IEVQSNYGFV
WDVSLKKVIW KDGVSFVGVG ADYRHASCPI DYIIANSQAN PEVFIADSDG
KLNFKEWSVC VGLTTYVNDY VLPYLAFSIG SVSRQAPDDS FKKLEDRFTN
LKFKVRKITS SHRGNICIGA TNYVADNFFY NVEGRWGSQR AVNVSGGFQF

The presence of PorB in the blebs means that the antigen can be mucosally administered more easily, and provides more effective protection than if administered alone.

The present invention additionally provides a Gram-negative bacterial bleb (preferably gonococcal) presenting on its surface one or more of the following proteins from Chlamydia trachomatis, or C. trachomatis PorB in combination with one or more of the following proteins. It will be clear to a skilled person that instead of the sequences below (and the PorB sequence above), the natural analogue of the sequences from other C. trachomatis serovars or serotypes could be used, as could genes encoding functional analogues of the proteins comprising insertions, deletions or substitutions from the recited sequences which unaffect the immunological properties of the encoded protein. Preferably a sequence from a serovar D strain should be selected. A bacterial strain capable of producing such a bleb is a further aspect of the invention.

```
>gi|6578118|gb|AAC68456.2| predicted Protease containing IRBP and DHR domains
[Chlamydia trachomatis][SEQ ID NO:3]
MKMNRIWLLLLTFSSAIHSPVQGESLVCKNALQDLSFLEHLLQVKYAPKTWKEQYLGWDLVQSSVSAQQK
LRTQENPSTSFCQQVLADFIGGLNDFHAGVTFFAIESAYLPYTVQKSSDGRFYFVDIMTFSSEIRVGDEL
LEVDGAPVQDVLATLYGSNHKGTAAEESAALRTLFSRMASLGHKVPSGRTTLKIRRPFGTTREVRVKWRY
VPEGVGDLATIAPSIRAPQLQKSMRSFFPKKDDAFHRSSSLFYSPMVPHFWAELRNHYATSGLKSGYNIG
STDGFLPVIGPVIWESEGLFRAYISSVTDGDGKSHKVGFLRIPTYSWQDMEDFDPSGPPPWEEFAKIIQV
FSSNTEALIIDQTNNPGGSVLYLYALLSMLTDRPLELPKHRMILTQDEVVDALDWLTLLENVDTNVESRL
ALGDNMEGYTVDLQVAEYLKSFGRQVLNCWSKGDIELSTPIPLFGFEKIHPHPRVQYSKPICVLINEQDF
SCADFFPVVLKDNDRALIVGTRTAGAGGFVFNVQFPNRTGIKTCSLTGSLAVREHGAFIENIGVEPHIDL
PFTANDIRYKGYSEYLDKVKKLVCQLINNDGTIILAEDGSF
>gi|3329331|gb|AE001359.1:101-1906[SEQ ID NO:4]
ATGAAAATGAATAGGATTTGGCTATTACTGCTTACCTTTTCTTCTGCCATACATTCTCCTGTACAAGGAG
AAAGCTTGGTTTGCAAGAATGCTCTTCAAGATTTGAGTTTTTTAGAGCATTTATTACAGGTTAAATATGC
TCCTAAAACATGGAAAGAGCAATACTTAGGATGGGATCTTGTTCAAAGCTCCGTTTCTGCACAGCAGAAG
CTTCGTACACAAGAAAATCCATCAACAAGTTTTTGCCAGCAGGTCCTTGCTGATTTTATCGGAGGATTAA
ATGACTTTCACGCTGGAGTAACTTTCTTTGCGATAGAAAGTGCTTACCTTCCTTATACCGTACAAAAAAG
TAGTGACGGCCGTTTCTACTTTGTAGATATCATGACTTTTTCTTCAGAGATCCGTGTTGGAGATGAGTTG
CTAGAGGTGGATGGGGCGCCTGTCCAAGATGTACTCGCTACTCTATATGGAAGCAATCACAAAGGGACTG
CAGCTGAAGAGTCGGCTGCTTTAAGAACACTATTTTCTCGCATGGCCTCTTTAGGGCACAAAGTACCTTC
TGGGCGCACTACTTTAAAGATTCGTCGTCCTTTTGGTACTACGAGAGAAGTTCGTGTGAAATGGCGTTAT
GTTCCTGAAGGTGTAGGAGATTTGGCTACCATAGCTCCTTCTATCAGGGCTCCACAGTTACAGAAATCGA
TGAGAAGCTTTTTCCCTAAGAAAGATGATGCGTTTCATCGGTCTAGTTCGCTATTCTACTCTCCAATGGT
TCCGCATTTTTGGGCAGAGCTTCGCAATCATTATGCAACGAGTGGTTTGAAAAGCGGGTACAATATTGGG
AGTACCGATGGGTTTCTCCCTGTCATTGGGCCTGTTATATGGGAGTCGGAGGGTCTTTTCCGCGCTTATA
TTTCTTCGGTGACTGATGGGGATGGTAAGAGCCATAAAGTAGGATTTCTAAGAATTCCTACATATAGTTG
GCAGGACATGGAAGATTTTGATCCTTCAGGACCGCCTCCTTGGGAAGAATTTGCTAAGATTATTCAAGTA
TTTTCTTCTAATACAGAAGCTTTGATTATCGACCAAACGAACAACCCAGGTGGTAGTGTCCTTTATCTTT
ATGCACTGCTTTCCATGTTGACAGACCGTCCTTTAGAACTTCCTAAACATAGAATGATTCTGACTCAGGA
TGAAGTGGTTGATGCTTTAGATTGGTTAACCCTGTTGGAAAACGTAGACACAAACGTGGAGTCTCGCCTT
GCTCTGGGAGACAACATGGAAGGATATACTGTGGATCTACAGGTTGCCGAGTATTTAAAAAGCTTTGGAC
GTCAAGTATTGAATTGTTGGAGTAAAGGGGATATCGAGTTATCAACGCCTATTCCTCTTTTTGGTTTTGA
GAAGATTCATCCACATCCTCGAGTTCAATACTCTAAACCGATTTGTGTTTTGATCAATGAGCAAGACTTT
TCTTGTGCTGACTTCTTCCCTGTAGTTTTGAAAGACAATGATCGAGCTCTTATTGTTGGTACTCGAACAG
CTGGAGCTGGAGGATTTGTCTTTAATGTGCAGTTCCCAAATAGAACTGGAATAAAAACTTGTTCTTTAAC
AGGATCATTAGCTGTTAGAGAGCATGGTGCCTTCATTGAGAACATCGGAGTCGAACCGCATATCGATCTG
CCTTTTACAGCGAATGATATTCGCTATAAAGGCTATTCCGAGTATCTTGATAAGGTCAAAAAATTGGTTT
GTCAGCTGATCAATAACGACGGTACCATTATTCTTGCGGAAGATGGTAGTTTTTAA
>gi|6578109|gb|AAC68227.2| CHLPN 76kDa Homolog [Chlamydia trachomatis][SEQ ID NO:5]
MKKYFYKGFVGALLLACGSTNLAFAQASSMDSQLWSVEDLDSYLSSKGFVETRKRDGVLRLAGDVRARWI
YAKEDLETTQTPAKPMLPTNRYRSEFNLYVDYTAANSWMTSKMNWVTIAGGESSAAGLDINRAFLGYRFY
KNPETQAEVFAEIGRSGLGDIFDSDVQFNSNFDGIHLYAARRISEKLPFTMIVHGGPFVVNMAEKEYAWV
VEAILNKLPGNFVVKTSVVDWNTLTAKTNDPADASAAQPAKPNTKYDYLVWQWLVGKSTAMPWFNGQTKN
LYTYGAYLFNPLAEIPENWKQSTTPTTKITNGKENHAWFIGCSLGGVRRAGDWSATVRYEYVEALAIPEI
DVAGIGRGNQMKYWFAQAIKQGLDPKESNGFTNYKGVSYQFVMGLTDSVSFRAYAAYSKPANDNLGSDFT
YRKYDLGLISSF
>gi|3329068|gb|AE001333.1:c3495-2197[SEQ ID NO:6]
ATGAAAAAATACTTTTATAAAGGGTTTGTAGGCGCGCTTTTATTAGCTTGTGGGTCTACAAACTTGGCTT
TTGCGCAGGCTAGTTCGATGGATAGCCAGCTATGGTCTGTTGAAGATTTAGATTCTTATTTGAGTTCCAA
AGGTTTTGTCGAGACTCGTAAGCGCGATGGAGTTCTACGTTTAGCTGGAGATGTCCGCGCTCGATGGATT
TATGCAAAAGAGGATCTTGAGACAACTCAGACTCCTGCTAAACCTATGTTACCTACCAATCGGTATCGTA
GTGAATTCAATTTGTATGTGGATTACACCGCTGCTAATAGTTGGATGACTTCGAAAATGAATTGGGTAAC
GATTGCTGGCGGAGAATCTTCTGCAGCAGGGTTAGATATTAATCGTGCCTTCTTAGGATACCGATTCTAC
AAAAACCCAGAAACGCAAGCAGAAGTATTTGCAGAGATTGGTCGCTCTGGATTGGGAGATATTTTTGATT
CCGACGTTCAGTTTAATAGTAATTTCGACGGAATTCATTTATACGCTGCGCGACGTATTAGTGAGAAACT
TCCTTTCACCATGATTGTTCATGGTGGTCCTTTTGTCGTGAATATGGCAGAGAAAGAGTATGCTTGGGTC
GTGGAAGCTATTTTGAATAAACTCCCAGGAAATTTCGTTGTGAAAACGAGTGTTGTTGACTGGAATACGT
TAACAGCAAAAACGAATGATCCAGCAGACGCAAGCGCTGCACAACCAGCTAAACCTAATACCAAGTACGA
TTATTTAGTATGGCAATGGTTGGTTGGGAAGAGCACAGCTATGCCATGGTTTAATGGACAAACAAAAAAT
CTTTACACTTACGGAGCCTATCTCTTTAATCCATTAGCGGAAATACCAGAGAACTGGAAACAATCAACAA
CTCCTACAACCAAAATTACAAATGGTAAGGAAAACCATGCTTGGTTCATCGGCTGCTCTCTAGGCGGTGT
TCGACGAGCTGGAGACTGGTCTGCAACAGTTCGTTATGAGTATGTTGAAGCTTTAGCGATTCCAGAAATT
GATGTCGCGGGTATTGGTCGCGGAAACCAAATGAAATATTGGTTTGCTCAAGCTATCAAACAAGGATTGG
ATCCTAAAGAATCTAACGGCTTTACTAACTATAAAGGAGTTTCCTATCAGTTTGTTATGGGTCTGACAGA
TTCGGTTTCTTTCCGAGCTTATGCTGCTTATTCTAAGCCTGCTAACGATAACCTTGGTAGCGACTTCACC
TATCGTAAGTATGACCTAGGTTTAATTTCTTCATTCTAA
>gi|3329350|gb|AAC68472.1| Putative Outer Membrane Protein I [Chlamydia
trachomatis][SEQ ID NO:7]
MRPDHMNFCCLCAAILSSTAVLFGQDPLGETALLTKNPNHVVCTFFEDCTMESLFPALCAHASQDDPLYV
LGNSYCWFVSKLHITDPKEALFKEKGDLSIQNFRFLSFTDCSSKESSPSIIHQKNGQLSLRNNGSMSFCR
NHAEGSGGAISADAFSLQHNYLFTAFEENSSKGNGGAIQAQTFSLSRNVSPISFARNRADLNGGAICCSN
LICSGNVNPLFFTGNSATNGGAICCISDLNTSEKGSLSLACNQETLFASNSAKEKGGAIYAKHMVLRYNG
PVSFINNSAKIGGAIAIQSGGSLSILAGEGSVLFQNNSQRTSDQGLVRNAIYLEKDAILSSLEARNGDIL
FFDPIVQESSSKESPLPSSLQASVTSPTPATASPLVIQTSANRSVIFSSERLSEEEKTPDNLTSQLQQPI
ELKSGRLVLKDRAVLSAPSLSQDPQALLIMEAGTSLKTSSDLKLATLSIPLHSLDTEKSVTIHAPNLSIQ
KIFLSNSGDENFYENVELLSKEQNNIPLLTLSKEQSHLHLPDGNLSSHFGYQGDWTFSWKDSDEGHSLIA
NWTPKNYVPHPERQSTLVANTLWNTYSDMQAVQSMINTIAHGGAYLFGTWGSAVSNLFYAHDSSGKPIDN
WHHRSLGYLFGISTHSLDDHSFCLAAGQLLGKSSDSFITSTETTSYIATVQAQLATPLMKISAQACYNES
IHELKTKYRSFSKEGFGSWHSVAVSGEVCASIPIVSNGSGLFSSFSIFSKLQGFSGTQDGFEESSGEIRS
FSASSFRNISLPMGITFEKKSQKTRNYYYFLGAYIQDLKRDVESGPVVLLKNAVSWDAPMANLDSRAYMF
RLTNQRALHRLQTLLNVSYVLRGQSHSYSLDLGTTYRF
>gi|3329348|gb|AE001361.1:c3451-815[SEQ ID NO:8]
ATGCGACCTGATCATATGAACTTCTGTTGTCTATGTGCTGCTATTTTGTCATCCACAGCGGTCCTCTTTG
GCCAGGATCCCTTAGGTGAAACCGCCCTCCTCACTAAAAATCCTAATCATGTCGTCTGTACATTTTTTGA
GGACTGTACCATGGAGAGCCTCTTTCCTGCTCTTTGTGCTCATGCATCACAAGATGATCCTTTGTATGTA
CTTGGAAATTCCTACTGTTGGTTCGTATCTAAACTCCATATCACGGACCCCAAAGAGGCTCTTTTTAAAG
AAAAAGGAGATCTTTCCATTCAAAATTTTCGCTTCCTTTCCTTCACAGATTGCTCTTCCAAGGAAAGCTC
TCCTTCTATTATTCATCAAAAGAATGGTCAGTTATCCTTGCGCAATAATGGTAGCATGAGTTTCTGTCGA
AATCATGCTGAAGGCTCTGGAGGAGCCATCTCTGCGGATGCCTTTTCTCTACAACACAACTATCTTTTCA
CAGCTTTTGAAGAGAATTCTTCTAAAGGAAATGGCGGAGCCATTCAGGCTCAAACCTTCTCTTTATCTAG
AAATGTGTCGCCTATTTCTTTCGCCCGTAATCGTGCGGATTTAAATGGCGGCGCTATTTGCTGTAGTAAT
CTTATTTGTTCAGGGAATGTAAACCCTCTCTTTTTCACTGGAAACTCCGCCACGAATGGAGGCGCTATTT
GTTGTATCAGCGATCTAAACACCTCAGAAAAAGGCTCTCTCTCTCTTGCTTGTAACCAAGAAACGCTATT
TGCAAGCAATTCTGCTAAAGAAAAAGGCGGGGCTATTTATGCCAAGCACATGGTATTGCGTTATAACGGT
CCTGTTTCCTTCATTAACAACAGCGCTAAAATAGGTGGAGCTATCGCCATCCAGTCCGGAGGGAGTCTCT
CTATCCTTGCAGGTGAAGGATCTGTTCTGTTCCAGAATAACTCCCAACGCACCTCCGACCAAGGTCTAGT
AAGAAACGCCATCTACTTAGAGAAAGATGCGATTCTTTCTTCCTTAGAAGCTCGCAACGGAGATATTCTT
TTCTTTGATCCTATTGTACAAGAAAGTAGCAGCAAAGAATCGCCTCTTCCCTCCTCTTTGCAAGCCAGCG
TGACTTCTCCCACCCCAGCCACCGCATCTCCTTTAGTTATTCAGACAAGTGCAAACCGTTCAGTGATTTT
CTCGAGCGAACGTCTTTCTGAAGAAGAAAAAACTCCTGATAACCTCACTTCCCAACTACAGCAGCCTATC
GAACTGAAATCCGGACGCTTAGTTTTAAAAGATCGCGCTGTCCTTTCCGCGCCTTCTCTCTCTCAGGATC
CTCAAGCTCTCCTCATTATGGAAGCGGGAACTTCTTTAAAAACTTCCTCTGATTTGAAGTTAGCTACGCT
AAGTATTCCCCTTCATTCCTTAGATACTGAAAAAAGCGTAACTATCCACGCCCCTAACCTTTCTATCCAA
AAGATCTTCCTCTCTAATTCTGGAGATGAGAATTTTTATGAAAATGTAGAGCTTCTCAGTAAAGAGCAAA
ACAATATTCCTCTCCTTACTCTCTCTAAAGAGCAATCTCATTTACATCTTCCTGATGGGAACCTCTCTTC
TCACTTTGGATATCAAGGAGATTGGACTTTTTCTTGGAAAGATTCTGATGAAGGGCATTCTCTGATTGCT
AATTGGACGCCTAAAAACTATGTGCCTCATCCAGAACGTCAATCTACACTCGTTGCGAACACTCTTTGGA
ACACCTATTCCGATATGCAAGCTGTGCAGTCGATGATTAATACAATAGCGCACGGAGGAGCCTATCTATT
TGGAACGTGGGGATCTGCTGTTTCTAATTTATTCTATGCTCACGACAGCTCTGGGAAACCTATCGATAAT
TGGCATCATAGAAGCCTTGGCTACCTATTCGGTATCAGTACTCACAGTTTAGATGACCATTCTTTCTGCT
TGGCTGCAGGACAATTACTCGGGAAATCGTCCGATTCCTTTATTACGTCTACAGAAACGACCTCCTATAT
AGCTACTGTACAAGCGCAACTCGCTACCCCTCTAATGAAAATCTCTGCACAGGCATGCTATAATGAAAGT
ATCCATGAGCTAAAAACAAAATATCGCTCCTTCTCTAAAGAAGGATTCGGATCCTGGCATAGCGTTGCAG
TATCCGGAGAAGTGTGCGCATCGATTCCTATTGTATCCAATGGTTCCGGACTGTTCAGCTCCTTCTCTAT
TTTCTCTAAACTGCAAGGATTTTCAGGAACACAGGACGGTTTTGAGGAGAGTTCGGGAGAGATTCGGTCC
TTTTCTGCCAGCTCTTTCAGAAATATTTCACTTCCTATGGGAATAACATTTGAAAAAAAATCCCAAAAAA
CACGAAACTACTATTACTTTCTGGGAGCCTACATCCAAGACCTAAAACGTGATGTGGAATCGGGACCTGT
AGTGTTACTCAAAAATGCCGTCTCCTGGGATGCTCCTATGGCGAACTTGGATTCGCGAGCCTACATGTTC
AGGCTTACGAATCAAAGAGCTCTGCATAGACTTCAGACGCTGTTAAATGTGTCTTACGTACTGCGCGGGC
AAAGCCATAGTTACTCCCTGGATCTGGGGACCACTTACAGGTTCTAG
>gi|3329346|gb|AAC68469.1| Putative Outer Membrane Protein G [Chlamydia
trachomatis][SEQ ID NO:9]
MQTSFHKFFLSMILAYSCCSLSGGGYAAEIMIPQGIYDGETLTVSFPYTVIGDPSGTTVFSAGELTLKNL
DNSIAALPLSCFGNLLGSFTVLGRGHSLTFENIRTSTNGAALSDSANSGLFTIEGFKELSFSNCNSLLAV
LPAATTNNGSQTPTTTSTPSNGTIYSKTDLLLLNNEKFSFYSNLVSGDGGAIDAKSLTVQGISKLCVFQE
NTAQADGGACQVVTSFSAMANEAPIAFIANVAGVRGGGIAAVQDGQQGVSSSTSTEDPVVSFSRNTAVEF
DGNVARVGGGIYSYGNVAFLNNGKTLFLNNVASPVYIAAEQPTNGQASNTSDNYGDGGAIFCKNGAQAAG
SNNSGSVSFDGEGVVFFSSNVAAGKGGAIYAKKLSVANCGPVQFLGNIANDGGAIYLGESGELSLSADYG
DIIFDGNLKRTAKENAADVNGVTVSSQAISMGSGGKITTLRAKAGHQILFNDPIEMANGNNQPAQSSEPL
KINDGEGYTGDIVFANGNSTLYQNVTIEQGRIVLREKAKLSVNSLSQTGGSLYMEAGSTLDFVTPQPPQQ
PPAANQLITLSNLHLSLSSLLANNAVTNPPTNPPAQDSHPAIIGSTTAGSVTISGPIFFEDLDDTAYDRY
DWLGSNQKIDVLKLQLGTQPSANAPSDLTLGNEMPKYGYQGSWKLAWDPNTANNGPYTLKATWTKTGYNP
GPERVASLVPNSLWGSILDIRSAHSAIQASVDGRSYCRGLWVSGVSNFFYHDRDALGQGYRYISGGYSLG
ANSYFGSSMFGLAFTEVFGRSKDYVVCRSNHHACIGSVYLSTKQALCGSYLFGDAFIRASYGFGNQHMKT
SYTFAEESDVRWDNNCLVGEIGVGLPIVITPSKLYLNELRPFVQAEFSYADHESFTEEGDQARAFRSGHL
MNLSVPVGVKFDRCSSTHPNKYSFMGAYICDAYRTISGTQTTLLSHQETWTTDAFHLARHGVIVRGSMYA
SLTSNIEVYGHGRYEYRDTSRGYGLSAGSKVRF
>gi|3329342|gb|AE001360.1:7736-10777[SEQ ID NO:10]
ATGCAAACGTCTTTCCATAAGTTCTTTCTTTCAATGATTCTAGCTTATTCTTGCTGCTCTTTAAGTGGGG
GGGGGTATGCAGCAGAAATCATGATTCCTCAAGGAATTTACGATGGGGAGACGTTAACTGTATCATTTCC
CTATACTGTTATAGGAGATCCGAGTGGGACTACTGTTTTTTCTGCAGGAGAGTTAACGTTAAAAAATCTT
GACAATTCTATTGCAGCTTTGCCTTTAAGTTGTTTTGGGAACTTATTAGGGAGTTTTACTGTTTTAGGGA
GAGGACACTCGTTGACTTTCGAGAACATACGGACTTCTACAAATGGAGCTGCACTAAGTGACAGCGCTAA
TAGCGGGTTATTTACTATTGAGGGTTTTAAAGAATTATCTTTTTCCAATTGCAACTCATTACTTGCCGTA
CTGCCTGCTGCAACGACTAATAATGGTAGCCAGACTCCGACGACAACATCTACACCGTCTAATGGTACTA
TTTATTCTAAAACAGATCTTTTGTTACTCAATAATGAGAAGTTCTCATTCTATAGTAATTTAGTCTCTGG
AGATGGGGGAGCTATAGATGCTAAGAGCTTAACGGTTCAAGGAATTAGCAAGCTTTGTGTCTTCCAAGAA
AATACTGCTCAAGCTGATGGGGGAGCTTGTCAAGTAGTCACCAGTTTCTCTGCTATGGCTAACGAGGCTC
CTATTGCCTTTATAGCGAATGTTGCAGGAGTAAGAGGGGGAGGGATTGCTGCTGTTCAGGATGGGCAGCA
GGGAGTGTCATCATCTACTTCAACAGAAGATCCAGTAGTAAGTTTTTCCAGAAATACTGCGGTAGAGTTT
GATGGGAACGTAGCCCGAGTAGGAGGAGGGATTTACTCCTACGGGAACGTTGCTTTCCTGAATAATGGAA
AAACCTTGTTTCTCAACAATGTTGCTTCTCCTGTTTACATTGCTGCTGAGCAACCAACAAATGGACAGGC
TTCTAATACGAGTGATAATTACGGAGATGGAGGAGCTATCTTCTGTAAGAATGGTGCGCAAGCAGCAGGA
TCCAATAACTCTGGATCAGTTTCCTTTGATGGAGAGGGAGTAGTTTTCTTTAGTAGCAATGTAGCTGCTG
GGAAAGGGGGAGCTATTTATGCCAAAAAGCTCTCGGTTGCTAACTGTGGCCCTGTACAATTCTTAGGGAA
TATCGCTAATGATGGTGGAGCGATTTATTTAGGAGAATCTGGAGAGCTCAGTTTATCTGCTGATTATGGA
GATATTATTTTCGATGGGAATCTTAAAAGAACAGCCAAAGAGAATGCTGCCGATGTTAATGGCGTAACTG
TGTCCTCACAAGCCATTTCGATGGGATCGGGAGGGAAAATAACGACATTAAGAGCTAAAGCAGGGCATCA
GATTCTCTTTAATGATCCCATCGAGATGGCAAACGGAAATAACCAGCCAGCGCAGTCTTCCGAACCTCTA
AAAATTAACGATGGTGAAGGATACACAGGGGATATTGTTTTTGCTAATGGAAACAGTACTTTGTACCAAA
ATGTTACGATAGAGCAAGGAAGGATTGTTCTTCGTGAAAAGGCAAAATTATCAGTGAATTCTCTAAGTCA
GACAGGTGGGAGTCTGTATATGGAAGCTGGGAGTACATTGGATTTTGTAACTCCACAACCACCACAACAG
CCTCCTGCCGCTAATCAGTTGATCACGCTTTCCAATCTGCATTTGTCTCTTTCTTCTTTGTTAGCAAACA
ATGCAGTTACGAATCCTCCTACCAATCCTCCAGCGCAAGATTCTCATCCTGCAATCATTGGTAGCACAAC
TGCTGGTTCTGTTACAATTAGTGGGCCTATCTTTTTTGAGGATTTGGATGATACAGCTTATGATAGGTAT
GATTGGCTAGGTTCTAATCAAAAAATCGATGTCCTGAAATTACAGTTAGGGACTCAGCCCTCAGCTAATG
CCCCATCAGATTTGACTCTAGGGAATGAGATGCCTAAGTATGGCTATCAAGGAAGCTGGAAGCTTGCGTG
GGATCCTAATACAGCAAATAATGGTCCTTATACTCTGAAAGCTACATGGACTAAAACTGGGTATAATCCT
GGGCCTGAGCGAGTAGCTTCTTTGGTTCCAAATAGTTTATGGGGATCCATTTTAGATATACGATCTGCGC
ATTCAGCAATTCAAGCAAGTGTGGATGGGCGCTCTTATTGTCGAGGATTATGGGTTTCTGGAGTTTCGAA
TTTCTTCTATCATGACCGCGATGCTTTAGGTCAGGGATATCGGTATATTAGTGGGGGTTATTCCTTAGGA
GCAAACTCCTACTTTGGATCATCGATGTTTGGTCTAGCATTTACCGAAGTATTTGGTAGATCTAAAGATT
ATGTAGTGTGTCGTTCCAATCATCATGCTTGCATAGGATCCGTTTATCTATCTACCAAACAAGCTTTATG
TGGATCCTATTTGTTCGGAGATGCGTTTATCCGTGCTAGCTACGGGTTTGGGAACCAGCATATGAAAACC
TCATACACATTTGCAGAGGAGAGCGATGTTCGTTGGGATAATAACTGTCTGGTTGGAGAGATTGGAGTGG
GATTACCGATTGTGATTACTCCATCTAAGCTCTATTTGAATGAGTTGCGTCCTTTCGTGCAAGCTGAGTT
TTCTTATGCCGATCATGAATCTTTTACAGAGGAAGGCGATCAAGCTCGGGCATTCAGGAGTGGACATCTC
ATGAATCTATCAGTTCCTGTTGGAGTAAAATTTGATCGATGTTCTAGTACACACCCTAATAAATATAGCT
TTATGGGGGCTTATATCTGTGATGCTTATCGCACCATCTCTGGGACTCAGACAACACTCCTATCCCATCA
AGAGACATGGACAACAGATGCCTTTCATTTGGCAAGACATGGAGTCATAGTTAGAGGGTCTATGTATGCT
TCTCTAACAAGCAATATAGAAGTATATGGCCATGGAAGATATGAGTATCGAGATACTTCTCGAGGTTATG
GTTTGAGTGCAGGAAGTAAAGTCCGGTTCTAA
>gi|3329345|gb|AAC68468.1| Putative Outer Membrane Protein F [Chlamydia
trachomatis][SEQ ID NO:11]
MIKRTSLSFACLSFFYLSTISILQANETDTLQFRRFTFSDREIQFVLDPASLITAQNIVLSNLQSNGTGA
CTISGNTQTQIFSNSVNTTADSGGAFDMVTTSFTASDNANLLFCNNYCTHNKGGGAIRSGGPIRFLNNQD
VLFYNNISAGAKYVGTGDHNEKNRGGALYATTITLTGNRTLAFINNMSGDCGGAISADTQISITDTVKGI
LFENNHTLNHIPYTQAENMARGGAICSRRDLCSISNNSGPIVFNYNQGGKGGAISATRCVIDNNKERIIF
SNNSSLGWSQSSSASNGGAIQTTQGFTLRNNKGSIYFDSNTATHAGGAINCGYIDIRDNGPVYFLNNSAA
WGAAFNLSKPRSATNYIHTGTGDIVFNNNVVFTLDGNLLGKRKLFHINNNEITPYTLSLGAKKDTRIYFY
DLFQWERVKENTSNNPPSPTSRNTITVNPETEFSGAVVFSYNQMSSDIRTLMGKEHNYIKEAPTTLKFGT
LAIEDDAELEIFNIPFTQNPTSLLALGSGATLTVGKHGKLNITNLGVILPIILKEGKSPPCIRVNPQDMT
QNTGTGQTPSSTSSISTPMIIFNGRLSIVDENYESVYDSMDLSRGKAEQLILSIETTNDGQLDSNWQSSL
NTSLLSPPHYGYQGLWTPNWITTTYTITLNNNSSAPTSATSIAEQKKTSETFTPSNTTTASIPNIKASAG
SGSGSASNSGEVTITKHTLVVNWAPVGYIVDPIRRGDLIANSLVHSGRNMTMGLRSLLPDNSWFALQGAA
TTLFTKQQKRLSYHGYSSASKGYTVSSQASGAHGHKFLLSFSQSSDKMKEKETNNRLSSRYYLSALCFEH
PMFDRIALIGAAACNYGTHNMRSFYGTKKSSKGKFHSTTLGASLRCELRDSMPLRSIMLTPFAQALFSRT
EPASIRESGDLARLFTLEQAHTAVVSPIGIKGAYSSDTWPTLSWEMELAYQPTLYWKRPLLNTLLIQNNG
SWVTTNTPLAKHSFYGRGSHSLKFSHLKLFANYQAEVATSTVSHYINAGGALVF
>gi|3329342|gb|AE001360.1:c7571-4467[SEQ ID NO:12]
ATGATTAAAAGAACTTCTCTATCCTTTGCTTGCCTCAGTTTTTTTTATCTTTCAACTATATCCATTTTGC
AAGCTAATGAAACGGATACGCTACAGTTCCGGCGATTTACTTTTTCGGATAGAGAGATTCAGTTCGTCCT
AGATCCCGCCTCTTTAATTACCGCCCAAAACATCGTTTTATCTAATTTACAGTCAAACGGAACCGGAGCC
TGTACCATTTCAGGCAATACGCAAACTCAAATCTTTTCTAATTCCGTTAACACCACCGCAGATTCTGGTG
GAGCCTTTGATATGGTTACTACCTCATTCACGGCCTCTGATAATGCTAATCTACTCTTCTGCAACAACTA
CTGCACACATAATAAAGGCGGAGGAGCTATTCGTTCCGGAGGACCTATTCGATTCTTAAATAATCAAGAC
GTGCTTTTTTATAATAACATATCGGCAGGGGCTAAATATGTTGGAACAGGAGATCACAACGAAAAAAATA
GGGGCGGTGCGCTTTATGCAACTACTATCACTTTGACAGGGAATCGAACTCTTGCCTTTATTAACAATAT
GTCTGGAGACTGCGGTGGAGCCATCTCTGCTGACACTCAAATATCAATAACTGATACCGTTAAAGGAATT
TTATTTGAAAACAATCACACGCTCAATCATATACCGTACACGCAAGCTGAAAATATGGCACGAGGAGGAG
CAATCTGTAGTAGAAGAGACTTGTGCTCAATCAGCAATAATTCTGGTCCCATAGTTTTTAACTATAACCA
AGGCGGGAAAGGTGGAGCTATTAGCGCTACCCGATGTGTTATTGACAATAACAAAGAAAGAATCATCTTT
TCAAACAATAGTTCCCTGGGATGGAGCCAATCTTCTTCTGCAAGTAACGGAGGAGCCATTCAAACGACAC
AAGGATTTACTTTACGAAATAATAAAGGCTCTATCTACTTCGACAGCAACACTGCTACACACGCCGGGGG
AGCCATTAACTGTGGTTACATTGACATCCGAGATAACGGACCCGTCTATTTTCTAAATAACTCTGCTGCC
TGGGGAGCGGCCTTTAATTTATCGAAACCACGTTCAGCGACAAATTATATCCATACAGGGACAGGCGATA
TTGTTTTTAATAATAACGTTGTCTTTACTCTTGACGGTAATTTATTAGGGAAACGGAAACTTTTTCATAT
TAATAATAATGAGATAACACCATATACATTGTCTCTCGGCGCTAAAAAAGATACTCGTATCTATTTTTAT
GATCTTTTCCAATGGGAGCGTGTTAAAGAAAATACTAGCAATAACCCACCATCTCCTACCAGTAGAAACA
CCATTACCGTTAACCCGGAAACAGAGTTTTCTGGAGCTGTTGTGTTCTCCTACAATCAAATGTCTAGTGA
CATACGAACTCTGATGGGTAAAGAACACAATTACATTAAAGAAGCCCCAACTACTTTAAAATTCGGAACG
CTAGCCATAGAAGATGATGCAGAATTAGAAATCTTCAATATCCCGTTTACCCAAAATCCGACTAGCCTTC
TTGCTTTAGGAAGCGGCGCTACGCTGACTGTTGGAAAGCACGGTAAGCTCAATATTACAAATCTTGGTGT
TATTTTACCCATTATTCTCAAAGAGGGGAAGAGTCCGCCTTGTATTCGCGTCAACCCACAAGATATGACC
CAAAATACTGGTACCGGCCAAACTCCATCAAGCACAAGTAGTATAAGCACTCCAATGATTATCTTTAATG
GGCGCCTCTCAATTGTAGACGAAAATTATGAATCAGTCTACGACAGTATGGACCTCTCCAGAGGGAAAGC
AGAACAACTAATTCTATCCATAGAAACCACTAATGATGGGCAATTAGACTCCAATTGGCAAAGTTCTCTG
AATACTTCTCTACTCTCTCCTCCACACTATGGCTATCAAGGTCTATGGACTCCTAATTGGATAACAACAA
CCTATACCATCACGCTTAATAATAATTCTTCAGCTCCAACATCTGCTACCTCCATCGCTGAGCAGAAAAA
AACTAGTGAAACTTTTACTCCTAGTAACACAACTACAGCTAGTATCCCTAATATTAAAGCTTCCGCAGGA
TCAGGCTCTGGATCGGCTTCCAATTCAGGAGAAGTTACGATTACCAAACATACCCTTGTTGTAAACTGGG
CACCAGTCGGCTACATAGTAGATCCTATTCGTAGAGGAGATCTGATAGCCAATAGCTTAGTACATTCAGG
AAGAAACATGACCATGGGCTTACGATCATTACTCCCGGATAACTCTTGGTTTGCTTTGCAAGGAGCTGCA
ACAACATTATTTACAAAACAACAAAAACGTTTGAGTTATCATGGCTACTCTTCTGCATCAAAGGGGTATA
CCGTCTCTTCTCAAGCATCAGGAGCTCATGGTCATAAGTTTCTTCTTTCCTTCTCCCAGTCATCTGATAA
GATGAAAGAAAAAGAAACAAATAACCGCCTTTCTTCTCGTTACTATCTTTCTGCTTTATGTTTCGAACAT
CCTATGTTTGATCGCATTGCTCTTATCGGAGCAGCAGCTTGCAATTATGGAACACATAACATGCGGAGTT
TCTATGGAACTAAAAAATCTTCTAAAGGGAAATTTCACTCTACAACCTTAGGAGCTTCTCTTCGCTGTGA
ACTACGCGATAGTATGCCTTTACGATCAATAATGCTCACCCCATTTGCTCAGGCTTTATTCTCTCGAACA
GAACCAGCTTCTATCCGAGAAAGCGGTGATCTAGCTAGATTATTTACATTAGAGCAAGCCCATACTGCCG
TTGTCTCTCCAATAGGAATCAAAGGAGCTTATTCTTCTGATACATGGCCAACACTCTCTTGGGAAATGGA
ACTAGCTTACCAACCCACCCTCTACTGGAAACGTCCTCTACTCAACACACTATTAATCCAAAATAACGGT
TCTTGGGTCACCACAAATACCCCATTAGCTAAACATTCCTTTTATGGGAGAGGTTCTCACTCCCTCAAAT
TTTCTCATCTGAAACTATTTGCTAACTATCAAGCAGAAGTGGCTACTTCCACTGTCTCACACTACATCAA
TGCAGGAGGAGCTCTGGTCTTTTAA
>gi|3329344|gb|AAC68467.1| Putative Outer Membrane Protein E [Chlamydia
trachomatis][SEQ ID NO:13]
MKKAFFFFLIGNSLSGLAREVPSRIFLMPNSVPDPTKESLSNKISLTGDTHNLTNCYLDNLRYILAILQK
TPNEGAAVTITDYLSFFDTQKEGIYFAKNLTPESGGAIGYASPNSPTVEIRDTIGPVIFENNTCCRLFTW
RNPYAADKIREGGAIHAQNLYINHNHDVVGFMKNFSYVQGGAISTANTFVVSENQSCFLFMDNICIQTNT
AGKGGAIYAGTSNSFESNNCDLFFINNACCAGGAIFSPICSLTGNRGNIVFYNNRCFKNVETASSEASDG
GAIKVTTRLDVTGNRGRIFFSDNITKNYGGAIYAPVVTLVDNGPTYFINNIANNKGGAIYIDGTSNSKIS
ADRHAIIFNENIVTNVTNANGTSTSANPPRRNAITVASSSGEILLGAGSSQNLIFYDPIEVSNAGVSVSF
NKEADQTGSVVFSGATVNSADFHQRNLQTKTPAPLTLSNGFLCIEDHAQLTVNRFTQTGGVVSLGNGAVL
SCYKNGTGDSASNASITLKHIGLNLSSILKSGAEIPLLWVEPTNNSNNYTADTAATFSLSDVKLSLIDDY
GNSPYESTDLTHALSSQPMLSISEASDNQLQSENIDFSGLNVPHYGWQGLWTWGWAKTQDPEPASSATIT
DPQKANRFHRTLLLTWLPAGYVPSPKHRSPLIANTLWGNMLLATESLKNSAELTPSGHPFWGITGGGLGM
MVYQDPRENHPGFHMRSSGYSAGMIAGQTHTFSLKFSQTYTKLNERYAKNNVSSKNYSCQGEMLFSLQEG
FLLTKLVGLYSYGDHNCHHFYTQGENLTSQGTFRSQTMGGAVFFDLPMKPFGSTHILTAPFLGALGIYSS
LSHFTEVGAYPRSFSTKTPLINVLVPIGVKGSFMNATHRPQAWTVELAYQPVLYRQEPGIAAQLLASKGI
WFGSGSPSSRHAMSYKISQQTQPLSWLTLHFQYHGFYSSSTFCNYLNGEIALRF
>gi|3329342|gb|AE001360.1:c4464-1570[SEQ ID NO:14]
ATGAAAAAAGCGTTTTTCTTTTTCCTTATCGGAAACTCCCTATCAGGACTAGCTAGAGAGGTTCCTTCTA
GAATCTTTCTTATGCCCAACTCAGTTCCAGATCCTACGAAAGAGTCGCTATCAAATAAAATTAGTTTGAC
AGGAGACACTCACAATCTCACTAACTGCTATCTCGATAACCTACGCTACATACTGGCTATTCTACAAAAA
ACTCCCAATGAAGGAGCTGCTGTCACAATAACAGATTACCTAAGCTTTTTTGATACACAAAAAGAAGGTA
TTTATTTTGCAAAAAATCTCACCCCTGAAAGTGGTGGTGCGATTGGTTATGCGAGTCCCAATTCTCCTAC
CGTGGAGATTCGTGATACAATAGGTCCTGTAATCTTTGAAAATAATACTTGTTGCAGACTATTTACATGG
AGAAATCCTTATGCTGCTGATAAAATAAGAGAAGGCGGAGCCATTCATGCTCAAAATCTTTACATAAATC
ATAATCATGATGTGGTCGGATTTATGAAGAACTTTTCTTATGTCCAAGGAGGAGCCATTAGTACCGCTAA
TACCTTTGTTGTGAGCGAGAATCAGTCTTGTTTTCTCTTTATGGACAACATCTGTATTCAAACTAATACA
GCAGGAAAAGGTGGCGCTATCTATGCTGGAACGAGCAATTCTTTTGAGAGTAATAACTGCGATCTCTTCT
TCATCAATAACGCCTGTTGTGCAGGAGGAGCGATCTTCTCCCCTATCTGTTCTCTAACAGGAAATCGTGG
TAACATCGTTTTCTATAACAATCGCTGCTTTAAAAATGTAGAAACAGCTTCTTCAGAAGCTTCTGATGGA
GGAGCAATTAAAGTAACTACTCGCCTAGATGTTACAGGCAATCGTGGTAGGATCTTTTTTAGTGACAATA
TCACAAAAAATTATGGCGGAGCTATTTACGCTCCTGTAGTTACCCTAGTGGATAATGGCCCTACCTACTT
TATAAACAATATCGCCAATAATAAGGGGGGCGCTATCTATATAGACGGAACCAGTAACTCCAAAATTTCT
GCCGACCGCCATGCTATTATTTTTAATGAAAATATTGTGACTAATGTAACTAATGCAAATGGTACCAGTA
CGTCAGCTAATCCTCCTAGAAGAAATGCAATAACAGTAGCAAGCTCCTCTGGTGAAATTCTATTAGGAGC
AGGGAGTAGCCAAAATTTAATTTTTTATGATCCTATTGAAGTTAGCAATGCAGGGGTCTCTGTGTCCTTC
AATAAGGAAGCTGATCAAACAGGCTCTGTAGTATTTTCAGGAGCTACTGTTAATTCTGCAGATTTTCATC
AACGCAATTTACAAACAAAAACACCTGCACCCCTTACTCTCAGTAATGGTTTTCTATGTATCGAAGATCA
TGCTCAGCTTACAGTGAATCGATTCACACAAACTGGGGGTGTTGTTTCTCTTGGGAATGGAGCAGTTCTG
AGTTGCTATAAAAATGGTACAGGAGATTCTGCTAGCAATGCCTCTATAACACTGAAGCATATTGGATTGA
ATCTTTCTTCCATTCTGAAAAGTGGTGCTGAGATTCCTTTATTGTGGGTAGAGCCTACAAATAACAGCAA
TAACTATACAGCAGATACTGCAGCTACCTTTTCATTAAGTGATGTAAAACTCTCACTCATTGATGACTAC
GGGAACTCTCCTTATGAATCCACAGATCTGACCCATGCTCTGTCATCACAGCCTATGCTATCTATTTCTG
AAGCTAGCGATAACCAGCTACAATCAGAAAATATAGATTTTTCGGGACTAAATGTCCCTCATTATGGATG
GCAAGGACTTTGGACTTGGGGCTGGGCAAAAACTCAAGATCCAGAACCAGCATCTTCAGCAACAATCACT
GATCCACAAAAAGCCAATAGATTTCATAGAACCTTACTACTAACATGGCTTCCTGCCGGGTATGTTCCTA
GCCCAAAACACAGAAGTCCCCTCATAGCTAACACCTTATGGGGGAATATGCTGCTTGCAACAGAAAGCTT
AAAAAATAGTGCAGAGCTGACACCTAGTGGTCATCCTTTCTGGGGAATTACAGGAGGAGGACTAGGCATG
ATGGTTTACCAAGATCCTCGAGAAAATCATCCTGGATTCCATATGCGCTCTTCCGGATACTCTGCGGGGA
TGATAGCAGGGCAGACACACACCTTCTCATTGAAATTCAGTCAGACCTACACCAAACTCAATGAGCGTTA
CGCAAAAAACAACGTATCTTCTAAAAATTACTCATGCCAAGGAGAAATGCTCTTCTCATTGCAAGAAGGT
TTCTTGCTGACTAAATTAGTTGGGCTTTACAGCTATGGAGACCATAACTGTCACCATTTCTATACTCAAG
GAGAAAATCTAACATCTCAAGGGACGTTCCGCAGTCAAACGATGGGAGGTGCTGTCTTTTTTGATCTCCC
TATGAAACCCTTTGGATCAACGCATATACTGACAGCTCCCTTTTTAGGTGCTCTTGGTATTTATTCTAGC
CTGTCTCACTTTACTGAGGTGGGAGCCTATCCGCGAAGCTTTTCTACAAAGACTCCTTTGATCAATGTCC
TAGTCCCTATTGGAGTTAAAGGTAGCTTTATGAATGCTACCCACAGACCTCAAGCCTGGACTGTAGAATT
GGCATACCAACCCGTTCTGTATAGACAAGAACCAGGGATCGCAGCCCAGCTCCTAGCCAGTAAGGGTATT
TGGTTCGGTAGTGGAAGCCCCTCATCGCGTCATGCCATGTCCTATAAAATCTCACAGCAAACACAACCTT
TGAGTTGGTTAACTCTCCATTTCCAGTATCATGGATTCTACTCCTCTTCAACCTTCTGTAATTATCTCAA
TGGGGAAATTGCTCTGCGATTCTAG
>gi|3329279|gb|AAC68408.1| Putative Outer Membrane Protein D [Chlamydia
trachomatis][SEQ ID NO:15]
MSSEKDIKSTCSKFSLSVVAAILASVSGLASCVDLHAGGQSVNELVYVGPQAVLLLDQIRDLFVGSKDSQ
AEGQYRLIVGDPSSFQEKDADTLPGKVEQSTLFSVTNPVVFQGVDQQDQVSSQGLICSFTSSNLDSPRDG
ESFLGIAFVGDSSKAGITLTDVKASLSGAALYSTEDLIFEKIKGGLEFASCSSLEQGGACAAQSILIHDC
QGLQVKHCTTAVNAEGSSANDHLGFGGGAFFVTGSLSGEKSLYMPAGDMVVANCDGAISFEGNSANFANG
GAIAASGKVLFVANDKKTSFIENRALSGGAIAASSDIAFQNCAELVFKGNCAIGTEDKGSLGGGAISSLG
TVLLQGNHGITCDKNESASQGGAIFGKNCQISDNEGPVVFRDSTACLGGGAIAAQEIVSIQNNQAGISFE
GGKASFGGGIACGSFSSAGGASVLGTIDISKNLGAISFSRTLCTTSDLGQMEYQGGGALFGENISLSENA
GVLTFKDNIVKTFASNGKILGGGAILATGKVEITNNSEGISFTGNARAPQALPTQEEFPLFSKKEGRPLS
SGYSGGGAILGREVAILHNAAVVFEQNRLQCSEEEATLLGCCGGGAVHGMDSTSIVGNSSVRFGNNYAMG
QGVSGGALLSKTVQLAGNGSVDFSRNIASLGGGALQASEGNCELVDNGYVLFRDNRGRVYGGAISCLRGD
VVISGNKGRVEFKDNIATRLYVEETVEKVEEVEPAPEQKDNNELSFLGRAEQSFITAANQALFASEDGDL
SPESSISSEELAKRRECAGGAIFAKRVRIVDNQEAVVFSNNFSDIYGGAIFTGSLREEDKLDGQIPEVLI
SGNAGDVVFSGNSSKRDEHLPHTGGGAICTQNLTISQNTGNVLFYNNVACSGGAVRIEDHGNVLLEAFGG
DIVFKGNSSFRAQGSDAIYFAGKESHITALNATEGHAIVFHDALVFENLEERKSAEVLLINSRENPGYTG
SIRFLEAESKVPQCIHVQQGSLELLNGATLCSYGFKQDAGAKLVLAAGAKLKILDSGTPVQQGHAISKPE
AEIESSSEPEGAHSLWIAKNAQTTVPMVDIHTISVDLASFSSSQQEGTVEAPQVIVPGGSYVRSGELNLE
LVNTTGTGYENHALLKNEAKVPLMSFVASGDEASAEISNLSVSDLQIHVVTPEIEEDTYGHMGDWSEAKI
QDGTLVISWNPTGYRLDPQKAGALVFNALWEEGAVLSALKNARFAHNLTAQRMEFDYSTNVWGFAFGGFR
TLSAENLVAIDGYKGAYGGASAGVDIQLMEDFVLGVSGAAFLGKMDSQKFDAEVSRKGVVGSVYTGFLAG
SWFFKGQYSLGETQNDMKTRYGVLGESSASWTSRGVLADALVEYRSLVGPVRPTFYALHFNPYVEVSYAS
MKFPGFTEQGREARSFEDASLTNITIPLGMKFELAFIKGQFSEVNSLGISYAWEAYRKVEGGAVQLLEAG
FDWEGAPMDLPRQELRVALENNTEWSSYFSTVLGLTAFCGGFTSTDSKLGYEANTGLRLIF
>gi|3329271|gb|AE001353.1:9710-14305[SEQ ID NO:16]
ATGAGTTCCGAGAAAGATATAAAAAGCACCTGTTCTAAGTTTTCTTTGTCTGTAGTAGCAGCTATCCTTG
CCTCTGTTAGCGGGTTAGCTAGTTGCGTAGATCTTCATGCTGGAGGACAGTCTGTAAATGAGCTGGTATA
TGTAGGCCCTCAAGCGGTTTTATTGTTAGACCAAATTCGAGATCTATTCGTTGGGTCTAAAGATAGTCAG
GCTGAAGGACAGTATAGGTTAATTGTAGGAGATCCAAGTTCTTTCCAAGAGAAAGATGCGGATACTCTTC
CCGGGAAGGTAGAGCAAAGTACTTTGTTCTCAGTAACCAATCCCGTGGTTTTCCAAGGTGTGGACCAACA
GGATCAAGTCTCTTCCCAAGGGTTAATTTGTAGTTTTACGAGCAGCAACCTTGATTCTCCTCGTGACGGA
GAATCTTTTTTAGGTATTGCTTTTGTTGGGGATAGTAGTAAGGCTGGAATCACATTAACTGACGTGAAAG
CTTCTTTGTCTGGAGCGGCTTTATATTCTACAGAAGATCTTATCTTTGAAAAGATTAAGGGTGGATTGGA
ATTTGCATCATGTTCTTCTCTAGAACAGGGGGGAGCTTGTGCAGCTCAAAGTATTTTGATTCATGATTGT
CAAGGATTGCAGGTTAAACACTGTACTACAGCCGTGAATGCTGAGGGGTCTAGTGCGAATGATCATCTTG
GATTTGGAGGAGGCGCTTTCTTTGTTACGGGTTCTCTTTCTGGAGAGAAAAGTCTCTATATGCCTGCAGG
AGATATGGTAGTTGCGAATTGTGATGGGGCTATATCTTTTGAAGGAAACAGCGCGAACTTTGCTAATGGA
GGAGCGATTGCTGCCTCTGGGAAAGTGCTTTTTGTCGCTAATGATAAAAAGACTTCTTTTATAGAGAACC
GAGCTTTGTCTGGAGGAGCGATTGCAGCCTCTTCTGATATTGCCTTTCAAAACTGCGCAGAACTAGTTTT
CAAAGGCAATTGTGCAATTGGAACAGAGGATAAAGGTTCTTTAGGTGGAGGGGCTATATCTTCTCTAGGC
ACCGTTCTTTTGCAAGGGAATCACGGGATAACTTGTGATAAGAATGAGTCTGCTTCGCAAGGAGGCGCCA
TTTTTGGCAAAAATTGTCAGATTTCTGACAACGAGGGGCCAGTGGTTTTCAGAGATAGTACAGCTTGCTT
AGGAGGAGGCGCTATTGCAGCTCAAGAAATTGTTTCTATTCAGAACAATCAGGCTGGGATTTCCTTCGAG
GGAGGTAAGGCTAGTTTCGGAGGAGGTATTGCGTGTGGATCTTTTTCTTCCGCAGGTGGTGCTTCTGTTT
TAGGGACCATTGATATTTCGAAGAATTTAGGCGCGATTTCGTTCTCTCGTACTTTATGTACGACCTCAGA
TTTAGGACAAATGGAGTACCAGGGAGGAGGAGCTCTATTTGGTGAAAATATTTCTCTTTCTGAGAATGCT
GGTGTGCTCACCTTTAAAGACAACATTGTGAAGACTTTTGCTTCGAATGGGAAAATTCTGGGAGGAGGAG
CGATTTTAGCTACTGGTAAGGTGGAAATTACTAATAATTCCGAAGGAATTTCTTTTACAGGAAATGCGAG
AGCTCCACAAGCTCTTCCAACTCAAGAGGAGTTTCCTTTATTCAGCAAAAAAGAAGGGCGACCACTCTCT
TCAGGATATTCTGGGGGAGGAGCGATTTTAGGAAGAGAAGTAGCTATTCTCCACAACGCTGCAGTAGTAT
TTGAGCAAAATCGTTTGCAGTGCAGCGAAGAAGAAGCGACATTATTAGGTTGTTGTGGAGGAGGCGCTGT
TCATGGGATGGATAGCACTTCGATTGTTGGCAACTCTTCAGTAAGATTTGGTAATAATTACGCAATGGGA
CAAGGAGTCTCAGGAGGAGCTCTTTTATCTAAAACAGTGCAGTTAGCTGGGAATGGAAGCGTCGATTTTT
CTCGAAATATTGCTAGTTTGGGAGGAGGAGCTCTTCAAGCTTCTGAAGGAAATTGTGAGCTAGTTGATAA
CGGCTATGTGCTATTCAGAGATAATCGAGGGAGGGTTTATGGGGGTGCTATTTCTTGCTTACGTGGAGAT
GTAGTCATTTCTGGAAACAAGGGTAGAGTTGAATTTAAAGACAACATAGCAACACGTCTTTATGTGGAAG
AAACTGTAGAAAAGGTTGAAGAGGTAGAGCCAGCTCCTGAGCAAAAAGACAATAATGAGCTTTCTTTCTT
AGGGAGAGCAGAACAGAGTTTTATTACTGCAGCTAATCAAGCTCTTTTCGCATCTGAAGATGGGGATTTA
TCACCTGAGTCATCCATTTCTTCTGAAGAACTTGCGAAAAGAAGAGAGTGTGCTGGAGGAGCTATTTTTG
CAAAACGGGTTCGTATTGTAGATAACCAAGAGGCCGTTGTATTCTCGAATAACTTCTCTGATATTTATGG
CGGCGCCATTTTTACAGGTTCTCTTCGAGAAGAGGATAAGTTAGATGGGCAAATCCCTGAAGTCTTGATC
TCAGGCAATGCAGGGGATGTTGTTTTTTCCGGAAATTCCTCGAAGCGTGATGAGCATCTTCCTCATACAG
GTGGGGGAGCCATTTGTACTCAAAATTTGACGATTTCTCAGAATACAGGGAATGTTCTGTTTTATAACAA
CGTGGCCTGTTCGGGAGGAGCTGTTCGTATAGAGGATCATGGTAATGTTCTTTTAGAAGCTTTTGGAGGA
GATATTGTTTTTAAAGGAAATTCTTCTTTCAGAGCACAAGGATCCGATGCTATCTATTTTGCAGGTAAAG
AATCGCATATTACAGCCCTGAATGCTACGGAAGGACATGCTATTGTTTTCCACGACGCATTAGTTTTTGA
AAATCTAGAAGAAAGGAAATCTGCTGAAGTATTGTTAATCAATAGTCGAGAAAATCCAGGTTACACTGGA
TCTATTCGATTTTTAGAAGCAGAAAGTAAAGTTCCTCAATGTATTCATGTACAACAAGGAAGCCTTGAGT
TGCTAAATGGAGCCACATTATGTAGTTATGGTTTTAAACAAGATGCTGGAGCTAAGTTGGTATTGGCTGC
TGGAGCTAAACTGAAGATTTTAGATTCAGGAACTCCTGTACAACAAGGGCATGCTATCAGTAAACCTGAA
GCAGAAATCGAGTCATCTTCTGAACCAGAGGGTGCACATTCTCTTTGGATTGCGAAGAATGCTCAAACAA
CAGTTCCTATGGTTGATATCCATACTATTTCTGTAGATTTAGCCTCCTTCTCTTCTAGTCAACAGGAGGG
GACAGTAGAAGCTCCTCAGGTTATTGTTCCTGGAGGAAGTTATGTTCGATCTGGAGAGCTTAATTTGGAG
TTAGTTAACACAACAGGTACTGGTTATGAAAATCATGCTTTATTGAAGAATGAGGCTAAAGTTCCATTGA
TGTCTTTCGTTGCTTCTGGTGATGAAGCTTCAGCCGAAATCAGTAACTTGTCGGTTTCTGATTTACAGAT
TCATGTAGTAACTCCAGAGATTGAAGAAGACACATACGGCCATATGGGAGATTGGTCTGAGGCTAAAATT
CAAGATGGAACTCTTGTCATTAGTTGGAATCCTACTGGATATCGATTAGATCCTCAAAAAGCAGGGGCTT
TAGTATTTAATGCATTATGGGAAGAAGGGGCTGTCTTGTCTGCTCTGAAAAATGCACGCTTTGCTCATAA
TCTCACTGCTCAGCGTATGGAATTCGATTATTCTACAAATGTGTGGGGATTCGCCTTTGGTGGTTTCCGA
ACTCTATCTGCAGAGAATCTGGTTGCTATTGATGGATACAAAGGAGCTTATGGTGGTGCTTCTGCTGGAG
TCGATATTCAATTGATGGAAGATTTTGTTCTAGGAGTTAGTGGAGCTGCTTTCCTAGGTAAAATGGATAG
TCAGAAGTTTGATGCGGAGGTTTCTCGGAAGGGAGTTGTTGGTTCTGTATATACAGGATTTTTAGCTGGA
TCCTGGTTCTTCAAAGGACAATATAGCCTTGGAGAAACACAGAACGATATGAAAACGCGTTATGGAGTAC
TAGGAGAGTCGAGTGCTTCTTGGACATCTCGAGGAGTACTGGCAGATGCTTTAGTTGAATACCGAAGTTT
AGTTGGTCCTGTGAGACCTACTTTTTATGCTTTGCATTTCAATCCTTATGTCGAAGTATCTTATGCTTCT
ATGAAATTCCCTGGCTTTACAGAACAAGGAAGAGAAGCGCGTTCTTTTGAAGACGCTTCCCTTACCAATA
TCACCATTCCTTTAGGGATGAAGTTTGAATTGGCGTTCATAAAAGGACAGTTTTCAGAGGTGAACTCTTT
GGGAATAAGTTATGCATGGGAAGCTTATCGAAAAGTAGAAGGAGGCGCGGTGCAGCTTTTAGAAGCTGGG
TTTGATTGGGAGGGAGCTCCAATGGATCTTCCTAGACAGGAGCTGCGTGTCGCTCTGGAAAATAATACGG
AATGGAGTTCTTACTTCAGCACAGTCTTAGGATTAACAGCTTTTTGTGGAGGATTTACTTCTACAGATAG
TAAACTAGGATATGAGGCGAATACTGGATTGCGATTGATCTTTTAA
>gi|3329169|gb|AAC68308.1| Outer Membrane Protein Analog [Chlamydia
trachomatis][SEQ ID NO:17]
MSSKLVNYLRLTFLSFLGIASTSLDAMPAGNPAFPVIPGINIEQKNACSFDLCNSYDVLSALSGNLKLCF
CGDYIFSEEAQVKDVPVVTSVTTAGVGPSPDITSTTKTRNFDLVNCNLNTNCVAVAFSLPDRSLSAIPLF
DVSFEVKVGGLKQYYRLPMNAYRDFTSEPLNSESEVTDGMIEVQSNYGFVWDVSLKKVIWKDGVSFVGVG
ADYRHASCPIDYIIANSQANPEVFIADSDGKLNFKEWSVCVGLTTYVNDYVLPYLAFSIGSVSRQAPDDS
FKKLEDRFTNLKFKVRKITSSHRGNICIGATNYVADNFFYNVEGRWGSQRAVNVSGGFQF
>gi|3329166|gb|AE001342.1:c4638-3616[SEQ ID NO:18]
ATGAGTAGCAAGCTAGTGAACTATCTCCGTTTGACTTTCCTATCTTTTTTAGGGATCGCATCTACTTCAT
TAGACGCTATGCCTGCGGGGAATCCGGCGTTTCCAGTCATCCCGGGGATTAATATTGAACAGAAAAATGC
CTGTTCTTTCGATTTATGTAATTCTTATGATGTACTATCCGCACTGTCCGGTAACCTGAAGCTCTGCTTC
TGCGGAGATTATATCTTTTCAGAAGAAGCTCAGGTAAAAGATGTCCCTGTCGTTACCTCTGTGACAACAG
CTGGGGTTGGTCCTTCTCCTGATATTACTTCGACAACCAAAACGCGAAATTTCGATCTCGTGAACTGTAA
TCTCAATACAAACTGTGTAGCTGTAGCTTTTTCCCTTCCTGATCGTTCGCTGAGCGCGATTCCTCTGTTT
GATGTGAGTTTCGAAGTGAAAGTAGGAGGACTGAAACAATACTACCGCCTTCCCATGAATGCCTATCGAG
ACTTCACCTCGGAACCTCTCAATTCTGAATCAGAAGTTACGGACGGGATGATTGAAGTACAGTCCAATTA
CGGATTTGTTTGGGATGTTAGCTTGAAAAAAGTCATATGGAAAGATGGCGTTTCCTTTGTAGGCGTCGGT
GCAGACTATCGCCATGCTTCTTGCCCTATTGACTACATCATTGCAAACAGTCAAGCTAATCCAGAAGTAT
TCATCGCTGACTCGGATGGGAAACTGAACTTCAAGGAGTGGAGTGTCTGCGTAGGTCTTACTACCTATGT
GAATGACTACGTTCTTCCTTACTTAGCGTTTTCTATAGGGAGTGTTTCTCGCCAAGCTCCGGACGACAGC
TTCAAAAAATTAGAAGATCGCTTCACTAACCTCAAATTTAAAGTTCGTAAAATTACCAGCTCTCATCGTG
GAAACATCTGCATCGGAGCGACAAACTATGTCGCCGATAACTTCTTCTACAACGTAGAAGGAAGATGGGG
AAGCCAGCGCGCTGTGAACGTCTCCGGAGGATTCCAATTCTAA
>gi|3328866|gb|AAC68034.1| Sulfite Reductase [Chlamydia trachomatis][SEQ ID NO:19]
MSLFSKFKAQWMFLHSRELCSSTSDIGNTCSDPVFQVLCNPVRSEISYKVGDSLGVFPTNPSILVDSVLD
ALQYGPRSPVVSRHADSVLPLHEFLTSYVDLDKIPKSLRPFFPGDLDDTWSLAEAILVYQPRIPFEEFIR
SAMPLLPRFYSIASSPTCSHGKLELLVRCVSFQGKTQLRYGLCSAFLCKDLQEGESFRGFIQPTRHFTLE
QKNFGKPLIMIGAGTGIAPYKGFLQHRIYHQDVGSNILFFGERFEKSNFYYRDFLQELIVSGKLQLFTAF
SRDSESKLYVQNVIEQQKELIQEVYEQEAFFFVCGKKILGTEVKRALEQILGPKAVRELIAQKRLVSDVY
>gi|3328863|gb|AE001317:c2573-1521[SEQ ID NO:20]
ATGTCTTTATTTTCTAAATTCAAAGCTCAGTGGATGTTTTTACATTCACGTGAGCTTTGTTCTTCCACAT
CGGATATTGGGAATACTTGTTCGGATCCTGTTTTTCAGGTTTTATGTAATCCGGTTCGTTCTGAGATTTC
CTATAAAGTTGGGGATTCTTTGGGGGTATTCCCAACAAATCCTTCCATATTAGTCGATTCAGTTCTAGAT
GCTTTACAGTATGGCCCCAGGTCTCCTGTCGTATCTCGGCATGCAGATTCTGTTCTCCCTCTTCACGAAT
TTCTTACTAGTTACGTAGACTTAGATAAAATTCCAAAATCGTTAAGACCTTTTTTCCCAGGGGATTTAGA
CGATACCTGGTCTTTAGCTGAAGCTATTTTGGTTTACCAGCCGCGTATTCCTTTTGAAGAGTTTATTCGG
AGTGCGATGCCTTTATTGCCTCGATTTTATTCTATAGCTTCTTCTCCAACATGTTCTCATGGGAAGCTAG
AGTTGCTCGTGCGCTGTGTTAGTTTCCAAGGTAAAACGCAGCTGCGCTATGGATTATGTTCGGCTTTTTT
ATGTAAGGACTTACAAGAGGGAGAGTCTTTTCGTGGGTTTATACAACCGACGCGGCATTTTACTTTGGAG
CAGAAAAATTTTGGGAAACCTTTAATTATGATCGGAGCAGGGACAGGTATCGCTCCGTACAAAGGGTTCT
TACAACATCGAATATACCATCAGGACGTAGGCTCCAATATTCTATTCTTTGGAGAGCGTTTTGAGAAAAG
TAACTTCTATTACCGGGATTTTCTCCAGGAGCTGATCGTTTCAGGAAAACTCCAGTTATTCACAGCCTTT
TCCAGAGATTCCGAGTCTAAATTGTATGTTCAGAATGTTATAGAGCAACAAAAAGAACTTATACAAGAAG
TCTACGAACAAGAAGCTTTCTTTTTTGTTTGTGGGAAAAAAATCCTTGGTACGGAAGTTAAACGTGCTTT
AGAGCAGATATTAGGTCCTAAGGCGGTACGAGAGCTGATTGCACAGAAGAGACTAGTTTCAGACGTATAC
TAA
>gi|3328843|gb|AAC68011.1| Putative outer membrane protein C [Chlamydia
trachomatis][SEQ ID NO:21]
MKFMSATAVFAAALSSVTEASSIQDQIKNTDCNVSKLGYSTSQAFTDMMLADNTEYRAADSVSFYDFSTS
SRLPRKHLSSSSEASPTTEGVSSSSSGETDEKTEEELDNGGIIYAREKLTISESQDSLSNQSIELHDNSI
FFGEGEVIFDHRVALKNGGAIYGEKEVVFENIKSLLVEVNIAVEKGGSVYAKERVSLENVTEATFSSNGG
EQGGGGIYSEQDMLISDCNNVHFQGNAAGATAVKQCLDEEMIVLLAECVDSLSEDTLDSTPETEQTESNG
NQDGSSETEDTQVSESPESTPSPDDVLGKGGGIYTEKSLTITGITGTIDFVSNIATDSGAGVFTKENLSC
TNTNSLQFLKNSAGQHGGGAYVTQTMSVTNTTSESITTPPLIGEVIFSENTAKGHGGGICTNKLSLSNLK
TVTLTKNSAKESGGAIFTDLASIPITDTPESSTPSSSSPASTPEVVASAKINRFFASTAKPAAPSLTEAE
SDQTDQTETSDTNSDIDVSIENILNVAINQNTSAKKGGAIYGKKAKLSRINNLELSGNSSQDVGGGLCLT
ESVEFDAIGSLLSHYNSAAKEGGAIHSKTVTLSNLKSTFTFADNTVKAIVESTPEAPEEIPPVEGEESTA
TEDPNSNTEGSSANTNLEGSQGDTADTGTGDVNNESQDTSDTGNAESEEQLQDSTQSNEENTLPNSNIDQ
SNENTDESSDSHTEEITDESVSSSSESGSSTPQDGGAASSGAPSGDQSISANACLAKSYAASTDSSPVSN
SSGSEEPVTSSSDSDVTASSDNPDSSSSGDSAGDSEEPTEPEAGSTTETLTLIGGGAIYGETVKIENFSG
QGIFSGNKAIDNTTEGSSSKSDVLGGAVYAKTLFNLDSGSSRRTVTFSGNTVSSQSTTGQVAGGAIYSPT
VTIATPVVFSKNSATNNANNTTDTQRKDTFGGAIGATSAVSLSGGAHFLENVADLGSAIGLVPGTQNTET
VKLESGSYYFEKNKALKRATIYAPVVSIKAYTATFNQNRSLEEGSAIYFTKEASIESLGSVLFTGNLVTL
TLSTTTEGTPATTSGDVTKYGAAIFGQIASSNGSQTDNLPLKLIASGGNICFRNNEYRPTSSDTGTSTFC
SIAGDVKLTMQAAKGKTISFFDAIRTSTKKTGTQATAYDTLDINKSEDSETVNSAFTGTILFSSELHENK
SYIPQNVVLHSGSLVLKPNTELHVISFEQKEGSSLVMTPGSVLSNQTVADGALVINNMTIDLSSVEKNGI
AEGNIFTPPELRIIDTTTGGSGGTPSTDSESNQNSDDTEEQNNNDASNQGESANGSSSPAVAAAHTSRTR
NFAAAATATPTTTPTATTTTSNQVILGGEIKLIDPNGTFFQNPALRSDQQISLLVLPTDSSKMQAQKIVL
TGDIAPQKGYTGTLTLDPDQLQNGTISVLWKFDSYRQWAYVPRDNHFYANSILGSQMLMVTVKQGLLNDK
MNLARFEEVSYNNLWISGLGTMLSQVGTPTSEEFTYYSRGASVALDAKPAHDVIVGAAFSKMIGKTKSLK
RENNYTHKGSEYSYQASVYGGKPFHFVINKKTEKSLPLLLQGVISYGYIKHDTVTHYPTIRERNKGEWED
LGWLTALRVSSVLRTPAQGDTKRITVYGELEYSSIRQKQFTETEYDPRYFDNCTYRNLAIPMGLAFEGEL
SGNDILMYNRFSVAYMLSIYRNSPTCKYQVLSSGEGGEIICGVPTRNSARGEYSTQLYLGPLWTLYGSYT
IEADAHTLAHMMNCGARMTF
>gi|3328842|gb|AE001315.1:120-5432[SEQ ID NO:22]
ATGAAATTTATGTCAGCTACTGCTGTATTTGCTGCAGCACTCTCCTCCGTTACTGAGGCGAGCTCGATCC
AAGATCAAATAAAGAATACCGACTGCAATGTTAGCAAATTAGGATATTCAACTTCTCAAGCATTTACTGA
TATGATGCTAGCAGACAACACAGAGTATCGAGCTGCTGATAGTGTTTCATTCTATGACTTTTCGACATCT
TCCAGATTACCTAGAAAACATCTTAGTAGTAGTAGTGAAGCTTCTCCAACGACAGAAGGAGTGTCTTCAT
CTTCATCTGGAGAAACTGATGAGAAAACAGAAGAAGAACTAGACAATGGCGGAATCATTTATGCTAGAGA
GAAACTAACTATCTCAGAATCTCAGGACTCTCTCTCTAATCAAAGCATAGAACTCCATGACAATAGTATT
TTCTTCGGAGAAGGTGAAGTTATCTTTGATCACAGAGTTGCCCTCAAAAACGGAGGAGCTATTTATGGAG
AGAAAGAGGTAGTCTTTGAAAACATAAAATCTCTACTAGTAGAAGTAAATATCGCGGTCGAGAAAGGGGG
TAGCGTCTATGCAAAAGAACGAGTATCTTTAGAAAATGTTACCGAAGCAACCTTCTCCTCCAATGGTGGG
GAACAAGGTGGTGGTGGAATCTATTCAGAACAGGATATGTTAATCAGTGATTGCAACAATGTACATTTCC
AAGGGAATGCTGCAGGAGCAACAGCAGTAAAACAATGTCTGGATGAAGAAATGATCGTATTGCTCGCAGA
ATGCGTTGATAGCTTATCCGAAGATACACTGGATAGCACTCCAGAAACGGAACAGACTGAGTCAAATGGA
AATCAAGACGGTTCGTCTGAAACAGAAGATACACAAGTATCAGAATCACCAGAATCAACTCCTAGCCCCG
ACGATGTTTTAGGTAAAGGTGGTGGTATCTATACAGAAAAATCTTTGACCATCACTGGAATTACACGGAC
TATAGATTTTGTCAGTAACATAGCTACCGATTCTGGAGCAGGTGTATTCACTAAAGAAAACTTGTCTTGC
ACCAACACGAATAGCCTACAGTTTTTGAAAAACTCGGCAGGTCAACATGGAGGAGGAGCCTACGTTACTC
AAACCATGTCTGTTACTAATACAACTAGTGAAAGTATAACTACTCCCCCTCTCATAGGACAAGTGATTTT
CTCTGAAAATACAGCTAAAGGGCACGGTGGTGGTATCTGCACTAACAAACTTTCTTTATCTAATTTAAAA
ACGGTGACTCTCACTAAAAACTCTGCAAAGGAGTCTGGAGGAGCTATTTTTACAGATCTGGCGTCTATAC
CAATAACAGATACCCCAGAATCTTCTACCCCCTCTTCCTCCTCGCCTGCAAGCACTCCTGAAGTAGTTGC
TTCTGCTAAAATAAATCGATTCTTTGCCTCTACGGCAAAACCGGCAGCCCCTTCTCTAACAGAGGCTGAG
TCTGATCAAACGGATCAAACAGAAACTTCTGATACTAATAGCGATATAGACGTGTCGATTGAGAACATTT
TGAATGTCGCTATCAATCAAAACACTTCTGCGAAAAAAGGAGGGGCTATTTACGGGAAAAAAGCTAAACT
TTCCCGTATTAACAATCTTGAACTTTCAGGGAATTCATCCCAGGATGTAGGAGGAGGTCTCTGTTTAACT
GAAAGCGTAGAATTTGATGCAATTGGATCGCTCTTATCCCACTATAACTCTGCTGCTAAAGAAGGTGGGG
CTATTCATTCTAAAACGGTTACTCTATCTAACCTCAAGTCTACCTTCACTTTTGCAGATAACACTGTTAA
AGCAATAGTAGAAAGCACTCCTGAAGCTCCAGAAGAGATTCCTCCAGTAGAAGGAGAAGAGTCTACAGCA
ACAGAAGATCCAAATTCTAATACAGAAGGAAGTTCGGCTAACACTAACCTTGAAGGATCTCAAGGGGATA
CTGCTGATACAGGGACTGGTGATGTTAACAATGAGTCTCAAGACACATCAGATACTGGAAACGCTGAATC
TGAAGAACAACTACAAGATTCTACACAATCTAATGAAGAAAATACCCTTCCCAATAGTAATATTGATCAA
TCTAACGAAAACACAGACGAATCATCTGATAGCCACACTGAGGAAATAACTGACGAGAGTGTCTCATCGT
CCTCTGAAAGTGGATCATCTACTCCTCAAGATGGAGGAGCAGCTTCTTCAGGGGCTCCCTCAGGAGATCA
ATCTATCTCTGCAAACGCTTGTTTAGCTAAAAGCTATGCTGCGAGTACTGATAGCTCCCCCGTATCTAAT
TCTTCAGGTTCAGAAGAGCCTGTCACTTCTTCTTCAGATTCAGACGTTACTGCATCTTCTGATAATCCAG
ACTCTTCCTCATCTGGAGATAGCGCTGGAGACTCTGAAGAACCGACTGAGCCAGAAGCTGGTTCTACAAC
AGAAACTCTTACTTTAATAGGAGGAGGTGCTATCTATGGAGAAACTGTTAAGATTGAGAACTTCTCTGGC
CAAGGAATATTTTCTGGAAACAAAGCTATCGATAACACCACAGAAGGCTCCTCTTCCAAATCTGACGTCC
TCGGAGGTGCGGTCTATGCTAAAACATTGTTTAATCTCGATAGCGGGAGCTCTAGACGAACTGTCACCTT
CTCCGGGAATACTGTCTCTTCTCAATCTACAACAGGTCAGGTTGCTGGAGGAGCTATCTACTCTCCTACT
GTAACCATTGCTACTCCTGTAGTATTTTCTAAAAACTCTGCAACAAACAATGCTAATAACACTACAGATA
CTCAGAGAAAAGACACCTTTGGAGGAGCTATCGGAGCTACTTCTGCTGTTTCTCTATCAGGAGGGGCTCA
TTTCTTAGAAAACGTTGCTGACCTCGGATCTGCTATTGGGTTGGTGCCAGGCACACAAAATACAGAAACA
GTGAAATTAGAGTCTGGCTCCTACTACTTTGAAAAAAATAAAGCTTTAAAACGAGCTACTATTTACGCAC
CTGTCGTTTCCATTAAAGCCTATACTGCGACATTTAACCAAAACAGATCTCTAGAAGAAGGAAGCGCGAT
TTACTTTACAAAAGAAGCATCTATTGAGTCTTTAGGCTCTGTTCTCTTCACAGGAAACTTAGTAACCCTA
ACGCTAAGCACAACTACAGAAGGCACACCAGCCACAACCTCAGGAGATGTAACAAAATATGGTGCTGCTA
TCTTTGGACAAATAGCAAGCTCAAACGGATCTCAGACGGATAACCTTCCCCTGAAACTCATTGCTTCAGG
AGGAAATATTTGTTTCCGAAACAATGAATACCGTCCTACTTCTTCTGATACCGGAACCTCTACTTTCTGT
AGTATTGCGGGAGATGTTAAATTAACCATGCAAGCTGCAAAAGGGAAAACGATCAGTTTCTTTGATGCAA
TCCGGACCTCTACTAAGAAAACAGGTACACAGGCAACTGCCTACGATACTCTCGATATTAATAAATCTGA
GGATTCAGAAACTGTAAACTCTGCGTTTACAGGAACGATTCTGTTCTCCTCTGAATTACATGAAAATAAA
TCCTATATTCCACAAAACGTAGTTCTACACAGTGGATCTCTTGTATTGAAGCCAAATACCGAGCTTCATG
TTATTTCTTTTGAGCAGAAAGAAGGCTCTTCTCTCGTTATGACACCTGGATCTGTTCTTTCGAACCAGAC
TGTTGCTGATGGAGCTTTGGTCATAAATAACATGACCATTGATTTATCCAGCGTAGAGAAAAATGGTATT
GCTGAAGGAAATATCTTTACTCCTCCAGAATTGAGAATCATAGACACTACTACAGGTGGAAGCGGTGGAA
CCCCATCTACAGATAGTGAAAGTAACCAGAATAGTGATGATACCGAGGAGCAAAATAATAATGACGCCTC
GAATCAAGGAGAAAGCGCGAATGGATCGTCTTCTCCTGCAGTAGCTGCTGCACACACATCTCGTACAAGA
AACTTTGCCGCTGCAGCTACAGCCACACCTACGACAACACCAACGGCTACAACTACAACAAGCAACCAAG
TAATCCTAGGAGGAGAAATTAAACTCATCGATCCTAATGGGACCTTCTTCCAGAACCCTGCATTAAGATC
CGACCAACAAATCTCCTTGTTAGTGCTCCCTACAGACTCATCAAAAATGCAAGCTCAGAAAATAGTACTG
ACGGGTGATATTGCTCCTCAGAAAGGATATACAGGAACACTCACTCTGGATCCTGATCAACTACAAAATG
GAACGATCTCAGTGCTCTGGAAATTTGACTCTTATAGACAATGGGCTTATGTACCTAGAGACAATCATTT
CTATGCGAACTCGATTCTGGGATCTCAAATGTTAATGGTCACAGTCAAACAAGGCTTGCTCAACGATAAA
ATGAATCTAGCTCGCTTTGAGGAAGTTAGCTATAACAACCTGTGGATATCAGGACTAGGAACGATGCTAT
CGCAAGTAGGAACACCTACTTCTGAAGAATTCACTTATTACAGCAGAGGAGCTTCTGTTGCCTTAGATGC
TAAACCAGCCCATGATGTGATTGTTGGAGCTGCATTTAGTAAGATGATCGGGAAAACAAAATCCTTGAAA
AGAGAGAATAACTACACTCACAAAGGATCCGAATATTCTTACCAAGCATCGGTATACGGAGGCAAACCAT
TCCACTTTGTAATCAATAAAAAAACGGAAAAATCGCTACCGCTATTGTTACAAGGAGTCATCTCTTACGG
ATATATCAAACATGATACAGTGACTCACTATCCAACGATCCGTGAACGAAACAAAGGAGAATGGGAAGAC
TTAGGATGGCTGACAGCTCTCCGTGTCTCCTCTGTCTTAAGAACTCCTGCACAAGGGGATACTAAACGTA
TCACTGTTTACGGAGAATTGGAATACTCCAGTATCCGTCAGAAACAATTCACAGAAACAGAATACGATCC
TCGTTACTTCGACAACTGCACCTATAGAAACTTAGCAATTCCTATGGGGTTAGCATTCGAAGGAGAGCTC
TCTGGTAACGATATTTTGATGTACAACAGATTCTCTGTAGCATACATGCTATCAATCTATCGAAATTCTC
CAACATGCAAATACCAAGTGCTCTCTTCAGGAGAAGGCGGAGAAATTATTTGTGGAGTACCGACAAGAAA
CTCAGCTCGCGGAGAATACAGCACGCAGCTGTACCTGGGACCTTTGTGGACTCTGTATGGATCCTACACG
ATAGAAGCAGACGCACATACACTAGCTCATATGATGAACTGCGGTGCTCGTATGACATTCTAA
>gi|3328815|gb|AAC67986.1| hypothetical protein [Chlamydia trachomatis][SEQ ID NO:23]
MMKPLRFGYFFCAIYFTLLQAAFAKEPNSCPDCQNNWKEVTHTDQLPENIIHADDACYHSGYVQALIDMH
FLDSCCQVIVENQTAYLFSLPTDDVTRNAIINLIKDLPFIHSVEICQASYQTCHHQGPHGKTSLPEQRSF
CTKVCGKEAIWLPQNTILFSPLVADPRQATNSAGIRFNDEVLGKRVGSATFGGDFIFLRLFDISRFHGDM
DIGLQGAVFSVFDLDHPEACMVNSDFFVAALCNFAVNKWSYRFRLWHLSSHLGDEFILANQLPPKKRYNR
SDEAVDFFASFRYTPQIRVYGGIGYIISRDLTFPEDPLYFEGGIELRPFGLREDNLHAQPVFAMHFRFWE
EHDFSIDQTYIVGMEWSKFQDVGRKVRAVLEYHQGFSHEGQFVREECDYYGFRLSYGF
>gi|3328812|gb|AE001312.1:2790-4016[SEQ ID NO:24]
ATGATGAAACCTCTACGTTTCGGTTATTTCTTTTGCGCAATCTATTTTACTTTGTTACAGGCAGCGTTTG
CTAAAGAACCGAATTCTTGTCCCGACTGCCAGAATAATTGGAAAGAAGTCACCCACACGGATCAACTCCC
AGAAAACATCATTCATGCTGATGATGCTTGTTATCACTCTGGTTATGTACAGGCTCTCATTGATATGCAT
TTCTTAGATAGCTGCTGCCAGGTCATCGTTGAAAACCAAACTGCTTACTTATTTTCTCTTCCTACAGATG
ATGTTACGCGCAACGCCATTATCAACCTAATTAAAGACCTTCCATTCATTCACTCCGTAGAAATCTGCCA
AGCATCCTATCAAACCTGTCATCATCAAGGCCCTCATGGAAAGACTTCTCTTCCAGAACAACGTTCTTTC
TGTACAAAGGTCTGTGGAAAAGAAGCTATTTGGTTACCACAGAATACCATCCTATTCTCGCCTCTTGTAG
CAGATCCTAGACAAGCAACTAATAGTGCAGGTATCCGTTTTAACGACGAAGTCTTAGGAAAACGTGTTGG
CTCTGCTACCTTCGGTGGAGATTTCATCTTCTTACGATTATTTGATATCTCCCGATTCCATGGAGACATG
GATATTGGTCTCCAAGGAGCTGTATTCTCTGTTTTCGACCTGGATCATCCAGAAGCTTGCATGGTCAACT
CTGACTTTTTTGTCGCCGCTTTGTGCAACTTTGCAGTGAACAAATGGAGCTACCGCTTCAGACTATGGCA
TCTTTCTTCTCATCTTGGCGACGAATTTATTCTTGCCAACCAGTTACCTCCTAAAAAACGTTATAATCGA
AGCGATGAAGCCGTCGATTTCTTTGCTTCTTTTCGTTACACTCCACAGATCCGTGTTTATGGAGGTATTG
GGTATATCATTAGTCGAGATTTAACATTCCCTGAAGATCCTCTTTACTTTGAAGGAGGTATCGAACTACG
TCCTTTCGGATTACGGGAAGACAACCTTCATGCCCAACCCGTCTTTGCTATGCATTTTCGCTTTTGGGAA
GAGCATGACTTTTCTATAGACCAAACTTATATAGTAGGCATGGAGTGGTCCAAATTCCAGGATGTAGGGA
GAAAAGTGCGCGCTGTATTGGAATACCACCAAGGTTTCTCCCACGAAGGACAATTTGTCCGAGAAGAATG
CGATTATTATGGCTTTCGATTAAGTTATGGCTTCTAG
>gi|3328651|gb|AAC67834.1| Omp85 Analog [Chlamydia trachomatis][SEQ ID NO:25]
MLGIRKKTILQLAVLLLLTFSRSSFCSTSEGRMVVESITITTQGENTQNKRAIPKIKTKQGTLFSQADFD
EDLRTLSKDFDRVEPIVEFRNGQAVISLILTAKPVIREINISGNEAIPTHKILKTLELYKNDLFDRELFF
KNFDALRTLYLKRGYYDSQLSYSHNHNEKEGFIDISIEIKEGRHGRIKKLTISGITRTEASDLGDIVLTK
QYSTTTSWFTGAGVYHPDMVEQDLFAITNYFQNKGYADAKVSKEVSTDAKGNITLLIVVDKGPLYTLGHV
HIEGFTALSKRLLDKQLLVGPNSLYCPDKIWTGAQKIRSAYARYGYVNTNVDVSFSAHPTLPVYDVTYRV
SEGSPYKIGLIKIKGNTHTKHDVILHETSLFPGDTFDRLKLEGTETRLRNTGYFKSVSVYTVRSQLDPLD
SNDLYRDVFIEVKETETGNLGLFLGFSSIDHLFGGAEIAESNFDLFGARNFLKKGFKSLRGGGEYLFLKA
NLGDKVTDYTVKWTKPHFLNTPWILGVELDKSINKALSKDYSVDTYGGNISTTYILNDKLKYGMYYRGSQ
TSLSLRKKTSSSNRPGPDLDSNKGFVSAAGLNVLYDSIDNPRKPTMGIRSSLNFELSGLGGTYQFTKLTA
SGSIYRLLTKKGVLKVRAEAKFIKPFGTTTAQGIPVSERFFLGGETTVRGYKPFIIGPKFSPTEPQGGLS
SLLLTEEFQYPLISQPCINAFVFLDSGFIGIEEYTIRLKDLCSSAGFGLRFDMMNNVPIMLGWGWPFRPT
EILNNEKIDVSQRFFFALGGVF
>gi|3328646|gb|AE001297.1:4000-6378[SEQ ID NO:26]
ATGCTTGGAATACGCAAAAAAACGATTCTGCAACTCGCTGTTTTACTGTTGCTCACCTTTTCACGAAGTT
CTTTCTGTTCAACTTCAGAAGGACGTATGGTCGTAGAGTCTATCACCATTACGACTCAAGGAGAGAATAC
TCAAAATAAACGAGCTATTCCTAAAATAAAAACAAAGCAGGGGACGTTGTTCTCTCAAGCAGATTTTGAT
GAAGATCTAAGAACACTTTCGAAAGATTTTGATCGAGTAGAGCCTATCGTAGAGTTTCGTAATGGACAAG
CTGTGATCTCTCTGATTCTGACGGCAAAACCTGTTATCAGAGAGATCAATATTTCAGGAAATGAAGCTAT
CCCCACTCATAAAATTCTGAAAACTTTAGAGCTTTATAAAAATGATCTTTTTGATCGGGAATTATTCTTT
AAAAATTTTGATGCGCTAAGAACTCTTTATTTGAAACGAGGGTACTACGATTCTCAACTCTCCTATTCTC
ATAATCATAATGAGAAAGAGGGCTTTATCGATATTTCCATCGAGATTAAAGAAGGACGTCACGGTCGCAT
AAAAAAATTAACGATTTCGGGAATTACGCGAACAGAAGCATCAGACTTAGGTGACATTGTTTTAACTAAA
CAATACTCCACAACAACGAGCTGGTTCACTGGTGCCGGAGTGTATCATCCGGACATGGTAGAGCAAGACT
TATTTGCTATCACAAATTACTTCCAAAATAAAGGATATGCTGATGCTAAAGTAAGCAAAGAGGTCTCTAC
AGATGCTAAAGGAAACATTACTTTGCTTATCGTTGTAGACAAAGGACCTTTATACACATTAGGTCACGTA
CATATAGAAGGATTCACAGCGTTATCCAAAAGACTGCTCGATAAACAACTATTGGTTGGACCTAACTCCT
TATATTGCCCAGATAAAATTTGGACTGGAGCACAAAAGATTCGTAGCGCATACGCTAGATATGGCTACGT
GAACACTAACGTTGATGTCTCCTTCTCAGCGCACCCCACTCTACCTGTTTACGATGTTACCTATCGAGTG
AGTGAAGGATCTCCCTACAAAATCGGGTTAATTAAAATCAAAGGGAACACTCATACTAAGCATGATGTGA
TTTTGCATGAGACTAGTCTTTTCCCTGGAGACACTTTTGATAGATTAAAACTGGAAGGTACAGAGACTCG
TTTACGCAACACCGGCTACTTTAAAAGTGTAAGTGTCTATACGGTTCGTTCCCAATTAGATCCTCTTGAT
TCTAACGACCTTTATCGAGATGTTTTTATTGAAGTCAAAGAGACTGAAACAGGAAATCTTGGGCTATTCT
TAGGATTCAGCTCCATTGACCATTTATTTGGAGGGGCAGAAATTGCAGAAAGCAACTTTGATTTATTTGG
AGCCCGAAACTTTCTCAAAAAAGGATTCAAATCTTTAAGAGGTGGTGGAGAATACCTCTTCCTAAAAGCT
AATTTAGGAGATAAGGTCACCGATTACACTGTTAAATGGACGAAACCACACTTCTTAAATACCCCTTGGA
TTCTTGGAGTAGAATTAGATAAATCAATTAATAAAGCTTTATCAAAAGACTACTCTGTGGATACCTATGG
AGGGAATATCAGTACCACCTACATTCTTAACGATAAGTTAAAATATGGGATGTATTACCGTGGTAGCCAA
ACAAGCTTAAGTTTGCGCAAAAAAACGTCCAGCTCTAATAGACCTGGACCAGATTTAGATAGTAATAAAG
GATTTGTTTCCGCAGCGGGACTCAATGTTCTCTATGATTCTATTGATAATCCTAGAAAACCTACTATGGG
AATCCGCAGCTCCTTAAACTTTGAATTATCTGGTTTAGGCGGAACTTACCAATTTACTAAACTAACAGCT
AGTGGTTCTATCTATCGCTTATTAACTAAAAAAGGTGTTTTGAAAGTCCGTGCAGAAGCTAAGTTTATCA
AACCTTTCGGAACAACAACTGCACAAGGCATTCCTGTCAGCGAACGGTTCTTCTTAGGAGGTGAAACCAC
TGTTCGCGGTTACAAACCTTTTATTATTGGACCGAAATTTTCTCCTACTGAACCACAAGGAGGCTTGTCT
TCCCTACTATTAACAGAAGAATTTCAATATCCTTTGATTTCTCAACCTTGCATTAATGCCTTTGTATTTC
TAGATTCCGGATTCATTGGGATAGAAGAGTACACTATTCGCCTGAAAGACCTTTGCAGTAGCGCCGGATT
TGGTCTACGCTTTGATATGATGAATAATGTGCCAATTATGCTAGGCTGGGGTTGGCCGTTCCGCCCAACA
GAAATCCTCAATAATGAAAAAATTGATGTATCTCAAAGATTCTTTTTTGCCTTGGGAGGAGTATTCTAG
>gi|3328587|gb|AAC67774.1| CMP-2-keto-3-deoxyoctulosonic acid synthetase [Chlamydia
trachomatis][SEQ ID NO:27]
MFAFLTSKKVGILPSRWGSSRFPGKPLAKILGKILVQRSYENALSSQSLDCVVVATDDQRIFDHVVEFGG
LCVMTSTSCANGTERVEEVVSRHFPQAEIVVNIQGDEPCLSPTVIDGLVSTLENNPAADMVTSVTETTDP
EAILTDHKVKCVFDKNGKALYFSRSAIPHNFKHPTPIYLHIGVYAFRKAFLSEYVKIPPSSLSLAEDLEQ
LRVLEIGRSIYVHVVQNATGPSVDYPEDITKVEQYLLCLSKASF
>gi|3328586|gb|AE001292.1:216-980[SEQ ID NO:28]
GTGTTTGCGTTTTTAACCAGCAAAAAAGTCGGCATTCTCCCCTCTAGATGGGGAAGCTCCCGCTTCCCCG
GAAAACCTCTAGCAAAAATTCTAGGGAAAACCCTTGTTCAAAGATCCTATGAAAATGCCTTAAGCAGTCA
ATCTCTAGATTGCGTTGTTGTGGCAACAGATGATCAACGAATTTTTGACCATGTCGTTGAATTTGGGGGG
CTCTGTGTCATGACTAGCACATCTTGCGCTAACGGAACTGAGCGAGTAGAAGAGGTTGTGTCTCGACATT
TTCCTCAAGCAGAGATTGTTGTGAACATCCAAGGAGACGAGCCCTGTTTATCTCCTACCGTCATAGATGG
GCTTGTGAGCACGCTAGAGAACAATCCTGCTGCAGATATGGTCACATCTGTTACAGAAACAACAGACCCC
GAAGCGATATTGACAGATCACAAAGTGAAGTGTGTTTTCGATAAGAATGGCAAAGCTCTTTACTTTAGCA
GAAGCGCTATTCCTCACAACTTTAAACACCCAACGCCTATTTATCTGCATATTGGTGTTTATGCTTTTAG
AAAAGCTTTTCTAAGTGAATATGTTAAAATTCCTCCTTCCTCGTTAAGCCTAGCCGAAGATCTTGAACAA
TTACGAGTATTAGAAATAGGTCGTTCTATCTACGTTCATGTCGTTCAGAATGCAACGGGCCCTTCTGTTG
ATTATCCCGAAGATATAACCAAAGTGGAGCAGTATTTATTATGTCTTTCAAAAGCATCTTTTTGA
>gi|3329039|gb|AAC68197.1| Thio:disulfide Interchange Protein [Chlamydia
trachomatis][SEQ ID NO:29]
MIRQWYGFFLCLLFSYTSCFGVEENSGRATPTVELVSESEQAVEGEVLRIGVLIAIPEGEHIYWKNPGKL
GMPLRISWDLPSGCRLLEEHWPTPEIFEEDGVVYFGYKHSTMVVADIRVSKEIETRPLEIKAQVEWLSCG
ASCLPGSSSRVLVIPIDQGPLIPNSKETFTFSRALAAQPRPLDAAIKISYQPDGLDVFVPAGKADRATQA
WFIAENTRDFAYAQEVPLEQATTYIWKLKHPEGNMPKGIGLSGILIFKDDAGKVVASYQVEENQVEQLSA
LSWRFLSILLMAFIGGILLNIMPCVLPLITLKVFSLIKSAADHHSSSVIGGIGFTLGAIVSFWGLAFCAF
LLKVLGQNIGWGFQLQEPMFVAVLIIVFFLFALSSLGVFEMGIICLSLGKKLQEEGGASVRKNQIWGAFF
NGMLTTLVTTPCTGPFLGSVFGLVMAVSFVKQLAIFTAIGLGMASPYLLFASFPKMLAILPKPGPWMSTF
KQLTGFMLLATATWLIWIFGVETSATAVTILLVGLWLAAVGAWILGRWGTLVSPRNQRLLASVVFIFCIL
SSLVITSIGVRYFDENVPPAHSSDWQSFSPEKLADLREKGIPVFVNFTAKWCLTCQLNKPLLHANMQAFA
AKGVVTLEADWTKKDPKITEELARLGRASVPSYVYYPAGNKAPLILPERLSQSALEEMVFSQ
>gi|3329034|gb|AE001330.1:c6695-4617[SEQ ID NO:30]
ATGATTCGGCAATGGTATGGATTTTTTCTTTGCTTGCTGTTCAGCTATACGTCTTGTTTTGGTGTAGAAG
AAAATAGTGGAAGAGCTACGCCTACAGTAGAACTTGTTAGTGAAAGCGAACAAGCTGTTGAAGGAGAAGT
GCTTCGTATCGGAGTATTGATTGCTATTCCAGAAGGAGAGCATATCTACTGGAAAAATCCAGGGAAGCTT
GGAATGCCTTTGCGCATTTCTTGGGATTTGCCATCAGGATGTAGGTTGCTGGAGGAACATTGGCCGACTC
CAGAGATTTTCGAAGAGGATGGGGTTGTTTATTTTGGTTATAAACATTCTACAATGGTGGTTGCGGATAT
TCGCGTTTCTAAAGAGATAGAAACGCGTCCATTGGAGATAAAAGCGCAAGTTGAATGGTTGTCTTGCGGT
GCATCTTGTCTCCCAGGTTCTTCGTCAAGGGTTCTTGTGATTCCTATAGATCAGGGGCCGTTAATTCCTA
ATAGTAAAGAGACATTCACTTTTTCCCGTGCGTTAGCGGCTCAACCTCGACCTTTGGATGCTGCCATAAA
GATTTCTTATCAGCCTGATGGCTTAGATGTTTTTGTGCCAGCAGGGAAAGCGGATCGGGCAACCCAGGCA
TGGTTCATTGCTGAAAACACGCGAGATTTTGCTTATGCTCAAGAGGTTCCTCTTGAGCAAGCGACTACGT
ACATATGGAAGTTGAAACATCCTGAAGGAAATATGCCTAAGGGTATTGGGTTGTCGGGGATTCTTATATT
CAAGGATGATGCAGGGAAAGTAGTCGCTTCGTATCAAGTAGAAGAGAATCAAGTCGAACAGCTTTCGGCA
TTGAGCTGGAGGTTTCTCTCTATTCTTCTTATGGCTTTCATTGGTGGAATCTTATTAAACATCATGCCCT
GTGTATTGCCTCTGATTACTTTGAAAGTATTTAGTTTAATTAAATCGGCGGCAGATCACCATTCTTCCTC
TGTGATTGGAGGGATTGGGTTTACTTTAGGGGCTATTGTAAGCTTTTGGGGACTCGCTTTTTGTGCGTTT
TTGTTAAAGGTTTTAGGGCAAAATATTGGATGGGGATTCCAGCTTCAAGAACCCATGTTTGTTGCCGTTT
TAATTATTGTCTTCTTCTTATTTGCTCTGAGTTCGTTAGGCGTTTTTGAGATGGGAATAATTTGTCTGAG
CCTAGGGAAAAAATTGCAAGAAGAGGGAGGGGCATCGGTAAGGAAGAATCAGATCTGGGGAGCTTTTTTC
AATGGGATGTTGACTACCCTGGTTACAACTCCTTGCACTGGGCCTTTTCTTGGCTCTGTATTTGGATTAG
TTATGGCAGTGTCTTTTGTTAAGCAGCTGGCAATTTTTACTGCTATAGGATTAGGAATGGCAAGTCCCTA
TCTATTATTTGCTTCTTTTCCGAAGATGCTAGCCATTTTACCTAAACCTGGTCCTTGGATGAGTACGTTT
AAACAGTTGACTGGGTTTATGTTGCTTGCTACTGCAACTTGGCTTATCTGGATTTTTGGGGTAGAGACGA
GTGCAACCGCTGTAACTATTCTTCTTGTAGGATTGTGGTTGGCTGCTGTAGGTGCATGGATTCTAGGGAG
ATGGGGAACCCTTGTATCTCCGCGTAATCAGCGGCTTCTTGCTTCCGTTGTATTCATTTTCTGTATTTTA
AGTTCCTTAGTGATTACCTCTATAGGTGTCCGTTATTTTGATGAGAACGTCCCTCCTGCACATAGCTCTG
ATTGGCAATCTTTTTCTCCCGAAAAGCTAGCTGATTTACGCGAAAAAGGGATTCCAGTTTTTGTAAATTT
CACTGCAAAGTGGTGTTTAACGTGTCAACTCAATAAGCCTCTTCTTCATGCCAATATGCAAGCTTTTGCT
GCTAAGGGCGTAGTTACTTTAGAAGCAGATTGGACGAAAAAAGATCCAAAAATTACAGAAGAACTCGCTC
GTTTAGGCCGAGCCAGTGTACCTTCTTATGTGTATTACCCTGCGGGGAACAAAGCTCCGCTTATTCTTCC
AGAAAGATTATCGCAATCTGCTTTGGAAGAGATGGTTTTTTCTCAGTAG
>gi|3329000|gb|AAC68161.1| Yop proteins translocation lipoprotein J [Chlamydia
trachomatis][SEQ ID NO:31]
MFRYTLSRSLFFILALFFCSACDSRSMITHGLSGRDANEIVVLLVSKGVAAQKVPQAASSTGGSGEQLWD
ISVPAAQITEALAILNQAGLPRMKGTSLLDLFAKQGLVPSEMQEKIRYQEGLSEQMATTIRKMDGIVDAS
VQISFSPEEEDQRPLTASVYIKHRGVLDNPNSIMVSKIKRLVASAVPGLCPENVSVVSDRASYSDITING
PWGLSDEMNYVSVWGIILAKHSLTKFRLVFYFLILLLFILSCGLLWVIWKTHTLISALGGTKGFFDPAPY
SQLSFTQNKPAPKETPGAAEGAEAQTASEQPSKENAEKQEENNEDA
>gi|3328999|gb|AE001327.1:84-1064[SEQ ID NO:32]
ATGTTTCGTTATACTCTTTCTCGATCCTTATTTTTCATTTTGGCTCTTTTCTTCTGCTCGGCTTGTGATA
GTCGTTCCATGATTACACACGGCTTGTCAGGACGTGATGCTAATGAAATCGTAGTGCTTCTAGTCAGTAA
AGGGGTCGCTGCACAGAAAGTTCCCCAAGCAGCGTCCTCAACAGGAGGATCTGGAGAACAACTCTGGGAT
ATTTCGGTTCCTGCAGCACAAATTACAGAGGCTCTAGCTATTCTGAACCAAGCTGGGCTTCCAAGAATGA
AAGGAACCAGCCTTCTTGATCTATTCGCTAAACAAGGGCTGGTCCCTTCTGAAATGCAAGAAAAAATCCG
CTACCAAGAAGGTCTTTCAGAACAAATGGCTACGACCATTAGAAAGATGGACGGTATCGTCGATGCGAGC
GTACAGATTTCCTTTTCTCCTGAAGAAGAAGATCAACGGCCGCTAACAGCCTCTGTATATATCAAACACA
GAGGGGTATTAGACAACCCTAACAGTATTATGGTGTCTAAGATTAAACGTTTAGTTGCGAGTGCTGTCCC
AGGACTATGTCCCGAGAACGTTTCCGTAGTCAGTGACCGAGCTTCTTATAGTGACATTACTATTAATGGC
CCTTGGGGACTCTCCGATGAAATGAATTATGTTTCTGTATGGGGGATCATTCTAGCTAAGCATTCCCTTA
CTAAATTCCGCCTTGTTTTCTATTTCTTAATTCTCCTTCTCTTCATTCTTTCCTGTGGGCTACTCTGGGT
CATTTGGAAAACACACACACTGATTTCTGCTCTGGGTGGAACAAAAGGATTCTTTGATCCTGCTCCTTAC
TCACAGCTCTCTTTCACTCAGAATAAGCCAGCTCCAAAAGAAACTCCTGGAGCAGCAGAAGGTGCAGAAG
CGCAAACCGCTTCGGAACAACCCTCTAAAGAAAACGCAGAAAAACAAGAAGAGAATAACGAGGACGCTTA
A
>gi|3328905|gb|AAC68071.1| hypothetical protein [Chlamydia trachomatis][SEQ ID NO:33]
MEKRGVIVHILVCLLTIFGTFSLPAFGAHFLAEEEQFYMDRFVFSGQYPDMETMEIHAERKKRVQFDVTG
SFPKLESVVYKGSFGLLRSKIKGECPELSSVNLSCTSCRMDLDFRGEWKKNASIYIRNEQEPITIMLPKD
IGVVVYTQVDMNSKVVAEGSLIKRGRGFWKKTFRNSLVGESPVTLTFHVETRNGGVIFLR
>gi|3328891|gb|AE001320.1:c11104-10502[SEQ ID NO:34]
ATGGAGAAGAGAGGCGTTATTGTGCATATACTAGTTTGTTTGTTGACAATCTTCGGAACGTTCAGTTTAC
CCGCTTTCGGCGCGCATTTTCTCGCGGAAGAAGAGCAGTTTTATATGGATCGGTTTGTTTTCTCTGGGCA
GTATCCAGATATGGAAACTATGGAAATCCATGCAGAAAGAAAAAAACGTGTACAATTTGATGTGACGGGA
AGCTTCCCTAAGTTGGAGAGCGTGGTTTATAAGGGATCTTTTGGATTGCTGCGTTCGAAAATAAAGGGAG
AGTGTCCAGAACTGTCTTCTGTAAATCTTTCTTGTACCTCCTGCAGAATGGATTTAGATTTTCGAGGGGA
GTGGAAAAAGAATGCGTCTATTTATATTCGTAATGAGCAAGAGCCAATTACAATTATGTTGCCTAAAGAC
ATTGGTGTAGTTGTCTATACGCAGGTTGATATGAATAGTAAAGTAGTTGCAGAGGGATCACTAATCAAGA
GAGGAAGAGGTTTTTGGAAGAAAACTTTTCGGAATTCTTTGGTAGGAGAATCCCCTGTGACGCTAACTTT
TCATGTAGAGACTCGTAATGGAGGAGTTATTTTTCTCCGTTAG
>gi|3328884|gb|AAC68051.1| Phosphatidate Cytidylytransferase [Chlamydia
trachomatis][SEQ ID NO:35]
MFDSDHNSIFQSDLCQRLVVHSILLTFLVILLCTSLYPSSAFIVGLLSSACAALGTYEMGAMVRIKFPFS
FTRYSALGSAIFIALTCLTARCKMCFPEHIDLLPWFFLFFWTIRLVFKSRHYKLGPIGSTGLALFCMLYV
SVPIRLFLHILYGFVHTDTPFVGIWWAIFLIATTKSSDIFGYFFGKAFGKKRIAPVISPNKTVVGFIAGC
CGSILVSLLFYSHLPKAFADQIAVPWILIALGTVLGVSGFFGDIIESTFKRDAQIKNSSDLESIGGMLDV
LDSLLLSTPIVYAILLITQNRTFLG
>gi|3328881|gb|AE001319.1:1804-2721[SEQ ID NO:36]
ATGTTCGATTCGGATCATAATTCCATTTTTCAAAGCGATTTGTGTCAGCGTCTGGTCGTCCACTCGATTC
TTCTTACTTTCCTTGTCATTCTTCTCTGTACATCTTTATATCCCAGCTCAGCCTTTATTGTAGGGCTTCT
TTCCTCCGCTTGCGCAGCTCTAGGAACATATGAGATGGGGGCTATGGTTAGAATCAAGTTTCCATTTTCT
TTCACACGCTATAGTGCATTAGGATCCGCTATTTTCATTGCTCTGACCTGTCTTACAGCTCGTTGTAAAA
TGTGTTTTCCAGAGCATATAGACCTACTTCCTTGGTTCTTTCTCTTCTTTTGGACGATTCGCTTAGTATT
TAAAAGTCGCCATTATAAACTTGGTCCCATAGGCTCAACTGGGCTCGCGTTGTTTTGTATGCTTTATGTA
TCAGTCCCTATCCGCTTGTTCCTCCACATTTTGTATGGGTTTGTGCATACCGATACTCCATTTGTAGGAA
TTTGGTGGGCGATTTTTCTTATCGCTACAACAAAAAGCTCTGATATTTTTGGTTACTTCTTTGGAAAAGC
TTTTGGGAAAAAACGCATTGCACCAGTCATTAGCCCGAACAAAACAGTAGTAGGCTTCATTGCTGGTTGC
TGTGGATCTATCTTGGTTAGCCTTCTTTTCTACTCCCATCTTCCTAAAGCCTTTGCTGATCAGATTGCGG
TGCCTTGGATCCTCATTGCTTTAGGTACTGTGTTGGGCGTTAGTGGATTCTTTGGAGACATTATCGAATC
TACGTTCAAACGGGATGCACAGATCAAGAACAGCAGTGATCTGGAGTCTATCGGAGGAATGCTAGATGTG
CTAGACTCCTTGCTTCTTTCGACTCCTATCGTTTACGCTATCCTCCTTATCACTCAAAATAGGACATTTT
TAGGATGA
>gi|3328855|gb|AAC68022.1| hypothetical protein [Chlamydia trachomatis][SEQ ID NO:37]
MRRSVCYVTPSVARAGQISTWRFEYSSANFLPEGTLLKFDLGIDGRPIDWEIPSTDLSQPCNTIYLETPS
EDIVAAKAVYAPGGYIPTFEFTLPCDVEAGDTFSIILGSSPNFPQEDSSGNGAQLFTQRRKPFSLYVDPS
GKGSFEDPDIFTMDIRGNVLKNIRIFAPSYVIKNKRFDITVRFEDEFGNLTNFSPEETHIELSYEHLREN
LNWQLFIPETGFVILPNLYFNEPGIYRIQLRNQATKEVFTSAPIKCFAETSSHLLWGLLHGESERVDSEG
NIESCLRYFRDDCALNFFATSSFEIQDGLTPETIKTINQTVADFNEEDRFIALSGAQYLSEEPGEGIREV
LLMKEPKSPGKHKECKLFPLSKLYKQSTSHELISIPSFTASKKFGYNFNNFHPEFERVVEIYNAWGCSER
TEAEGNPFPIKGSIDSENPEGTVLSALKRNLRFGFVAGGLDDRNLYNHFFDSDQQQYSPGLTAVICNKYS
RDSLLEALYQRQCYATTGQRIIVNFQITSAPMGSELSTAIKPGLVINRHISGYVAGTAKIASIEIIRNED
ILHTFHPDGNNFEYEYDDLSPFAQVTLKDPQNGAPFAFYYLRVTQENGAMAWSSPIWIDLN
>gi|3328850|gb|AE001316.1:4105-5970[SEQ ID NO:38]
ATGCGCAGATCTGTTTGTTACGTTACTCCTTCAGTTGCTAGGGCTGGTCAAATTTCTACCTGGCGTTTCG
AATATTCTTCAGCTAATTTCCTTCCCGAAGGCACATTGCTAAAATTTGACCTGGGAATAGACGGACGCCC
TATAGACTGGGAGATTCCTTCTACAGATCTTTCTCAACCATGTAATACAATTTATTTAGAAACGCCTTCC
GAGGATATTGTGGCTGCAAAAGCTGTGTATGCTCCCGGAGGCTATATCCCTACTTTCGAATTTACTCTCC
CTTGTGATGTGGAAGCTGGGGACACTTTCTCTATTATTCTTGGCTCCTCTCCCAACTTCCCTCAAGAGGA
CTCTTCAGGTAATGGTGCTCAATTATTTACTCAACGCCGTAAACCTTTCTCTCTTTATGTTGACCCATCA
GGGAAAGGATCTTTTGAAGATCCCGATATCTTCACAATGGATATCAGAGGAAATGTATTAAAAAATATCC
GGATTTTTGCTCCTTCTTATGTGATCAAAAACAAACGCTTTGATATTACAGTGCGCTTCGAAGATGAATT
TGGGAACTTAACCAATTTCTCCCCAGAAGAGACCCATATCGAGCTTTCGTACGAACATCTTCGCGAAAAC
CTCAATTGGCAATTGTTCATCCCTGAAACAGGCTTTGTGATCCTTCCAAACCTGTATTTCAATGAACCAG
GTATTTATCGTATTCAACTACGCAATCAAGCAACAAAAGAGGTCTTTACATCAGCGCCTATCAAATGTTT
TGCAGAAACCTCCTCTCATCTTTTGTGGGGGCTTCTACATGGAGAATCTGAACGTGTCGACTCTGAAGGT
AATATCGAGTCTTGCTTGCGTTATTTTCGTGATGACTGCGCGTTAAACTTTTTTGCAACATCCTCTTTCG
AAATTCAAGATGGCCTGACCCCAGAAACCATTAAAACCATTAACCAAACCGTTGCTGATTTTAATGAAGA
AGATCGTTTCATTGCCTTATCCGGAGCACAGTACCTTTCTGAAGAGCCTGGCGAGGGAATTCGTGAAGTA
TTGCTGATGAAGGAACCCAAATCCCCAGGGAAACATAAAGAATGCAAACTATTTCCTTTATCTAAGCTAT
ATAAGCAATCAACTAGTCATGAGTTAATCTCAATCCCCAGCTTCACTGCTTCAAAGAAATTTGGATACAA
TTTTAATAATTTCCATCCTGAATTTGAAAGAGTTGTTGAAATTTATAATGCCTGGGGATGCTCTGAAAGA
ACTGAAGCTGAAGGAAACCCTTTCCCTATTAAAGGTTCTATCGACTCAGAAAATCCAGAGGGAACTGTTC
TATCTGCTTTAAAGAGAAACCTGCGTTTTGGATTCGTAGCCGGTGGTCTTGATGATAGAAATCTATACAA
TCACTTTTTTGATTCCGATCAACAGCAATACTCCCCTGGATTAACAGCTGTGATCTGCAATAAATATTCT
CGGGATTCCTTACTCGAGGCATTATACCAACGACAATGCTATGCTACAACCGGCCAAAGAATTATCGTGA
ATTTCCAGATTACATCTGCTCCTATGGGCTCCGAACTCTCCACAGCCATTAAACCAGGGCTCGTGATCAA
TAGACATATTTCGGGATATGTAGCAGGAACTGCCAAGATTGCGTCGATCGAAATCATCCGCAATGAGGAT
ATTCTCCATACCTTCCACCCAGATGGAAATAACTTTGAGTATGAGTACGACGATCTCTCTCCTTTTGCAC
AAGTCACTCTAAAAGATCCTCAAAATGGAGCTCCTTTTGCTTTTTACTACTTACGAGTCACTCAAGAGAA
TGGAGCTATGGCTTGGAGCTCTCCTATTTGGATAGATCTTAACTAA
>gi|3328772|gb|AAC67946.1| hypothetical protein [Chlamydia trachomatis][SEQ ID NO:39]
MKRFFPLFIGVLLAHTLPSEGLSHQQAVQKKISYLSHFKGITGIMDVEDGVLHIHDDLRLQANKAYVENR
TDCGIKIVAHGNVMVNYRGKILICDYLEYYEDTDSCLLTNGRCSLYPWFIGGSTITISPSSIIIHKGYIS
TSEGPQKHICLSGDYLKYSSDSVLSMGPSRLSICNTPVLLLPQISIMPMEIPKPPITFRGGSGGFLGSYL
GVSYSPISKKHCSTTLFLDGFFKHGIGLGYNMRFSSQENPSNAINIKSYYAHRLAIDSSGAKDRYRLHGD
FTFSKERAHLAGEFHLSDSWETVVDIFPNNFSLKNTGPTEVSLSWRDNNLFGKMTSSVKVNSFQNVKQEL
PQAILHHRPVRIRRSRIFLENRLEAGFLDFHFSSNIPGSNFSSWRFSSAHKVYRGLVLPIGTLTPSLSGT
AIYYTRMLSPNAAHCQLSGSLSFDYRVALQKEYRHARHIVEPFCSFLKTTRPVLSSDEPHIFSIKDAFHS
INLLHVGLESKVLNKHSTPSHLKLWTTYIFDEPHAKDTFPKTACWFSLPLTLQNTLSLDAEWIWKKSRWD
HLNVIWEWILNDNLGLTLEFLHRSKYGFIKCAKDNYTLDVSRSLDTLLASPLSDRRNLITGKLFVRPHPH
WNYNLNLRYGWHRPDSPSYLEYQMILGHKIFEHWQLFSVYEKREADKRCFFYLKLDKRKQKHRHPFG
>gi|3328766|gb|AE001308.1:6085-8178[SEQ ID NO:40]
GTGAAACGATTTTTCCCACTTTTTATTGGAGTGCTGCTCGCGCACACTTTGCCGTCAGAAGGTCTTTCTC
ATCAACAAGCTGTCCAAAAAAAAATTTCTTATCTGAGCCATTTTAAAGGCATTACAGGAATTATGGATGT
TGAGGATGGGGTATTACATATCCATGATGATCTACGTCTTCAAGCCAATAAAGCCTATGTTGAAAATCGC
ACGGATTGTGGGATCAAAATCGTTGCTCATGGCAACGTTATGGTCAATTATCGCGGGAAAATTTTAATCT
GTGATTATCTTGAGTACTATGAAGATACAGATTCTTGTTTACTCACCAATGGCCGCTGTTCGTTATACCC
ATGGTTCATTGGAGGATCCACTATAACGATCTCACCATCTTCTATTATCATTCATAAAGGGTATATCTCG
ACTTCTGAAGGTCCTCAGAAACATATTTGTTTATCCGGAGATTATTTAAAATACTCTTCAGACAGCGTAT
TATCTATGGGACCCTCACGTCTTTCTATCTGTAATACGCCTGTGTTATTGCTTCCTCAAATCTCCATTAT
GCCTATGGAGATTCCTAAGCCTCCGATTACTTTTCGAGGTGGGAGTGGAGGATTTCTGGGATCCTACTTA
GGTGTTAGTTATTCCCCTATATCTAAAAAGCATTGTTCTACGACTTTGTTCTTGGATGGTTTTTTTAAAC
ATGGAATAGGTCTCGGCTATAACATGCGCTTTTCCTCTCAGGAAAATCCAAGCAATGCCATAAATATTAA
AAGCTATTACGCACATCGATTAGCTATTGATTCATCAGGAGCAAAAGATCGCTATCGATTACATGGAGAC
TTCACTTTTTCCAAAGAACGAGCCCATCTTGCTGGTGAATTCCATTTAAGTGATAGCTGGGAAACAGTTG
TGGATATCTTCCCAAATAACTTCTCTTTAAAAAATACAGGCCCTACAGAAGTTAGCCTATCATGGCGCGA
TAACAATTTATTTGGGAAAATGACTTCCTCTGTCAAAGTCAACTCCTTTCAAAATGTTAAACAAGAATTG
CCTCAAGCAATTCTTCATCACCGACCAGTACGTATCAGGCGCTCTCGCATTTTCCTAGAGAATCGCTTAG
AAGCTGGTTTTTTAGATTTTCATTTCAGTAGTAATATTCCAGGCTCTAACTTCTCATCATGGAGGTTCTC
ATCCGCTCACAAAGTCTACCGTGGGCTTGTTCTTCCTATAGGAACGTTAACCCCTTCGCTATCTGGAACT
GCTATCTACTATACCCGCATGCTCTCCCCAAATGCAGCCCATTGTCAATTATCTGGATCGCTATCTTTTG
ATTATCGCGTTGCTTTACAAAAAGAATATCGGCATGCAAGACATATTGTAGAGCCTTTTTGCTCCTTTTT
AAAAACCACTCGTCCTGTATTATCCTCTGATGAGCCTCATATTTTCTCGATTAAAGATGCTTTTCACTCT
ATCAACCTTCTACATGTAGGATTGGAGTCAAAAGTCTTAAACAAACATTCCACTCCTTCGCATCTGAAAC
TATGGACGACCTATATCTTTGATGAACCTCACGCTAAGGACACTTTCCCTAAAACTGCTTGCTGGTTCTC
TCTTCCTCTTACACTCCAAAATACTTTATCCTTAGATGCGGAATGGATTTGGAAAAAAAGCCGATGGGAT
CATCTCAATGTAATCTGGGAATGGATTTTGAATGATAATCTCGGTCTTACTTTAGAATTTTTACATAGAA
GTAAGTATGGCTTTATTAAGTGCGCTAAAGATAACTACACACTCGATGTAAGCCGATCTTTAGACACATT
ACTAGCCTCTCCTCTTTCCGATCGAAGAAATTTGATTACTGGCAAACTTTTTGTTCGTCCACATCCTCAT
TGGAATTATAATCTTAATCTTCGTTATGGATGGCATCGTCCAGACTCTCCATCCTATTTAGAATACCAGA
TGATTCTGGGTCATAAAATCTTTGAGCACTGGCAGCTATTCTCTGTCTACGAAAAACGTGAAGCTGATAA
GCGCTGCTTCTTTTATCTAAAATTAGATAAACGAAAACAGAAACACCGCCATCCTTTTGGATAA
>gi|3329347|gb|AAC68470.| Putative Outer Membrane Protein H [Chlamydia
trachomatis][SEQ ID NO:41]
MPFSLRSTSFCFLACLCSYSYGFASSPQVLTPNVTTPFKGDDVYLNGDCAFVNVYAGAENGSIISANGDN
LTITGQNHTLSFTDSQGPVLQNYAFISAGETLTLKDFSSLMFSKNVSCGEKGMISGKTVSISGAGEVIFW
DNSVGYSPLSIVPASTPTPPAPAPAPAASSSLSPTVSDARKGSIFSVETSLEISGVKKGVMFDNNAGNFG
TVFRGNSNNNAGSGGSGSATTPSFTVKNCKGKVSFTDNVASCGGGVVYKGTVLFKDNEGGIFFRGNTAYD
DLGILAATSRDQNTETGGGGGVICSPDDSVKFEGNKGSIVFDYNFAKGRGGSILTKEFSLVADDSVVFSN
NTAEKGGGAIYAPTIDISTNGGSILFERNRAAEGGAICVSEASSGSTGNLTLSASDGDIVFSGNMTSDRP
GERSAARILSDGTTVSLNASGLSKLIFYDPVVQNNSAAGASTPSPSSSSMPGAVTINQSGNGSVIFTAES
LTPSEKLQVLNSTSNFPGALTVSGGELVVTEGATLTTGTITATSGRVTLGSGASLSAVAGAANNNYTCTV
SKLGIDLESFLTPNYKTAILGADGTVTVNSGSTLDLVMESEAEVYDNPLFVGSLTIPFVTLSSSSASNGV
TKNSVTINDADAAHYGYQGSWSADWTKPPLAPDAKGMVPPNTNNTLYLTWRPASNYGEYRLDPQRKGELV
PNSLWVAGSALRTFTNGLKEHYVSRDVGFVASLHALGDYILNYTQDDRDGFLARYGGFQATAASHYENGS
IFGVAFGQLYGQTKSRMYYSKDAGNMTMLSCFGRSYVDIKGTETVMYWETAYGYSVHRMHTQYFNDKTQK
FDHSKCHWHNNNYYAFVGAEHNFLEYCIPTRQFARDYELTGFMRFEMAGGWSSSTRETGSLTRYFARGSG
HNMSLPIGIVAHAVSHVRRSPPSKLTLNMGYRPDIWRVTPHCNMEIIANGVKTPIQGSPLARHAFFLEVH
DTLYIHHFGRAYMNYSLDARRRQTAHFVSMGLNRIF
>gi|3329342|gb|AE001360.1:10808-13858[SEQ ID NO:42]
ATGCCTTTTTCTTTGAGATCTACATCATTTTGTTTTTTAGCTTGTTTGTGTTCCTATTCGTATGGATTCG
CGAGCTCTCCTCAAGTGTTAACACCTAATGTAACCACTCCTTTTAAGGGGGACGATGTTTACTTGAATGG
AGACTGCGCTTTTGTCAATGTCTATGCAGGGGCAGAGAACGGCTCAATTATCTCAGCTAATGGCGACAAT
TTAACGATTACCGGACAAAACCATACATTATCATTTACAGATTCTCAAGGGCCAGTTCTTCAAAATTATG
CCTTCATTTCAGCAGGAGAGACACTTACTCTGAAAGATTTTTCGAGTTTGATGTTCTCGAAAAATGTTTC
TTGCGGAGAAAAGGGAATGATCTCAGGGAAAACCGTGAGTATTTCCGGAGCAGGCGAAGTGATTTTTTGG
GATAACTCTGTGGGGTATTCTCCTTTGTCTATTGTGCCAGCATCGACTCCAACTCCTCCAGCACCAGCAC
CAGCTCCTGCTGCTTCAAGCTCTTTATCTCCAACAGTTAGTGATGCTCGGAAAGGGTCTATTTTTTCTGT
AGAGACTAGTTTGGAGATCTCAGGCGTCAAAAAAGGGGTCATGTTCGATAATAATGCCGGGAATTTTGGA
ACAGTTTTTCGAGGTAATAGTAATAATAATGCTGGTAGTGGGGGTAGTGGGTCTGCTACAACACCAAGTT
TTACAGTTAAAAACTGTAAAGGGAAAGTTTCTTTCACAGATAACGTAGCCTCCTGTGGAGGCGGAGTAGT
CTACAAAGGAACTGTGCTTTTCAAAGACAATGAAGGAGGCATATTCTTCCGAGGGAACACAGCATACGAT
GATTTAGGGATTCTTGCTGCTACTAGTCGGGATCAGAATACGGAGACAGGAGGCGGTGGAGGAGTTATTT
GCTCTCCAGATGATTCTGTAAAGTTTGAAGGCAATAAAGGTTCTATTGTTTTTGATTACAACTTTGCAAA
AGGCAGAGGCGGAAGCATCCTAACGAAAGAATTCTCTCTTGTAGCAGATGATTCGGTTGTCTTTAGTAAC
AATACAGCAGAAAAAGGCGGTGGAGCTATTTATGCTCCTACTATCGATATAAGCACGAATGGAGGATCGA
TTCTATTTGAAAGAAACCGAGCTGCAGAAGGAGGCGCCATCTGCGTGAGTGAAGCAAGCTCTGGTTCAAC
TGGAAATCTTACTTTAAGCGCTTCTGATGGGGATATTGTTTTTTCTGGGAATATGACGAGTGATCGTCCT
GGAGAGCGCAGCGCAGCAAGAATCTTAAGTGATGGAACGACTGTTTCTTTAAATGCTTCCGGACTATCGA
AGCTGATCTTTTATGATCCTGTAGTACAAAATAATTCAGCAGCGGGTGCATCGACACCATCACCATCTTC
TTCTTCTATGCCTGGTGCTGTCACGATTAATCAGTCCGGTAATGGATCTGTGATTTTTACCGCCGAGTCA
TTGACTCCTTCAGAAAAACTTCAAGTTCTTAACTCTACTTCTAACTTCCCAGGAGCTCTGACTGTGTCAG
GAGGGGAGTTGGTTGTGACGGAAGGAGCTACCTTAACTACTGGGACCATTACAGCCACCTCTGGACGAGT
GACTTTAGGATCCGGAGCTTCGTTGTCTGCCGTTGCAGGTGCTGCAAATAATAATTATACTTGTACAGTA
TCTAAGTTGGGGATTGATTTAGAATCCTTTTTAACTCCTAACTATAAGACGGCCATACTGGGTGCGGATG
GAACAGTTACTGTTAACAGCGGCTCTACTTTAGACCTAGTGATGGAGAGTGAGGCAGAGGTATATGATAA
TCCGCTTTTTGTGGGATCGCTGACAATTCCTTTTGTTACTCTATCTTCTAGTAGTGCTAGTAACGGAGTT
ACAAAAAATTCTGTCACTATTAATGATGCAGACGCTGCGCACTATGGGTATCAAGGCTCTTGGTCTGCAG
ATTGGACGAAACCGCCTCTGGCTCCTGATGCTAAGGGGATGGTACCTCCTAATACCAATAACACTCTGTA
TCTGACATGGAGACCTGCTTCGAATTACGGTGAATATCGACTGGATCCTCAGAGAAAGGGAGAACTAGTA
CCCAACTCTCTTTGGGTAGCGGGATCTGCATTAAGAACCTTTACTAATGGTTTGAAAGAACACTATGTTT
CTAGAGATGTTGGATTTGTAGCATCTCTGCATGCTCTCGGGGATTATATTTTGAATTATACGCAAGATGA
TCGGGATGGCTTTTTAGCTAGATATGGGGGATTCCAGGCGACCGCAGCCTCCCATTATGAAAATGGGTCA
ATATTTGGAGTGGCTTTTGGACAACTCTATGGTCAGACAAAGAGCAGAATGTATTACTCTAAAGATGCTG
GGAACATGACGATGTTGTCCTGTTTCGGAAGAAGTTACGTAGATATTAAAGGAACAGAAACTGTTATGTA
TTGGGAGACGGCTTATGGCTATTCTGTGCACAGAATGCATACGCAGTATTTTAATGACAAAACGCAGAAG
TTCGATCATTCGAAATGTCATTGGCACAACAATAACTATTATGCGTTTGTGGGTGCCGAGCATAATTTCT
TAGAGTACTGCATTCCTACTCGTCAGTTCGCTAGAGATTATGAGCTTACAGGGTTTATGCGTTTTGAAAT
GGCCGGAGGATGGTCCAGTTCTACACGAGAAACTGGCTCCCTAACTAGATATTTCGCTCGCGGGTCAGGG
CATAATATGTCGCTTCCAATAGGAATTGTAGCTCATGCAGTTTCTCATGTGCGAAGATCTCCTCCTTCTA
AACTGACACTAAATATGGGATATAGACCAGACATTTGGCGTGTCACTCCACATTGCAATATGGAAATTAT
TGCTAACGGAGTGAAGACACCTATACAAGGATCTCCGCTGGCACGGCATGCCTTCTTCTTAGAAGTGCAT
GATACTTTGTATATTCATCATTTTGGAAGAGCCTATATGAACTATTCGCTGGATGCTCGTCGTCGACAAA
CGGCACATTTTGTATCCATGGGCTTGAATAGAATCTTTTAA
>gi|3328874|gb|AAC68042.1|60kDa Cysteine-Rich OMP [Chlamydia trachomatis][SEQ ID NO:43]
MRIGDPMNKLIRRAVTIFAVTSVASLFASGVLETSMAESLSTNVISLADTKAKDNTSHKSKKARKNHSKE
TPVDRKEVAPVHESKATGPKQDSCFGRMYTVKVNDDRNVEITQAVPEYATVGSPYPIEITATGKRDCVDV
IITQQLPCEAEFVRSDPATTPTADGKLVWKIDRLGQGEKSKITVWVKPLKEGCCFTAATVCACPEIRSVT
KCGQPAICVKQEGPENACLRCPVVYKINIVNQGTATARNVVVENPVPDGYAHSSGQRVLTFTLGDMQPGE
HRTITVEFCPLKRGRATNIATVSYCGGHKNTASVTTVINEPCVQVSIAGADWSYVCKPVEYVISVSNPGD
LVLRDVVVEDTLSPGVTVLEAAGAQISCNKVVWTVKELNPGESLQYKVLVRAQTPGQFTNNVVVKSCSDC
GTCTSCAEATTYWKGVAATHMCVVDTCDPVCVGENTVYRICVTNRGSAEDTNVSLMLKFSKELQPVSFSG
PTKGTITGNTVVFDSLPRLGSKETVEFSVTLKAVSAGDARGEAILSSDTLTVPVSDTENTHIY
>gi|3328863|gb|AE001317.1:c11039-9378[SEQ ID NO:44]
ATGCGAATAGGAGATCCTATGAACAAACTCATCAGACGAGCAGTGACGATCTTCGCGGTGACTAGTGTGG
CGAGTTTATTTGCTAGCGGGGTGTTAGAGACCTCTATGGCAGAGTCTCTCTCTACAAACGTTATTAGCTT
AGCTGACACCAAAGCGAAAGACAACACTTCTCATAAAAGCAAAAAAGCAAGAAAAAACCACAGCAAAGAG
ACTCCCGTAGACCGTAAAGAGGTTGCTCCGGTTCATGAGTCTAAAGCTACAGGACCTAAACAGGATTCTT
GCTTTGGCAGAATGTATACAGTCAAAGTTAATGATGATCGCAATGTTGAAATCACACAAGCTGTTCCTGA
ATATGCTACGGTAGGATCTCCCTATCCTATTGAAATTACTGCTACAGGTAAAAGGGATTGTGTTGATGTT
ATCATTACTCAGCAATTACCATGTGAAGCAGAGTTCGTACGCAGTGATCCAGCGACAACTCCTACTGCTG
ATGGTAAGCTAGTTTGGAAAATTGACCGCTTAGGACAAGGCGAAAAGAGTAAAATTACTGTATGGGTAAA
ACCTCTTAAAGAAGGTTGCTGCTTTACAGCTGCAACAGTATGCGCTTGTCCAGAGATCCGTTCGGTTACA
AAATGTGGACAACCTGCTATCTGTGTTAAACAAGAAGGCCCAGAGAATGCTTGTTTGCGTTGCCCAGTAG
TTTACAAAATTAATATAGTGAACCAAGGAACAGCAACAGCTCGTAACGTTGTTGTTGAAAATCCTGTTCC
AGATGGTTACGCTCATTCTTCTGGACAGCGTGTACTGACGTTTACTCTTGGAGATATGCAACCTGGAGAG
CACAGAACAATTACTGTAGAGTTTTGTCCGCTTAAACGTGGTCGTGCTACCAATATAGCAACGGTTTCTT
ACTGTGGAGGACATAAAAATACAGCAAGCGTAACAACTGTGATCAACGAGCCTTGCGTACAAGTAAGTAT
TGCAGGAGCAGATTGGTCTTATGTTTGTAAGCCTGTAGAATATGTGATCTCCGTTTCCAATCCTGGAGAT
CTTGTGTTGCGAGATGTCGTCGTTGAAGACACTCTTTCTCCCGGAGTCACAGTTCTTGAAGCTGCAGGAG
CTCAAATTTCTTGTAATAAAGTAGTTTGGACTGTGAAAGAACTGAATCCTGGAGAGTCTCTACAGTATAA
AGTTCTAGTAAGAGCACAAACTCCTGGACAATTCACAAATAATGTTGTTGTGAAGAGCTGCTCTGACTGT
GGTACTTGTACTTCTTGCGCAGAAGCGACAACTTACTGGAAAGGAGTTGCTGCTACTCATATGTGCGTAG
TAGATACTTGTGACCCTGTTTGTGTAGGAGAAAATACTGTTTACCGTATTTGTGTCACCAACAGAGGTTC
TGCAGAAGATACAAATGTTTCTTTAATGCTTAAATTCTCTAAAGAACTGCAACCTGTATCCTTCTCTGGA
CCAACTAAAGGAACGATTACAGGCAATACAGTAGTATTCGATTCGTTACCTAGATTAGGTTCTAAAGAAA
CTGTAGAGTTTTCTGTAACATTGAAAGCAGTATCAGCTGGAGATGCTCGTGGGGAAGCGATTCTTTCTTC
CGATACATTGACTGTTCCAGTTTCTGATACAGAGAATACACACATCTATTAA
>gi|3328841|gb|AAC68010.1| Putative outer membrane protein B [Chlamydia
trachomatis][SEQ ID NO:45]
MKWLSATAVFAAVLPSVSGFCFPEPKELNFSRVGTSSSTTFTETVGEAGAEYIVSGNASFTKFTNIPTTD
TTTPTNSNSSSSNGETASVSEDSDSTTTTPDPKGGGAFYNAHSGVLSFMTRSGTEGSLTLSEIKITGEGG
AIFSQGELLFTDLTGLTIQNNLSQLSGGAIFGESTISLSGITKATFSSNSAEVPAPVKKPTEPKAQTASE
TSGSSSSSGNDSVSSPSSSRAEPAAANLQSHFICATATPAAQTDTETSTPSHKPGSGGAIYAKGDLTIAD
SQEVLFSINKATKDGGAIFAEKDVSFENITSLKVQTNGAEEKGGAIYAKGDLSIQSSKQSLFNSNYSKQG
GGALYVEGDINFQDLEEIRIKYNKAGTFETKKITLPKAQASAGNADAWASSSPQSGSGATTVSNSGDSSS
GSDSDTSETVPATAKGGGLYTDKNLSITNITGIIEIANNKATDVGGGAYVKGTLTCENSHRLQFLKNSSD
KQGGGIYGEDNITLSNLTGKTLFQENTAKEEGGGLFIKGTDKALTMTGLDSFCLINNTSEKHGGGAFVTK
EISQTYTSDVETIPGITPVHGETVITGNKSTGGNGGGVCTKRLALSNLQSISISGNSAAENGGGAHTCPD
SFPTADTAEQPAAASAATSTPESAPVVSTALSTPSSSTVSSLTLLAASSQASPATSNKETQDPNADTDLL
IDYVVDTTISKNTAKKGGGIYAKKAKMSRIDQLNISENSATEIGGGICCKESLELDALVSLSVTENLVGK
EGGGLHAKTVNISNLKSGFSFSNNKANSSSTGVATTASAPAAAAASLQAAAAAVPSSPATPTYSGVVGGA
IYGEKVTFSQCSGTCQFSGNQAIDNNPSQSSLNVQGGAIYAKTSLSIGSSDAGTSYIFSGNSVSTGKSQT
TGQIAGGAIYSPTVTLNCPATFSNNTASMATPKTSSEDGSSGNSIKDTIGGAIAGTAITLSGVSRFSGNT
ADLGAAIGTLANANTPSATSGSQNSITEKITLENGSFIFERNQANKRGAIYSPSVSIKGNNITFNQNTST
HDGSAIYFTKDATIESLGSVLFTGNNVTATQASSATSGQNTNTANYGAAIFGDPGTTQSSQTDAILTLLA
SSGNITFSNNSLQNNQGDTPASKFCSIAGYVKLSLQAAKGKTISFFDCVHTSTKKIGSTQNVYETLDINK
EENSNPYTGTIVFSSELHENKSYIPQNAILHNGTLVLKEKTELHVVSFEQKEGSKLIMKPGAVLSNQNIA
NGALVINGLTIDLSSMGTPQAGEIFSPPELRIVATTSSASGGSGVSSSIPTNPKRISAAAPSGSAATTPT
MSENKVFLTGDLTLIDPNGNFYQNPMLGSDLDVPLIKLPTNTSDVQVYDLTLSGDLFPQKGYMGTWTLDS
NPQTGKLQARWTFDTYRRWVYIPRDNHFYANSILGSQNSMIVVKQGLINNMLNNARFDDIAYNNFWVSGV
GTFLAQQGTPLSEEFSYYSRGTSVAIDAKPRQDFILGAAFSKMVGKTKAIKKMHNYFHKGSEYSYQASVY
GGKFLYFLLNKQHGWALPFLIQGVVSYGHIKHDTTTLYPSIHERNKGDWEDLGWLADLRISMDLKEPSKD
SSKRITVYGELEYSSIRQKQFTEIDYDPRHFDDCAYRNLSLPVGCAVEGAIMNCNILMYNKLALAYMPSI
YRNNPVCKYRVLSSNEAGQVICGVPTRTSARAEYSTQLYLGPFWTLYGNYTIDVGMYTLSQMTSCGARMI
F
>gi|3328833|gb|AE001314.1:9601-14856[SEQ ID NO:46]
ATGAAATGGCTGTCAGCTACTGCGGTGTTTGCTGCTGTTCTCCCCTCAGTTTCAGGGTTTTGCTTCCCAG
AACCTAAAGAATTAAATTTCTCTCGCGTAGGAACTTCTTCCTCTACCACTTTTACTGAAACAGTTGGAGA
AGCTGGGGCAGAATATATCGTCTCTGGTAACGCATCTTTCACAAAATTTACCAACATTCCTACTACCGAT
ACAACAACTCCCACGAACTCAAACTCCTCTAGCTCTAACGGAGAGACTGCTTCCGTTTCTGAGGATAGTG
ACTCTACAACAACGACTCCTGATCCTAAAGGTGGCGGCGCCTTTTATAACGCGCACTCCGGAGTTTTATC
CTTTATGACACGATCAGGAACAGAAGGTTCCTTAACTCTGTCTGAGATAAAAATAACTGGTGAAGGCGGT
GCTATCTTCTCTCAAGGAGAGCTGCTATTTACAGATCTGACAGGTCTAACCATCCAAAATAACTTATCCC
AGCTATCCGGAGGAGCGATTTTTGGAGAATCTACAATCTCCCTATCAGGGATTACTAAAGCGACTTTCTC
CTCCAACTCTGCAGAAGTTCCTGCTCCTGTTAAGAAACCTACAGAACCTAAAGCTCAAACAGCAAGCGAA
ACGTCGGGTTCTAGTAGTTCTAGCGGAAATGATTCGGTGTCTTCCCCCAGTTCCAGTAGAGCTGAACCCG
CAGCAGCTAATCTTCAAAGTCACTTTATTTGTGCTACAGCTACTCCTGCTGCTCAAACCGATACAGAAAC
ATCAACTCCCTCTCATAAGCCAGGATCTGGGGGAGCTATCTATGCTAAAGGCGACCTTACTATCGCAGAC
TCTCAAGAGGTACTATTCTCAATAAATAAAGCTACTAAAGATGGAGGAGCGATCTTTGCTGAGAAAGATG
TTTCTTTCGAGAATATTACATCATTAAAAGTACAAACTAACGGTGCTGAAGAAAAGGGAGGAGCTATCTA
TGCTAAAGGTGACCTCTCAATTCAATCTTCTAAACAGAGTCTTTTTAATTCTAACTACAGTAAACAAGGT
GGTGGGGCTCTATATGTTGAAGGAGATATAAACTTCCAAGATCTTGAAGAAATTCGCATTAAGTACAATA
AAGCTGGAACGTTCGAAACAAAAAAAATCACTTTACCAAAAGCTCAAGCATCTGCAGGAAATGCAGATGC
TTGGGCCTCTTCCTCTCCTCAATCTGGTTCTGGAGCAACTACAGTCTCCAACTCAGGAGACTCTAGCTCT
GGCTCAGACTCGGATACCTCAGAAACAGTTCCAGCCACAGCTAAAGGCGGTGGGCTTTATACTGATAAGA
ATCTTTCGATTACTAACATCACAGGAATTATCGAAATTGCAAATAACAAAGCGACAGATGTTGGAGGTGG
TGCTTACGTAAAAGGAACCCTTACTTGTGAAAACTCTCACCGTCTACAATTTTTGAAAAACTCTTCCGAT
AAACAAGGTGGAGGAATCTACGGAGAAGACAACATCACCCTATCTAATTTGACAGGGAAGACTCTATTCC
AAGAGAATACTGCCAAAGAAGAGGGCGGTGGACTCTTCATAAAAGGTACAGATAAAGCTCTTACAATGAC
AGGACTGGATAGTTTCTGTTTAATTAATAACACATCAGAAAAACATGGTGGTGGAGCCTTTGTTACCAAA
GAAATCTCTCAGACTTACACCTCTGATGTGGAAACAATTCCAGGAATCACGCCTGTACATGGTGAAACAG
TCATTACTGGCAATAAATCTACAGGAGGTAATGGTGGAGGCGTGTGTACAAAACGTCTTGCCTTATCTAA
CCTTCAAAGCATTTCTATATCCGGGAATTCTGCAGCTGAAAATGGTGGTGGAGCCCACACATGCCCAGAT
AGCTTCCCAACGGCGGATACTGCAGAACAGCCCGCAGCAGCTTCTGCCGCGACGTCTACTCCCGAGTCTG
CCCCAGTGGTCTCAACTGCTCTAAGCACACCTTCATCTTCTACCGTCTCTTCATTAACCTTACTACCAGC
CTCTTCACAAGCCTCTCCTGCAACCTCTAATAAGGAAACTCAAGATCCTAATGCTGATACAGACTTATTG
ATCGATTATGTAGTTGATACGACTATCAGCAAAAACACTGCTAAGAAAGGCGGTGGAATCTATGCTAAAA
AAGCCAAGATGTCCCGCATAGACCAACTGAATATCTCTGAGAACTCCGCTACAGAGATAGGTGGAGGTAT
CTGCTGTAAAGAATCTTTAGAACTAGATGCCCTAGTCTCCTTATCTGTAACAGAGAACCTTGTTGGGAAA
GAAGGTGGAGGCTTACATGCTAAAACTGTAAATATTTCTAATCTGAAATCAGGCTTCTCTTTCTCGAACA
ACAAAGCAAACTCCTCATCCACAGGAGTCGCAACAACAGCTTCAGCACCTGCTGCAGCTGCTGCTTCCCT
ACAAGCAGCCGCAGCAGCCGTACCATCATCTCCAGCAACACCAACTTATTCAGGTGTAGTAGGAGGAGCT
ATCTATGGAGAAAAGGTTACATTCTCTCAATGTAGCGGGACTTGTCAGTTCTCTGGGAACCAAGCTATCG
ATAACAATCCCTCCCAATCATCGTTGAACGTACAAGGAGGAGCCATCTATGCCAAAACCTCTTTGTCTAT
TGGATCTTCCGATGCTGGAACCTCCTATATTTTCTCGGGGAACAGTGTCTCCACTGGGAAATCTCAAACA
ACAGGGCAAATAGCGGGAGGAGCGATCTACTCCCCTACTGTTACATTGAATTGTCCTGCGACATTCTCTA
ACAATACAGCCTCTATGGCTACACCAAAGACTTCTTCTGAAGATGGATCCTCAGGAAATTCTATTAAAGA
TACCATTGGAGGAGCCATTGCAGGGACAGCCATTACCCTATCTGGAGTCTCTCGATTTTCAGGGAATACG
GCTGATTTAGGAGCTGCAATAGGAACTCTAGCTAATGCAAATACACCCAGTGCAACTAGCGGATCTCAAA
ATAGCATTACAGAAAAAATTACTTTAGAAAACGGTTCTTTTATTTTTGAAAGAAACCAAGCTAATAAACG
TGGAGCGATTTACTCTCCTAGCGTTTCCATTAAAGGGAATAATATTACCTTCAATCAAAATACATCCACT
CATGATGGAAGTGCTATCTACTTTACAAAAGATGCTACGATTGAGTCTTTAGGATCTGTTCTTTTTACAG
GAAATAACGTTACAGCTACACAAGCTAGTTCTGCAACATCTGGACAAAATACAAATACTGCCAACTATGG
GGCAGCCATCTTTGGAGATCCAGGAACCACTCAATCGTCTCAAACAGATGCCATTTTAACCCTTCTTGCT
TCTTCTGGAAACATTACTTTTAGCAACAACAGTTTACAGAATAACCAAGGTGATACTCCCGCTAGCAAGT
TTTGTAGTATTGCAGGATACGTCAAACTCTCTCTACAAGCCGCTAAAGGGAAGACTATTAGCTTTTTCGA
TTGTGTGCACACCTCTACCAAAAAAATAGGTTCAACACAAAACGTTTATGAAACTTTAGATATTAATAAA
GAAGAGAACAGTAATCCATATACAGGAACTATTGTGTTCTCTTCTGAATTACATGAAAACAAATCTTACA
TCCCACAGAATGCAATCCTTCACAACGGAACTTTAGTTCTTAAAGAGAAAACAGAACTCCACGTAGTCTC
TTTTGAGCAGAAAGAAGGGTCTAAATTAATTATGAAACCCGGAGCTGTGTTATCTAACCAAAACATAGCT
AACGGAGCTCTAGTTATCAATGGGTTAACGATTGATCTTTCCAGTATGGGGACTCCTCAAGCAGGGGAAA
TCTTCTCTCCTCCAGAATTACGTATCGTTGCCACGACCTCTAGTGCATCCGGAGGAAGCGGGGTCAGCAG
TAGTATACCAACAAATCCTAAAAGGATTTCTGCAGCAGCGCCTTCAGGTTCTGCCGCAACTACTCCAACT
ATGAGCGAGAACAAAGTTTTCCTAACAGGAGACCTTACTTTAATAGATCCTAATGGAAACTTTTACCAAA
ACCCTATGTTAGGAAGCGATCTAGATGTACCACTAATTAAGCTTCCGACTAACACAAGTGACGTCCAAGT
CTATGATTTAACTTTATCTGGGGATCTTTTCCCTCAGAAAGGGTACATGGGAACCTGGACATTAGATTCT
AATCCACAAACAGGGAAACTTCAAGCCAGATGGACATTCGATACCTATCGTCGCTGGGTATACATACCTA
GGGATAAATCATTTTTATGCGAACTCTATCTTAGGCTCCCAAACTCAATGATTGTTGTGAAGCAAGGGCT
TATCAACAACATGTTGAATAATGCCCGCTTCGATGATATCGCTTACAATAACTTCTGGGTTTCAGGAGTA
GGAACTTTCTTAGCTCAACAAGGAACTCCTCTTTCCGAAGAATTCAGTTACTACAGCCGCGGAACTTCAG
TTGCCATCGATGCCAAACCTAGACAAGATTTTATCCTAGGAGCTGCATTTAGTAAGATGGTGGGGAAAAC
CAAAGCCATCAAAAAAATGCATAATTACTTCCATAAGGGCTCTGAGTACTCTTACCAAGCTTCTGTCTAT
GGAGGTAAATTCCTGTATTTCTTGCTCAATAAGCAACATGGTTGGGCACTTCCTTTCCTAATACAAGGAG
TCGTGTCCTATGGACATATTAAACATGATACAACAACACTTTACCCTTCTATCCATGAAAGAAATAAAGG
AGATTGGGAAGATTTAGGATGGTTAGCGGATCTTCGTATCTCTATGGATCTTAAAGAACCTTCTAAAGAT
TCTTCTAAACGGATCACTGTCTATGGGGAACTTGAGTATTCCAGCATTCGCCAGAAACAGTTCACAGAAA
TCGATTACGATCCAAGACACTTCGATGATTGTGCTTACAGAAATCTGTCGCTTCCTGTGGGATGCGCTGT
CGAAGGAGCTATCATGAACTGTAATATTCTTATGTATAATAAGCTTGCATTAGCCTACATGCCTTCTATC
TACAGAAATAATCCTGTCTGTAAATATCGGGTATTGTCTTCGAATGAAGCTGGTCAAGTTATCTGCGGAG
TGCCAACTAGAACCTCTGCTAGAGCAGAATACAGTACTCAACTATATCTTGGTCCCTTCTGGACTCTCTA
CGGAAACTATACTATCGATGTAGGCATGTATACGCTATCGCAAATGACTAGCTGCGGTGCTCGCATGATC
TTCTAA
>gi|3328840|gb|AAC68009.1| Putative outer membrane protein A [Chlamydia
trachomatis][SEQ ID NO:47]
MNRVIEIHAHYDQRQLSQSPNTNFLVHHPYLTLIPKFLLGALIVYAPYSFAEMELAISGHKQGKDRDTFT
MISSCPEGTNYIINRKLILSDFSLLNKVSSGGAFRNLAGKISFLGKNSSASIHFKHININGFGAGVFSES
SIEFTDLRKLVAFGSESTGGIFTAKEDISFKNNHHIAFRNNITKGNGGVIQLQGDMKGSVSFVDQRGAII
FTNNQAVTSSSMKHSGRGGAISGDFAGSRILFLNNQQITFEGNSAVHGGAIYNKNGLVEFLGNAGPLAFK
ENTTIANGGAIYTSNFKANQQTSPILFSQNHANKKGGAIYAQYVNLEQNQDTIRFEKNTAKEGGGAITSS
QCSITAHNTIIFSDNAAGDLGGGAILLEGKKPSLTLIAHSGNIAFSGNTMLHITKKASLDRHNSILIKEA
PYKIQLAANKNHSIHFFDPVMALSASSSPIQINAPEYETPFFSPKGMIVFSGANLLDDAREDVANRTSIF
NQPVHLYNGTLSIENGAHLIVQSFKQTGGRISLSPGSSLALYTMNSFFHGNISSKEPLEINGLSFGVDIS
PSNLQAEIRAGNAPLRLSGSPSIHDPEGLFYENRDTAASPYQMEILLTSDKIVDISKFTTDSLVTNKQSG
FQGAWHFSWQPNTINNTKQKILRASWLPTGEYVLESNRVGRAVPNSLWSTFLLLQTASHNLGDHLCNNRS
LIPTSYFGVLIGGTGAEMSTHSSEEESFISRLGATGTSIIRLTPSLTLSGGGSHMFGDSFVADLPEHITS
EGIVQNVGLTHVWGPLTVNSTLCAALDHNAMVRICSKKDHTYGKWDTFGMRGTLGASYTFLEYDQTMRVF
SFANIEATNILQRAFTETGYNPRSFSKTKLLNIAIPIGIGYEFCLGNSSFALLGKGSIGYSRDIKRENPS
TLAHLAMNDFAWTTNGCSVPTSAHTLANQLILRYKACSLYITAYTINREGKNLSNSLSCGGYVGF
>gi|3328833|gb|AE001314.1:6535-9462[SEQ ID NO:48]
ATGAATCGAGTTATAGAAATCCATGCTCACTACGATCAAAGACAACTTTCTCAATCTCCAAATACAAACT
TCTTAGTACATCATCCTTATCTTACTCTTATTCCCAAGTTTCTACTAGGAGCTCTAATCGTCTATGCTCC
TTATTCGTTTGCAGAAATGGAATTAGCTATTTCTGGACATAAACAAGGTAAAGATCGAGATACCTTTACC
ATGATCTCTTCCTGTCCTGAAGGCACTAATTACATCATCAATCGCAAACTCATACTCAGTGATTTCTCGT
TACTAAATAAAGTTTCATCAGGGGGAGCCTTTCGGAATCTAGCAGGGAAAATTTCCTTCTTAGGAAAAAA
TTCTTCTGCGTCCATTCATTTTAAACACATTAATATCAATGGTTTTGGAGCCGGAGTCTTTTCTGAATCC
TCTATTGAATTTACTGATTTACGAAAACTTGTTGCTTTTGGATCTGAAAGCACAGGAGGAATTTTTACTG
CGAAAGAGGACATCTCTTTTAAAAACAACCACCACATTGCCTTCCGCAATAATATCACCAAAGGGAATGG
TGGCGTTATCCAGCTCCAAGGAGATATGAAAGGAAGCGTATCCTTTGTAGATCAACGTGGAGCTATCATC
TTTACCAATAACCAAGCTGTAACTTCTTCATCAATGAAACATAGTGGTCGTGGAGGAGCAATTAGCGGTG
ACTTCGCAGGATCCAGAATTCTTTTTCTTAATAACCAACAAATTACTTTCGAAGGCAATAGCGCTGTGCA
TGGAGGTGCTATCTACAATAAGAATGGCCTTGTCGAGTTCTTAGGAAATGCAGGACCTCTTGCCTTTAAA
GAGAACACAACAATAGCTAACGGGGGAGCTATATACACAAGTAATTTCAAAGCGAATCAACAAACATCCC
CCATTCTATTCTCTCAAAATCATGCGAATAAGAAAGGCGGAGCGATTTACGCGCAATATGTGAACTTAGA
ACAGAATCAAGATACTATTCGCTTTGAAAAAAATACCGCTAAAGAAGGCGGTGGAGCCATCACCTCTTCT
CAATGCTCAATTACTGCTCATAATACCATCATTTTTTCCGATAATGCTGCCGGAGATCTTGGAGGAGGAG
CAATTCTTCTAGAAGGGAAAAAACCTTCTCTAACCTTGATTGCTCATAGTGGTAATATTGCATTTAGCGG
CAATACCATGCTTCATATCACCAAAAAAGCTTCCCTAGATCGACACAATTCTATCTTAATCAAAGAAGCT
CCCTATAAAATCCAACTTGCAGCGAACAAAAACCATTCTATTCATTTCTTTGATCCTGTCATGGCATTGT
CAGCATCATCTTCCCCTATACAAATCAATGCTCCTGAGTATGAAACTCCCTTCTTCTCACCTAAGGGTAT
GATCGTTTTCTCGGGTGCGAATCTTTTAGATGATGCTAGGGAAGATGTTGCAAATAGAACATCGATTTTT
AACCAACCCGTTCATCTATATAATGGCACCCTATCTATCGAAAATGGAGCCCATCTGATTGTCCAAAGCT
TCAAACAGACCGGAGGACGTATCAGTTTATCTCCAGGATCCTCCTTGGCTCTATACACGATGAACTCGTT
CTTCCATGGCAACATATCCAGCAAAGAACCCCTAGAAATTAATGGTTTAAGCTTTGGAGTAGATATCTCT
CCTTCTAATCTTCAAGCAGAGATCCGTGCCGGCAACGCTCCTTTACGATTATCCGGATCCCCATCTATCC
ATGATCCTGAAGGATTATTCTACGAAAATCGCGATACTGCAGCATCACCATACCAAATGGAAATCTTGCT
CACCTCTGATAAAATTGTAGATATCTCCAAATTTACTACTGATTCTCTAGTTACGAACAAACAATCAGGA
TTCCAAGGAGCCTGGCATTTTAGCTGGCAGCCAAATACTATAAACAATACTAAACAAAAAATATTAAGAG
CTTCTTGGCTCCCAACAGGAGAATATGTCCTTGAATCCAATCGAGTGGGGCGTGCCGTTCCTAATTCCTT
ATGGAGCACATTTTTACTTTTACAGACAGCCTCTCATAACTTAGGCGATCATCTATGTAATAATCGATCT
CTTATTCCTACTTCATACTTCGGAGTTTTAATTGGAGGAACTGGAGCAGAAATGTCTACCCACTCCTCAG
AAGAAGAAAGCTTTATATCTCGTTTAGGAGCTACAGGAACCTCTATCATACGCTTAACTCCCTCCCTGAC
ACTCTCTGGAGGAGGCTCACATATGTTCGGAGATTCGTTCGTTGCAGACTTACCAGAACACATCACTTCA
GAAGGAATTGTTCAGAATGTCGGTTTAACCCATGTCTGGGGACCCCTTACTGTCAATTCTACATTATGTG
CAGCCTTAGATCACAACGCGATGGTCCGCATATGCTCCAAAAAAGATCACACCTATGGGAAATGGGATAC
ATTCGGTATGCGAGGAACATTAGGAGCCTCTTATACATTCCTAGAATATGATCAAACTATGCGCGTATTC
TCATTCGCCAACATCGAAGCCACAAATATCTTGCAAAGAGCTTTTACTGAAACAGGCTATAACCCAAGAA
GTTTTTCCAAGACAAAACTTCTAAACATCGCCATCCCCATAGGGATTGGTTATGAATTCTGCTTAGGGAA
TAGCTCTTTTGCTCTACTAGGTAAGGGATCCATCGGTTACTCTCGAGATATTAAACGAGAAAACCCATCC
ACTCTTGCTCACCTGGCTATGAATGATTTTGCTTGGACTACCAATGGCTGTTCAGTTCCAACCTCTGCAC
ACACATTGGCAAATCAATTGATTCTTCGCTATAAAGCATGTTCCTTATACATCACGGCATATACTATCAA
CCGTGAAGGGAAGAACCTCTCCAATAGCTTATCCTGCGGAGGCTATGTTGGCTTCTAA
>gi|3328763|gb|AAC67938.1| O-Sialoglycoprotein Endopeptidase family [Chlamydia
trachomatis][SEQ ID NO:49]
MYKYFIVDTSGSQPFLAYVDCRDVLEVWSLPTGPDQGVVLNFIFNSLDLPFQGIGVSVGPGGFSATRVGV
AFAQGLSLAKNVPLVGYSSLEGYLSLGQEEEALLLPLGKKGGIVALNSELSLDGFLLTDTTSTPGILLSY
SEALEYCLDKGCCHVISPDPTYFVELFSSRISVRKVVPCIDRIRKYVVSQFVLSQNLPLCLDYRSISSFF
>gi|3328757|gb|AE001307.1:c6730-6098[SEQ ID NO:50]
GTGTACAAATATTTTATTGTAGACACTTCCGGTTCTCAGCCGTTTTTGGCCTATGTCGATTGTCGAGATG
TTTTAGAAGTATGGTCTTTGCCTACAGGGCCAGATCAAGGAGTTGTGTTAAATTTCATTTTCAACAGCCT
GGATTTGCCTTTCCAAGGTATTGGAGTCTCTGTTGGTCCTGGGGGATTTTCTGCAACTAGAGTGGGAGTT
GCTTTTGCTCAAGGGCTCTCTCTGGCTAAAAATGTCCCTTTAGTTGGCTATAGCTCTTTAGAAGGATATC
TTTCTTTGGGTCAAGAAGAGGAGGCTTTGCTTTTGCCTCTAGGGAAAAAGGGTGGGATTGTAGCTTTAAA
CTCAGAGCTTTCTCTTGATGGTTTTCTGCTTACAGATACTACTTCTACTCCGGGAATTTTATTGTCTTAT
TCTGAAGCTCTAGAGTATTGTTTAGATAAGGGATGTTGTCATGTGATCTCTCCGGATCCAACGTATTTCG
TAGAACTATTTTCTTCGCGTATTTCGGTAAGGAAGGTGGTTCCTTGTATCGATCGAATCCGTAAGTACGT
TGTTTCGCAGTTTGTTCTGTCTCAAAATCTTCCGTTGTGTTTAGATTACCGGAGCATCTCTTCCTTTTTT
TAG
>gi|6578102|gb|AAC67897.2| ATP Synthase Subunit K [Chlamydia trachomatis][SEQ ID NO:51]
MIDVSVVGPVLAMALAMIGSAVGCGMAGVASHAVMSRIDEGHGKIIGLSAMPSSQSIYGLIFMLLLNDAI
KDGKVSAVSGIVMGIAVGSALLLSAFMQGKCCVSAIQAYARSSAIYGKSFASIGIVESFALFAFVFALLL
F
>gi|3328718|gb|AE001303.1:c956-531[SEQ ID NO:52]
ATGATAGATGTATCAGTAGTGGGGCCTGTATTGGCTATGGCTTTGGCAATGATTGGTAGCGCTGTTGGAT
GTGGAATGGCTGGAGTCGCTTCTCACGCAGTGATGTCTCGAATCGATGAAGGACACGGGAAGATTATTGG
TCTGTCTGCTATGCCCTCATCACAATCCATTTACGGGTTGATTTTCATGTTACTGCTGAATGATGCAATT
AAGGATGGAAAAGTCTCTGCAGTCAGTGGTATCGTAATGGGTATAGCTGTAGGATCTGCGTTATTGCTTT
CTGCTTTTATGCAAGGGAAGTGCTGTGTGAGTGCTATTCAAGCCTATGCGCGTTCCTCTGCAATATATGG
TAAATCATTTGCTTCGATTGGGATTGTTGAGTCTTTTGCGTTATTTGCTTTCGTTTTTGCGCTATTGTTA
TTCTAA
>gi|3329252|gb|AAC68382.1| S14 Ribosomal Protein [Chlamydia trachomatis][SEQ ID NO:53]
MAKKSAVAREVKRRKLVEANFQKRAELRKLAKSLSVSEEERERAREALNKMRRDTSPSRLHNRCLLTGRP
RGYLRKFAISRICFRQMASMGDIPGVVKASW
>gi|3522908|gb|AE001351.1:2436-2741[SEQ ID NO:54]
ATGGCGAAGAAGTCAGCAGTAGCTAGAGAAGTTAAACGTCGAAAGTTAGTAGAAGCTAATTTTCAGAAGA
GAGCAGAGCTTCGAAAACTTGCGAAGAGTTTATCTGTTAGCGAGGAAGAAAGAGAAAGAGCTCGGGAAGC
TCTCAATAAAATGAGAAGAGATACTTCTCCTTCTCGTTTACATAATAGATGCCTATTAACAGGCCGTCCT
CGTGGATACCTTAGAAAGTTTGCTATCTCAAGAATTTGTTTTAGACAAATGGCTTCTATGGGAGATATCC
CAGGCGTTGTGAAAGCAAGTTGGTAG
>gi|3329133|gb|AAC68276.1| Major Outer Membrane Protein [Chlamydia
trachomatis][SEQ ID NO:55]
MKKLLKSVLVFAALSSASSLQALPVGNPAEPSLMIDGILWEGFGGDPCDPCATWCDAISMRVGYYGDFVF
DRVLKTDVNKEFQMGAKPTTDTGNSAAPSTLTARENPAYGRHMQDAEMFTNAACMALNIWDRFDVFCTLG
ATSGYLKGNSASFNLVGLFGDNENQKTVKAESVPNMSFDQSVVELYTDTTFAWSVGARAALWECGCATLG
ASFQYAQSKPKVEELNVLCNAAEFTINKPKGYVGKEFPLDLTAGTDAATGTKDASIDYHEWQASLALSYR
LNMFTPYIGVKWSRASFDADTIRIAQPKSATAIFDTTTLNPTIAGAGDVKTGAEGQLGDTMQIVSLQLNK
MKSRKSCGIAVGTTIVDADKYAVTVETRLIDERAAHVNAQFRF
>gi|3329126|gb|AE001338.1:c6759-5578[SEQ ID NO:56]
ATGAAAAAACTCTTGAAATCGGTATTAGTATTTGCCGCTTTGAGTTCTGCTTCCTCCTTGCAAGCTCTGC
CTGTGGGGAATCCTGCTGAACCAAGCCTTATGATCGACGGAATTCTGTGGGAAGGTTTCGGCGGAGATCC
TTGCGATCCTTGCGCCACTTGGTGTGACGCTATCAGCATGCGTGTTGGTTACTACGGAGACTTTGTTTTC
GACCGTGTTTTGAAAACTGATGTGAATAAAGAATTTCAGATGGGTGCCAAGCCTACAACTGATACAGGCA
ATAGTGCAGCTCCATCCACTCTTACAGCAAGAGAGAATCCTGCTTACGGCCGACATATGCAGGATGCTGA
GATGTTTACAAATGCCGCTTGCATGGCATTGAATATTTGGGATCGTTTTGATGTATTCTGTACATTAGGA
GCCACCAGTGGATATCTTAAAGGAAACTCTGCTTCTTTCAATTTAGTTGGATTGTTTGGAGATAATGAAA
ATCAAAAAACGGTCAAAGCGGAGTCTGTACCAAATATGAGCTTTGATCAATCTGTTGTTGAGTTGTATAC
AGATACTACTTTTGCGTGGAGCGTCGGCGCTCGCGCAGCTTTGTGGGAATGTGGATGTGCAACTTTAGGA
GCTTCATTCCAATATGCTCAATCTAAACCTAAAGTAGAAGAATTAAACGTTCTCTGCAATGCAGCAGAGT
TTACTATTAATAAACCTAAAGGGTATGTAGGTAAGGAGTTTCCTCTTGATCTTACAGCAGGAACAGATGC
TGCGACAGGAACTAAGGATGCCTCTATTGATTACCATGAATGGCAAGCAAGTTTAGCTCTCTCTTACAGA
CTGAATATGTTCACTCCCTACATTGGAGTTAAATGGTCTCGAGCAAGCTTTGATGCCGATACGATTCGTA
TAGCCCAGCCAAAATCAGCTACAGCTATTTTTGATACTACCACGCTTAACCCAACTATTGCTGGAGCTGG
CGATGTGAAAACTGGCGCAGAGGGTCAGCTCGGAGACACAATGCAAATCGTTTCCTTGCAATTGAACAAG
ATGAAATCTAGAAAATCTTGCGGTATTGCAGTAGGAACAACTATTGTGGATGCAGACAAATACGCAGTTA
CAGTTGAGACTCGCTTGATCGATGAGAGAGCAGCTCACGTAAATGCACAATTCCGCTTCTAA
>gi|3328987|gb|AAC68150.1| hypothetical protein [Chlamydia trachomatis][SEQ ID NO:57]
MLKMFWLNSLVFFSLLLSACGYTVLSPHYVEKKFSLSEGIYVCPIEGDSLGDLVSSLSYELEKRGLHTRS
QGTSSGYVLKVSLFNETDENIGFAYTPQKPDEKPVKHFIVSNEGRLALSAKVQLIKNRTQEILVEKCLRK
SVTFDFQPDLGTANAHQLALGQFEMHNEAIKSASRILYSQLAETIVQQVYYDLF
>gi|3328980|gb|AE001325.1:10880-11464[SEQ ID NO:58]
ATGCTGAAAATGTTTTGGTTGAATAGCCTCGTTTTCTTCTCGTTACTACTATCAGCCTGCGGCTATACAG
TGCTCTCCCCCCACTATGTAGAAAAGAAATTCTCGCTTTCCGAAGGCATCTATGTCTGCCCTATCGAAGG
AGATTCATTAGGAGATCTCGTATCCTCTCTTTCTTACGAATTAGAAAAGCGAGGACTCCACACACGATCT
CAAGGAACCTCTTCTGGTTATGTACTCAAAGTCTCTCTTTTCAATGAGACTGATGAAAATATTGGATTCG
CATACACTCCCCAAAAACCTGATGAAAAACCTGTAAAACACTTCATTGTCTCTAATGAAGGGCGCTTAGC
GTTATCAGCAAAAGTCCAACTAATCAAAAACCGCACACAAGAAATATTAGTGGAGAAATGCCTGAGAAAA
TCGGTTACTTTTGATTTTCAACCTGACCTCGGAACCGCGAATGCTCATCAGCTAGCTCTCGGACAATTTG
AAATGCATAATGAAGCAATAAAAAGCGCTTCTCGTATATTGTATTCGCAATTAGCAGAGACTATTGTACA
ACAGGTATACTATGACCTTTTCTGA
>gi|3328972|gb|AAC68136.1| Apolipoprotein N-Acetyltransferase [Chlamydia
trachomatis][SEQ ID NO:59]
MFKLVSYIILSWVLVCLAQPDVSVVASVVSCICGYSLLWAGLFALVEQLSWKKVWCIAFIWTWTVEGAHF
SWMLEDLYVGTSIYFVWGILLSYLATLFASFSCLVVWCCRKQYRGALVWLPGVWVAIEAIRYYGLLSGVS
FDFIGWPLTATAYGRQFGSFFGWAGQSFLVIAANICCFAVCLLKHSFSKGLWLTLCAFPYLLGGAHYEYL
KKHFSDSEVLRVAIVQPGYSPHMHAGRTASAIWRGLVSLCQTIQTPVDVIVFPEVSVPFGLHRQAYTLHE
NQPVLESLLPNKSWGEFFTNLDWIQAIAERYQCTVIMGMERWENKGGILHLYNAAECVSREGEITSYDKR
ILVPGGEYIPGGKIGFSLCQTFFPEFALPFQRLPGEFSGVVNITERIKAGISICYEETFGYAIRPYKRQQ
ADILVNLTNDGWYPRSRLPLVHFYHGMLRNQELGIPCIRACRTGVSAAVDSLGRIVGILPWESRTCPVST
GVLQVSVPLYSYHTVYARLGDAPLLLIAVCSVIGAIAYFYRKKKETPPQTFF
>gi|3328966|gb|AE001324.1:c6152-4524[SEQ ID NO:60]
GTGTTTAAACTTGTGTCATACATCATCCTTTCTTGGGTGCTGGTCTGTTTGGCTCAGCCGGATGTAAGTG
TTGTAGCTTCTGTTGTTAGTTGTATTTGCGGTTACAGCTTACTTTGGGCTGGGCTTTTTGCTTTAGTAGA
GCAATTATCTTGGAAGAAAGTTTGGTGCATCGCTTTTATTTGGACTTGGACTGTCGAAGGCGCTCATTTC
TCTTGGATGCTTGAAGATCTTTATGTAGGGACAAGCATCTATTTTGTTTGGGGTATACTGCTTTCTTATC
TCGCCACCCTATTTGCTAGTTTTTCTTGTTTGGTTGTGTGGTGTTGTCGCAAGCAATATAGGGGAGCTCT
TGTTTGGCTTCCAGGGGTTTGGGTGGCGATAGAAGCAATACGCTATTATGGGTTGCTTTCAGGAGTTTCT
TTTGATTTTATTGGCTGGCCTCTTACAGCGACAGCCTATGGCCGGCAATTCGGCAGCTTTTTTGGATGGG
CTGGACAAAGCTTTCTAGTTATTGCTGCCAATATATGCTGTTTTGCAGTATGTTTATTAAAACACTCTTT
TTCCAAAGGTTTGTGGTTGACGTTGTGCGCGTTCCCTTATCTGTTAGGCGGAGCGCATTACGAATACCTA
AAGAAGCATTTTTCCGACTCTGAAGTGCTTCGAGTTGCCATCGTGCAGCCTGGATATAGTCCTCATATGC
ATGCAGGGAGGACGGCTAGTGCTATTTGGAGAGGTTTGGTTTCTTTGTGCCAGACTATTCAAACTCCTGT
AGATGTGATCGTTTTCCCAGAAGTAAGTGTTCCTTTTGGCTTACATAGACAAGCCTATACTCTTCATGAA
AATCAGCCTGTATTAGAAAGTTTGCTTCCTAACAAATCTTGGGGCGAGTTTTTCACAAATTTGGATTGGA
TCCAAGCGATAGCTGAACGTTATCAATGCACCGTTATCATGGGAATGGAACGATGGGAAAATAAAGGGGG
AATACTGCATTTGTATAATGCTGCTGAATGCGTATCGCGAGAAGGGGAAATAACTAGCTATGATAAGCGG
ATTCTTGTTCCTGGAGGTGAGTACATCCCTGGAGGGAAAATAGGTTTTTCCTTGTGTCAAACCTTTTTCC
CAGAATTTGCTCTTCCCTTTCAACGTTTGCCAGGAGAGTTTTCTGGAGTTGTGAATATAACAGAGCGAAT
AAAAGCTGGGATCTCTATTTGTTATGAGGAGACATTTGGGTATGCAATTCGCCCTTACAAAAGGCAACAA
GCCGATATTTTAGTAAATCTTACTAATGACGGTTGGTATCCGCGTTCAAGGCTGCCTCTAGTACATTTTT
ATCATGGCATGTTACGTAATCAAGAGTTGGGTATACCTTGTATTCGCGCCTGTCGCACAGGAGTTTCTGC
TGCAGTGGATTCTTTGGGTAGAATTGTCGGCATACTTCCCTGGGAATCGAGAACTTGCCCAGTTTCTACA
GGAGTACTCCAAGTTTCCGTCCCTCTTTACAGTTATCATACTGTATATGCAAGGCTGGGTGATGCTCCTC
TGTTACTGATTGCAGTTTGTTCGGTTATCGGAGCGATTGCCTATTTTTATAGGAAAAAGAAAGAGACCCC
ACCACAAACATTTTTTTGA
>gi|3328612|gb|AAC67797.1| Fructose-6-P Phosphotransferase [Chlamydia
trachomatis][SEQ ID NO:61]
MSSNKHASLCQKTPSLCRELQKAPALLLTEDIRFKALLNERIDSVAELFPCTYNSPYYKFISKSDLSAET
SPLKVGVMLSGGPAPGGHNVILGLLHSIKKLHPNSQLLGFIRNGEGLLNNNTVEITDEFIEEFRNSGGFN
CIGTGRTNIITEENKARCLQTANELDLDGLVIIGGDGSNTATAILAEYFAKHQAKTVLVGVPKTIDGDLQ
HLFLDLTFGFDTATKFYSSIISNISRDALSCKGHYHFIKLMGRSSSHITLECALQTHPNIALIGEEIAEK
SISLETLIHDICETIADRAAMGKYHGVILIPEGVIEFIPEIQSLVKEIESIPEQENLYQALSLSSQQLLC
QFPEDICHQLLYNRDAHGNVYVSKISVDKLLIHLVRQHLETHFRQVPFNAISHFLGYEGRSGTPTHFDNV
YSYNLGYGAGVLVFNRCNGYLSTIEGLTSPIEKWRLRALPIVRMLTTKQGKDSKHYPLIKKRLVDIASPV
FNKFSLYRKIWALEDSYRFVGPLQIHSPEDAHSDDFPPLILFLNHNEWQKRCSICLEIPDQDY
>gi|3328609|gb|AE001294.1:2452-4113[SEQ ID NO:62]
ATGTCGTCGAATAAACATGCTTCTCTTTGTCAAAAGACGCCTTCTTTGTGTCGGGAGCTTCAAAAAGCTC
CTGCTCTTCTATTAACAGAAGACATAAGGTTTAAAGCTCTTCTTAATGAACGCATTGACTCTGTTGCAGA
ACTATTTCCATGCACTTATAACTCTCCCTACTACAAATTTATTTCGAAGTCCGATCTTTCCGCTGAGACC
TCTCCCCTTAAAGTGGGCGTTATGCTTTCTGGAGGCCCAGCTCCTGGTGGGCACAATGTCATCTTAGGAT
TGCTACACAGTATTAAAAAGCTCCATCCGAATAGTCAGCTTTTAGGATTTATTCGCAATGGAGAAGGACT
TCTCAATAATAATACTGTAGAAATCACAGATGAATTCATTGAAGAGTTTCGTAACTCTGGAGGCTTTAAT
TGCATAGGAACAGGTCGCACTAATATCATAACCGAAGAAAATAAAGCGCGCTGTTTACAAACAGCAAATG
AACTCGATTTAGATGGATTAGTGATTATTGGAGGCGATGGTTCGAATACAGCCACGGCGATTCTTGCTGA
ATATTTTGCTAAGCATCAAGCAAAAACGGTATTAGTTGGTGTTCCCAAAACTATTGATGGAGATTTGCAG
CACCTATTTTTAGACCTCACATTTGGGTTTGATACTGCTACTAAATTTTATTCATCCATCATCAGCAACA
TTTCTAGAGACGCATTATCGTGTAAAGGCCACTATCATTTTATTAAACTAATGGGCCGGTCTTCTTCTCA
TATCACGCTAGAATGCGCACTACAGACTCACCCAAATATTGCTCTTATAGGCGAAGAGATTGCAGAAAAA
AGCATCTCCTTAGAAACATTAATCCATGATATTTGTGAAACAATAGCAGATCGAGCTGCTATGGGGAAAT
ACCATGGCGTTATTCTCATCCCTGAAGGAGTCATTGAGTTTATTCCTGAAATACAGTCTCTGGTTAAAGA
AATTGAATCCATTCCAGAGCAGGAGAATCTTTACCAAGCTTTATCCTTATCTTCTCAGCAACTTTTATGC
CAATTTCCGGAAGATATTTGCCATCAGCTCTTGTATAATAGAGATGCTCATGGCAACGTCTATGTATCAA
AAATTAGTGTTGATAAACTTCTGATTCATCTAGTTCGTCAACATTTAGAAACACATTTTAGACAAGTTCC
CTTCAATGCAATCTCCCATTTTTTAGGTTATGAAGGGCGTTCAGGAACTCCTACACATTTTGATAATGTG
TATAGCTATAACTTAGGATATGGTGCTGGGGTTCTCGTTTTTAACCGCTGTAATGGGTATTTATCCACGA
TCGAAGGTCTAACTAGCCCTATTGAAAAATGGCGATTGCGCGCTTTACCCATTGTTCGAATGTTGACGAC
CAAGCAGGGGAAAGACAGTAAACATTATCCTCTGATAAAAAAAAGATTGGTAGATATTGCTAGTCCTGTT
TTTAATAAGTTCTCACTGTATCGGAAAATCTGGGCTTTAGAAGACTCCTATCGCTTTGTAGGGCCATTAC
AAATACATTCTCCGGAGGATGCTCATTCTGATGATTTTCCTCCTTTAATTTTGTTTTTGAATCATAATGA
ATGGCAAAAACGCTGTTCTATTTGTTTAGAAATCCCCGATCAGGATTATTAA
>gi|3328517|gb|AAC67709.1| hypothetical protein [Chlamydia trachomatis][SEQ ID NO:63]
MICCDKVLSSVQSMPVIDKCSVTKCLQTAKQAAVLALSLFAVFASGSLSILSAAVLFSGTAAVLPYLLIL
TTALLGFVCAVIVLLRNLSAVVQSCKKRSPEEIEGAARPSDQQESGGRLSEESASPQASPTSSTFGLESA
LRSIGDSVSGAFDDINKDNSRSRSHSF
>gi|3328516|gb|AE001286.1:75-578[SEQ ID NO:64]
ATGATCTGCTGTGACAAAGTCTTGTCGAGCGTACAATCAATGCCTGTTATAGATAAATGCTCTGTAACGA
AATGCTTACAAACGGCTAAGCAAGCAGCTGTTCTTGCGTTGTCTTTGTTTGCGGTGTTTGCTTCAGGAAG
TTTATCCATATTATCAGCGGCGGTACTGTTTAGTGGCACTGCTGCTGTTCTTCCATATCTGCTGATATTA
ACAACAGCTCTTCTAGGATTTGTTTGTGCTGTTATTGTGCTTTTAAGAAATTTATCAGCAGTTGTTCAGA
GTTGTAAAAAGAGATCACCTGAAGAAATTGAAGGGGCTGCTCGTCCCTCTGATCAGCAGGAATCAGGAGG
ACGTTTGTCCGAGGAGAGCGCTTCACCACAAGCATCTCCTACTTCGTCTACTTTTGGTCTTGAATCCGCT
TTGCGCTCAATAGGAGATA
>gi|3328482|gb|AAC67677.1| L28 Ribosomal Protein [Chlamydia trachomatis][SEQ ID NO:65]
MSKKCALTGRKPRRGYSYAIRGISKKKKGIGLKVTGRTKRRFFPNMMTKRLWSTEENRFLKLKISAAALR
LVDKLGLDQVVARAKSKGF
>gi|3328480|gb|AE001283.1:c2251-1982[SEQ ID NO:66]
ATGTCGAAAAAATGTGCGCTTACAGGAAGAAAGCCTCGTCGCGGTTATAGCTATGCTATCCGAGGGATTT
CTAAAAAGAAAAAAGGGATCGGTTTGAAAGTTACAGGAAGAACAAAACGTCGATTCTTCCCTAATATGAT
GACTAAGAGACTATGGTCTACTGAGGAAAATCGCTTCCTCAAACTCAAAATTTCTGCAGCAGCTTTACGC
CTTGTTGATAAACTAGGGTTAGATCAGGTTGTTGCTAGAGCTAAAAGCAAGGGTTTTTAG
>gi|3328436|gb|AAC67635.1| SS DNA Binding Protein [Chlamydia trachomatis][SEQ ID NO:67]
MLFGYLVGFLAADPEERMTSGGKRVVVLRLGVKSRVGSKDETVWCRCNIWNNRYDKMLPYLKKGSSVIVA
GELSLESYVGRDGSPQASISVSVDTLKFNSGSSRPDARGSDEGRQRANDNVSIGFDGESLDTDSALDKEV
YAGFGEDQQYASEDVPF
>gi|3328434|gb|AE001279.1:1060-1533[SEQ ID NO:68]
ATGTTGTTCGGATATTTGGTAGGATTTCTAGCTGCCGATCCTGAAGAAAGAATGACATCCGGAGGTAAAC
GGGTTGTTGTTTTACGTTTGGGTGTAAAATCTCGTGTAGGATCTAAAGATGAAACAGTGTGGTGCAGATG
CAATATCTGGAACAACCGTTATGATAAGATGCTTCCTTATTTGAAGAAAGGTTCTTCAGTCATTGTTGCT
GGAGAGCTTTCTTTAGAAAGCTATGTAGGTAGAGACGGTTCTCCACAAGCTTCTATTTCTGTAAGCGTAG
ATACATTAAAATTTAATTCCGGATCTTCTCGTCCTGATGCTAGAGGTTCAGATGAAGGTCGTCAGAGAGC
TAATGATAATGTCTCTATTGGATTTGATGGAGAAAGTTTAGATACAGACTCTGCGCTTGATAAGGAAGTC
TATGCAGGGTTTGGAGAAGACCAACAGTATGCTAGTGAGGATGTTCCTTTTTAG
>gi|3328411|gb|AAC67611.1| hypothetical protein [Chlamydia trachomatis][SEQ ID NO:69]
MKKQEKMHPQNLLKVFIFFLAFFISYPSAEAHSPLQSSIQEKILTARPGDYAVLSRGSQKFFFLIRQSSS
EATWVEMSEFASLTQQEKKLVEQSSWKNAFHQLQSSKKVYLLRISKNPLMIFVLKNAQWMPLSEKDPLPF
FVKILRLPLSPAPSHLIKYKGKERTPWSPRTSLNGELITLPSSAWISVWPKDSSPLSEKNILIYFSNNER
LAFPLWTSIDTPTGTVIIKTIEMGHQAASSYPALPNF
>gi|3522886|gb|AE001277.1:c6191-5448[SEQ ID NO:70]
ATGAAAAAGCAAGAAAAAATGCACCCTCAAAACCTTCTTAAAGTTTTTATTTTTTTCTTGGCATTTTTCA
TATCCTATCCCTCGGCTGAAGCCCATTCTCCTCTCCAATCATCAATCCAAGAAAAAATTCTAACTGCCCG
CCCCGGAGACTATGCCGTCTTAAGCCGAGGATCTCAAAAATTTTTCTTTTTAATTCGCCAAAGTTCTTCG
GAAGCGACTTGGGTCGAAATGTCTGAATTTGCCTCCCTAACACAGCAAGAAAAAAAATTAGTAGAACAGT
CTTCCTGGAAGAATGCCTTCCATCAACTCCAATCTTCAAAAAAAGTGTACTTGTTACGAATTTCCAAAAA
TCCTCTTATGATTTTTGTTCTCAAAAATGCGCAATGGATGCCTCTCTCAGAAAAAGATCCTTTGCCTTTC
TTTGTAAAAATCCTTCGACTCCCTTTATCTCCAGCCCCCTCTCACTTAATTAAATACAAAGGGAAAGAAC
GCACCCCCTGGTCTCCGCGAACATCTTTGAATGGAGAACTCATAACCCTTCCTTCCAGTGCTTGGATTTC
TGTTTGGCCAAAAGATTCTTCTCCTCTATCAGAAAAAAATATTCTCATATATTTTTCTAACAATGAACGT
TTAGCGTTTCCTCTATGGACTAGTATTGATACTCCTACAGGGACAGTGATTATTAAGACTATTGAAATGG
GGCACCAAGCCGCCTCCTCCTATCCAGCTCTTCCCAATTTCTAG
crpA, CHLTR 15 kD cysteine-rich protein (Chlamydia trachomatis serovar D (D/UW-
3/Cx)
DNA sequence[SEQ ID NO:71]
AATATGAGCACTGTACCCGTTGTTCAAGGAGCTGGATCTTCCAATTCGGCACAGGATATTTCCACTAGACCAT
TAACACTGAAAGAGCGTATATCGAATCTTCTATCTTCCACTGCATTTAAGGTGGGATTAGTGGTGATAGGACT
ACTTTTGTGATTGCTACTTTGATATTCCTAGTTTCGGCAGCTTCGTTTGTAAATGCCATCTATCTAGTAGCTA
TTCCTGCTATTTGGGATGCGTGAATATCTGCGTAGGAATTTTATCCATGGAAGGACACTGTTCTCCGGAGAGA
TGGATCTTATGTAAGAAGTATTAAAGACTTCAGAAGATATCATCGATGATGGGCAGATAAACAACTCTAATAA
AGTGTTTACTGATGAGAGGTTGAAGCCATAGGTGGGGTAGTGGAATCTCTATCTAGAAGAAATAGTCTGGTGG
ATCAGACCCAATGA
Translated amino acid sequence[SEQ ID NO:72]
          NMS TVPVVQGAGS SNSAQDISTR PLTLKERISN LLSSTAFKVG
LVVIGLLLVI ATLIFLVSAA SFVNAIYLVA IPAILGCVNI CVGILSMEGH
CSPERWILCK KVLKTSEDII DDGQINNSNK VFTDERLNAI GGVVESLSRR
NSLVDQTQ*
OmcA, CHLTR 9 kD cystein-rich outer membrane complex lipoprotein (Chlamydia
trachomatis serovar D (D/UW-3/Cx)
DNA sequence[SEQ ID NO:73]
GGGCTAGTTTCTTTTATTGTTAAAAGAATTGCTTTTATCGATAAAAGAAACTTCAAGAGCCCTTTTCTAG
AAAGGAGTCTGGAAGTTATGAAAAAAACTGCTTTACTCGCTGCTTTATGTAGTGTTGTTTCTTTAAGTAG
TTGTTGTCGATCGTTGACTGTTGCTTCGAAGATCCATGCGCACCTATCCAATGTTCACCTTGTGAATCTA
AGAAGAAAGACGTAGACGTGGTTGCAACTCTTGTAACGGGTATGTCCCAGCTTGCAAACCTTGCGGAGGG
GATACGCACCAAGATGCTAAACATGGCCTCAAGCTAGAGGAATTCCAGTTGACGGCAAATGCAGACAATA
G
Translated amino acid sequence[SEQ ID NO:74]
                                                            GL VSFIVKRIAF IDKRNFKSPF
LERSLEVMKK TALLAALCSV VSLSSCCRIV DCCFEDPCAP IQCSPCESKK
KDVDGGCNSC NGYVPACKPC GGDTHQDAKH GPQARGIPVD GKCRQ*
cutE, apolipoprotein N-acyltransferase (Chlamydia trachomatis serovar D (D/UW-
3/Cx)
DNA sequence[SEQ ID NO:75]
GCTAGTAAGGGAGCCCCTTTAGTGTTTAAACTTGTGTCATACATCATCCTTTCTTGGGTGCTGGTCTGTTTG
GCTCAGCCGGATGTAAGTGTTGTAGCTTCTGTTGTTAGTTGTATTTGCGGTTACAGCTTACTTTGGGCTGGG
CTTTTTGTTTAGTAGAGCAATTATCTTGGAAGAAAGTTTGGTGCATCGCTTTTATTTGCACTTGGACTGTCG
AAGGCGCTCATTTCCTTGGATGCTTGAAGATCTTTATGTAGGGACAAGCATCTATTTTGTTTGGGGTATACT
GCTTTCTTATCTCGCCACCCTTTTGCTAGTTTTTCTTGTTTGGTTGTGTGGTGTTGTCGCAAGCAATATAGG
GGAGCTCTTGTTTGGCTTCCAGGGGTTTGGTGGCGATAGAAGCAATACGCTATTATGGGTTGCTTTCAGGAG
TTTCTTTTGATTTTATTGGCTGGCCTCTTACAGCGCAGCCTATGGCCGGCAATTCGGCAGCTTTTTTGGATG
GGCTGGACAAAGCTTTCTAGTTATTGCTGCCAATATATGCTGTTTGCAGTATGTTTATTAAAACACTCTTTT
TCCAAAGGTTTGTGGTTGACGTTGTGCGCGTTCCCTTATCTGTTAGGCGAGCGCATTACGATACCTAAATGA
AGCATTTTTCCGACTCTGAAGTGCTTCGAGTTGCCATCGTGCAGCCTGGATATAGTCTCATATGCATGCAGG
GAGGACGGCTAGTGCTATTTGGAGAGGTTTGGTTTCTTTGTGCCAGACTATTCAAACTCCTGTGATGTGATC
GTTTTCCCAGAAGTAAGTGTTCCTTTTGGCTTACATAGACAAGCCTATACTCTTCATGAAAATCAGCCTGAT
TAGAAAGTTTGCTTCCTAACAAATCTTGGGGCGAGTTTTTCACAAATTTGGATTGGATCCAAGCGATAGCTG
AACGTATCAATGCACCGTTATCATGGGAATGGAACGATGGGAAAATAAAGGGGGAATACTGCATTTGTATAA
TGCTGCTGAATGGTATCGCGAGAAGGGGAAATAACTAGCTATGATAAGCGGATTCTTGTTCCTGGAGGTGAG
TACATCCCTGGAGGGAAAAAGGTTTTTCCTTGTGTCAAACCTTTTTCCCAGAATTTGCTCTTCCCTTTCAAC
GTTTGCCAGGAGAGTTTTCTGGAGTTTGAATATAACAGAGCGAATAAAAGCTGGGATCTCTATTTGTTATGA
GGAGACATTTGGGTATGCAATTCGCCCTTACAAAGGCAACAAGCCGATATTTTAGTAAATCTTACTAATGAC
GGTTGGTATCCGCGTTCAAGGCTGCCTCTAGTACATTTTTTCATGGCATGTTACGTAATCAAGAGTTGGGTA
TACCTTGTATTCGCGCCTGTCGCACAGGAGTTTCTGCTGCAGTGGATCTTTGGGTAGAATTGTCGGCATACT
TCCCTGGGAATCGAGAACTTGCCCAGTTTCTACAGGAGTACTCCAAGTTTCCGTCCTCTTTACAGTTATCAT
ACTGTATATGCAAGGCTGGGTGATGCTCCTCTGTTACTGATTGCAGTTTGTTCGGTTATCGAGCGATTGCCT
ATTTTTATAGGAAAAAGAAAGAGACCCCACCACAAACATTTTTTTGA
Translated amino acid sequence[SEQ ID NO:76]
                                                                                                    ASKG
APLVFKLVSY IILSWVLVCL AQPDVSVVAS VVSCICGYSL LWAGLFALVE
QLSWKKVWCI AFIWTWTVEG AHFSWMLEDL YVGTSIYFVW GILLSYLATL
FASFSCLVVW CCRKQYRGAL VWLPGVWVAI EAIRYYGLLS GVSFDFIGWP
LTATAYGRQF GSFFGWAGQS FLVIAANICC FAVCLLKHSF SKGLWLTLCA
FPYLLGGAHY EYLKKHFSDS EVLRVAIVQP GYSPHMHAGR TASAIWRGLV
SLCQTIQTPV DVIVFPEVSV PFGLHRQAYT LHENQPVLES LLPNKSWGEF
FTNLDWIQAI AERYQCTVIM GMERWENKGG ILHLYNAAEC VSREGEITSY
DKRILVPGGE YIPGGKIGFS LCQTFFPEFA LPFQRLPGEF SGVVNITERI
KAGISICYEE TFGYAIRPYK RQQADILVNL TNDGWYPRSR LPLVHFYHGM
LRNQELGIPC IRACRTGVSA AVDSLGRIVG ILPWESRTCP VSTGVLQVSV
PLYSYHTVYA RLGDAPLLLI AVCSVIGAIA YFYRKKKETP PQTFF*
pal, peptidoglycan-associated lipoprotein (Chlamydia trachomatis serovar D (D/UW-
3/Cx)
DNA sequence[SEQ ID NO:77]
                                                              G AAAATTGTTA TAGGATCAGG
AGAGAAACGT TTCCCATGCT GGGGAGCATT TCCTTCACAA CATATAAAGA
AAACCTCATG AGAAAGACTA TTTTTAAAGC GTTTAATTTA TTATTCTCCC
TTCTTTTTCT TTCTTCATGC TCTTATCCTT GCAGAGATTG GGAATGCCAT
GGTTGCGACT CCGCAAGACC TCGTAAATCC TCTTTTGGAT TCGTACCTTT
CTACTCCGAT GAAGAAATTC AACAAGCTTT TGTTGAAGAT TTTGATTCCA
AAGAAGAGCA GCTGTACAAA ACGAGCGCAC AGAGTACCTC TTTCCGAAAT
ATCACTTTCG CTACAGATAG TTATTCTATT AAAGGAGAGG ATAACCTCAC
GATTCTTGCA AGCTTAGTTC GTCATTTGCA TAAATCTCCT AAAGCTACGC
TATATATAGA GGGCCATACA GATGAACGTG GAGCTGCAGC TTATAACCTA
GCTTTAGGAG CTCGTCGTGC GAATGCTGTA AAACAATACC TCATCAAACA
GGGAATCGCT GCAGACCGCT TATTCACTAT TTCTTACGGA AAAGAACATC
CTGTTCATCC AGGCCATAAT GAATTAGCTT GGCAACAAAA TCGTCGTACT
GAATTTAAGA TCCATGCTCG CTAA
Translated amino acid sequence[SEQ ID NO:78]
                                                                                              ENCYRIR
RETFPMLGSI SFTTYKENLM RKTIFKAFNL LFSLLFLSSC SYPCRDWECH
GCDSARPRKS SFGFVPFYSD EEIQQAFVED FDSKEEQLYK TSAQSTSFRN
ITFATDSYSI KGEDNLTILA SLVRHLHKSP KATLYIEGHT DERGAAAYNL
ALGARRANAV KQYLIKQGIA ADRLFTISYG KEHPVHPGHN ELAWQQNRRT
EFKIHAR*
```

The following Chlamydia trachomatis outer membrane proteins (full sequences above) are disclosed for the first time as being useful in a C. trachomatis vaccine. A vaccine comprising one or more of these proteins (or native or functional analogues thereof) is a further aspect of this invention (particularly in the context of being presented on the surface of a bleb).

>gi|6578118|gb|AAC68456.2| predicted Protease containing IRBP
and DHR domains [Chlamydia trachomatis][SEQ ID NO: 3]
>gi|6578109|gb|AAC68227.2| CHLPN 76kDa Homolog [Chlamydia
trachomatis][SEQ ID NO: 5]
>gi|3328866|gb|AAC68034.1| Sulfite Reductase [Chlamydia
trachomatis][SEQ ID NO: 19]
>gi|3328815|gb|AAC67986.1| hypothetical protein [Chlamydia
trachomatis][SEQ ID NO: 23]
>gi|3328587|gb|AAC67774.1| CMP-2-keto-3-deoxyoctulosonic acid
synthetase [Chlamydia trachomatis][SEQ ID NO: 27]
>gi|3329039|gb|AAC68197.1| Thio: disulfide Interchange Protein
[Chlamydia trachomatis][SEQ ID NO: 29]
>gi|3329000|gb|AAC68161.1| Yop proteins translocation lipoprotein
J [Chlamydia trachomatis][SEQ ID NO: 31]
>gi|3328905|gb|AAC68071.1| hypothetical protein [Chlamydia
trachomatis][SEQ ID NO: 33]
>gi|3328884|gb|AAC68051.1| Phosphatidate Cytidylytransferase
[Chlamydia trachomatis][SEQ ID NO: 35]
>gi|3328855|gb|AAC68022.1| hypothetical protein [Chlamydia
trachomatis][SEQ ID NO: 37]
>gi|3328772|gb|AAC67946.1| hypothetical protein [Chlamydia
trachomatis][SEQ ID NO: 39]
>gi|3328763|gb|AAC67938.1| O-Sialoglycoprotein Endopeptidase
family [Chlamydia trachomatis][SEQ ID NO: 49]
>gi|6578102|gb|AAC67897.2| ATP Synthase Subunit K [Chlamydia
trachomatis][SEQ ID NO: 51]
>gi|3329252|gb|AAC68382.1| S14 Ribosomal Protein [Chlamydia
trachomatis][SEQ ID NO: 53]
>gi|3328987|gb|AAC68150.1| hypothetical protein [Chlamydia
trachomatis][SEQ ID NO: 57]
>gi|3328972|gb|AAC68136.1| Apolipoprotein N-Acetyltransferase
[Chlamydia trachomatis][SEQ ID NO: 59]
>gi|3328612|gb|AAC67797.1| Fructose-6-P Phosphotransferase
[Chlamydia trachomatis][SEQ ID NO: 61]
>gi|3328517|gb|AAC67709.1| hypothetical protein [Chlamydia
trachomatis][SEQ ID NO: 63]
>gi|3328482|gb|AAC67677.1| L28 Ribosomal Protein [Chlamydia
trachomatis][SEQ ID NO: 65]
>gi|3328436|gb|AAC67635.1| SS DNA Binding Protein [Chlamydia
trachomatis][SEQ ID NO: 67]
>gi|3328411|gb|AAC67611.1| hypothetical protein [Chlamydia
trachomatis][SEQ ID NO: 69]

Amino Acid Sequences:

>gi|6578118|gb|AAC68456.2| predicted Protease containing IRBP and DHR domains [Chlamydia trachomatis][SEQ ID NO:3]

>gi|6578109|gb|AAC68227.2| CHLPN 76 kDa Homolog [Chlamydia trachomatis][SEQ ID NO:5]

>gi|3328866|gb|AAC68034.1| Sulfite Reductase [Chlamydia trachomatis][SEQ ID NO:19]

>gi|3328815|gb|AAC67986.1| hypothetical protein [Chlamydia trachomatis][SEQ ID NO:23]

>gi|3328587|gb|AAC67774.1| CMP-2-keto-3-deoxyoctulosonic acid synthetase [Chlamydia trachomatis][SEQ ID NO:27]

>gi|3329039| gb|AAC68197.1| Thio:disulfide Interchange Protein [Chlamydia trachomatis][SEQ ID NO:29]

>gi|3329000|gb|AAC68161.1| Yop proteins translocation lipoprotein J [Chlamydia trachomatis][SEQ ID NO:31]

>gi|3328905|gb|AAC68071.1| hypothetical protein [Chlamydia trachomatis][SEQ ID NO:33]

>gi|3328884|gb|AAC68051.1| Phosphatidate Cytidylytransferase [Chlamydia trachomatis][SEQ ID NO:35]

>gi|3328855|gb|AAC68022.1| hypothetical protein [Chlamydia trachomatis][SEQ ID NO:37]

>gi|3328772|gb|AAC67946.1| hypothetical protein [Chlamydia trachomatis][SEQ ID NO:39]

>gi|3328763|gb|AAC67938.1| O-Sialoglycoprotein Endopeptidase family [Chlamydia trachomatis][SEQ ID NO:49]

>gi|6578102|gb|AAC67897.2| ATP Synthase Subunit K [Chlamydia trachomatis][SEQ ID NO:51]

>gi|3329252|gb|AAC68382.1| S14 Ribosomal Protein [Chlamydia trachomatis][SEQ ID NO:53]

>gi|3328987|gb|AAC68150.1| hypothetical protein [Chlamydia trachomatis][SEQ ID NO:57]

>gi|3328972|gb|AAC68136.1| Apolipoprotein N-Acetyltransferase [Chlamydia trachomatis][SEQ ID NO:59]

>gi|3328612|gb|AAC67797.1| Fructose-6-P Phosphotransferase [Chlamydia trachomatis][SEQ ID NO:61]

>gi|3328517|gb|AAC67709.1| hypothetical protein [Chlamydia trachomatis][SEQ ID NO:63]

>gi|3328482|gb|AAC67677.1| L28 Ribosomal Protein [Chlamydia trachomatis][SEQ ID NO:65]

>gi|3328436|gb|AAC67635.1| SS DNA Binding Protein [Chlamydia trachomatis][SEQ ID NO:67]

>gi|3328411|gb|AAC67611.1| hypothetical protein [Chlamydia trachomatis][SEQ ID NO:69]

Again, when such blebs are present in a vaccine formulation they may be more protective against Chlamydia trachomatis infection than the use of the protein in isolation.

Particularly beneficial pairs of Chlamydia trachomatis antigens are further preferred embodiments of this invention. Thus in a further aspect a Gram-negative bleb (preferably from gonococcus) is provided presenting on its surface both the PorB and PmpG outer membrane proteins from Chlamydia trachomatis. Furthermore, a Gram-negative bleb (preferably from gonococcus) is provided presenting on its surface both the PorB and MOMP (from one or more serovars) outer membrane proteins from Chlamydia trachomatis. Lastly, a Gram-negative bleb (preferably from gonococcus) is provided presenting on its surface both the PmpG and MOMP (from one or more serovars) outer membrane proteins from Chlamydia trachomatis.

By MOMP (or OMP1 or OMP I) from one or more (1, 2, 3, 4, 5, 6, 7, 8, 9, 10 or more) serovars it is preferred that one or more should be selected from a list serovars consisting of: B, Ba, D, Da, E, L1, L2, L2a, F, G, K, L3, A, C, H, I, Ia, & J; more preferably from a list consisting of D, E, F, G, K, H, I, & J. Most preferably one or more MOMPs should at least comprise MOMP from serovar D or E (most preferably D). A further preferred strategy is the selection of one or more MOMP from each of the following 3 serogroups: B-serogroup (consisting of serovars B, Ba, D, Da, E, L1, L2 and L2a, and preferably selected from serovars D, Da, & E); F-G-serogroup (consisting of serovars F and G); and C-serogroup (consisting of servars A, C, H, I, Ia, J, K, and L3, and preferably selected from serovars H, I, Ia, J and K).

Most preferably the genes for the Chlamydia trachomatis antigens should be inserted at the PorA locus of Neisseria (preferably gonococcus).

Such a prepartion formulated as a vaccine may give enhanced protection to a host against Chlamydia trachomatis than when a single antigen is administered.

Preferably the bleb has been derived from a strain (preferably gonococcus) which has been modified to upregulate one or more protective outer membrane antigens (as described below).

Preferably the bleb has been derived from a strain (preferably gonococcus) which has been modified to downregulate one or more immunodominant variable or non-protective outer membrane antigens (as described below).

Preferably the blebs are derived from a strain (preferably gonococcus) which has a detoxified lipid A portion of bacterial LPS, due to the strain having been engineered to reduce or switch off expression of one or more genes which cause LPS to be toxic (preferably selected from the following genes, or homologues thereof htrB, msbB and lpxK; see section below).

Preferably the blebs are derived from a strain (preferably gonococcus) which has a detoxified lipid A portion of bacterial LPS, due to the strain having been engineered to express at a higher level of one or more genes producing a gene product that is capable of detoxifying LPS (preferably selected from the following genes, or homologues thereof: pmrA, pmrB, pmrE and pmrF; see section below).

Vaccine compositions comprising the bleb of the invention and a pharmaceutically suitable excipient or carrier is also envisaged. Preferably the vaccine additionally comprises a mucosal adjuvant. Mucosal adjuvants are well known in the art (see Vaccine Design “The subunit and adjuvant approach” (eds Powell M. F. & Newman M. J.) (1995) Plenum Press New York). A preferred mucosal adjuvant is LT2 (or LTII, which can be split into LTIIa and LTIIb—see Martin et al. Infection and Immunity, 2000, 68:281-287). Preferably such vaccines should be formulated and administered as described below in “vaccine formulations”.

The content of blebs per dose in the vaccine will typically be in the range 1-100 μg, preferably 5-50 μg, most typically in the range 5-25 μg.

Optimal amounts of components for a particular vaccine can be ascertained by standard studies involving observation of appropriate immune responses in subjects. Following an initial vaccination, subjects may receive one or several booster immunisations adequately spaced.

A method of preventing Chlamydia trachomatis infection in a host is also provided comprising the steps of administering an effective amount of the above vaccine to a host in need thereof. Preferably the vaccine is mucosally administered via either a intranasal, oral, intradermal or intravaginal route.

Chlamydia pneumoniae Antigens Integrated into a Gram Negative Bacterial bleb

In a further aspect, the invention provides a Gram-negative bleb presenting on its surface a protective antigen from Chlamydia pneumoniae. Neisseria meningitidis, Moraxella catharralis, and Haemophilus influenzae are preferred species for the production of said bleb. A bacterial strain capable of producing such a bleb is a further aspect of the invention. Such protective antigens are preferably one or more of those listed below:

1) Cell Envelope: Membrane Proteins, Lipoproteins and Porins

| Gene: | Protein Function: |
|---|---|
| yaeT | OMP85 homolog |
| 60IM | 60 kD inner membrane protein |
| lgt | prolipoprotein diacylglyceryl transferase |
| crpA | CHLTR 15 kD cysteine-rich protein |
| omcB | 60 kD cysteine-rich outer membrane complex protein |
| omcA | 9 kD cysteine-rich outer membrane complex lipoprotein |
| cutE | apolipoprotein N-acetyltransferase |
| ompA | major outer membrane protein |
| pal | peptidoglycan-associated lipoprotein |
| porB | outer membrane protein analog |

2) Coding Genes (Not in C. trachomatis)

| Gene: | Protein Function: |
|---|---|
| yqfF | conserved hypothetical inner membrane protein |
| yxjG | hypothetical protein |
| guaA | GMP synthase |
| guaB | inosine 5′-monophosphate dehydrogenase |
| argR | similarity to arginine repressor |
| CPn0232 | similarity to 5′-methylthioadenosine nucleosidase |
| CPn0251 | conserved hypothetical protein |
| CPn0278 | conserved outer membrane lipoprotein protein/a> |
| CPn0279 | possible ABC transporter permease |
| yxjG | hypothetical protein |
| yqeV | hypothetical protein |
| CPn0486 | hypothetical proline permease |
| CPn0505 | 3-methyladenine DNA glycosylase |
| CPn0562 | CHLPS 43 kDa protein |
| CPn0585 | similarity to CHLPS IncA |
| yvyD | conserved hypothetical protein |
| CPn0608 | uridine 5′-monophosphate synthase |
| CPn0735 | uridine kinase |
| CPn0907 | CutA-like periplasmic divalent cation tolerance protein |
| CPn0927 | CHLPS 43 kDa protein |
| CPn0928 | CHLPS 43 kDa protein |
| CPn0929 | CHLPS 43 kDa protein |
| CPn0980 | similar to S. cerevisiae 52.9 kDa protein |
| bioA | adenosylmethionine-8-amino-7-oxononanoate |
| | aminotransferase |
| bioD | dethiobiotin synthetase |
| bioB | biotin synthase |
| CPn1045 | conserved hypothetical membrane protein |
| CPn1046 | tryptophan hydroxylase |

3) Chlamydia-Specific Proteins

| Gene: | Protein Function: |
|---|---|
| pmp_1 | polymorphic outer membrane protein |
| pmp_2 | polymorphic outer membrane protein |
| pmp_3 | polymorphic outer membrane protein |
| pmp_3 | polymorphic outer membrane protein |
| pmp_4 | polymorphic outer membrane protein |
| pmp_4 | polymorphic outer membrane protein |
| pmp_5 | polymorphic outer membrane protein |
| pmp_5 | polymorphic outer membrane protein |
| CPn0133 | CHLPS hypothetical protein |
| CPn0186 | similarity to IncA |
| incB | inclusion membrane protein B |
| incC | inclusion membrane protein C |
| CPn0332 | CHLTR T2 protein |
| ltuB | LtuB protein |
| pmp_6 | polymorphic outer membrane protein |
| pmp_7 | polymorphic outer membrane protein |
| pmp_8 | polymorphic outer membrane protein |
| pmp_9 | polymorphic outer membrane protein |
| pmp_10 | polymorphic outer membrane protein |
| pmp_10 | polymorphic outer membrane protein |
| pmp_11 | polymorphic outer membrane protein |
| pmp_12 | polymorphic outer membrane protein |
| pmp_13 | polymorphic outer membrane protein |
| pmp_14 | polymorphic outer membrane protein |
| pmp_15 | polymorphic outer membrane protein |
| pmp_16 | polymorphic outer membrane protein |
| pmp_17 | polymorphic outer membrane protein |
| pmp_17 | polymorphic outer membrane protein |
| pmp_17 | polymorphic outer membrane protein |
| pmp_18 | polymorphic outer membrane protein |
| pmp_19 | polymorphic outer membrane protein |
| pmp_20 | polymorphic outer membrane protein |
| euo | CHLPS Euo protein |
| CPn0562 | CHLPS 43 kDa protein homolog |
| CPn0585 | similar to CHLPS inclusion membrane protein A |
| CPn0728 | CHLPN 76 kDa protein homolog |
| CPn0729 | CHLPN 76 kDa protein homolog |
| gp6D | CHLTR plasmid protein |
| CPn0927 | CHLPS 43 kDa protein homolog |
| CPn0928 | CHLPS 43 kDa protein homolog |
| CPn0929 | CHLPS 43 kDa protein homolog |
| pmp_21 | polymorphic outer membrane protein |
| ltuA | LtuA protein |

(Full sequence information has been published at the Chlamydia Genome Project web site: http://chlamydia-www.berkeley.edu:4231/index.html).

Additional Chlamydia Genes, and Encoded Proteins, Suitable for Expression in a Gram-Negative Bacteria for OMV Vaccine Preparation:

| | |
|---|---|
| Chlamydia pneumoniae 98 kD putative outer membrane | WO2000262 |
| protein gene. | 37-A2 |
| Patent InventorsDUNN PL | |
| OOMEN RP | |
| MURDIN AD | |
| Chlamydia POMP91B precursor gene. | WO2000262 |
| Patent InventorsDUNN PL | 39-A2 |
| OOMEN RP | |
| MURDIN AD | |
| Chlamydia antigen CPN100634 full length coding | WO2000327 |
| sequence. | 94-A2 |
| Patent InventorsOOMEN RP | |
| WANG J | |
| MURDIN AD | |
| Chlamydia antigen CPN100634 gene open reading frame. | WO2000327 |
| Patent InventorsOOMEN RP | 94-A2 |
| WANG J | |
| MURDIN AD | |
| Chlamydia antigen CPN100635 full length coding | WO2000327 |
| sequence. | 94-A2 |
| Patent InventorsOOMEN RP | |
| WANG J | |
| MURDIN AD | |
| Chlamydia antigen CPN100635 gene open reading frame. | WO2000327 |
| Patent InventorsOOMEN RP | 94-A2 |
| WANG J | |
| MURDIN AD | |
| Chlamydia antigen CPN100638 full length coding | WO2000327 |
| sequence. | 94-A2 |
| Patent InventorsOOMEN RP | |
| WANG J | |
| MURDIN AD | |
| Chlamydia antigen CPN100638 gene open reading frame. | WO2000327 |
| Patent InventorsOOMEN RP | 94-A2 |
| WANG J | |
| MURDIN AD | |
| Chlamydia antigen CPN100639 full length coding | WO2000327 |
| sequence. | 94-A2 |
| Patent InventorsOOMEN RP | |
| WANG J | |
| MURDIN AD | |
| Chlamydia antigen CPN100639 gene open reading frame. | WO2000327 |
| Patent InventorsOOMEN RP | 94-A2 |
| WANG J | |
| MURDIN AD | |
| Chlamydia antigen CPN100708 full length coding | WO2000327 |
| sequence. | 94-A2 |
| Patent InventorsOOMEN RP | |
| WANG J | |
| MURDIN AD | |
| Chlamydia antigen CPN100708 gene open reading frame. | WO2000327 |
| Patent InventorsOOMEN RP | 94-A2 |
| WANG J | |
| MURDIN AD | |
| C. pneumoniae ATP/ADP translocase coding sequence. | WO2000391 |
| Patent InventorsDUNN P | 57-A1 |
| OOMEN RP | |
| WANG J | |
| MURDIN AD | |
| Chlamydia pneumoniae 98 kDa outer membrane protein | WO2000327 |
| CPN100640 gene. | 84-A1 |
| Patent InventorsDUNN P | |
| OOMEN RP | |
| WANG J | |
| MURDIN AD | |
| Chlamydia pneumoniae 98 kDa outer membrane protein | WO2000327 |
| coding region. | 84-A1 |
| Patent InventorsDUNN P | |
| OOMEN RP | |
| WANG J | |
| MURDIN AD | |
| DNA encoding a 9 kDa cysteine-rich membrane protein. | WO2000537 |
| Patent InventorsDUNN P | 64-A1 |
| OOMEN RP | |
| WANG J | |
| MURDIN AD | |
| DNA encoding a 60 kDa cysteine-rich membrane protein. | WO2000553 |
| Patent InventorsDUNN P | 26-A1 |
| OOMEN RP | |
| WANG J | |
| MURDIN AD | |
| A 9 kDa cysteine-rich membrane protein. | WO2000537 |
| Patent InventorsDUNN P | 64-A1 |
| OOMEN RP | |
| WANG J | |
| MURDIN AD | |
| A 60 kDa cysteine-rich membrane protein of Chlamydia | WO2000553 |
| pneumoniae. | 26-A1 |
| Patent InventorsDUNN P | |
| OOMEN RP | |
| WANG J | |
| MURDIN AD | |
| C. pneumoniae mip (outer membrane protein). | WO2000067 |
| Patent InventorsDUNN PL | 41-A1 |
| OOMEN RP | |
| MURDIN AD | |
| C. pneumoniae mip (outer membrane protein) truncated | WO2000067 |
| protein. | 41-A1 |
| Patent InventorsDUNN PL | |
| OOMEN RP | |
| MURDIN AD | |
| C. pneumoniae omp protein sequence. | WO2000067 |
| Patent InventorsDUNN PL | 43-A2 |
| OOMEN RP | |
| MURDIN AD | |
| C. pneumoniae omp protein truncated sequence. | WO2000067 |
| Patent InventorsDUNN PL | 43-A2 |
| OOMEN RP | |
| MURDIN AD | |
| Amino acid sequence of the CPN100111 polypeptide. | WO2000111 |
| Patent InventorsOOMEN RP | 83-A2 |
| MURDIN AD | |
| Amino acid sequence of the CPN100224 polypeptide. | WO2000111 |
| Patent InventorsOOMEN RP | 83-A2 |
| MURDIN AD | |
| Amino acid sequence of the CPN100230 polypeptide. | WO2000111 |
| Patent InventorsOOMEN RP | 83-A2 |
| MURDIN AD | |
| Amino acid sequence of the CPN100231 polypeptide. | WO2000111 |
| Patent InventorsOOMEN RP | 83-A2 |
| MURDIN AD | |
| Amino acid sequence of the CPN100232 polypeptide. | WO2000111 |
| Patent InventorsOOMEN RP | 83-A2 |
| MURDIN AD | |
| Amino acid sequence of the CPN100233 polypeptide. | WO2000111 |
| Patent InventorsOOMEN RP | 83-A2 |
| MURDIN AD | |
| Amino acid sequence of the CPN100394 polypeptide. | WO2000111 |
| Patent InventorsOOMEN RP | 83-A2 |
| MURDIN AD | |
| Amino acid sequence of the CPN100395 polypeptide. | WO2000111 |
| Patent InventorsOOMEN RP | 83-A2 |
| MURDIN AD | |
| Amino acid sequence of the POMP91A protein of | WO2000111 |
| Chlamydia pneumoniae. | 80-A1 |
| Patent InventorsDUNN PL | |
| OOMEN RP | |
| MURDIN AD | |
| Chlamydia pneumoniae antigen CPN100202 protein | WO2000067 |
| sequence. | 39-A2 |
| Patent InventorsOOMEN RP | |
| MURDIN AD | |
| Chlamydia pneumoniae antigen CPN100149 protein | WO2000067 |
| SEQ ID NO: 2. | 40-A1 |
| Patent InventorsOOMEN RP | |
| MURDIN AD | |
| Chlamydia pneumoniae antigen CPN100605 protein | WO2000067 |
| SEQ ID NO: 2. | 42-A2 |
| Patent InventorsOOMEN RP | |
| MURDIN AD | |
| Chlamydia antigen CPN100634. | WO2000327 |
| Patent InventorsOOMEN RP | 94-A2 |
| WANG J | |
| MURDIN AD | |
| Chlamydia antigen CPN100635. | WO2000327 |
| Patent InventorsOOMEN RP | 94-A2 |
| WANG J | |
| MURDIN AD | |
| Mature Chlamydia antigen CPN100635. | WO2000327 |
| Patent InventorsOOMEN RP | 94-A2 |
| WANG J | |
| MURDIN AD | |
| Chlamydia antigen CPN100638. | WO2000327 |
| Patent InventorsOOMEN RP | 94-A2 |
| WANG J | |
| MURDIN AD | |
| Chlamydia antigen CPN100639. | WO2000327 |
| Patent InventorsOOMEN RP | 94-A2 |
| WANG J | |
| MURDIN AD | |
| Chlamydia antigen CPN100708. | WO2000327 |
| Patent InventorsOOMEN RP | 94-A2 |
| WANG J | |
| MURDIN AD | |
| C. pneumoniae ATP/ADP translocase protein sequence. | WO2000391 |
| Patent InventorsDUNN P | 57-A1 |
| OOMEN RP | |
| WANG J | |
| MURDIN AD | |
| Chlamydia pneumoniae 98 kD putative outer membrane | WO2000262 |
| protein. | 37-A2 |
| Patent InventorsDUNN PL | |
| OOMEN RP | |
| MURDIN AD | |
| Chlamydia POMP91B precursor protein. | WO2000262 |
| Patent InventorsDUNN PL | 39-A2 |
| OOMEN RP | |
| MURDIN AD | |
| Chlamydia pneumoniae 98 kDa outer membrane protein | WO2000327 |
| CPN100640. | 84-A1 |
| Patent InventorsDUNN P | |
| OOMEN RP | |
| WANG J | |
| MURDIN AD | |
| Chlamydia pneumoniae processed 98 kDa outer membrane | WO2000327 |
| protein CPN100640. | 84-A1 |
| Patent InventorsDUNN P | |
| OOMEN RP | |
| WANG J | |
| MURDIN AD | |
| C. pneumoniae mip (outer membrane protein) encoding | WO2000067 |
| DNA. | 41-A1 |
| Patent InventorsDUNN PL | |
| OOMEN RP | |
| MURDIN AD | |
| C. pneumoniae omp protein encoding DNA. | WO2000067 |
| Patent InventorsDUNN PL | 43-A2 |
| OOMEN RP | |
| MURDIN AD | |
| DNA encoding the CPN100111 polypeptide. | WO2000111 |
| Patent InventorsOOMEN RP | 83-A2 |
| MURDIN AD | |
| DNA encoding the CPN100224 polypeptide. | WO2000111 |
| Patent InventorsOOMEN RP | 83-A2 |
| MURDIN AD | |
| DNA encoding the CPN100230 polypeptide. | WO2000111 |
| Patent InventorsOOMEN RP | 83-A2 |
| MURDIN AD | |
| DNA encoding the CPN100231 polypeptide. | WO2000111 |
| Patent InventorsOOMEN RP | 83-A2 |
| MURDIN AD | |
| DNA encoding the CPN100232 polypeptide. | WO2000111 |
| Patent InventorsOOMEN RP | 83-A2 |
| MURDIN AD | |
| DNA encoding the CPN100233 polypeptide. | WO2000111 |
| Patent InventorsOOMEN RP | 83-A2 |
| MURDIN AD | |
| DNA encoding the CPN100394 polypeptide. | WO2000111 |
| Patent InventorsOOMEN RP | 83-A2 |
| MURDIN AD | |
| DNA encoding the CPN100395 polypeptide. | WO2000111 |
| Patent InventorsOOMEN RP | 83-A2 |
| MURDIN AD | |
| Nucleotide sequence of the POMP91A gene of Chlamydia | WO2000111 |
| pneumoniae. | 80-A1 |
| Patent InventorsDUNN PL | |
| OOMEN RP | |
| MURDIN AD | |
| Chlamydia pneumoniae antigen CPN100202 nucleotide | WO2000067 |
| sequence. | 39-A2 |
| Patent InventorsOOMEN RP | |
| MURDIN AD | |
| Chlamydia pneumoniae antigen CPN100149 protein | WO2000067 |
| encoding DNA SEQ ID NO: 1. | 40-A1 |
| Patent InventorsOOMEN RP | |
| MURDIN AD | |
| Chlamydia pneumoniae antigen CPN100605 protein | WO2000067 |
| encoding DNA SEQ ID NO: 1. | 42-A2 |
| Patent InventorsOOMEN RP | |
| MURDIN AD | |

When such blebs are present in a vaccine formulation they may be more protective against Chlamydia pneumoniae infection than the use of the protein/antigen in isolation.

Particularly beneficial pairs of Chlamydia pneumoniae antigens have also been found. Thus in a further aspect a Gram-negative bleb (preferably from meningococcus) is provided presenting on its surface both the PorB and MOMP outer membrane proteins from Chlamydia pneumoniae. Furthermore, a Gram-negative bleb (preferably from meningococcus) is provided presenting on its surface both MOMP and one or more Pmp outer membrane proteins from Chlamydia pneumoniae. A Gram-negative bleb (preferably from meningococcus) is additionally provided presenting on its surface both the PorB and one or more Pmp outer membrane proteins from Chlamydia pneumoniae. A Gram-negative bleb (preferably from meningococcus) is also provided presenting on its surface both the PorB and Npt1 proteins from Chlamydia pneumoniae. A Gram-negative bleb (preferably from meningococcus) is additionally provided presenting on its surface both the Npt1 and one or more Pmp proteins from Chlamydia pneumoniae. Lastly, a Gram-negative bleb (preferably from meningococcus) is provided presenting on its surface both the Npt1 and MOMP proteins from Chlamydia pneumoniae. Bacterial strains from which these blebs are derived are further aspects of this invention.

Such prepartions formulated as a vaccine can give enhanced protection to a host against Chlamydia than when a single antigen is administered.

Preferably the bleb has been derived from a strain which has been modified to upregulate one or more protective outer membrane antigens (see below; for instance for meningocococcal protective outer membrane antigens see section “Neisserial bleb preparations” for those antigens that should preferably be upregulated).

Preferably the bleb has been derived from a strain which has been modified to downregulate one or more immunodominant variable or non-protective outer membrane antigens (as described below; for instance for meningocococcal variable/non-protective outer membrane antigens see section “Neisserial bleb preparations” for those antigens that should preferably be downregulated).

Preferably the blebs are derived from a strain which has a detoxified lipid A portion of bacterial LPS, due to the strain having been engineered to reduce or switch off expression of one or more genes which cause LPS to be toxic (preferably selected from the following genes, or homologues thereof htrB, msbB and lpxK; see section below).

Preferably the blebs are derived from a strain which has a detoxified lipid A portion of bacterial LPS, due to the strain having been engineered to express at a higher level of one or more genes producing a gene product that is capable of detoxifying LPS (preferably selected from the following genes, or homologues thereof: pmrA, pmrB, pmrE and pmrF; see section below).

Vaccine compositions comprising the bleb of the invention and a pharmaceutically suitable excipient or carrier are also envisaged. Preferably the vaccine additionally comprising a mucosal adjuvant. Mucosal adjuvants are well known in the art (see Vaccine Design “The subunit and adjuvant approach” (eds Powell M. F. & Newman M. J.) (1995) Plenum Press New York). A preferred mucosal adjuvant is LT2 (or LTII, which can be split into LTIIa and LTIIb—see Martin et al. Infection and Immunity, 2000, 68:281-287). Preferably such vaccines should be formulated and administered as described below in “Vaccine formulations”.

The content of blebs per dose in the vaccine will typically be in the range 1-100 μg, preferably 5-50 μg, most typically in the range 5-25 μg.

Optimal amounts of components for a particular vaccine can be ascertained by standard studies involving observation of appropriate immune responses in subjects. Following an initial vaccination, subjects may receive one or several booster immunisations adequately spaced.

The efficacy of a C. pneumoniae vaccine can be evaluated in a mouse model of infection such as the one described by Murdin et al., 2000, J. Infect. Dis. 181 (suppl 3):S5444-5 1. The protection elicited by a vaccine formulation can be assessed by reduction of the bacterial load in the lung after a challenge infection with C. pneumoniae.

A method of preventing Chlamydia pneumoniae infection in a host is also provided comprising the steps of administering an effective amount of the above vaccine to a host in need thereof. Preferably the vaccine is mucosally administered via either an intranasal, intradermal or oral route.

Further Improvements in the Bacteria and blebs of the Invention

The Gram-negative bacterium of the invention may be further genetically engineered by one or more processes selected from the following group: (a) a process of down-regulating expression of immunodominant variable or non-protective antigens, (b) a process of upregulating expression of protective OMP antigens, (c) a process of down-regulating a gene involved in rendering the lipid A portion of LPS toxic, (d) a process of upregulating a gene involved in rendering the lipid A portion of LPS less toxic, and (e) a process of down-regulating synthesis of an antigen which shares a structural similarity with a human structure and may be capable of inducing an auto-immune response in humans. These processes are described in detail in WO 01/09350 (incorporated by reference herein).

Such bleb vaccines of the invention are designed to focus the immune response on a few protective (preferably conserved) antigens or epitopes—formulated in a multiple component vaccine. Where such antigens are integral OMPs, the outer membrane vesicles of bleb vaccines will ensure their proper folding. This invention provides methods to optimize the OMP and LPS composition of OMV (bleb) vaccines by deleting immunodominant variable as well as non protective OMPs, by creating conserved OMPs by deletion of variable regions, by upregulating expression of protective OMPs, and by eliminating control mechanisms for expression (such as iron restriction) of protective OMPs. In addition the invention provides for the reduction in toxicity of lipid A by modification of the lipid portion or by changing the phosphoryl composition whilst retaining its adjuvant activity or by masking it. Each of these new methods of improvement individually improve the bleb vaccine, however a combination of one or more of these methods work in conjunction so as to produce an optimised engineered bleb vaccine which is immuno-protective and non-toxic—particularly suitable for paediatric use.

(a) A Process of Down-Regulating Expression of Immunodominant Variable or Non-Protective Antigens

Many surface antigens are variable among bacterial strains and as a consequence are protective only against a limited set of closely related strains. An aspect of this invention covers the reduction in expression, or, preferably, the deletion of the gene(s) encoding variable surface protein(s) which results in a bacterial strain producing blebs which, when administered in a vaccine, have a stronger potential for cross-reactivity against various strains due to a higher influence exerted by conserved proteins (retained on the outer membranes) on the vaccinee's immune system. Examples of such variable antigens include: for Neisseria—pili (PilC) which undergoes antigenic variations, PorA, Opa, TbpB, FrpB; for H. influenzae—P2, P5, pilin, IgA1-protease; and for Moraxella—CopB, OMP106.

Other types of gene that could be down-regulated or switched off are genes which, in vivo, can easily be switched on (expressed) or off by the bacterium. As outer membrane proteins encoded by such genes are not always present on the bacteria, the presence of such proteins in the bleb preparations can also be detrimental to the effectiveness of the vaccine for the reasons stated above. A preferred example to down-regulate or delete is Neisseria Opc protein. Anti-Opc immunity induced by an Opc containing bleb vaccine would only have limited protective capacity as the infecting organism could easily become Opc⁻. H. influenzae HgpA and HgpB are other examples of such proteins.

In process a), these variable or non-protective genes are down-regulated in expression, or terminally switched off. This has the surprising advantage of concentrating the immune system on better antigens that are present in low amounts on the outer surface of blebs.

The strain can be engineered in this way by a number of strategies including transposon insertion to disrupt the coding region or promoter region of the gene, or point mutations or deletions to achieve a similar result. Homologous recombination may also be used to delete a gene from a chromosome (where sequence X comprises part (preferably all) of the coding sequence of the gene of interest). It may additionally be used to change its strong promoter for a weaker (or no) promoter. All these techniques are described in WO 01/09350 (published by WIPO on Aug. 2, 2001 and incorporated by reference herein).

(b) A Process of Upregulating Expression of Protective OMP Antigens

This may be done by inserting a copy of such a protective OMP into the genome (preferably by homologous recombination), or by upregulating expression of the native gene by replacing the native promoter for a stronger promoter, or inserting a strong promoter upstream of the gene in question (also by homologous recombination). Such methods can be accomplished using the techniques described in WO 01/09350 (published by WIPO on Aug. 2, 2001 and incorporated by reference herein).

Such methods are particularly useful for enhancing the production of immunologically relevant Bleb components such as outer-membrane proteins and lipoproteins (preferably conserved OMPs, usually present in blebs at low concentrations).

(c) A Process of Down-Regulating a Gene Involved in Rendering the Lipid A Portion of LPS Toxic

The toxicity of bleb vaccines presents one of the largest problems in the use of blebs in vaccines. A further aspect of the invention relates to methods of genetically detoxifying the LPS present in Blebs. Lipid A is the primary component of LPS responsible for cell activation. Many mutations in genes involved in this pathway lead to essential phenotypes. However, mutations in the genes responsible for the terminal modifications steps lead to temperature-sensitive (htrB) or permissive (msbB) phenotypes. Mutations resulting in a decreased (or no) expression of these genes result in altered toxic activity of lipid A. Indeed, the non-lauroylated (htrB mutant) [also defined by the resulting LPS lacking both secondary acyl chains] or non-myristoylated (msbB mutant) [also defined by the resulting LPS lacking only a single secondary acyl chain] lipid A are less toxic than the wild-type lipid A. Mutations in the lipid A 4′-kinase encoding gene (lpxK) also decreases the toxic activity of lipid A.

Process c) thus involves either the deletion of part (or preferably all) of one or more of the above open reading frames or promoters. Alternatively, the promoters could be replaced with weaker promoters. Preferably the homologous recombination techniques are used to carry out the process. Preferably the methods described in WO 01/09350 (published by WIPO on Aug. 2, 2001 and incorporated by reference herein) are used. The sequences of the htrB and msbB genes from Neisseria meningitidis B, Moraxella catarrhalis, and Haemophilus influenzae are provided in WO 01/09350 for this purpose.

(d) A Process of Upregulating a Gene Involved in Rendering the Lipid A Portion of LPS less Toxic

LPS toxic activity could also be altered by introducing mutations in genes/loci involved in polymyxin B resistance (such resistance has been correlated with addition of arninoarabinose on the 4′ phosphate of lipid A). These genes/loci could be pmrE that encodes a UDP-glucose dehydrogenase, or a region of antimicrobial peptide-resistance genes common to many enterobacteriaciae which could be involved in aminoarabinose synthesis and transfer. The gene pmrF that is present in this region encodes a dolicol-phosphate manosyl transferase (Gunn J. S., Kheng, B. L., Krueger J., Kim K., Guo L., Hackett M., Miller S. I. 1998. Mol. Microbiol. 27: 1171-1182).

Mutations in the PhoP-PhoQ regulatory system, which is a phospho-relay two component regulatory system (f.i. PhoP constitutive phenotype, PhoP^{c}), or low Mg⁺⁺ environmental or culture conditions (that activate the PhoP-PhoQ regulatory system) lead to the addition of aminoarabinose on the 4′-phosphate and 2-hydroxymyristate replacing myristate (hydroxylation of myristate). This modified lipid A displays reduced ability to stimulate E-selectin expression by human endothelial cells and TNF-α secretion from human monocytes.

Process d) involves the upregulation of these genes using a strategy as described in WO 01/09350 (published by WIPO on Aug. 2, 2001 and incorporated by reference herein).

(e) A Process of Down-Regulating Synthesis of an Antigen which Shares a Structural Similarity with a Human Structure and may be Capable of Inducing an Auto-Immune Response in Humans

The isolation of bacterial outer-membrane blebs from encapsulated Gram-negative bacteria often results in the co-purification of capsular polysaccharide. In some cases, this “contaminant” material may prove useful since polysaccharide may enhance the immune response conferred by other bleb components. In other cases however, the presence of contaminating polysaccharide material in bacterial bleb preparations may prove detrimental to the use of the blebs in a vaccine. For instance, it has been shown at least in the case of N. meningitidis that the serogroup B capsular polysaccharide does not confer protective immunity and is susceptible to induce an adverse auto-immune response in humans. Consequently, process e) of the invention is the engineering of the bacterial strain for bleb production such that it is free of capsular polysaccharide. The blebs will then be suitable for use in humans. A particularly preferred example of such a bleb preparation is one from N. meningitidis serogroup B devoid of capsular polysaccharide.

This may be achieved by using modified bleb production strains in which the genes necessary for capsular biosynthesis and/or export have been impaired as described in WO 01/09350 (published by WIPO on Aug. 2, 2001 and incorporated by reference herein). A preferred method is the deletion of some or all of the Neisseria meningitidis cps genes required for polysaccharide biosynthesis and export. For this purpose, the replacement plasmid pMF121 (described in Frosh et al. 1990, Mol. Microbiol. 4:1215-1218) can be used to deliver a mutation deleting the cpsCAD (+galE) gene cluster. Alternatively the siaD gene could be deleted, or down-regulated in expression (the meningococcal siaD gene encodes alpha-2,3-sialyltransferase, an enzyme required for capsular polysaccharide and LOS synthesis). Such mutations may also remove host-similar structures on the saccharide portion of the LPS of the bacteria.

Combinations of Methods a)-e)

It may be appreciated that one or more of the above processes may be used to produce a modified strain from which to make improved bleb preparations of the invention. Preferably one such process is used, more preferably two or more (2, 3, 4, or 5) of the processes are used in order to manufacture the bleb vaccine. As each additional method is used in the manufacture of the bleb vaccine, each improvement works in conjunction with the other methods used in order to make an optimised engineered bleb preparation.

A preferred meningococcal (particularly N. meningitidis B) bleb preparation comprises the use of processes b), c) and e) (optionally combined with process a)). Such bleb preparations are safe (no structures similar to host structures), non-toxic, and structured such that the host immune response will be focused on high levels of protective (and preferably conserved) antigens. All the above elements work together in order to provide an optimised bleb vaccine.

Similarly for M. catarrhalis, non-typeable H. influenzae, gonococcus, and non serotype B meningococcal strains (e.g. serotype A, C, Y or W), preferred bleb preparations comprise the use of processes b) and c), optionally combined with process a).

Preferred Neisserial bleb Preparations

One or more of the following genes (encoding protective antigens) are preferred for upregulation via process b) when carried out on a Neisserial strain, including gonococcus, and meningococcus (particularly N. meningitidis B): NspA (WO 96/29412), Hsf-like (WO 99/31132), Hap (PCT/EP99/02766), PorA, PorB, OMP85 (WO 00/23595), PilQ (PCT/EP99/03603), PldA (PCT/EP99/06718), FrpB (WO 96/31618), TbpA (U.S. Pat. No. 5,912,336), TbpB, FrpA/FrpC (WO 92/01460), LbpA/LbpB (PCT/EP98/05117), FhaB (WO 98/02547), HasR (PCT/EP99/05989), lipo02 (PCT/EP99/08315), Thp2 (WO 99/57280), MltA (WO 99/57280), and ctrA (PCT/EP00/00135). They are also preferred as genes which may be heterologously introduced into other Gram-negative bacteria.

One or more of the following genes are preferred for downregulation via process a): PorA, PorB, PilC, ThpA, TbpB, LbpA, LbpB, Opa, and Opc (most preferably PorA).

One or more of the following genes are preferred for downregulation via process c): htrB, msbB and lpxK (most preferably msbB which removes only a single secondary acyl chain from the LPS molecule).

One or more of the following genes are preferred for upregulation via process d): pmrA, pmrB, pmrE, and pmrF.

One or more of the following genes are preferred for downregulation via process e): galE, siaA, siaB, siaC, siaD, ctrA, ctrB, ctrC, and ctrD (the genes are described in described in WO 01/09350—published by WIPO on Aug. 2, 2001 and incorporated by reference herein).

Many of the above open reading frames and upstream regions are described in WO 01/09350 (incorporated by reference herein).

Preferred gonococcal genes to upregulate via process b) include one or more of the following:

```
[SEQ ID NO:79]
Neisseria gonorrheae lactoferrin receptor precursor (lbpA) gene,
complete cds.
ACCESSION U16260
VERSION U16260.1 GI:915277
Source: Neisseria gonorrhoeae/strain=“FA19”
gene=“lbpA” nucleotides: 278..3109
protein_id=“AAC13780.1”/db_xref=“GI:915278”
                        /translation=
“MNKKHGFPLTLTALAIATAFPAYAAQAGAAALDAAQSQSLKEVT
VPAAKVGRRSKEATGLGKIVKTSETLNKEQVLGIRDLTRYDPGVAVVEQGNGASGGYS
IRGVDKNRVAVSVDGVAQIQAFTVQGSLSGYGGRGGSGAINEIEYENISTVEIDKGAG
SSDHGSGALGGAVAFRTKEAADLISDGKSWGIQAKTAYGSKNRQFMKSLGAGFSKDGW
EGLLIRTERQGRETRPHGDIADGVEYGIDRLDAFRQTYDIKRKTTEPFFLVEGENTLK
PVAKLAGYGIYLNRQLNRWVKERIEQNQPLSAEEEAQVREAQARHENLSAQAYTGGGR
ILPDPMDYRSGSWLAKLGYRFGGRHYVGGVFEDTKQRYDIRDMTEKQYYGTDEAEKFR
DKSGVYDGDDFRDGLYFVPNIEEWKGDKNLVKGIGLKYSRTKFIDEHHRRRRMGLLYR
YENEKYSDNWADKAVLSFDKQGVATDNNTLKLNCAVYPAVDKSCRASADKPYSYDSSD
RFHYREQHNVLNASFEKSLKNKWTKHHLTLGFGYDASKAVSRPEQLSHNAARISESTG
FDEKNQDKYRLGKPEVVEGSVCGYIETLRSRKCVPRKINGSNIHISLNDRFSIGKYFD
FSLGGRYDRKNFTTSEELVRSGRYADRSWNSGIVFKPNRHFSVSYRASSGFRTPSFQE
LFGIDIYHDYPKGWQRPALKSEKAANREIGLQWKGDFGFLEISSFRNRYTDMIAVADQ
KTKLPDSAGRLTEIDIRDYYNAQNMSLQGINILGKIDWNGVYGKLPEGLYTTLAYNRI
KPKSVSNRPDLSLRSYALDAVQPSRYVLGFGYDQPEGKWGANIMLTYSKGKNPDELAY
LAGDQKRYSAGRVTSSWKTADVSAYLNLKKRLTLRAAIYNIGNYRYVTWESLRQTAES
                                          TANRHGGDSNYGRYAAPGRNFSLALEMKF”
[SEQ ID NO:80]
      1 ctcgggataa cggcatcaat ctttcgggaa atggttcgac taatcctcaa agtttcaaag
    61 ccgacaatct tcttgtaacg ggcggctttt acggcccgca ggcggcggaa ttgggcggca
  121 ctattttcaa taaggatggg aaatctcttg gtataactga agatattgaa aatgaagttg
  181 aaaatgaagc tgatgttggc gaacagttag aacctgaagt taaaccccaa ttcggcgtgg
  241 tattcggtgc gaagaaagat aataaagagg tggaaaaatg aataagaaac acggttttcc
  301 gctgactttg acggcgttgg ccattgcaac cgcttttccg gcttatgctg cccaagcggg
  361 ggcggcggca cttgatgcgg cgcaaagtca atcattgaaa gaggttaccg tccgtgccgc
  421 caaagtggga cggcgatcga aagaggcgac aggtttgggc aaaatcgtca aaacgtcgga
  481 aacgttgaac aaagaacagg tactcggtat ccgcgacctg acgcgctacg atccgggcgt
  541 ggcggttgtc gaacagggca acggcgcgag cggcggctac tcgatacgcg gcgtagataa
  601 aaaccgtgtg gcggtttcgg ttgacggcgt tgcccaaata caggcgttta ccgtgcaggg
  661 atcgttgagc ggatacggcg gacgcggcgg cagcggcgca atcaacgaaa tcgaatatga
  721 aaacatcagc acggtggaaa tcgacaaagg cgccggttcg tccgatcacg gcagcggcgc
  781 actcggcggc gcggtcgcct tccgcaccaa agaggcggca gacctgattt cagacggcaa
  841 aagctggggg atacaggcaa aaaccgctta cggcagtaaa aaccgccaat ttatgaagtc
  901 gctcggcgcg gggttcagca aagacggttg ggaagggctg ctaatccgaa ccgaacgcca
  961 agggcgggaa acgcgcccgc acggcgatat tgcggacggg gtggaatacg gcatagaccg
1021 tttggacgcg ttccgccaga catacgatat taaacgcaag acaacagagc catttttctt
1081 agtagagggc gagaatacac tcaagcccgt ggcaaaattg gcgggctacg ggatatattt
1141 gaaccgccag ctcaaccgct gggtaaaaga acgtattgaa caaaatcagc ctttaagtgc
1201 tgaagaagag gcgcaggtgc gggaggcgca ggcgcgccac gaaaacctgt ccgcccaagc
1261 ctacacgggc ggcggcagga tattgcccga tccgatggat taccgcagcg gctcttggct
1321 tgccaagctg ggctaccgct tcggcggcag gcattatgtc ggcggcgtgt ttgaggatac
1381 caaacagcgt tacgacatcc gcgatatgac ggaaaaacag tattacggta cggacgaggc
1441 ggaaaagttt agagacaaga gcggggtgta cgacggcgac gatttccgcg acggcttgta
1501 ttttgtgccg aatatagaag agtggaaggg cgataaaaat ttggtcaagg gcataggttt
1561 gaaatattcc cgcaccaaat ttattgacga acatcaccgc cgccgccgta tgggtttgct
1621 gtaccgttat gaaaatgaga aatactcgga caactgggcg gataaggcgg tgttgtcgtt
1681 tgacaaacag ggcgtggcaa ccgacaacaa cacgctgaag ctgaattgcg ccgtgtatcc
1741 tgccgtggac aaatcctgcc gcgcgtcggc ggacaaaccg tattcctacg acagcagcga
1801 ccgtttccac taccgcgaac agcacaatgt tttgaatgcc tcgtttgaga agtcgctgaa
1861 aaacaaatgg acgaaacacc atctgacttt gggcttcggt tacgatgctt ccaaagcagt
1921 atcccgccca gaacagcttt cccacaatgc ggcaaggatt tcggaatcca cgggattcga
1981 tgaaaagaat caagataagt accgtttggg taagcccgaa gtcgtcgaag ggtcggtctg
2041 cggctatatc gaaaccctgc gttcccgcaa atgcgtgcca agaaaaatca acggcagcaa
2101 tatccacatt tctttgaacg accgtttttc aatcggcaaa tattttgatt tcagcttggg
2161 cggcaggtac gaccggaaaa acttcaccac gtcggaagaa ctcgtccgca gcgggcggta
2221 tgccgaccgt tcgtggaaca gcggcatcgt gttcaaaccg aaccggcatt tttccgtgtc
2281 ttaccgcgcc tccagcggct tcagaacgcc ttccttccaa gaacttttcg ggatagacat
2341 ttatcacgat tatccgaaag gctggcagcg tcccgccctg aaatcggaaa aggcagccaa
2401 ccgggaaatc ggtttgcagt ggaagggcga tttcggcttt ttggaaatca gcagtttccg
2461 caaccgttat accgatatga ttgccgttgc cgatcaaaaa accaaattgc cggattcagc
2521 aggacgattg acagagattg atatacgcga ttattacaat gcccaaaata tgtcgcttca
2581 aggcatcaac atcttgggga aaatcgactg gaacggcgta tacggcaaac tgcccgaagg
2641 cctgtacacc acattggcgt acaaccgtat caaaccgaaa tcggtatcca accggccgga
2701 cttgtccctc cgcagctatg ctttggatgc ggtacagccg tcgcgttatg ttttggggtt
2761 cggatacgac cagcccgagg ggaaatgggg cgcaaacatt atgctgacct attccaaagg
2821 gaaaaaccct gacgagcttg cttatctggc aggcgatcaa aaacgatatt cggcaggaag
2881 ggttacgtct tcttggaaaa cggcagatgt ttccgcttat ctgaatctga aaaaacggct
2941 gaccttgagg gcggctatct acaatatcgg caactaccgc tacgttactt gggaatcctt
3001 gcgccagact gcggaaagca cggcaaaccg gcacggcggc gacagcaact atggaaggta
3061 tgccgcaccg ggcaggaact tcagcctcgc gctcgaaatg aagttttaaa ggaaatgccg
3121 tctggaagct tgatctgcac cccaaaagtc ggactaaacc gccaactgat taaggtgcag
3181 gtttttttga ttcaatataa acaagatttc cgccgtcatt cccgcgcagg cgggaatccg
3241 gacattcaat gctaaggcaa tttatcggaa atgactgaaa ctcaaaaaac cggattccca
//
[SEQ ID NO:81]
Neisseria gonorrhoeae lactoferrin binding protein B precursor.
Source: Neisseria gonorrhoeae”/strain=“FA19”
ACCESSION AAD08809
PID g4106393
VERSION AAD08809.1 GI:4106393
/gene=“lbpB” coding sequence: 1..728, “AF072890.1:310..2496”
      1 mrklnyygia llplmlascg gnfgvqpvve stptaypvtf kskdvptspp paepsvettp
    61 vnrpavgaam rllrrntafh redgtaipds kqaeeklsfk egdvlflygs kgnklqqlks
  121 eihkrdsdve irtsekenkk ygyefvdagy vytkngkdei eqnsggkrft hrfgydgfvy
  181 ysgerpsqsl psagtvkyfg nwqymtdakr hrtgkavasd dlgyitfygn digatsyaak
  241 daddrekhpa eytvdfdkki lkgeliknqy vqkkndpkkp ltiynitadl ngnrftgsak
  301 vntevktrha dkeylffhtd adqrleggff gdngeelagr fisndngvfg vfagkqktna
  361 sgtnpampfg khtkildslk isvdeatden prpfevstmp dfghpdkllv egreiplvsk
  421 ektidladgr kmtvsaccdf ltyvklgrik terpavkpka qdeedsginn geesedeeei
  481 aeesedevse ddngededei veeeadeaee ieeeaeeeep eeespeegng vsdgippape
  541 alkgrdidlf lkgirtaead ipktgtahyt gtwearigep iqwdnkadka akaefdvdfg
  601 nksisgtlte qngvepafri engviegngf hptartrdng inlsgngstn pqsfkadnll
  661 vtggfygpqa aelggtifnk dgkslgited ienevenead vgeqlepevk pqfgvvfgak
  721 kdnkevek
[SEQ ID NO:82]
Neisseria gonorrhoeae transferrin-binding protein A (tbpA) gene, complete
cds.
ACCESSION AF241227
VERSION AF241227.1 GI:9719361
source :Neisseria gonorrhoeae/strain=“Pgh3-2”
gene “tbpA” coding sequence: 223..2946
/protein_id=“AAF97766.1”
/db_xref=“GI:9719362”
/translation=
“MQQQHLFRFNILCLSLMTALPAYAENVQAGQAQEKQLDTIQVKA
KKQKTRRDNEVTGLGKLVKTADTLSKEQVLDIRDLTRYDPGIAVVEQGRGASSGYSIR
GMDKNRVSLTVDGLAQIQSYTAQAALGGTRTAGSSGAINEIEYENVKAVEISKGSNSV
EQGSGALAGSVAFQTKTADDVIGEGRQWGIQSKTAYSGKNRGLTQSIALAGRIGGAEA
LLIRTGRHAGEIRAHEAAGRGVQSFNRLAPVEDGSDYAYFVVEGECPDGYAACKDKPK
KDVVGEDKRQTVSTRDYTGPNRFLADPLSYESRSWLFRPGFRFENKRHYIGGILERTQ
QTFDTRDMTVPAFLTKAVFDANSKQAGSLRGNGKYAGNHKYGGLFTNGENNAPVGAEY
GTGVFYDETHTKSRYGLEYVYTNADKDTWADYARLSYDRQGIGLDNHFQQTHCSADGS
DKYCRPSADKPFSYYKSDRVIYGESHRLLQAAFKKSFDTAKIRHNLSVNLGYDRFGSN
LRHQDYYYQSANRAYSLKTPPQNNGKKTSPYWVSIGRGNVVTGQICRSGNNTYTDCTP
RSINGKSYYAAVRDNVRLGRWADVGAGLRYDYRSTHSDDGSVSTGTHRTLSWNTGIVL
KPADWLDLTYRTSTGFRLPSFAEMYGWRSGDKIKAVKIDPEKSFNKEAGIVFKGDFGN
LEASWFDNAYRDLIVRGYEAEIKNGKEQAKGAPAYLNAQSARITGINILGKIDWNGVW
DKLPEGWYSTFAYNRVRVRDIKKRADRTDIQSHLFDAIQPSRYVVGSGYDQPEGKWGV
NGMLTYSKAKEITELLGSRALLNGNSRNTKATARRTRPWYIVDVSGYYTVKKHFTLRA
                                          GVYNLLNHRYVTWENVRQTAAGAVNQHKNVGVYNRYAAPGRNYTFSLEMKF”
[SEQ ID NO:83]
      1 cgaagagttg ggcggatggt ttgcctatcc gggcaatgaa caaacgaaaa atgcgcaagc
    61 ttcatccggc aatggaaatt cagcaggcag cgcgaccgtg gtattcggtg cgaaacgcca
  121 aaagcttgtg caataagcac ggctgccgaa caatcgagaa taaggcttca gacggcatcg
  181 ttcctgccga ttccgtctga aagcgaagat tagggaaaca ctatgcaaca gcaacatttg
  241 ttccgattca atattttatg cctgtcttta atgactgcgc tgcccgctta tgcagaaaat
  301 gtgcaagccg gacaagcaca ggaaaaacag ttggacacca tacaggtaaa agccaaaaaa
  361 cagaaaaccc gccgcgataa cgaagtaacc ggtttgggca aattggtcaa aaccgccgac
  421 acactcagca aagaacaggt actcgacatc cgcgacctga cgcgttacga ccccggcatc
  481 gccgtcgtcg aacaggggcg cggcgcaagc tcgggctact cgatacgcgg tatggacaaa
  541 aaccgcgtct ccttgacggt ggacggcttg gcgcaaatac agtcctacac cgcgcaggcg
  601 gcattgggcg ggacgaggac ggcgggcagc agcggcgcaa tcaatgaaat cgagtatgaa
  661 aacgttaagg ctgtcgaaat cagcaaaggc tcaaactcgg tcgaacaagg cagcggcgca
  721 ttggcgggtt cggtcgcatt tcaaaccaaa accgcagacg atgttatcgg ggaaggcagg
  781 cagtggggca ttcagagtaa aaccgcctat tccggcaaaa accgggggct tacccaatcc
  841 atcgcgctgg cggggcgcat cggcggtgcg gaggctttgc tgatccgcac cggccggcac
  901 gcgggggaaa tccgcgccca cgaagccgcc ggacgcggcg ttcagagctt taacaggctg
  961 gcgccggttg aagacggcag tgactatgcc tattttgtgg tcgaaggaga atgccctgat
1021 ggatatgcgg cttgtaaaga caaaccgaaa aaagatgttg tcggcgaaga caaacgtcaa
1081 acggtttcca cccgagacta cacgggcccc aaccgcttcc ttgccgatcc gctttcatac
1141 gaaagccggt cgtggctgtt ccgcccgggt tttcgttttg agaataagcg gcactacatc
1201 ggcggcatac tcgaacgcac gcaacaaact ttcgacacgc gcgatatgac ggttccggca
1261 ttcctgacca aggcggtttt tgatgcaaat tcaaaacagg cgggttcttt gcgcggcaac
1321 ggcaaatacg cgggcaacca caaatacggc gggctgttta ccaacggcga aaacaatgcg
1381 ccggtgggcg cggaatacgg tacgggcgtg ttttacgacg agacgcacac caaaagccgc
1441 tacggtttgg aatatgtcta taccaatgcc gataaagaca cttgggcgga ttatgcccgc
1501 ctctcttacg accggcaggg catcggtttg gacaaccatt ttcagcagac gcactgttct
1561 gccgacggtt cggacaaata ttgccgcccg agtgccgaca agccgttttc ctattacaaa
1621 tccgaccgcg tgatttacgg ggaaagccac aggctcttgc aggcggcatt caaaaaatcc
1681 ttcgataccg ccaaaatccg ccacaacctg agcgtgaatc tcggttacga ccgcttcggc
1741 tctaatctgc gccatcagga ttattattat caaagtgcca accgcgccta ttcgttgaaa
1801 acgccccctc aaaacaacgg caaaaaaacc agcccctatt gggtcagcat aggcagggga
1861 aatgtcgtta cggggcaaat ctgccgctcg ggcaacaata cttatacgga ctgcacgccg
1921 cgcagcatca acggcaaaag ctattacgcg gcggtccggg acaatgtccg tttgggcagg
1981 tgggcggatg tcggcgcggg cttgcgctac gactaccgca gcacgcattc ggacgacggc
2041 agcgtttcca ccggcacgca ccgcaccctg tcctggaaca ccggcatcgt cctcaaacct
2101 gccgactggc tggatttgac ttaccgcact tcaaccggct tccgcctgcc ctcgtttgcg
2161 gaaatgtacg gctggcggtc gggcgataaa ataaaagccg tcaaaatcga tccggaaaaa
2221 tcgttcaaca aagaagccgg catcgtgttt aaaggcgatt tcggcaactt ggaggcaagt
2281 tggttcgaca atgcctaccg cgatttgatt gtccggggtt atgaagcgga aattaaaaac
2341 ggcaaagaac aagccaaagg cgccccggct tacctcaatg cccaaagcgc gcggattacc
2401 ggcatcaata ttttgggcaa aatcgattgg aacggcgtat gggataaatt gcccgaaggt
2461 tggtattcta catttgccta taatcgtgtc cgtgtccgcg acatcaaaaa acgcgcagac
2521 cgcaccgata ttcaatcaca cctgtttgat gccatccaac cctcgcgcta tgtcgtcggc
2581 tcgggctatg accaaccgga aggcaaatgg ggcgtgaacg gtatgctgac ttattccaaa
2641 gccaaggaaa tcacagagtt gttgggcagc cgggctttgc tcaacggcaa cagccgcaat
2701 acaaaagcca ccgcgcgccg tacccgccct tggtatattg tggatgtgtc cggttattac
2761 acggttaaaa aacacttcac cctccgtgcg ggcgtgtaca acctcctcaa ccaccgctat
2821 gttacttggg aaaatgtgcg gcaaactgcc gccggcgcag tcaaccaaca caaaaatgtc
2881 ggcgtttaca accgatatgc cgcccccggc cgcaactaca catttagctt ggaaatgaag
2941 ttctaaacgt ccgaacgccg caaatgccgt ctgaaaggct tcagacggcg ttttttacac
3001 aatccccacc gtttcccatc cttcccgata caccg
[SEQ ID NO:84]
Neisseria gonorrhoeae strain UU1008 transferrin-binding protein 2
(tbpB) gene, complete cds.
ACCESSION U65222
VERSION U65222.1 GI:2286066
Source: Neisseria gonorrhoeae/strain=“UU1008”
gene=“tbpB” coding sequence: 1..2052
/protein_id=“AAB64243.1”
/db_xref=“GI:2286067”
/translation=
“MNNPLVNQAAMVLPVFLLSACLGGGGSFDLDSVDTEAPRAAPKY
QDVPSKKPEARKDQGGYGFAMRFKRRNWYRAANENEVKLKESDWEQTDDDEIKNPFKQ
KNIINALPGNEGELLQDSSQQGKGTSKVRDHHDFKYVWSGFFYKRIKITTKKDESHKI
IEARSGPDGYIFYKGRNPSRKLPVSGEVTYKGTWDFLTDVKANQKFTDLGNASTKSGD
QYSAFSGELDYIVKKEEDKKEKHKGLGLTTEITVDFEKKTLIGKLIKNNMLINNNTKP
TTQYYSLEAQVTGNRFSGKAMATEKGENKQHPFVSDSSSLSGGFFGPQGEELGFRFLS
DDGKVAVVGSAKTKDETASSGGTSGGASVSTSNGAAGTSSENKLTTVLDAVELTPNGK
KIKDLDNFSNAAQLVVDGIMIPLLPKDSESGGSHTDKGENGKTAFIYETTYTPESDKE
DAQTGMATNGVQTVSNTAGGTSGKTKTHYEVQACCSNLNYLKYGLLTRKNSSQADAKM
GQVEQSMFLQGERTDEKEIPQEQNVVYSGTWYGHIATNGTSWTREASDQENGNRANFD
VNFKDKRITGTLTAENRSEATFTIEAMIEGNGFKGTAKTGNGGFAPDQNSSTGTHKVH
ITNAAVQGGFYGPNAEELGGWFAYPGNGQTKNAQTSSGNGNSAGSATVVFGAKRQQLV
                                          K”
[SEQ ID NO:85]
      1 atgaacaatc cattggtgaa tcaggctgct atggtgctgc ccgtgttttt gttgagcgct
    61 tgtctgggcg gaggcggcag tttcgatctt gattctgtcg ataccgaagc cccgcgtgcc
  121 gcgccaaagt atcaagatgt tccttccaaa aaaccggaag cccgaaaaga ccaaggcgga
  181 tacggtttcg cgatgcgctt caagcggcgg aattggtatc gggcggcaaa cgaaaacgag
  241 gttaaactga aagagagtga ttgggaacaa acggatgatg atgagatcaa aaaccctttc
  301 aaacaaaaaa atattattaa tgccttacct ggaaatgagg gggaattatt gcaagattcc
  361 agtcaacaag gtaagggtac atctaaggtt agggaccatc acgattttaa atacgtatgg
  421 tcgggttttt tttataaacg gattaagatt acaactaaaa aagacgaatc tcataaaata
  481 atcgaagcca gaagcggtcc tgacggttat attttttata aaggcagaaa tccctcgaga
  541 aaacttcctg tttcagggga ggttacgtac aaaggtactt gggatttttt aactgatgtg
  601 aaagcaaatc agaaatttac agatttagga aatgcttcta cgaaatccgg agaccaatat
  661 agtgcttttt ccggggagtt ggattatata gtcaaaaaag aggaggataa aaaagaaaag
  721 cacaaaggtt tgggattaac aacggaaata acggttgatt ttgagaaaaa aaccctgatc
  781 ggaaaattaa ttaaaaacaa catgttaatc aataataaca ctaaacccac cacccaatat
  841 tacagccttg aggctcaagt aacaggcaac cgcttcagcg gcaaggcgat ggcaaccgaa
  901 aaaggcgaaa acaaacaaca tccctttgtt tccgactcgt cttctctgag cggcggcttt
  961 ttcggcccgc agggtgagga attgggtttc cgctttttga gcgacgatgg aaaagttgcc
1021 gttgtcggca gcgcgaaaac caaagacgaa accgcaagca gtggcggcac ttcgggcggt
1081 gcaagcgttt ccacatcaaa cggtgcggca ggcacgtcgt ctgaaaacaa gctgaccacg
1141 gttttggatg cggttgaatt gacaccaaac ggcaagaaaa tcaaagatct cgacaacttc
1201 agcaacgccg cccaactggt tgtcgacggc attatgattc cgctcctgcc caaggattcc
1261 gaaagcgggg gcagtcatac agataaaggt gaaaacggca aaacagcctt tatctacgaa
1321 acaacctaca cgccggaaag tgataaagaa gacgctcaaa caggtatggc gaccaatggc
1381 gtgcaaaccg tttcaaatac ggcaggcggc acaagtggca aaacaaaaac ccattatgaa
1441 gtccaagcct gctgttccaa cctcaattat ctgaaatacg ggttgctgac gcgtaaaaac
1501 agtagtcaag ctgacgctaa aatgggacaa gttgaacaaa gtatgttcct ccaaggcgag
1561 cgcaccgatg aaaaagaaat tccacaagaa caaaatgtcg tttattcagg cacttggtac
1621 gggcatattg ccaccaacgg cacaagttgg acccgcgaag cctccgatca ggaaaatggt
1681 aatcgggcaa attttgacgt gaatttcaaa gacaaaagaa ttaccggcac gttaaccgct
1741 gaaaacaggt cggaggcaac ctttaccatt gaagccatga ttgagggcaa cggctttaaa
1801 ggtacggcga aaaccggtaa tggcggcttt gcgccggatc aaaacagcag caccggtaca
1861 cataaagtgc acatcacaaa tgccgcggtg cagggcggtt tttacgggcc taacgccgaa
1921 gagttgggcg gttggtttgc ctatccgggc aatggacaaa cgaaaaatgc gcaaacttca
1981 tccggcaatg gaaattcagc aggcagcgcg accgtggtat tcggtgcgaa acgccaacag
2041 cttgtgaaat aa
[SEQ ID NO:86]
Neisseria gonorrhoeae pilus biogenesis gene cluster, pilO, pilP and
pilQ genes, complete cds.
ACCESSION U40596
VERSION U40596.1 GI:1173872
source : Neisseria gonorrhoeae/strain=“MS11”
gene=“pilO” coding sequence 22..669
/protein_id=“AAC43601.1”
/db_xref=“GI:1173873”
                                          /translation=
“MASKSSKTNLDLNNLHLLNLPARLFIALLVVAAVLGLGYAGLFK
SQMESLEEYEAKETELKNTYKQKSIDAASLNNLRDELASIRSAFDIMLKQLPTDAEIP
NLVQELHQAGSSNGLRLDSVMPQPPVDDGPIKKLPYSISITGNYEQISQFTRDVGSLS
RIITLESLKIAQSPENGGNPDGKSSILNLSAIATTYQAKSIEELAAEAAQNAEQK”
[SEQ ID NO:87]
gene=“pilP” coding sequence 687..1229
/protein_id=“AAC43602.1”
/db_xref=“GI:1173874”
/translation=
“MKHYALLISFLALSACSQSSEDLNEWMAQTRREAKAEIIPFQAP
TLPVAPVYSPPQLTGPNAFDFRRMETAKKGENAPDTKRIKETLEKFSLENMRYVGILK
SGQKVSGFIEAEGYVYTVGVGNYLGQNYGRIESITDDSIILNELIEDSTGNWVSRKAE
                                          LLLNSSDKNTEQAAQPEEQN”
[SEQ ID NO:88]
gene=“pilQ” coding sequence 1248..3410
/protein_id=“AAC43603.1”
/db_xref=“GI:1173875”
/translation=
“MNTKLTKIISGLFVATAAFQTASAGNITDIKVSSLPNKQKIVKV
SFDKEIVNPTGFVTSSPARIALDFEQTGISMDQQVLEYADPLLSKISAAQNSSRARLV
LNLNKPGQYNTEVRGNKVWIFINESDDTVSAPARPAVKAAPAAPAKQQAAAPFTESVV
SVSAPFSPAKQQAAASAKQQAATPAKQTNIDFRKDGKNAGIIELAALGFAGQPDISQQ
HDHIIVTLKNHTLPTALQRSLDVADFKTPVQKVTLKRLNNDTQLIITTTGNWELVNKS
AAPGYFTFQVLPKKQNLESGGVNNAPKTFTGRKISLDFQDVEIRTILQILAKESGMNI
VASDSVSGKMTLSLKDVPWDQALDLVMQARNLDMRQQGNIVNMAPRRAACQRQSLLTS
GKRHCRSGRAVFPKLPIEIQKCGRIPQHPALDNADTTGNRNTLVSGRGSVLIDPATNT
LIVTDTRSVIEKFRKLIDELDVPAQQVMIEARIVEAADGFSRDLGVKFGATGRKKLKN
ETSAFGWGVNSGFGGGDKWEAKPKSTCRLPCRKQHFAGARDFSGALNLELSASESLSK
TKTLANPRVLTQNRKEAKIESGYEIPFTVTTRSGGGNSTNTELKKAVLGLTVTANITP
DGQIIMTVKINKDSPRQCASGNNTILCISTKSLNTQAMVENGGTLIVGGIYEENNGNT
                                          LTKVPLLATSPLSATSLKHSGKNRPPRTADFQLPPREL”
[SEQ ID NO:89]
      1 aaacgcacag gaggaaactg aatggcttct aaatcatcta aaaccaactt ggatctcaac
    61 aaccttcacc tgctcaacct tcctgccagg ctttttatcg ccctgctggt cgttgccgcc
  121 gtgctggggc tcggttatgc cggattgttc aaaagccaga tggaatccct tgaggaatat
  181 gaagcaaaag aaaccgaact gaaaaacacc tacaaacaga aaagtatcga cgcggccagc
  241 ctgaacaacc ttagggacga acttgcctca atccgctctg ccttcgatat catgttgaaa
  301 cagctgccga cagatgcaga aattcccaat ttggttcaag agcttcatca ggcgggttcg
  361 agcaacggtc tgcgcttgga cagcgttatg ccccaacctc ccgtagatga cggtcccatc
  421 aaaaaattac cctattccat ttccattacc ggaaattacg aacagatcag ccaatttacc
  481 cgcgatgtcg gcagtctctc ccgaatcatt acccttgagt cgctgaaaat cgcccaatct
  541 ccggaaaacg gcggcaatcc tgacggcaag agcagtatcc tgaacctcag cgccattgcc
  601 accacctacc aagcaaaatc catagaagag cttgccgcag aagcggcaca aaatgccgag
  661 caaaaataac ttacgttagg gaaaccatga aacactatgc cttactcatc agctttctgg
  721 ctctctccgc gtgttcccaa agttctgaag acctaaacga atggatggca caaacgcgac
  781 gcgaagccaa agcagaaatc atacctttcc aagcacctac cctgccggtt gcgccggtat
  841 acagcccgcc gcagcttaca gggccgaacg cattcgactt ccgccgcatg gaaaccgcca
  901 aaaaagggga aaatgccccc gacaccaagc gtattaaaga aacgctggaa aaattcagtt
  961 tggaaaatat gcgttatgtc ggcattttga agtccggaca gaaagtctcc ggcttcatcg
1021 aggctgaagg ttatgtctac actgtcggtg tcggcaacta tttgggacaa aactacggta
1081 gaatcgaaag cattaccgac gacagcatca tcctgaacga gctgatagaa gacagcacgg
1141 gcaactgggt ttcccgtaaa gcagaactgc tgttgaattc ttccgacaaa aacaccgaac
1201 aagcggcaca gcctgaggaa caaaattaag aagaggatta ctccattatg aataccaaac
1261 tgacaaaaat catttccggt ctctttgtcg caaccgccgc ctttcagacg gcatcggcag
1321 gaaacattac agacatcaaa gtttcctccc tgcccaacaa acagaaaatc gtcaaagtca
1381 gctttgacaa agagattgtc aacccgaccg gcttcgtaac ctcctcaccg gcccgcatcg
1441 ccttggactt tgaacaaacc ggcatttcca tggatcaaca ggtactcgaa tatgccgatc
1501 ctctgttgag caaaatcagt gccgcacaaa acagcagccg tgcgcgtctg gttctgaatt
1561 tgaacaaacc gggccaatac aataccgaag tacgcgggaa caaagtttgg atattcatta
1621 acgaatcgga cgataccgtg tccgcccccg cccgcccagc cgtaaaagcc gcgcctgccg
1681 caccggcaaa acaacaggct gccgcaccgt ttaccgagtc cgtagtatcc gtatccgcac
1741 cgttcagccc ggcaaaacaa caggcagcgg catcggcaaa acaacaggcg gcgacaccgg
1801 caaaacaaac caatatcgat ttccgcaaag acggcaaaaa tgccggcatt atcgaattgg
1861 cggcattggg ctttgccggg cagcccgaca tcagccaaca gcacgaccac atcatcgtta
1921 cgctgaaaaa ccataccctg ccgaccgcgc tccaacgcag tttggatgtg gcagacttca
1981 aaacaccggt tcaaaaggtt acgctgaaac gcctcaataa cgacacccag ctgattatca
2041 caacaaccgg caactgggaa ctcgtcaaca aatccgccgc gcccggatac tttaccttcc
2101 aagtcctgcc gaaaaaacaa aacctcgagt caggcggcgt gaacaatgcg cccaaaacct
2161 tcacaggccg gaaaatctcc cttgacttcc aagatgtcga aatccgcacc atcctgcaga
2221 ttttggcaaa agaatccggg atgaacattg ttgccagcga ctccgtcagc ggcaaaatga
2281 ccctctccct caaagacgta ccttgggatc aggctttgga tttggttatg caggcgcgca
2341 acctcgatat gcgccagcaa gggaacatcg tcaacatggc cccgcgacga gctgcttgcc
2401 aaagacaaag ccttcttaca agcggaaaaa gacattgccg atctgggcgc gctgtattcc
2461 caaaacttcc aattgaaata caaaaatgtg gaagaattcc gcagcatcct gctttggaca
2521 atgccgacac gaccggaaac cgcaacacgc ttgtcagcgg caggggcagc gtgctgatcg
2581 atcccgccac caacaccctg attgttaccg atacccgcag cgtcatcgaa aaattccgca
2641 aactgattga cgaattggac gtacccgcgc aacaagtgat gattgaggcg cgtatcgtcg
2701 aagcggcaga cggcttctcg cgcgatttgg gcgttaagtt cggcgcgaca ggcaggaaaa
2761 aactgaaaaa tgagacgagc gcattcggct ggggcgtgaa ctccggcttc gggggcggcg
2821 ataaatggga ggccaaacca aaatcaacct gccggttgcc gtgccgcaaa cagcatttcg
2881 ctggtgcgcg cgatttctcc ggcgcgttga atttggaatt gtccgcatcc gagtcgcttt
2941 caaaaaccaa aacgcttgcc aatccgcgcg tgctgaccca aaaccgcaaa gaggccaaaa
3001 tcgaatccgg ttacgaaatt ccttttaccg taactacacg ctcgggcggc ggcaactcta
3061 ccaacacgga actcaaaaaa gccgtcttgg ggctgaccgt tacggcgaac atcacgcccg
3121 acggacaaat catcatgacc gtcaaaatca acaaagactc gcctcgacaa tgtgcttcag
3181 gcaacaacac aatcctatgt atttcgacca aaagcctgaa tacgcaggct atggttgaaa
3241 acggcggcac tttgattgtc ggcggtattt atgaagaaaa caacggcaat acgctgacca
3301 aagtccccct gttggctaca tccccgttat cggcaacctc tttaaaacac tcgggaaaaa
3361 accgaccgcc gcgaactgct gattttcaat tacccccgag ggaattatag atacggcgca
3421 acagcctgcg ctattgatgc gtcaaaataa gggcatatgt tttacagcat atgccctttc
3481 tttatgcttt ttgccgcgac cgaaatgccg tcattcccgc gagcgaatcc aacttgtccg
3541 gtttcggttg tttttcgtct cgtaactttt gagccgtcat tcccgcgaaa tcggaaatcc
3601 agtccgttca gtttcggtca tttccgataa attcctgttg cttttcattt ctagattccc
3661 actttcgtgg aataacggcg gaagggataa atcctcgcaa tccaaagcct gctcatttcc
3721 acaaaaaaca gcaacccgaa acaccccgtc attcccgagc aggcggaatc tagaaccgca
3781 acgccaggaa tctgtcggat acggctgaaa ccgaacgact ggattcccg
[SEQ ID NO:90]
NspA
Neisseria gonorrhoeae outer membrane protein gene, complete cds
ACCESSION U52069
VERSION U52069.1 GI:1808968
source Neisseria gonorrhoeae/strain=“B2”
Gene “NspA” coding sequence : 141..665
/protein_id=“AAB41581.1”
/db_xref=“GI:1808969”
                                                                            /translation
“MKKALAALIALALPAAALAEGASGFYVQADAAHAKASSSLGSAK
GFSPRISAGYRINDLRFAVDYTRYKNYKAPSTDFKLYSIGASVIYDFDTQSPVKPYFG
ARLSLNRASAHLGGSDSFSKTSAGLGVLAGVSYAVTPNVDLDAGYRYNYVGKVNTVKN
                                          VRSGELSAGVRVKF”
[SEQ ID NO:91]
      1 cggcaaagca gccggatgcc gccgcgtatc ttgaggcatt gaaaatatta cgatgcaaaa
    61 agaaaatttc agtataatac ggcaggattc tttaacggat tattaacaat ttttctccct
  121 gaccataaag gaaccaaaat atgaaaaaag cacttgccgc actgattgcc ctcgcactcc
  181 cggccgccgc actggcggaa ggcgcatccg gcttttacgt ccaagccgat gccgcacacg
  241 ccaaagcctc aagctcttta ggttctgcca aaggcttcag cccgcgcatc tccgcaggct
  301 accgcatcaa cgacctccgc ttcgccgtcg attacacgcg ctacaaaaac tataaagccc
  361 catccaccga tttcaaactt tacagcatcg gcgcgtccgt catttacgac ttcgacaccc
  421 aatcgcccgt caaaccgtat ttcggcgcgc gcttgagcct caaccgcgct tccgcccact
  481 tgggcggcag cgacagcttc agcaaaacct ccgccggcct cggcgtattg gcgggcgtaa
  541 gctatgccgt taccccgaat gtcgatttgg atgccggcta ccgctacaac tacgtcggca
  601 aagtcaacac tgtcaaaaac gtccgttccg gcgaactgtc cgccggcgtg cgcgtcaaat
  661 tctgatatac gcgttattcc gcaaaccgcc gagccttcgg cggttttttg
[SEQ ID NO:92]
Neisseria gonorrhoeae outer membrane protein (omp85) gene, complete
cds.
ACCESSION U81959
VERSION U81959.1 GI:1766041
Source: Neisseria gonorrhoeae/strain=“FA19”
gene=“omp85” coding sequence 1..2379
/protein_id=“AAC17600.1”
/db_xref=“GI:1766042”
/translation
“MKLKQIASALMMLGISPLAFADFTIQDIRVEGLQRTEPSTVFNY
LPVKVGDTYNDTHGSAIIKSLYATGFFDDVRVETADGLLLLTVIVCPTIGSLNITGAK
MLQNDAIKKNLESFGLAQSQYFNQATLNQAVAGLKEEYLGRGKLNIQITPKVTKLARN
RVDIDITIDEGKSAKITDIEFEGNQVYSDRKLMRQMSLTEGGIWTWLTRSDRFDRQKF
AQDMEKVTDFYQNNGYFDFRILDTDIQTNEDKTRQTIKITVHEGGRFRWGKVSIEGDT
NEVPKAELEKLLTMKPGKWYERQQMTAVLGEIQNRMGSAGYAYSEISVQPLPNAGTKT
VDFVLHIEPGRKIYVNEIHITGNNKTRDEVVRRELRQMESAPYDTSKLQRSKERVELL
GYFDNVQFDAVPLAGTPDKVDLNMSLTERSTGSLDLSAGWVQDTGLVMSAGVSQDNLF
GTGKSAALRASRSKTTLNGSLSFTDPYFTADGVSLGYDIYGKAFDPRKASTSVKQYKT
TTAGGGVRMGIPVTEYDRVNFGLAAEHLTVNTYNKAPKRYADFIKQYGKTDGADGSFK
GLLYKGTVGWGRNKTDSALWPTRGYLTGVNAEIALPGSKLQYYSATHNQTWFFPLSKT
FTLMLGGEVGIAGGYGRTKEIPFFENFYGGGLGSVRGYESGTLGPKVYDEYGEKISYG
GNKKANVSAELLFPMPGAKDARTVRLSLFADAGSVWDGRTYTAAENGNNKSVYSENAH
KSTFTNELRYSAGGAVTWLSPLGPMKFIYAYPLKKKPEDEIQRFQFQLGTTF”
[SEQ ID NO:93]
      1 atgaaactga aacagattgc ctccgcactg atgatgttgg gcatatcgcc tttggcattt
    61 gccgacttca ccatccaaga catccgtgtc gaaggcttgc agcgtaccga gccgagcacc
  121 gtattcaact acctgcccgt caaagtcggc gacacctaca acgacacaca cggcagtgcc
  181 atcatcaaaa gcctgtacgc caccggtttc tttgacgacg tacgagtcga aactgcggac
  241 gggctgcttc tgctgaccgt tatcgtatgc cctaccatcg gctcgctcaa catcaccggc
  301 gccaaaatgc tgcagaacga cgccatcaag aaaaacctcg aatcgttcgg gctggcgcag
  361 tcgcaatact ttaatcaggc gacactcaac caggcagtcg ccggcctgaa agaagaatat
  421 ctcgggcgcg gcaaactcaa tatccaaatc acgcccaaag taaccaaact cgcccgcaac
  481 cgcgtcgaca tcgacatcac gattgacgag ggcaaatccg ccaaaatcac cgacatcgaa
  541 tttgaaggca accaagtcta ttccgaccgc aaactgatgc ggcagatgtc gctgaccgaa
  601 ggcggcattt ggacatggct gacacgaagc gaccggttcg accgccagaa attcgcccaa
  661 gacatggaaa aagtaaccga cttctaccag aacaacggct acttcgattt ccgtatcctc
  721 gataccgaca tccaaaccaa cgaagacaaa accaggcaga ccatcaaaat caccgtccac
  781 gaaggcggac gtttccgctg gggcaaagtg tcgattgaag gcgacaccaa cgaagtcccc
  841 aaggccgaac tggaaaaact gctgaccatg aagcccggca aatggtacga acgccagcag
  901 atgaccgccg ttttgggtga gattcagaac cgcatgggct cggcaggcta cgcatacagc
  961 gaaatcagcg tacagccgct gccgaacgcc ggaaccaaaa ccgtcgattt cgtcctgcac
1021 atcgaaccgg gcagaaaaat ctacgtcaac gaaatccaca tcaccggcaa caacaaaacc
1081 cgcgacgaag tcgtgcgccg cgaattgcgc caaatggaat ccgcgcctta cgacacctcc
1141 aagctgcaac gctccaaaga gcgcgtcgag cttttgggct acttcgacaa cgtacagttt
1201 gatgccgtcc cgcttgccgg tacgcccgac aaagtcgatt tgaacatgag cctgaccgaa
1261 cgttccaccg gctcgctcga cttgagcgcg ggctgggttc aggataccgg cttggtcatg
1321 tccgccggcg tatcgcagga caacctgttc ggtacgggca agtcggccgc cctgcgcgcc
1381 tcgcgaagca aaaccacgct caacggctcg ctgtcgttta ccgacccgta cttcacggca
1441 gacggggtca gcctgggcta cgatatttac ggaaaagcct tcgacccgcg caaagcatcg
1501 accagcgtca aacaatataa aaccaccacc gccggcggcg gcgtaaggat gggtatcccc
1561 gttaccgaat acgaccgcgt caatttcggg ctggcggcgg aacacctgac cgtcaacacc
1621 tacaacaaag cacccaaacg ctatgccgac tttatcaaac aatacggcaa aaccgacggc
1681 gcagacggca gcttcaaagg cctgctgtac aaaggcactg tcggctgggg gcgcaacaag
1741 accgacagcg ccttatggcc gacgcgcggc tacctgaccg gcgtaaatgc cgaaatcgcc
1801 ctgcccggca gcaaactgca atactactcc gccacccaca accaaacctg gttcttcccc
1861 ttaagcaaaa ccttcacgct gatgctcggc ggcgaagtcg gcattgcggg cggctacggc
1921 agaaccaaag aaatcccctt ctttgaaaac ttctacggcg gcggcctggg ttcggtgcgc
1981 ggctacgaaa gcggcacgct cggcccgaaa gtgtatgacg aatacggcga aaaaatcagc
2041 tacggcggca acaaaaaagc caacgtctcc gccgagctgc tcttcccgat gcccggtgcg
2101 aaagacgcac gcaccgtccg cctgagcctg tttgccgacg caggcagcgt gtgggacggc
2161 agaacctata ccgccgccga aaacggtaac aacaaatcgg tttactcgga aaacgcgcat
2221 aaatccacct ttaccaacga attgcgctat tccgccggcg gcgcggttac ctggctctcg
2281 cctttgggcc cgatgaaatt catctacgcc tacccgctga agaaaaaacc ggaagacgaa
2341 atccaacgct tccaattcca gctcggcacg acgttctaa
[SEQ ID NO:94]
PldA1 homolog in Neisseria gonorrhoeae
Source: U. of Oklahoma sequencing project
PldA1-like coding sequence:
>GONOCTG01_15 Continuation (15 of 22) of gonoctg01 from base 1400001
ATGAATACACGAAATATGCGCTATATTCTTTTGACAGGACTGTTGCCGACGGCATCCGCT
TTTGGAGAGACCGCGCTGCAATGCGCCGCTTTGACGGACAATGTTACGCGTTTGGCGTGT
TACGACAGGATTTTTGCGGCACAGCTTCCGTCTTCGGCAGGGCAGGAAGGGCAGGAGTCG
AAAGCCGTACTCAATCTGACGGAAACCGTCCGCAGCAGCTTGGATAAGGGCGAGGCGGTC
ATTGTTGTTGAAAAAGGCGGGGATGCGCTTCCTGCCGACAGTGCGGGCGAAACCGCCGAT
ATCTATACGCCTTTGAGCCTGATGTACGACTTGGACAAAAACGATTTGCGCGGGCTGTTG
GGCGTACGCGAACACAATCCGATGTACCTTATGCCGTTTTGGTATAACAATTCGCCCAAC
TATGCCCCGAGTTCGCCGACGCGCGGTACGACTGTACAGGAAAAATTCGGACAGCAGAAA
CGTGCGGAAACCAAATTGCAGGTTTCGTTCAAAAGCAAAATTGCCGAAAATTTGTTTAAA
ACCCGCGCGGATCTGTGGTTCGGCTACACCCAAAGATCCGATTGGCAGATTTACAACCAA
GGCAGGAAATCCGCGCCGTTCCGCAATACGGATTACAAACCTGAAATTTTCCTGACCCAG
CCTGTGAAGGCGGATTTGCCGTTCGGCGGCAGGCTGCGTATGCTCGGTGCGGGTTTTGTC
CACCAGTCCAACGGACAGAGCCGTCCCGAATCGCGTTCGTGGAACAGGATTTATGCCATG
GCAGGCATGGAATGGGGCAAATTGACGGTGATTCCGCGCGTGTGGGTGCGTGCGTTCGAT
CAGAGCGGCGATAAAAACGACAATCCCGATATTGCCGACTATATGGGGTATGGCGACGTG
AAGCTGCAGTACCGCCTGAACGACAGGCAGAATGTGTATTCCGTATTGCGCTACAACCCC
AAAACGGGCTACGGCGCGATTGAAGCCGCCTACACGTTTCCGATTAAGGGCAAACTCAAA
GGCGTGGTACGCGGATTCCACGGTTACGGCGAGAGCCTGATCGACTACAACCACAAGCAG
AACGGTATCGGTATCGGGTTGATGTTCAACGACTGGGACGGCATCTGA
[SEQ ID NO:95]
PldA1-like amino acid sequence
MNTRNMRYILLTGLLPTASAFGETALQCAALTDNVTRLACYDRIFAAQLPSSAGQEGQESKAVLNLTETVRSS
LDKGEAVIVVEKGGDALPADSAGETADIYTPLSLMYDLDKNDLRGLLGVREHNPMYLMPFWYNNSPNYAPSSP
TRGTTVQEKFGQQKRAETKLQVSFKSKIAENLFKTRADLWFGYTQRSDWQIYNQGRKSAPFRNTDYKPEIFLT
QPVKADLPFGGRLRMLGAGFVHQSNGQSRPESRSWNRIYAMAGMEWGKLTVIPRVWVRAFDQSGDKNDNPDIA
DYMGYGDVKLQYRLNDRQNVYSVLRYNPKTGYGAIEAAYTFPIKGKLKGVVRGFHGYGESLIDYNHKQNGIGI
GLMFNDWDGI.
[SEQ ID NO:96]
1000 base pairs upstream PldA1-like sequence (usuable for replacing the promoter for a
stronger sequence)
>GONOCTG01_15 Continuation (15 of 22) of gonoctg01 from base
1400001TTTTGGCTTCCAGCGTTTCGTTGTTTTCGTACAAGTCGTAAGTCAGCTTCAGATTGTTGG
CTTTTTTAAAGTCTTCGACCGTACTCTCGTCAACATAATTCGACCAGTTGTAGATGTTCA
GAGTATCGGTGGCAGCGGCTTCGGCATTGGCAGCAGGTGCGCTGCCTGCTTGAGGCTGCA
CGGCGTTTTTTTCGCTGCCGCCGCAGGCTGCCAGAGACAGCGCGGCCAAAACGGCTAATA
CGGATTTTTTCATACGGGCAGATTCCTGATGAAAGAGGTTGGAAAAAAAGAAAACCCCGC
GCCCCATAAACACCCCGGCGCAAGGTTTGGGTATTGTAAAGTAAATTTGTGCAAACTCAA
AGCGATATTGGCCTGATTTTCCTAAAAAATTACCCTGTTTCCAAAAAAGGGGGGGAAACG
GCCGCCCGATTTTGCCGTTTTTTTGCGCCGTCAGGGTGTCCGACGGGCGGATAGAGAAAA
AAGGCTTGCATATAATGTAAACCCCCTTTAAAATTGCGCGTTTACAGAATTTATTTTTCT
TTCAGGAGATTCCAATATGGCAAACAGCGCACAAGCACGCAAACGTGCCCGCCAGTCCGT
CAAACAACGCGCCCACAACGCTAGCCTGCGTACCGCATTCCGCACCGCAGTGAAAAAAGT
ATTGAAAGCAGTCGAAGCAGGCGATAAAGCTGCCGCACAAGCGGTTTACCAAGAGTCCGT
CAAAGTCATCGACCGCATCGCCGACAAAGGCGTGTTTCATAAAAACAAAGCGGCTCGCCA
CAAAAGCCGCCTGTCTGCAAAAGTAAAAGCACTGGCTTGATTTTTGCAAAACCGCCAAGG
CGGTTGATACGCGATAAGCGGAAAACCCTGAAGCCCGACGGTTTCGGGGTTTTCTGTATT
TCGGGGGTAAAGTTCGAAATGGCGGAAAGGGTGCGGTTTTTTATCCGAATCCGCTATAAA
ATGCCGTTTGAAAACCAATATGCCGACAATGGGGGCGGAG
Preferred gonococcal genes to downregulate via process a) include one or more of
the following:
[SEQ ID NO:97]
Neisseria gonorrhoeae iron-regulated outer membrane protein preFrpB
(frpB) gene, complete cds.
ACCESSION U13980
VERSION U13980.1 GI:833694
Source: Neisseria gonorrhoeae/strain=“FA19”
gene=“frpB” coding sequence: 318..2459
/protein_id=“AAC43332.1”
/db_xref=“GI:833695”
                                          /translation=
“MNAPFFRLSLLSLTLAAGFAHAAENNANVALDTVTVKGDRQGSK
IRTNIVTLQQKDESTATDMRELLKEEPSIDFGGGNGTSQFLTLRGMGQNSVDIKVDNA
YSDSQILYHQGRFIVDPALVKVVSVQKGAGSASAGIGATNGAIIAKTVDAQDLLKGLD
KNWGVRLNSGFAGNNGASYGASVFGKEGNFDGLFSYNRNDEKDYEAGKGFRNDNGGKT
VPYSALDKRSYLAKIGTTFGDGDHRIVLSHMKDQHRGIRTVREEFAVSEKNSRITIKR
QAPSYRETTQSNTNLAYTGKDLGFVEKLDANAYVLEKKRYSADDKDNGYAGNVKGPNH
TRIATRSMNFNFDSRLAEQTLLKYGINYRHQEIKPQAFLNSEFEIKDKEKATNEEKKK
NRENEKIAKAYRLTNPTKTDTGAYIEAIHEIDGFTLTGGLRYDRFKVKTHDGKTVSSS
SLNPSFGVIWQPREHWSFSASHNYAGRSPRLYDALQTHGKRGIISIADGTKAERARNT
EIGFNYNDGTFAANGSYFRQTIKDALANPQNRHDSVAVREAVNAGYIKNHGYELGASY
RTGGLTAKVGVSHSKPRFYDTHKDKLLSANPEFGAQVGRTWTASLAYRFKNPNLEIGW
RGRYVQKAVGSILAAGQKDRDGKLENVVRQGFGVNDVFANWKPLGKDTLNVNLSVNNV
                                          FDKFYYPHSQRWTNTLPGVGRDVRLGVNYKF”
[SEQ ID NO:98]
      1 aaaccggtac ggcgttgccc cgccttagct caaagagaac gattccctaa ggtgctgaag
    61 caccgagtga atcggttccg tactatttgt actgtctgcg gcttcgccgc cttgtcctga
  121 tttttgttag tccacatata catttccgac aaaacctgtc aacaaaaaac aacgcttcgc
  181 aaataaaaac gataatcagc tttacacaac ccccccccgc taatataaac aaaaataatt
  241 attattattt tttcttatcc tgccaaacct taacggtttg gcttaacttc ccttcataca
  301 ctcaaaagga cgaacaaatg aacgccccgt ttttccgcct cagcctgctc tcgctcacac
  361 ttgccgccgg ctttgcccac gcggcagaaa ataatgccaa tgtcgcattg gataccgtta
  421 ccgtaaaagg cgaccgccaa ggcagcaaaa tccgtaccaa catcgttacg cttcaacaaa
  481 aagacgaaag caccgcaacc gatatgcgcg aactcttaaa agaagagccc tccatcgatt
  541 tcggcggcgg caacggcacg tcccaattcc tgacgctgcg cggtatgggt cagaactctg
  601 tcgacatcaa ggtggacaac gcctattccg acagccaaat cctttaccac caaggcagat
  661 ttattgtcga tcccgctttg gttaaagtcg tttccgtaca gaaaggcgcg ggttccgcct
  721 ctgccggtat cggcgcgacc aacggcgcga tcatcgccaa aaccgtcgat gcccaagacc
  781 tgctcaaagg cttggataaa aactggggcg tgcgcctcaa cagcggcttt gccggcaaca
  841 acggcgcaag ctacggcgca agcgtattcg gaaaagaggg caacttcgac ggtttgttct
  901 cttacaaccg caacgatgaa aaagattacg aagccggcaa aggtttccgc aatgacaacg
  961 gcggcaaaac cgtaccgtac agcgcgctgg acaaacgcag ctacctcgcc aaaatcggaa
1021 caaccttcgg cgacggcgac caccgcatcg tgttgagcca tatgaaagac caacaccggg
1081 gcatccgcac tgtgcgtgaa gagtttgccg tcagcgaaaa aaattcacgg ataactatta
1141 aacgccaagc cccatcctac cgcgaaacca ctcaatccaa caccaacttg gcgtacaccg
1201 gcaaagattt gggctttgtc gaaaaactgg atgccaacgc ctatgtgttg gaaaagaaac
1261 gctattccgc cgatgacaaa gataacggct acgcaggcaa tgtaaaaggc cccaaccata
1321 cccgaatcgc cactcggagt atgaacttca acttcgacag ccgccttgcc gaacaaaccc
1381 tgttgaaata cggcatcaac taccgccatc aggaaatcaa accgcaagcg tttttgaact
1441 cggaatttga aataaaagat aaagaaaaag caactaatga agagaaaaag aagaaccgtg
1501 aaaatgaaaa aattgccaaa gcctaccgcc tgaccaaccc gaccaaaacc gataccggcg
1561 cgtatatcga agccattcac gagattgacg gctttaccct gaccggcggg ctgcgttacg
1621 accgcttcaa ggtgaaaacc cacgacggca aaaccgtttc aagcagcagc ctcaacccga
1681 gtttcggcgt gatttggcag ccgcgcgaac actggagctt cagcgcgagc cacaactacg
1741 ccggccgcag cccgcgcctg tatgacgctc tgcaaaccca cggcaagcgc ggcatcatct
1801 cgattgccga cggcacgaaa gccgaacgcg cgcgcaatac cgaaatcggc ttcaactaca
1861 acgacggcac gtttgccgca aacggcagct acttccggca gaccatcaaa gacgcgcttg
1921 ccaatccgca aaaccgccac gactccgtcg ccgtccgcga agccgtcaac gccggctaca
1981 tcaaaaacca cggttacgaa ttgggcgcgt cctaccgcac cggcggcctg accgccaaag
2041 tcggcgtaag ccacagcaaa ccgcgctttt acgatacgca caaagacaag ctgttgagcg
2101 cgaaccctga atttggcgca caagtcggcc gcacttggac ggcctccctt gcctaccgct
2161 tcaaaaaccc gaatctggaa atcggctggc gcggtcgtta tgttcaaaaa gccgtgggtt
2221 cgatattggc ggcaggtcaa aaagaccgcg acggcaaatt ggaaaacgtt gtacgccaag
2281 gtttcggtgt gaacgatgtc ttcgccaact ggaaaccgct gggcaaagac acgctcaatg
2341 ttaatctttc ggttaacaac gtgttcgaca agttctacta tccgcacagc caacgctgga
2401 ccaataccct gccgggcgtg ggacgtgatg tacgcctggg cgtgaactac aagttctaaa
2461 acgcacatcc cgaaaaaatg ccgtctgaaa gcctttcaga cggcatctgt cctgataatt
2521 tgatatatag tggattaaca aaaaccggta cggcgttgcc ccgccttagc tcaaagggaa
2581 cgattcccta aggtgctgaa
[SEQ ID NO:99]
N. gonorrhoeae structural gene for gonococcal protein III (PIII).
ACCESSION X05105
VERSION X05105.1 GI:44889
source:Neisseria gonorrhoeae/db_xref=“taxon:485”
Gene PIII coding sequence: 103..813
/protein_id=“CAA28752.1“
/db_xref=“GI:44890”
/db_xref=“SWISS-PROT:P07050”
/translation=
“MTKQLKLSALFVALLASGTAVAGEASVQGYTVSGQSNEIVRNNY
GECWKNAYFDKASQGRVECGDAVAVPEPEPAPVAVVEQAPQYVDETISLSAKTLFGFD
KDSLRAEAQDNLKVLAQRLSRTNVQSVRVEGHTDFMGSEKYNQALSERRAYVVANNLV
SNGVPASRISAVGLGESQAQMTQVCQAEVAKLGAKASKAKKREALIACIEPDRRVDVK
                                          IRSIVTRQVVPARNHHQH”
[SEQ ID NO:100]
      1 gaattcctat ccgatttgcc gccatgtttc tacagcggcc tgtatgttgg caattcagca
    61 gttgcttctg tatctgctgt acaaatctaa tgagggaata aaatgaccaa acagctgaaa
  121 ttaagcgcat tattcgttgc attgctcgct tccggcactg ctgttgcggg cgaggcgtcc
  181 gttcagggtt acaccgtaag cggccagtcg aacgaaatcg tacgcaacaa ctatggagaa
  241 tgctggaaaa acgcctactt tgataaagca agccaaggtc gcgtagaatg cggcgatgcg
  301 gttgccgtcc ccgagcccga acccgcgcct gtcgccgttg tggagcaggc tcctcaatat
  361 gttgatgaaa ccatttccct gtctgccaaa accctgttcg gtttcgataa ggattcattg
  421 cgcgccgaag ctcaagacaa cctgaaagta ttggcgcaac gcctgagtcg aaccaatgtc
  481 caatctgtcc gcgtcgaagg ccataccgac tttatgggtt ctgaaaaata caatcaggct
  541 ctgtccgaac gccgcgcata cgtagtggca aacaacctgg tcagcaacgg cgtacctgct
  601 tctagaattt ctgctgtcgg cttgggcgaa tctcaagcgc aaatgactca agtttgtcaa
  661 gccgaagttg ccaaactggg tgcgaaagcc tctaaagcca aaaaacgtga ggctctgatt
  721 gcatgtatcg aacctgaccg ccgcgtagat gtgaaaatcc gcagcatcgt aacccgtcag
  781 gttgtgccgg cacgcaatca tcaccaacac taaggctagg taatatcttg ccgatgcatg
  841 aggttagcgg attttgtacc gggtactgtt gcaatattcg tgaaacgtcg gccggtatcg
  901 atgatgtgaa acaaaccccg cttttgcggg gtttgttttt ttgggtggtt ttctgaaacg
  961 gctatcgtca gaatcggggt gcaggttcgg attcggattc agattcatgt ttgtgtccca
1021 ttgccgcgct ttatagtgga ttaacaaaaa tcaggacaag gcgacgaagc cgcagacagt
1081 acaatagtac ggcaaggcga ggcaacgccg taccggttta aatttaatcc actatatcgg
1141 ttgaaactct gattttaagg cggtaggatg tgggtttgcc catagcaagg gaatcctttc
1201 tgtatcaagc cccgaaaggg ataattcata caaattcacg cctttccccc tcattgggaa
1261 atggatggaa tcgtgcccga tgtgtgcggc actgtatgcc ggatatggtt ttatcatcat
1321 cccttttcgg ttgaaacccc gcggaattc
```

Preferred Pseudomonas aeruginosa bleb Preparations

One or more of the following genes (encoding protective antigens) are preferred for upregulation via process b): PcrV, OprF, OprI. They are also preferred as genes which may be heterologously introduced into other Gram-negative bacteria.

Preferred Moraxella catarrhalis bleb Preparations

One or more of the following genes (encoding protective antigens) are preferred for upregulation via process b): OMP106 (WO 97/41731 & WO 96/34960), HasR (PCT/EP99/03824), PilQ (PCT/EP99/03823), OMP85 (PCT/EP00/01468), lipo06 (GB 9917977.2), lipo10 (GB 9918208.1), lipo11 (GB 9918302.2), lipo18 (GB 9918038.2), P6 (PCT/EP99/03038), ompCD, CopB (Helminen Me., et al (1993) Infect. Immun. 61:2003-2010), D15 (PCT/EP99/03822), OmplA1 (PCT/EP99/06781), Hly3 (PCT/EP99/03257), LbpA and LbpB (WO 98/55606), ThpA and TbpB (WO 97/13785 & WO 97/32980), OmpE, UspA1 and UspA2 (WO 93/03761), FhaB (WO 99/58685) and Omp21. They are also preferred as genes which may be heterologously introduced into other Gram-negative bacteria.

One or more of the following genes are preferred for downregulation via process a): CopB, OMP106, OmpB1, ThpA, TbpB, LbpA, and LbpB.

One or more of the following genes are preferred for downregulation via process c): htrB, msbB and lpxK (most preferably msbB).

One or more of the following genes are preferred for upregulation via process d): pmrA, pmrB, pmrE, and pmrF.

Many of the above open reading frames and upstream regions are described in WO 01/09350 (incorporated by reference herein).

Preferred Haemophilus influenzae bleb Preparations

One or more of the following genes (encoding protective antigens) are preferred for upregulation via process b): D15 (WO 94/12641), P6 (EP 281673), TbpA, TbpB, P2, P5 (WO 94/26304), OMP26 (WO 97/01638), HMW1, HMW2, HMW3, HMW4, Hia, Hsf, Hap, Hin47, Iomp1457 (GB 0025493.8), YtfN (GB 0025488.8), VirG (GB 0026002.6), Iomp1681 (GB 0025998.6), OstA (GB 0025486.2) and Hif (all genes in this operon should be upregulated in order to upregulate pilin). They are also preferred as genes which may be heterologously introduced into other Gram-negative bacteria.

One or more of the following genes are preferred for downregulation via process a): P2, P5, Hif, IgA1-protease, HgpA, HgpB, HMW1, HMW2, Hxu, TbpA, and TbpB.

One or more of the following genes are preferred for downregulation via process c): htrB, msbB and lpxK (most preferably msbB).

One or more of the following genes are preferred for upregulation via process d): pmrA, pmrB, pmrE, and pmrF.

Many of the above open reading frames and upstream regions are described in WO 01/09350 (incorporated by reference herein).

Preparations of Membrane Vesicles (blebs) of the Invention

The manufacture of bleb preparations from any of the aforementioned modified strains may be achieved by harvesting blebs naturally shed by the bacteria, or by any of the methods well known to a skilled person (e.g. as disclosed in EP 301992, U.S. Pat. No. 5,597,572, EP 11243 or U.S. Pat. No. 4,271,147). For Neisseria, the method described in the Example below is preferably used

A preparation of membrane vesicles obtained from the bacterium of the invention is a further aspect of this invention. Preferably, the preparation of membrane vesicles is capable of being filtered through a 0.22 μm membrane.

A sterile (preferably homogeneous) preparation of membrane vesicles obtainable by passing the membrane vesicles from the bacterium of the invention through a 0.22 μm membrane is also envisaged.

Vaccine Formulations

A preferred embodiment of the invention is the formulation of the bleb preparations of the invention in a vaccine which may also comprise a pharmaceutically acceptable excipient.

Vaccine preparation is generally described in Vaccine Design (“The subunit and adjuvant approach” (eds Powell M. F. & Newman M. J.) (1995) Plenum Press New York).

The bleb preparations of the present invention may be adjuvanted in the vaccine formulation of the invention. Suitable adjuvants include an aluminium salt such as aluminum hydroxide gel (alum) or aluminium phosphate, but may also be a salt of calcium (particularly calcium carbonate), iron or zinc, or may be an insoluble suspension of acylated tyrosine, or acylated sugars, cationically or anionically derivatised polysaccharides, or polyphosphazenes.

Suitable Th1 adjuvant systems that may be used include, Monophosphoryl lipid A, particularly 3-de-O-acylated monophosphoryl lipid A, and a combination of monophosphoryl lipid A, preferably 3-de-O-acylated monophosphoryl lipid A (3D-MPL) together with an aluminium salt. An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO96/33739. A particularly potent adjuvant formulation involving QS21 3D-MPL and tocopherol in an oil in water emulsion is described in WO95/17210 and is a preferred formulation.

The vaccine may comprise a saponin, more preferably QS21. It may also comprise an oil in water emulsion and tocopherol. Unmethylated CpG containing oligo nucleotides (WO 96/02555) are also preferential inducers of a TH1 response and are suitable for use in the present invention.

The vaccine preparation of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Thus one aspect of the present invention is a method of immunizing a human host against a disease caused by infection of a gram-negative bacteria, which method comprises administering to the host an immunoprotective dose of the bleb preparation of the present invention.

The amount of antigen in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-100 μg of protein antigen, preferably 5-50 μg, and most typically in the range 5-25 μg.

An optimal amount for a particular vaccine can be ascertained by standard studies involving observation of appropriate immune responses in subjects. Following an initial vaccination, subjects may receive one or several booster immunisations adequately spaced.

Ghost or Killed Whole Cell Vaccines

The inventors envisage that the above modified bacterial strains may not only be useful in generating bleb preparations useful in vaccines—they may also be easily used to make ghost or killed whole cell preparations and vaccines (with identical advantages). Methods of making ghost preparations (empty cells with intact envelopes) from Gram-negative strains are well known in the art (see for example WO 92/01791). Methods of killing whole cells to make inactivated cell preparations for use in vaccines are also well known. The terms ‘bleb preparations’ and ‘bleb vaccines’ as well as the processes described throughout this document are therefore applicable to the terms ‘ghost preparation’ and ‘ghost vaccine’, and ‘killed whole cell preparation’ and ‘killed whole cell vaccine’, respectively, for the purposes of this invention.

### EXAMPLES

The examples below are carried out using standard techniques, which are well known and routine to those of skill in the art, except where otherwise described in detail. The examples are illustrative, but do not limit the invention.

### Example 1

### Previously Reported Examples

Examples describing: Construction of a Neisseiria meningitidis serogroup B strain lacking capsular polysaccharides; Construction of versatile gene delivery vectors (the pCMK series) targeting integration in the porA locus of Neisseiria meningitidis; Construction of a Neisseiria meningitidis serogroup B strain lacking both capsular polysaccharides and the major immunodominant antigen PorA; Up-regulation of the NspA outer membrane protein production in blebs derived from a recombinant Neisseiria meningitidis serogroup B strain lacking functional porA and cps genes; Up-regulation of the D15/Omp85 outer membrane protein antigen in blebs derived from a recombinant Neisseiria meningitidis serogroup B strain lacking functional cps genes but expressing PorA; Construction of versatile promoter delivery vectors; Fermentation processes for producing recombinant blebs; Identification of bacterial promoters suitable for up-regulation antigens-coding genes; Up-regulation of the N. meningitidis serogroup B Omp85 gene by promoter replacement; Up-regulation of the Hsf protein antigen in a recombinant Neisseiria meningitidis serogroup B strain lacking functional cps genes but expressing PorA; Expression of the Green Fluorescent Protein in a recombinant Neisseria meningitidis serogroup B strain lacking functional cps genes but expressing PorA; Up-regulation of the N. meningitidis serogroup B NspA gene by promoter replacement; Up-regulation of the N. meningitidis serogroup B pldA (omplA) gene by promoter replacement; Up-regulation of the N. meningitidis serogroup B tbpA gene by promoter replacement; Up-regulation of the N. meningitidis serogroup B pilQ gene by promoter replacement; Construction of a kanR/sacB cassette for introducing “clean”, unmarked mutations in the N. meningitidis chromosome; Use of small recombinogenic sequences (43 bp) to allow homologous recombination in the chromosome of Neisseria meningitidis; Active protection of mice immunized with WT and recombinant Neisseria meningitidis blebs; and Immunogenicity of recombinant blebs measured by whole cell & specific ELISA methods have been described in WO 01/09350 (incorporated by reference herein).

### Example 2

### Gonococcal blebs Expressing Chlamydia trachomatis Proteins on its Surface for use in a Vaccine Composition

Both Chlamydia trachomatis and N. gonorrhoeae cause sexually transmitted diseases, including urethritis, cervicitis, salpingitis and pelvic inflammatory disease. Mixed infection with both CT and GC does occur. Therefore, in the design of a vaccine targeting one, or more of these diseases, the possibility to afford protection against both organisms with one single formulation creates a technical advantage.

Protection Against N. gono.

A N. gonorrhoeae OMV vaccine can be obtained from bleb producing strain(s) in which the expression of one or several genes have been up and/or down regulated. A list of genes encoding N. gonorrhoeae proteins for which it is particularly useful to up/down regulate expression is provided above.

A successful vaccine for the prevention of infection by N. gono may require more than one of the following elements: generation of serum and/or mucosal antibodies to facilitate complement mediated killing of the gonococcus, and/or to enhance phagocytosis and microbial killing by leukocytes such as polymorphonuclear leukocytes, and/or to prevent attachment of the gonococci to the host tissues; induction of a cell mediated immune response may also participate to protection.

The potential of a bleb gono vaccine preparation can be evaluated by analyzing the induced immune response for serum and/or mucosal antibodies that have antiadherence, and/or opsonizing properties, and/or bactericidal activity, as described by others (McChesney D et al, Infect. Immun. 36: 1006, 1982; Boslego J et al: Efficacy trialof a purified gonococcl pilus vaccine, in Program and Abstracts of the 24^{th} Interscience Conference on Antimicrobial Agents and Chemotherapy, Whashington, American Society for Microbiology, 1984; Siegel M et al, J. Infect. Dis 145: 300, 1982; de la Pas, Microbiology, 141 (Pt4): 913-20, 1995).

A mouse model of genital infection by N. gono has recently been described (Plante M, J. Infect. Dis., 182: 848-55, 2000). The efficiency of a bleb gono vaccine could also be evaluated by its ability to prevent or to reduce colonization by N. gono in this mouse model of infection.

Protection Against CT

A GC/CT bleb vaccine can be obtained from a strain expressing one or several Chlamydia genes, preferably selected from the above list of genes encoding predicted outer membrane proteins.

Other genes of interest for overexpression in Neisseria are C. trachomatis genes for which no homolog has been found in C. pneumoniae. Such a set of genes has been described in Richard S.; p:9-27, Stephens Stephens Ed. ASM Press, Washington D.C., Chlamydia: Intracellular Biology, Pathogenesis, and Immunity ISBN: 1-55581-155-8 pages: 380.

Most preferred combinations of Chlamydia trachomatis genes are as follows:

Major outer membrane protein MOMP (from one or several different serovars) and the Outer membrane Protein Analog (also known as PorB), MOMP (from one or several different serovars) and the Putative Outer Membrane Protein G (pmpG); & PorB and pmpG.

Although the immunity to CT is not fully understood, there is evidence that Ab play a role in protection. Ab to CT in genital fluids have been associated with immunity to CT (Brunham R C, Infect Immun. March 1983;39(3):1491-4.). A protective role of serum antibody in immunity to chlamydial genital infection has also been shown (Rank R G, Infect Immun. January 1989;57(1):299-301.). Antibodies, e.g. MOMP specific antibodies, have been shown to be capable to neutralize CT infection in vitro and in vivo (Caldwell et al. 1982 Infect. Immun. 38: 745-54, Lucero et al, 1985, Infect. Immun. 50: 595-97, Zhang et al. 1987 J. Immunol. 138: 575-581). The MOMP surface antigen of CT has been shown to bear non linear surface epitopes which are target of neutralizing antibodies (Fan J, J. Infect Dis 1997, 176(3):713-21).

Thus, an important objective in the design of a protective chlamydia vaccine includes the identification of formulation(s) of the CT antigens able to optimize the induction of a chlamydia specific antibody responses. Optimization of the Ab response includes targeting to the genital mucosa, and/or presentation of properly folded Chlamydia antigens, and/or combination of several antibody targets.

Mucosal targeting of the immune response to Chlamydia antigen can be achieved by mucosal administration of the vaccine. Intranasal administration of a outer membrane vesicle vaccine can induce persistent local mucosal antibodies and serum antibodies with strong bactericidal activity in humans.

For certain B cell epitopes, such as non linear epitopes, the presentation of the antigen to the immune system in a properly folded manner is critical. A bleb vaccine prepared from a strain expressing Chlamydia antigen(s) offers to chlamydia OMP an outer membrane environment which can be critical to maintain these antigens in a properly folded structure.

Combination of several antibody targets can create an increased efficacy by tackling the infection at different steps of the life cycle of the bacteria, such as adhesion to the host cell, internalization by the host cell and/or interference with further steps of the intracellular development.

The induction and recruitement of Th1 cells into the local genital mucosae are important for immunity against Chlamydia. Thus, an important objective in designing a protective anti-chlamydia vaccine includes the identification of formulation(s) of CT antigen(s) able to optimize the induction of chlamydia speicific Th1 cells, and preferably recruitment of these cells into the genital mucosae. A bleb vaccine prepared from a strain expressing chlamydia antigen(s) can induce a chlamydia specific CMI response. Antigen-specific T-cell responses can be induced in humans after intranasal immunization with an outer membrane vesicle vaccine.

A particular advantage of a GC/CT bleb vaccine is its capability to induce both Ab and CMI responses.

The efficacy of the GC/CT bleb vaccine can be evaluated by its ability to elicit Ag or Chlamydia-specific Ab and/or CMI responses. Ab responses can be evaluated by classical techniques such as ELISA or western blot. Preferably, the induced antibodies can neutralize the infectivity of Chlamydia in an in vitro assay (Byrne G. et al. (J Infect Dis. August 1993;168(2):415-20). Preferably, the CMI response is biased toward the Th1 phenotype. A Th1 biased immune response can be assessed by elevated antigen-specific IgG2a/ IgG1 ratios in mice (Snapper et al. 1987, Science 236:944-47). Elevated ratio of Th1/Th2 cytokine, e.g. elevated IFN-gamma/IL-5, ratio upon in vitro restimulation of immune T cells with the antigen(s) can also indicate such a biased Th1 response.

The ability of the formulation to elicit Ag specific mucosal Ab is of particular interest, and can be demonstrated by detection of antibodies, such as IgG and/or IgA in mucosal fluids, such as genital tract secretions, vaginal lavages. To this end, certain route of administration of the vaccine may be particularly desired such as intranasal, oral, intravaginal, intradermal, deliveries.

The efficacy of the GC/CT bleb vaccine can be evaluated by its ability to induce protection against a Chlamydia challenge in animal model(s). Examples of such animal models have been described in the literature: genital infection with MoPn in mice (Barron et al. J. Infect. Dis. 1143:63-66), genital infection with human strains in mice (Igietseme et al.2000, Infect. Immun. 68:6798-806, Tuffrey et al. 1992 J. Gen. Microbiol. 138: 1707-1715), Tuffrey), genital infection with GPIC strain in guinea pigs (Rank et al. 1992 Am. J. Pathol. 140:927-936). Protection against infection can be assessed by reduction of shed Chlamydia from the infected site and/or reduction of the histopathological reactions after a challenge infection in immunized animals.

The advantage of combining two or more Chlamydia antigens (as described above) can be evaluated by one or more of the following techniques:

Ability to elicit a multi-target Ab and/or T cell protective response

Ability to elicit Ab titers in an in vitro neutralizing assay, and/or neutralizing Ab against multiple strains (antigenically distinct)

Ability to elicit a protective immune response against Chlamydia in a mouse model of genital infection as assessed by reduced shedding of bacteria and/or pathology after challenge.

### Example 3

### Expression of Heterologous Antigens (Clamydia trachomatis MOMP and PorB) in blebs Derived from a Recombinant Neisseiria meningitidis Serogroup B Strain Lacking Functional porA and cps Genes

Other genes of interest for over-expression in Neisseria are Chlamydia trachomatis genes for which no homologue has been found in Chlamydia pneumoniae. Among those, the major outer membrane protein (MOMP) and the outer membrane protein analog (PorB) have been shown to play a protective role against chlamydial genital infection. Optimization of the Ab response could be achieved by presentation of properly folded proteins.

MenB bleb vesicles may be used as delivery vectors to express heterologous membrane protein antigens under the control of the engineered porA-lacO promoter described in WO 01/09350. Expressed in the bleb context, recombinant MOMP and PorB from Chlamydia trachomatis serovar D and K can be correctly folded in the membrane and exposed at the surface. Neisseiria meningitidis strains lacking functional cps genes are advantageously used as recipient strains to express the heterologous antigens (WO 01/09350).

PCR Amplifications of the Genes Coding for MOMP (Chlamydia trachomatis).

Murine McCoy cells (ATCC) infected either, with Chlamydia trachomatis Serovar K (UW31-CX-serK), or Serovar D (UW31-CX-serD), were lysed in 400 μl of lysis buffer: 50 mM KCl, 10 mM Tris-HCl pH 8.3, 2.5 mM MgCl2, 0.45% Nonidet P40, 0.45% Tween 20 containing 60 μg/ml proteinase K, 3 hours at 56° C. Ten μl of the lysate were used as template to amplify the corresponding genes. The gene coding for MOMP (Serovar K) (SEQID N^{o} 1 below) was PCR amplified using the CYK/OMP/5/NDE and CYKD/OMP/3/BG oligonucleotide primers (see table 1). The gene coding for MOMP (Serovar D) (SEQID N^{o} 2 below) was PCR amplified using the CYD/OMP/5/NRU and CYKD/OMP/3/BG oligonucleotide primers (see table 1). The conditions used for PCR amplification were those described by the supplier (HiFi DNA polymerase, Boehringer Mannheim, GmbH). Thermal cycling was the following: 25 times (94° C. 1 min., 52° C. 1 min., 72° C. 3 min.) and 1 time (72° C. 10 min., 4° C. up to recovery). The corresponding amplicons (1194 bp) were digested with either NdeI/BglII or NruI/BglII restriction enzymes and can be cloned in the corresponding restriction sites of pCMK (+) delivery vector (as described in WO 01/09350).

PCR Amplifications of the Genes Coding for PorB (Chlamydia trachomatis).

Murine McCoy cells (ATCC) infected either, with Chlamydia trachomatis Serovar K (UW31-CX-serK), or Serovar D (UW31-CX-serD), were lysed in 400 μl of lysis buffer: 50 mM KCl, 10 mM Tris-HCl pH 8.3, 2.5 mM MgCl2, 0.45% Nonidet P40, 0.45% Tween 20 containing 60 μg/ml proteinase K, 3 hours at 56° C. Ten μl of the lysate were used as template to amplified the corresponding genes.

PorB sequences are highly conserved amongst serovar D and K (SEQID N^{o} 3 below). The same primers were used to amplify the corresponding genes in both serovars: CYD/PORB/5/NRU and CYD/PORB/3/BG (see table 1). The conditions used for PCR amplification were those described by the supplier (HiFi DNA polymerase, Boehringer Mannheim, GmbH). Thermal cycling was the following: 25 times (94° C. 1 min., 52° C. 1 min., 72° C. 3 min.) and 1 time (72° C. 10 min., 4° C. up to recover corresponding amplicons (1035 bp) were digested with NruI/BglII restriction enzymes and can be cloned in the corresponding restriction sites of pCMK (+) delivery vector (as described in WO 01/09350).

Transformation

Linearized recombinant pCMK plasmids can be transformed within a Neisseria meningitidis serogroup B strain lacking functional cps genes (described in WO 01/09350). Integration resulting from a double crossing-over between the pCMK vectors and the chromosomal porA locus can be selected by a combination of PCR and Western Blot screening as described in WO 01/09350.

TABLE 1
Oligonucleotides used in this work

| | | |
|---|---|---|
| Oligonucleotides | Sequence | Remark(s) |

| | | | |
|---|---|---|---|
| CYK/OMP/5/NDE | 5′-GGG AAT CCA TAT GAA AAA ACT CTT GAA | NdeI | cloning site |
| | ATC GG-3′[SEQ ID NO:101] | | |
| CYKD/OMP/3/BG | 5′-GGA AGA TCT TTA GAA GCG GAA TTG TGC | Bgl II | cloning site |
| | AT-3′[SEQ ID NO:102] | | |
| CYD/OMP/5/NRU | 5′-CTG CAG AAT CGC GAA TGA AAA AAC TCT | NruI | cloning site |
| | TGA AAT CGG-3′[SEQ ID NO:103] | | |
| CYD/POR/5/NRU | 5′-CTG CAG AAT CGC GAA TGA GTA GCA AGC | NruI | cloning site |
| | TAG TGA AC-3′[SEQ ID NO:104] | | |
| CYD/POR/3/BG | 5′-AGG AGA TCT TTA GAA TTG GAA TCC TCC | Bgl II | cloning site |
| | GG-3′[SEQ ID NO:105] | | |

[SEQ ID NO:106]
Nucleotide sequence of DNA coding for Chlamydia trachomatis MOMP
serovar K protein.
atgaaaaaactcttgaaatcggtattagtatttgccgctttgagttctgcttcctccttgcaagctct
gcctgtggggaatcctgctgaaccaagccttatgatcgacggaattctgtgggaaggtttcggcggag
atccttgcgatccttgcaccacttggtgtgacgctatcagcatgcgcgttggttactacggagacttt
gttttcgaccgtgttttgaaaactgatgtgaataaagaatttcagatgggagcggcgcctactaccag
cgatgtagaaggcttacaaaacgatccaacaacaaatgttgctcgtccaaatcccgcttatggcaaac
acatgcaagatgctgaaatgtttacgaacgctgcttacatggcattaaatatctgggatcgttttgat
gtattttgtacattgggagcaactaccggttatttaagaggaaactccgcttccttcaacttagttgg
attattcggaacaaaaacacaatattctaagtttaatacagcgaatcttgttcctaacactgctttgg
atcgagctgtggttgagctttatacagacaccacctttgcttggagcgtaggtgctcgtgcagctctc
tgggaatgtgggtgtgcaacgttaggagcttctttccaatatgctcaatctaaacctaaagtagaaga
gttaaatgttctttgtaatgcatccgaatttactattaataagccgaaaggatatgttggggtggaat
ttccacttgatattaccgcaggaacagaagctgcgacagggactaaggatgcctctattgactaccat
gagtggcaagcaagtttagccctttcttacagattaaatatgttcactccttacattggagttaaatg
gtctagagtaagttttgatgccgacacgatccgtatcgctcagcctaaattggctgaagcaatcttgg
atgtcactactctaaacccgaccatcgctggtaaaggagctgtggtctcttccggaagcgataacgaa
ctggctgatacaatgcaaatcgtttccttgcagttgaacaagctgaaatctagaaaatcttgcggtat
tgcagtaggaacgactattgtagatgcagataaatacgcagttacagttgagactcgcttgatcgatg
agagagcagctcacgtaaatgcacaattccgcttctaa
[SEQ ID NO:107]
Nucleotide sequence of DNA coding for Chlamydia trachomatis MOMP serovar
D protein.
atgaaaaaactcttgaaatcggtattagtatttgccgctttgagttctgcttcctccttgcaagctct
gcctgtggggaatcctgctgaaccaagccttatgatcgacggaattctgtgggaaggtttcggcggag
atccttgcgatccttgcgccacttggtgtgacgctatcagcatgcgtgttggttactacggagacttt
gttttcgaccgtgttttgaaaactgatgtgaataaagaatttcagatgggtgccaagcctacaactga
tacaggcaatagtgcagctccatccactcttacagcaagagagaatcctgcttacggccgacatatgc
aggatgctgagatgtttacaaatgccgcttgcatggcattgaatatttgggatcgttttgatgtattc
tgtacattaggagccaccagtggatatcttaaaggaaactctgcttctttcaatttagttggattgtt
tggagataatgaaaatcaaaaaacggtcaaagcggagtctgtaccaaatatgagctttgatcaatctg
ttgttgagttgtatacagatactacttttgcgtggagcgtcggcgctcgcgcagctttgtgggaatgt
ggatgtgcaactttaggagcttcattccaatatgctcaatctaaacctaaagtagaagaattaaacgt
tctctgcaatgcagcagagtttactattaataaacctaaagggtatgtaggtaaggagtttcctcttg
atcttacagcaggaacagatgctgcgacaggaactaaggatgcctctattgattaccatgaatggcaa
gcaagtttagctctctcttacagactgaatatgttcactccctacattggagttaaatggtctcgagc
aagctttgatgccgatacgattcgtatagcccagccaaaatcagctacagctatttttgatactacca
cgcttaacccaactattgctggagctggcgatgtgaaaactggcgcagagggtcagctcggagacaca
atgcaaatcgtttccttgcaattgaacaagatgaaatctagaaaatcttgcggtattgcagtaggaac
aactattgtggatgcagacaaatacgcagttacagttgagactcgcttgatcgatgagagagcagctc
acgtaaatgcacaattccgcttctaa
[SEQ ID NO:108]
Nucleotide sequence of DNA coding for Chlamydia trachomatis PorB serovar D
protein.
atgagtagcaagctagtgaactatctccgtttgactttcctatcttttttagggatcgcatctacttc
attagacgctatgcctgcggggaatccggcgtttccagtcatcccggggattaatattgaacagaaaa
atgcctgttctttcgatttatgtaattcttatgatgtactatccgcactgtccggtaacctgaagctc
tgcttctgcggagattatatcttttcagaagaagctcaggtaaaagatgtccctgtcgttacctctgt
gacaacagctggggttggtccttctcctgatattacttcgacaaccaaaacgcgaaatttcgatctcg
tgaactgtaatctcaatacaaactgtgtagctgtagctttttcccttcctgatcgttcgctgagcgcg
attcctctgtttgatgtgagtttcgaagtgaaagtaggaggactgaaacaatactaccgccttcccat
gaatgcctatcgagacttcacctcggaacctctcaattctgaatcagaagttacggacgggatgattg
aagtacagtccaattacggatttgtttgggatgttagcttgaaaaaagtcatatggaaagatggcgtt
tcctttgtaggcgtcggtgcagactatcgccatgcttcttgccctattgactacatcattgcaaacag
tcaagctaatccagaagtattcatcgctgactcggatgggaaactgaacttcaaggagtggagtgtct
gcgtaggtcttactacctatgtgaatgactacgttcttccttacttagcgttttctatagggagtgtt
tctcgccaagctccggacgacagcttcaaaaaattagaagatcgcttcactaacctcaaatttaaagt
tcgtaaaattaccagctctcatcgtggaaacatctgcatcggagcgacaaactatgtcgccgataact
tcttctacaacgtagaaggaagatggggaagccagcgcgctgtgaacgtctccggaggattccaattc
taa

### Example 4

### Isolation and Purification of blebs from meningococci Devoid of Capsular polysaccharide

Recombinant blebs can be purified as described below. The cell paste (42 gr) is suspended in 211 ml of 0.1M Tris-Cl buffer pH 8.6 containing 10 mM EDTA and 0.5% Sodium Deoxycholate (DOC). The ratio of buffer to biomass should be 5/1 (V/W). The biomass is extracted by magnetic stirring for 30 minutes at room temperature. Total extract is then centrifuged at 20,000 g for 30 minutes at 4° C. (13,000 rpm in a JA-20 rotor, Beckman J2-HS centrifuge). The pellet should be discarded. The supernatant is ultracentrifuged at 125,000 g for 2 hours at 4° C. (40,000 rpm in a 50.2 Ti rotor, Beckman L8-70M ultracentrifuge) in order to concentrate vesicles. The supernatant should be discarded. The pellet is gently suspended in 25 ml of 50 mM Tris-Cl buffer pH 8.6 containing 2 mM EDTA, 1.2% DOC and 20% sucrose. After a second ultracentrifugation step at 125,000 g for 2 hours at 4° C., vesicles are gently suspended in 44 ml of 3% sucrose and stored at 4° C. All solutions used for bleb extraction and purification contained 0.01% thiomersalate. As illustrated in WO 01/019350, this procedure yields protein preparations highly enriched in outer-membrane proteins.

### Example 5

### Models for Testing Protection Against gonococcal and C. trachomatis Infection

This can be done as described above in Example 2. In addition Whittum-Hudson et al. (Vaccine Jul. 16, 2001;19(28-29):4061-71) “The anti-idiotypic antibody to chlamydial glycolipid exoantigen (GLXA) protects mice against genital infection with a human biovar of Chlamydia trachomatis” is a vaginal inoculation model for C. trachomatis (incorporated by reference herein) which can also be used to test vaccine efficacy.

1. A Gram-negative bacterial bleb not derived from Chlamydia presenting on its surface the PorB outer membrane protein from Chlamydia trachomatis, wherein the combination of the Chlamydia antigen with the native Gram-negative bacterial bleb antigens interact in the prevention or treatment of salpingitis when present in a vaccine formulation.

2. The Gram-negative bleb of claim 1 further presenting on its surface the PmpG outer membrane proteins from Chlamydia trachomatis.

3. The Gram-negative bleb of claim 1 further presenting on its surface MOMP from one or more serovars from Chlamydia trachomatis.

4. A Gram-negative bleb not derived from Chlamydia, presenting on its surface both the PmpG and MOMP (from one or more serovars) outer membrane proteins from Chlamydia trachomatis, wherein the combination of the Chlamydia antigens with the native Gram-negative bacterial bleb antigens interact in the prevention or treatment of salpingitis when present in a vaccine formulation.

5. The bleb of claims 1-4 which are gonococcal blebs.

6. The bleb of claim 5 which has been derived from a gonococcal strain which has been modified to upregulate one or more protective gonococcal outer membrane antigens.

7. The bleb of claims 5 and 6 derived from a gonococcal strain which has been modified to downregulate one or more immunodominant variable or non-protective gonococcal outer membrane antigens.

8. The bleb of claims 5-7 derived from a strain which has a detoxified lipid A portion of bacterial LPS, due to the strain having been engineered to reduce or switch off expression of one or more genes selected from the group consisting of: htrB, msbB and lpxK.

9. The bleb of claims 5-8 wherein the bleb preparation is derived from a strain which has a detoxified lipid A portion of bacterial LPS, due to the strain having been engineered to express at a higher level one or more genes selected from the group consisting of: pmrA, pmrB, pmrE and pmrF.

10. A vaccine composition comprising the bleb of claims 1-9 and a pharmaceutically suitable excipient or carrier.

11. The vaccine of claim 10, additionally comprising a mucosal adjuvant.

12. A method of preventing Chlamydia trachomatis infection in a host comprising the steps of administering an effective amount of the vaccine of claim 10 or 11 to a host in need thereof.

13. The method of claim 12 where in the vaccine is mucosally administered via either a intranasal, oral, or intravaginal route.

14. A Gram-negative bleb produced from Neisseria meningitidis, Moraxella catharralis or Haemophilus influenzae presenting on its surface a protective antigen from Chlamydia pneumoniae.

15. The Gram-negative bleb of claim 14 presenting on its surface a PorB outer membrane protein from Chlamydia pneumoniae.

16. The Gram-negative bleb of claim 15 further_presenting on its surface MOMP outer membrane protein from Chlamydia pneumoniae.

17. The Gram-negative bleb of claim 15 further_presenting on its surface one or more Pmp outer membrane proteins from Chlamydia pneumoniae.

18. The Gram-negative bleb of claim 15 further_presenting on its surface Npt1 protein from Chlamydia pneumoniae.

19. The Gram-negative bleb of claim 14 presenting on its surface one or more Pmp proteins from Chlamydia pneumoniae.

20. The Gram-negative bleb of claim 19 further_presenting on its surface Npt1 protein from Chlamydia pneumoniae.

21. The Gram-negative bleb of claim 19 further presenting on its surface MOMP protein from Chlamydia pneumoniae.

22. The Gram-negative bleb of claim 14 presenting on its surface MOMP protein from Chlamydia pneumoniae.

23. The Gram-negative bleb of claim 22 further presenting on its surface Npt1 protein from Chlamydia pneumoniae.

24. The bleb of claims 14-23 which are meningococcal blebs.

25. The bleb of claim 24 derived from a meningococcal strain that has been modified to upregulate one or more protective meningococcal outer membrane antigens.

26. The bleb of claim 24 or 25 derived from a meningococcal strain that has been modified to downregulate one or more immunodominant variable or non-protective meningococcal outer membrane antigens.

27. The bleb of claims 24-26 derived from a strain which has a detoxified lipid A portion of bacterial LPS, due to the strain having been engineered to reduce or switch oft expression of one or more genes selected from the group consisting of: htrB, msbB and lpxK.

28. The bleb of claims 24-27 wherein the bleb preparation is derived from a strain which has a detoxified lipid A portion of bacterial LPS, due to the strain having been engineered to express at a higher level one or more genes selected from the group consisting of: pmrA, pmrB, pmrE and pmrF.

29. A vaccine composition comprising the bleb of claims 14-28 and a pharmaceutically suitable excipient or carrier.

30. The vaccine of claim 29, additionally comprising a mucosal adjuvant.

31. A method of preventing Chlamydia pneumoniae infection in a host comprising the steps of administering an effective amount of the vaccine of claim 29 or 30 to a host in need thereof.

32. The method of claim 31 where in the vaccine is mucosally administered via either an intranasal, or oral route.


SEQUENCE LISTING (108 sequences)

SEQ ID NO: 1 - DNA, 1023 bp, Chlamydia trachomatis
atgagtagcaagctagtgaactatctccgtttgactttcctatcttttttagggatcgcatctacttcattagacgctatgcctgcggggaatccggcgtttccagtcatcccggggattaatattgaacagaaaaatgcctgttctttcgatttatgtaattcttatgatgtactatccgcactgtccggtaacctgaagctctgcttctgcggagattatatcttttcagaagaagctcaggtaaaagatgtccctgtcgttacctctgtgacaacagctggggttggtccttctcctgatattacttcgacaaccaaaacgcgaaatttcgatctcgtgaactgtaatctcaatacaaactgtgtagctgtagctttttcccttcctgatcgttcgctgagcgcgattcctctgtttgatgtgagtttcgaagtgaaagtaggaggactgaaacaatactaccgccttcccatgaatgcctatcgagacttcacctcggaacctctcaattctgaatcagaagttacggacgggatgattgaagtacagtccaattacggatttgtttgggatgttagcttgaaaaaagtcatatggaaagatggcgtttcctttgtaggcgtcggtgcagactatcgccatgcttcttgccctattgactacatcattgcaaacagtcaagctaatccagaagtattcatcgctgactcggatgggaaactgaacttcaaggagtggagtgtctgcgtaggtcttactacctatgtgaatgactacgttcttccttacttagcgttttctatagggagtgtttctcgccaagctccggacgacagcttcaaaaaattagaagatcgcttcactaacctcaaatttaaagttcgtaaaattaccagctctcatcgtggaaacatctgcatcggagcgacaaactatgtcgccgataacttcttctacaacgtagaaggaagatggggaagccagcgcgctgtgaacgtctccggaggattccaattctaa

SEQ ID NO: 2 - protein, 340 aa, Chlamydia trachomatis
MSSKLVNYLRLTFLSFLGIASTSLDAMPAGNPAFPVIPGINIEQKNACSFDLCNSYDVLSALSGNLKLCFCGDYIFSEEAQVKDVPVVTSVTTAGVGPSPDITSTTKTRNFDLVNCNLNTNCVAVAFSLPDRSLSAIPLFDVSFEVKVGGLKQYYRLPMNAYRDFTSEPLNSESEVTDGMIEVQSNYGFVWDVSLKKVIWKDGVSFVGVGADYRHASCPIDYIIANSQANPEVFIADSDGKLNFKEWSVCVGLTTYVNDYVLPYLAFSIGSVSRQAPDDSFKKLEDRFTNLKFKVRKITSSHRGNICIGATNYVADNFFYNVEGRWGSQRAVNVSGGFQF

SEQ ID NO: 3 - protein, 601 aa, Chlamydia trachomatis
MKMNRIWLLLLTFSSAIHSPVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 4 - DNA, 1806 bp, Chlamydia trachomatis
atgaaaatgaataggatttggctattactgcttaccttttcttctgccatacattctcctgtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttaa

SEQ ID NO: 5 - protein, 432 aa, Chlamydia trachomatis
MKKYFYKGFVGALLLACGSTNLAFAQASSMDSQLWSVEDLDSYLSSKGFVETRKRDGVLRLAGDVRARWIYAKEDLETTQTPAKPMLPTNRYRSEFNLYVDYTAANSWMTSKMNWVTIAGGESSAAGLDINRAFLGYRFYKNPETQAEVFAEIGRSGLGDIFDSDVQFNSNFDGIHLYAARRISEKLPFTMIVHGGPFVVNMAEKEYAWVVEAILNKLPGNFVVKTSVVDWNTLTAKTNDPADASAAQPAKPNTKYDYLVWQWLVGKSTAMPWFNGQTKNLYTYGAYLFNPLAEIPENWKQSTTPTTKITNGKENHAWFIGCSLGGVRRAGDWSATVRYEYVEALAIPEIDVAGIGRGNQMKYWFAQAIKQGLDPKESNGFTNYKGVSYQFVMGLTDSVSFRAYAAYSKPANDNLGSDFTYRKYDLGLISSF

SEQ ID NO: 6 - DNA, 1299 bp, Chlamydia trachomatis
atgaaaaaatacttttataaagggtttgtaggcgcgcttttattagcttgtgggtctacaaacttggcttttgcgcaggctagttcgatggatagccagctatggtctgttgaagatttagattcttatttgagttccaaaggttttgtcgagactcgtaagcgcgatggagttctacgtttagctggagatgtccgcgctcgatggatttatgcaaaagaggatcttgagacaactcagactcctgctaaacctatgttacctaccaatcggtatcgtagtgaattcaatttgtatgtggattacaccgctgctaatagttggatgacttcgaaaatgaattgggtaacgattgctggcggagaatcttctgcagcagggttagatattaatcgtgccttcttaggataccgattctacaaaaacccagaaacgcaagcagaagtatttgcagagattggtcgctctggattgggagatatttttgattccgacgttcagtttaatagtaatttcgacggaattcatttatacgctgcgcgacgtattagtgagaaacttcctttcaccatgattgttcatggtggtccttttgtcgtgaatatggcagagaaagagtatgcttgggtcgtggaagctattttgaataaactcccaggaaatttcgttgtgaaaacgagtgttgttgactggaatacgttaacagcaaaaacgaatgatccagcagacgcaagcgctgcacaaccagctaaacctaataccaagtacgattatttagtatggcaatggttggttgggaagagcacagctatgccatggtttaatggacaaacaaaaaatctttacacttacggagcctatctctttaatccattagcggaaataccagagaactggaaacaatcaacaactcctacaaccaaaattacaaatggtaaggaaaaccatgcttggttcatcggctgctctctaggcggtgttcgacgagctggagactggtctgcaacagttcgttatgagtatgttgaagctttagcgattccagaaattgatgtcgcgggtattggtcgcggaaaccaaatgaaatattggtttgctcaagctatcaaacaaggattggatcctaaagaatctaacggctttactaactataaaggagtttcctatcagtttgttatgggtctgacagattcggtttctttccgagcttatgctgcttattctaagcctgctaacgataaccttggtagcgacttcacctatcgtaagtatgacctaggtttaatttcttcattctaa

SEQ ID NO: 7 - protein, 878 aa, Chlamydia trachomatis
MRPDHMNFCCLCAAILSSTAVLFGQDPLGETALLTKNPNHVVCTFFEDCTMESLFPALCAHASQDDPLYVLGNSYCWFVSKLHITDPKEALFKEKGDLSIQNFRFLSFTDCSSKESSPSIIHQKNGQLSLRNNGSMSFCRNHAEGSGGAISADAFSLQHNYLFTAFEENSSKGNGGAIQAQTFSLSRNVSPISFARNRADLNGGAICCSNLICSGNVNPLFFTGNSATNGGAICCISDLNTSEKGSLSLACNQETLFASNSAKEKGGAIYAKHMVLRYNGPVSFINNSAKIGGAIAIQSGGSLSILAGEGSVLFQNNSQRTSDQGLVRNAIYLEKDAILSSLEARNGDILFFDPIVQESSSKESPLPSSLQASVTSPTPATASPLVIQTSANRSVIFSSERLSEEEKTPDNLTSQLQQPIELKSGRLVLKDRAVLSAPSLSQDPQALLIMEAGTSLKTSSDLKLATLSIPLHSLDTEKSVTIHAPNLSIQKIFLSNSGDENFYENVELLSKEQNNIPLLTLSKEQSHLHLPDGNLSSHFGYQGDWTFSWKDSDEGHSLIANWTPKNYVPHPERQSTLVANTLWNTYSDMQAVQSMINTIAHGGAYLFGTWGSAVSNLFYAHDSSGKPIDNWHHRSLGYLFGISTHSLDDHSFCLAAGQLLGKSSDSFITSTETTSYIATVQAQLATPLMKISAQACYNESIHELKTKYRSFSKEGFGSWHSVAVSGEVCASIPIVSNGSGLFSSFSIFSKLQGFSGTQDGFEESSGEIRSFSASSFRNISLPMGITFEKKSQKTRNYYYFLGAYIQDLKRDVESGPVVLLKNAVSWDAPMANLDSRAYMFRLTNQRALHRLQTLLNVSYVLRGQSHSYSLDLGTTYRF

SEQ ID NO: 8 - DNA, 2637 bp, Chlamydia trachomatis
atgcgacctgatcatatgaacttctgttgtctatgtgctgctattttgtcatccacagcggtcctctttggccaggatcccttaggtgaaaccgccctcctcactaaaaatcctaatcatgtcgtctgtacattttttgaggactgtaccatggagagcctctttcctgctctttgtgctcatgcatcacaagatgatcctttgtatgtacttggaaattcctactgttggttcgtatctaaactccatatcacggaccccaaagaggctctttttaaagaaaaaggagatctttccattcaaaattttcgcttcctttccttcacagattgctcttccaaggaaagctctccttctattattcatcaaaagaatggtcagttatccttgcgcaataatggtagcatgagtttctgtcgaaatcatgctgaaggctctggaggagccatctctgcggatgccttttctctacaacacaactatcttttcacagcttttgaagagaattcttctaaaggaaatggcggagccattcaggctcaaaccttctctttatctagaaatgtgtcgcctatttctttcgcccgtaatcgtgcggatttaaatggcggcgctatttgctgtagtaatcttatttgttcagggaatgtaaaccctctctttttcactggaaactccgccacgaatggaggcgctatttgttgtatcagcgatctaaacacctcagaaaaaggctctctctctcttgcttgtaaccaagaaacgctatttgcaagcaattctgctaaagaaaaaggcggggctatttatgccaagcacatggtattgcgttataacggtcctgtttccttcattaacaacagcgctaaaataggtggagctatcgccatccagtccggagggagtctctctatccttgcaggtgaaggatctgttctgttccagaataactcccaacgcacctccgaccaaggtctagtaagaaacgccatctacttagagaaagatgcgattctttcttccttagaagctcgcaacggagatattcttttctttgatcctattgtacaagaaagtagcagcaaagaatcgcctcttccctcctctttgcaagccagcgtgacttctcccaccccagccaccgcatctcctttagttattcagacaagtgcaaaccgttcagtgattttctcgagcgaacgtctttctgaagaagaaaaaactcctgataacctcacttcccaactacagcagcctatcgaactgaaatccggacgcttagttttaaaagatcgcgctgtcctttccgcgccttctctctctcaggatcctcaagctctcctcattatggaagcgggaacttctttaaaaacttcctctgatttgaagttagctacgctaagtattccccttcattccttagatactgaaaaaagcgtaactatccacgcccctaacctttctatccaaaagatcttcctctctaattctggagatgagaatttttatgaaaatgtagagcttctcagtaaagagcaaaacaatattcctctccttactctctctaaagagcaatctcatttacatcttcctgatgggaacctctcttctcactttggatatcaaggagattggactttttcttggaaagattctgatgaagggcattctctgattgctaattggacgcctaaaaactatgtgcctcatccagaacgtcaatctacactcgttgcgaacactctttggaacacctattccgatatgcaagctgtgcagtcgatgattaatacaatagcgcacggaggagcctatctatttggaacgtggggatctgctgtttctaatttattctatgctcacgacagctctgggaaacctatcgataattggcatcatagaagccttggctacctattcggtatcagtactcacagtttagatgaccattctttctgcttggctgcaggacaattactcgggaaatcgtccgattcctttattacgtctacagaaacgacctcctatatagctactgtacaagcgcaactcgctacccctctaatgaaaatctctgcacaggcatgctataatgaaagtatccatgagctaaaaacaaaatatcgctccttctctaaagaaggattcggatcctggcatagcgttgcagtatccggagaagtgtgcgcatcgattcctattgtatccaatggttccggactgttcagctccttctctattttctctaaactgcaaggattttcaggaacacaggacggttttgaggagagttcgggagagattcggtccttttctgccagctctttcagaaatatttcacttcctatgggaataacatttgaaaaaaaatcccaaaaaacacgaaactactattactttctgggagcctacatccaagacctaaaacgtgatgtggaatcgggacctgtagtgttactcaaaaatgccgtctcctgggatgctcctatggcgaacttggattcgcgagcctacatgttcaggcttacgaatcaaagagctctgcatagacttcagacgctgttaaatgtgtcttacgtactgcgcgggcaaagccatagttactccctggatctggggaccacttacaggttctag

SEQ ID NO: 9 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 10 - DNA, 3042 bp, Chlamydia trachomatis
atgcaaacgtctttccataagttctttctttcaatgattctagcttattcttgctgctctttaagtgggggggggtatgcagcagaaatcatgattcctcaaggaatttacgatggggagacgttaactgtatcatttccctatactgttataggagatccgagtgggactactgttttttctgcaggagagttaacgttaaaaaatcttgacaattctattgcagctttgcctttaagttgttttgggaacttattagggagttttactgttttagggagaggacactcgttgactttcgagaacatacggacttctacaaatggagctgcactaagtgacagcgctaatagcgggttatttactattgagggttttaaagaattatctttttccaattgcaactcattacttgccgtactgcctgctgcaacgactaataatggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttatagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctgagcaaccaacaaatggacaggcttctaatacgagtgataattacggagatggaggagctatcttctgtaagaatggtgcgcaagcagcaggatccaataactctggatcagtttcctttgatggagagggagtagttttctttagtagcaatgtagctgctgggaaagggggagctatttatgccaaaaagctctcggttgctaactgtggccctgtacaattcttagggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccgaacctctaaaaattaacgatggtgaaggatacacaggggatattgtttttgctaatggaaacagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcaatcattggtagcacaactgctggttctgttacaattagtgggcctatcttttttgaggatttggatgatacagcttatgataggtatgattggctaggttctaatcaaaaaatcgatgtcctgaaattacagttagggactcagccctcagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttaccgaagtatttggtagatctaaagattatgtagtgtgtcgttccaatcatcatgcttgcataggatccgtttatctatctaccaaacaagctttatgtggatcctatttgttcggagatgcgtttatccgtgctagctacgggtttgggaaccagcatatgaaaacctcatacacatttgcagaggagagcgatgttcgttgggataataactgtctggttggagagattggagtgggattaccgattgtgattactccatctaagctctatttgaatgagttgcgtcctttcgtgcaagctgagttttcttatgccgatcatgaatcttttacagaggaaggcgatcaagctcgggcattcaggagtggacatctcatgaatctatcagttcctgttggagtaaaatttgatcgatgttctagtacacaccctaataaatatagctttatgggggcttatatctgtgatgcttatcgcaccatctctgggactcagacaacactcctatcccatcaagagacatggacaacagatgcctttcatttggcaagacatggagtcatagttagagggtctatgtatgcttctctaacaagcaatatagaagtatatggccatggaagatatgagtatcgagatacttctcgaggttatggtttgagtgcaggaagtaaagtccggttctaa

SEQ ID NO: 11 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 12 - DNA, 3105 bp, Chlamydia trachomatis
atgattaaaagaacttctctatcctttgcttgcctcagttttttttatctttcaactatatccattttgcaagctaatgaaacggatacgctacagttccggcgatttactttttcggatagagagattcagttcgtcctagatcccgcctctttaattaccgcccaaaacatcgttttatctaatttacagtcaaacggaaccggagcctgtaccatttcaggcaatacgcaaactcaaatcttttctaattccgttaacaccaccgcagattctggtggagcctttgatatggttactacctcattcacggcctctgataatgctaatctactcttctgcaacaactactgcacacataataaaggcggaggagctattcgttccggaggacctattcgattcttaaataatcaagacgtgcttttttataataacatatcggcaggggctaaatatgttggaacaggagatcacaacgaaaaaaataggggcggtgcgctttatgcaactactatcactttgacagggaatcgaactcttgcctttattaacaatatgtctggagactgcggtggagccatctctgctgacactcaaatatcaataactgataccgttaaaggaattttatttgaaaacaatcacacgctcaatcatataccgtacacgcaagctgaaaatatggcacgaggaggagcaatctgtagtagaagagacttgtgctcaatcagcaataattctggtcccatagtttttaactataaccaaggcgggaaaggtggagctattagcgctacccgatgtgttattgacaataacaaagaaagaatcatcttttcaaacaatagttccctgggatggagccaatcttcttctgcaagtaacggaggagccattcaaacgacacaaggatttactttacgaaataataaaggctctatctacttcgacagcaacactgctacacacgccgggggagccattaactgtggttacattgacatccgagataacggacccgtctattttctaaataactctgctgcctggggagcggcctttaatttatcgaaaccacgttcagcgacaaattatatccatacagggacaggcgatattgtttttaataataacgttgtctttactcttgacggtaatttattagggaaacggaaactttttcatattaataataatgagataacaccatatacattgtctctcggcgctaaaaaagatactcgtatctatttttatgatcttttccaatgggagcgtgttaaagaaaatactagcaataacccaccatctcctaccagtagaaacaccattaccgttaacccggaaacagagttttctggagctgttgtgttctcctacaatcaaatgtctagtgacatacgaactctgatgggtaaagaacacaattacattaaagaagccccaactactttaaaattcggaacgctagccatagaagatgatgcagaattagaaatcttcaatatcccgtttacccaaaatccgactagccttcttgctttaggaagcggcgctacgctgactgttggaaagcacggtaagctcaatattacaaatcttggtgttattttacccattattctcaaagaggggaagagtccgccttgtattcgcgtcaacccacaagatatgacccaaaatactggtaccggccaaactccatcaagcacaagtagtataagcactccaatgattatctttaatgggcgcctctcaattgtagacgaaaattatgaatcagtctacgacagtatggacctctccagagggaaagcagaacaactaattctatccatagaaaccactaatgatgggcaattagactccaattggcaaagttctctgaatacttctctactctctcctccacactatggctatcaaggtctatggactcctaattggataacaacaacctataccatcacgcttaataataattcttcagctccaacatctgctacctccatcgctgagcagaaaaaaactagtgaaacttttactcctagtaacacaactacagctagtatccctaatattaaagcttccgcaggatcaggctctggatcggcttccaattcaggagaagttacgattaccaaacatacccttgttgtaaactgggcaccagtcggctacatagtagatcctattcgtagaggagatctgatagccaatagcttagtacattcaggaagaaacatgaccatgggcttacgatcattactcccggataactcttggtttgctttgcaaggagctgcaacaacattatttacaaaacaacaaaaacgtttgagttatcatggctactcttctgcatcaaaggggtataccgtctcttctcaagcatcaggagctcatggtcataagtttcttctttccttctcccagtcatctgataagatgaaagaaaaagaaacaaataaccgcctttcttctcgttactatctttctgctttatgtttcgaacatcctatgtttgatcgcattgctcttatcggagcagcagcttgcaattatggaacacataacatgcggagtttctatggaactaaaaaatcttctaaagggaaatttcactctacaaccttaggagcttctcttcgctgtgaactacgcgatagtatgcctttacgatcaataatgctcaccccatttgctcaggctttattctctcgaacagaaccagcttctatccgagaaagcggtgatctagctagattatttacattagagcaagcccatactgccgttgtctctccaataggaatcaaaggagcttattcttctgatacatggccaacactctcttgggaaatggaactagcttaccaacccaccctctactggaaacgtcctctactcaacacactattaatccaaaataacggttcttgggtcaccacaaataccccattagctaaacattccttttatgggagaggttctcactccctcaaattttctcatctgaaactatttgctaactatcaagcagaagtggctacttccactgtctcacactacatcaatgcaggaggagctctggtcttttaa

SEQ ID NO: 13 - protein, 964 aa, Chlamydia trachomatis
MKKAFFFFLIGNSLSGLAREVPSRIFLMPNSVPDPTKESLSNKISLTGDTHNLTNCYLDNLRYILAILQKTPNEGAAVTITDYLSFFDTQKEGIYFAKNLTPESGGAIGYASPNSPTVEIRDTIGPVIFENNTCCRLFTWRNPYAADKIREGGAIHAQNLYINHNHDVVGFMKNFSYVQGGAISTANTFVVSENQSCFLFMDNICIQTNTAGKGGAIYAGTSNSFESNNCDLFFINNACCAGGAIFSPICSLTGNRGNIVFYNNRCFKNVETASSEASDGGAIKVTTRLDVTGNRGRIFFSDNITKNYGGAIYAPVVTLVDNGPTYFINNIANNKGGAIYIDGTSNSKISADRHAIIFNENIVTNVTNANGTSTSANPPRRNAITVASSSGEILLGAGSSQNLIFYDPIEVSNAGVSVSFNKEADQTGSVVFSGATVNSADFHQRNLQTKTPAPLTLSNGFLCIEDHAQLTVNRFTQTGGVVSLGNGAVLSCYKNGTGDSASNASITLKHIGLNLSSILKSGAEIPLLWVEPTNNSNNYTADTAATFSLSDVKLSLIDDYGNSPYESTDLTHALSSQPMLSISEASDNQLQSENIDFSGLNVPHYGWQGLWTWGWAKTQDPEPASSATITDPQKANRFHRTLLLTWLPAGYVPSPKHRSPLIANTLWGNMLLATESLKNSAELTPSGHPFWGITGGGLGMMVYQDPRENHPGFHMRSSGYSAGMIAGQTHTFSLKFSQTYTKLNERYAKNNVSSKNYSCQGEMLFSLQEGFLLTKLVGLYSYGDHNCHHFYTQGENLTSQGTFRSQTMGGAVFFDLPMKPFGSTHILTAPFLGALGIYSSLSHFTEVGAYPRSFSTKTPLINVLVPIGVKGSFMNATHRPQAWTVELAYQPVLYRQEPGIAAQLLASKGIWFGSGSPSSRHAMSYKISQQTQPLSWLTLHFQYHGFYSSSTFCNYLNGEIALRF

SEQ ID NO: 14 - DNA, 2895 bp, Chlamydia trachomatis
atgaaaaaagcgtttttctttttccttatcggaaactccctatcaggactagctagagaggttccttctagaatctttcttatgcccaactcagttccagatcctacgaaagagtcgctatcaaataaaattagtttgacaggagacactcacaatctcactaactgctatctcgataacctacgctacatactggctattctacaaaaaactcccaatgaaggagctgctgtcacaataacagattacctaagcttttttgatacacaaaaagaaggtatttattttgcaaaaaatctcacccctgaaagtggtggtgcgattggttatgcgagtcccaattctcctaccgtggagattcgtgatacaataggtcctgtaatctttgaaaataatacttgttgcagactatttacatggagaaatccttatgctgctgataaaataagagaaggcggagccattcatgctcaaaatctttacataaatcataatcatgatgtggtcggatttatgaagaacttttcttatgtccaaggaggagccattagtaccgctaatacctttgttgtgagcgagaatcagtcttgttttctctttatggacaacatctgtattcaaactaatacagcaggaaaaggtggcgctatctatgctggaacgagcaattcttttgagagtaataactgcgatctcttcttcatcaataacgcctgttgtgcaggaggagcgatcttctcccctatctgttctctaacaggaaatcgtggtaacatcgttttctataacaatcgctgctttaaaaatgtagaaacagcttcttcagaagcttctgatggaggagcaattaaagtaactactcgcctagatgttacaggcaatcgtggtaggatcttttttagtgacaatatcacaaaaaattatggcggagctatttacgctcctgtagttaccctagtggataatggccctacctactttataaacaatatcgccaataataaggggggcgctatctatatagacggaaccagtaactccaaaatttctgccgaccgccatgctattatttttaatgaaaatattgtgactaatgtaactaatgcaaatggtaccagtacgtcagctaatcctcctagaagaaatgcaataacagtagcaagctcctctggtgaaattctattaggagcagggagtagccaaaatttaattttttatgatcctattgaagttagcaatgcaggggtctctgtgtccttcaataaggaagctgatcaaacaggctctgtagtattttcaggagctactgttaattctgcagattttcatcaacgcaatttacaaacaaaaacacctgcaccccttactctcagtaatggttttctatgtatcgaagatcatgctcagcttacagtgaatcgattcacacaaactgggggtgttgtttctcttgggaatggagcagttctgagttgctataaaaatggtacaggagattctgctagcaatgcctctataacactgaagcatattggattgaatctttcttccattctgaaaagtggtgctgagattcctttattgtgggtagagcctacaaataacagcaataactatacagcagatactgcagctaccttttcattaagtgatgtaaaactctcactcattgatgactacgggaactctccttatgaatccacagatctgacccatgctctgtcatcacagcctatgctatctatttctgaagctagcgataaccagctacaatcagaaaatatagatttttcgggactaaatgtccctcattatggatggcaaggactttggacttggggctgggcaaaaactcaagatccagaaccagcatcttcagcaacaatcactgatccacaaaaagccaatagatttcatagaaccttactactaacatggcttcctgccgggtatgttcctagcccaaaacacagaagtcccctcatagctaacaccttatgggggaatatgctgcttgcaacagaaagcttaaaaaatagtgcagagctgacacctagtggtcatcctttctggggaattacaggaggaggactaggcatgatggtttaccaagatcctcgagaaaatcatcctggattccatatgcgctcttccggatactctgcggggatgatagcagggcagacacacaccttctcattgaaattcagtcagacctacaccaaactcaatgagcgttacgcaaaaaacaacgtatcttctaaaaattactcatgccaaggagaaatgctcttctcattgcaagaaggtttcttgctgactaaattagttgggctttacagctatggagaccataactgtcaccatttctatactcaaggagaaaatctaacatctcaagggacgttccgcagtcaaacgatgggaggtgctgtcttttttgatctccctatgaaaccctttggatcaacgcatatactgacagctccctttttaggtgctcttggtatttattctagcctgtctcactttactgaggtgggagcctatccgcgaagcttttctacaaagactcctttgatcaatgtcctagtccctattggagttaaaggtagctttatgaatgctacccacagacctcaagcctggactgtagaattggcataccaacccgttctgtatagacaagaaccagggatcgcagcccagctcctagccagtaagggtatttggttcggtagtggaagcccctcatcgcgtcatgccatgtcctataaaatctcacagcaaacacaacctttgagttggttaactctccatttccagtatcatggattctactcctcttcaaccttctgtaattatctcaatggggaaattgctctgcgattctag

SEQ ID NO: 15 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 16 - DNA, 4596 bp, Chlamydia trachomatis
atgagttccgagaaagatataaaaagcacctgttctaagttttctttgtctgtagtagcagctatccttgcctctgttagcgggttagctagttgcgtagatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcggatactcttcccgggaaggtagagcaaagtactttgttctcagtaaccaatcccgtggttttccaaggtgtggaccaacaggatcaagtctcttcccaagggttaatttgtagttttacgagcagcaaccttgattctcctcgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggtggtgcttctgttttagggaccattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattactaataattccgaaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctgggaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagagcagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctagaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagccacattatgtagttatggttttaaacaagatgctggagctaagttggtattggctgctggagctaaactgaagattttagattcaggaactcctgtacaacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttattgaagaatgaggctaaagttccattgatgtctttcgttgcttctggtgatgaagcttcagccgaaatcagtaacttgtcggtttctgatttacagattcatgtagtaactccagagattgaagaagacacatacggccatatgggagattggtctgaggctaaaattcaagatggaactcttgtcattagttggaatcctactggatatcgattagatcctcaaaaagcaggggctttagtatttaatgcattatgggaagaaggggctgtcttgtctgctctgaaaaatgcacgctttgctcataatctcactgctcagcgtatggaattcgattattctacaaatgtgtggggattcgcctttggtggtttccgaactctatctgcagagaatctggttgctattgatggatacaaaggagcttatggtggtgcttctgctggagtcgatattcaattgatggaagattttgttctaggagttagtggagctgctttcctaggtaaaatggatagtcagaagtttgatgcggaggtttctcggaagggagttgttggttctgtatatacaggatttttagctggatcctggttcttcaaaggacaatatagccttggagaaacacagaacgatatgaaaacgcgttatggagtactaggagagtcgagtgcttcttggacatctcgaggagtactggcagatgctttagttgaataccgaagtttagttggtcctgtgagacctactttttatgctttgcatttcaatccttatgtcgaagtatcttatgcttctatgaaattccctggctttacagaacaaggaagagaagcgcgttcttttgaagacgcttcccttaccaatatcaccattcctttagggatgaagtttgaattggcgttcataaaaggacagttttcagaggtgaactctttgggaataagttatgcatgggaagcttatcgaaaagtagaaggaggcgcggtgcagcttttagaagctgggtttgattgggagggagctccaatggatcttcctagacaggagctgcgtgtcgctctggaaaataatacggaatggagttcttacttcagcacagtcttaggattaacagctttttgtggaggatttacttctacagatagtaaactaggatatgaggcgaatactggattgcgattgatcttttaa

SEQ ID NO: 17 - protein, 340 aa, Chlamydia trachomatis; identical to SEQ ID NO: 2

SEQ ID NO: 18 - DNA, 1023 bp, Chlamydia trachomatis; identical to SEQ ID NO: 1

SEQ ID NO: 19 - protein, 350 aa, Chlamydia trachomatis
MSLFSKFKAQWMFLHSRELCSSTSDIGNTCSDPVFQVLCNPVRSEISYKVGDSLGVFPTNPSILVDSVLDALQYGPRSPVVSRHADSVLPLHEFLTSYVDLDKIPKSLRPFFPGDLDDTWSLAEAILVYQPRIPFEEFIRSAMPLLPRFYSIASSPTCSHGKLELLVRCVSFQGKTQLRYGLCSAFLCKDLQEGESFRGFIQPTRHFTLEQKNFGKPLIMIGAGTGIAPYKGFLQHRIYHQDVGSNILFFGERFEKSNFYYRDFLQELIVSGKLQLFTAFSRDSESKLYVQNVIEQQKELIQEVYEQEAFFFVCGKKILGTEVKRALEQILGPKAVRELIAQKRLVSDVY

SEQ ID NO: 20 - DNA, 1053 bp, Chlamydia trachomatis
atgtctttattttctaaattcaaagctcagtggatgtttttacattcacgtgagctttgttcttccacatcggatattgggaatacttgttcggatcctgtttttcaggttttatgtaatccggttcgttctgagatttcctataaagttggggattctttgggggtattcccaacaaatccttccatattagtcgattcagttctagatgctttacagtatggccccaggtctcctgtcgtatctcggcatgcagattctgttctccctcttcacgaatttcttactagttacgtagacttagataaaattccaaaatcgttaagaccttttttcccaggggatttagacgatacctggtctttagctgaagctattttggtttaccagccgcgtattccttttgaagagtttattcggagtgcgatgcctttattgcctcgattttattctatagcttcttctccaacatgttctcatgggaagctagagttgctcgtgcgctgtgttagtttccaaggtaaaacgcagctgcgctatggattatgttcggcttttttatgtaaggacttacaagagggagagtcttttcgtgggtttatacaaccgacgcggcattttactttggagcagaaaaattttgggaaacctttaattatgatcggagcagggacaggtatcgctccgtacaaagggttcttacaacatcgaatataccatcaggacgtaggctccaatattctattctttggagagcgttttgagaaaagtaacttctattaccgggattttctccaggagctgatcgtttcaggaaaactccagttattcacagccttttccagagattccgagtctaaattgtatgttcagaatgttatagagcaacaaaaagaacttatacaagaagtctacgaacaagaagctttcttttttgtttgtgggaaaaaaatccttggtacggaagttaaacgtgctttagagcagatattaggtcctaaggcggtacgagagctgattgcacagaagagactagtttcagacgtatactaa

SEQ ID NO: 21 - protein, 1770 aa, Chlamydia trachomatis
MKFMSATAVFAAALSSVTEASSIQDQIKNTDCNVSKLGYSTSQAFTDMMLADNTEYRAADSVSFYDFSTSSRLPRKHLSSSSEASPTTEGVSSSSSGETDEKTEEELDNGGIIYAREKLTISESQDSLSNQSIELHDNSIFFGEGEVIFDHRVALKNGGAIYGEKEVVFENIKSLLVEVNIAVEKGGSVYAKERVSLENVTEATFSSNGGEQGGGGIYSEQDMLISDCNNVHFQGNAAGATAVKQCLDEEMIVLLAECVDSLSEDTLDSTPETEQTESNGNQDGSSETEDTQVSESPESTPSPDDVLGKGGGIYTEKSLTITGITGTIDFVSNIATDSGAGVFTKENLSCTNTNSLQFLKNSAGQHGGGAYVTQTMSVTNTTSESITTPPLIGEVIFSENTAKGHGGGICTNKLSLSNLKTVTLTKNSAKESGGAIFTDLASIPITDTPESSTPSSSSPASTPEVVASAKINRFFASTAKPAAPSLTEAESDQTDQTETSDTNSDIDVSIENILNVAINQNTSAKKGGAIYGKKAKLSRINNLELSGNSSQDVGGGLCLTESVEFDAIGSLLSHYNSAAKEGGAIHSKTVTLSNLKSTFTFADNTVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGQGIFSGNKAIDNTTEGSSSKSDVLGGAVYAKTLFNLDSGSSRRTVTFSGNTVSSQSTTGQVAGGAIYSPTVTIATPVVFSKNSATNNANNTTDTQRKDTFGGAIGATSAVSLSGGAHFLENVADLGSAIGLVPGTQNTETVKLESGSYYFEKNKALKRATIYAPVVSIKAYTATFNQNRSLEEGSAIYFTKEASIESLGSVLFTGNLVTLTLSTTTEGTPATTSGDVTKYGAAIFGQIASSNGSQTDNLPLKLIASGGNICFRNNEYRPTSSDTGTSTFCSIAGDVKLTMQAAKGKTISFFDAIRTSTKKTGTQATAYDTLDINKSEDSETVNSAFTGTILFSSELHENKSYIPQNVVLHSGSLVLKPNTELHVISFEQKEGSSLVMTPGSVLSNQTVADGALVINNMTIDLSSVEKNGIAEGNIFTPPELRIIDTTTGGSGGTPSTDSESNQNSDDTEEQNNNDASNQGESANGSSSPAVAAAHTSRTRNFAAAATATPTTTPTATTTTSNQVILGGEIKLIDPNGTFFQNPALRSDQQISLLVLPTDSSKMQAQKIVLTGDIAPQKGYTGTLTLDPDQLQNGTISVLWKFDSYRQWAYVPRDNHFYANSILGSQMLMVTVKQGLLNDKMNLARFEEVSYNNLWISGLGTMLSQVGTPTSEEFTYYSRGASVALDAKPAHDVIVGAAFSKMIGKTKSLKRENNYTHKGSEYSYQASVYGGKPFHFVINKKTEKSLPLLLQGVISYGYIKHDTVTHYPTIRERNKGEWEDLGWLTALRVSSVLRTPAQGDTKRITVYGELEYSSIRQKQFTETEYDPRYFDNCTYRNLAIPMGLAFEGELSGNDILMYNRFSVAYMLSIYRNSPTCKYQVLSSGEGGEIICGVPTRNSARGEYSTQLYLGPLWTLYGSYTIEADAHTLAHMMNCGARMTF

SEQ ID NO: 22 - DNA, 5313 bp, Chlamydia trachomatis
atgaaatttatgtcagctactgctgtatttgctgcagcactctcctccgttactgaggcgagctcgatccaagatcaaataaagaataccgactgcaatgttagcaaattaggatattcaacttctcaagcatttactgatatgatgctagcagacaacacagagtatcgagctgctgatagtgtttcattctatgacttttcgacatcttccagattacctagaaaacatcttagtagtagtagtgaagcttctccaacgacagaaggagtgtcttcatcttcatctggagaaactgatgagaaaacagaagaagaactagacaatggcggaatcatttatgctagagagaaactaactatctcagaatctcaggactctctctctaatcaaagcatagaactccatgacaatagtattttcttcggagaaggtgaagttatctttgatcacagagttgccctcaaaaacggaggagctatttatggagagaaagaggtagtctttgaaaacataaaatctctactagtagaagtaaatatcgcggtcgagaaagggggtagcgtctatgcaaaagaacgagtatctttagaaaatgttaccgaagcaaccttctcctccaatggtggggaacaaggtggtggtggaatctattcagaacaggatatgttaatcagtgattgcaacaatgtacatttccaagggaatgctgcaggagcaacagcagtaaaacaatgtctggatgaagaaatgatcgtattgctcgcagaatgcgttgatagcttatccgaagatacactggatagcactccagaaacggaacagactgagtcaaatggaaatcaagacggttcgtctgaaacagaagatacacaagtatcagaatcaccagaatcaactcctagccccgacgatgttttaggtaaaggtggtggtatctatacagaaaaatctttgaccatcactggaattacagggactatagattttgtcagtaacatagctaccgattctggagcaggtgtattcactaaagaaaacttgtcttgcaccaacacgaatagcctacagtttttgaaaaactcggcaggtcaacatggaggaggagcctacgttactcaaaccatgtctgttactaatacaactagtgaaagtataactactccccctctcataggagaagtgattttctctgaaaatacagctaaagggcacggtggtggtatctgcactaacaaactttctttatctaatttaaaaacggtgactctcactaaaaactctgcaaaggagtctggaggagctatttttacagatctggcgtctataccaataacagataccccagaatcttctaccccctcttcctcctcgcctgcaagcactcctgaagtagttgcttctgctaaaataaatcgattctttgcctctacggcaaaaccggcagccccttctctaacagaggctgagtctgatcaaacggatcaaacagaaacttctgatactaatagcgatatagacgtgtcgattgagaacattttgaatgtcgctatcaatcaaaacacttctgcgaaaaaaggaggggctatttacgggaaaaaagctaaactttcccgtattaacaatcttgaactttcagggaattcatcccaggatgtaggaggaggtctctgtttaactgaaagcgtagaatttgatgcaattggatcgctcttatcccactataactctgctgctaaagaaggtggggctattcattctaaaacggttactctatctaacctcaagtctaccttcacttttgcagataacactgttaaagcaatagtagaaagcactcctgaagctccagaagagattcctccagtagaaggagaagagtctacagcaacagaagatccaaattctaatacagaaggaagttcggctaacactaaccttgaaggatctcaaggggatactgctgatacagggactggtgatgttaacaatgagtctcaagacacatcagatactggaaacgctgaatctgaagaacaactacaagattctacacaatctaatgaagaaaatacccttcccaatagtaatattgatcaatctaacgaaaacacagacgaatcatctgatagccacactgaggaaataactgacgagagtgtctcatcgtcctctgaaagtggatcatctactcctcaagatggaggagcagcttcttcaggggctccctcaggagatcaatctatctctgcaaacgcttgtttagctaaaagctatgctgcgagtactgatagctcccccgtatctaattcttcaggttcagaagagcctgtcacttcttcttcagattcagacgttactgcatcttctgataatccagactcttcctcatctggagatagcgctggagactctgaagaaccgactgagccagaagctggttctacaacagaaactcttactttaataggaggaggtgctatctatggagaaactgttaagattgagaacttctctggccaaggaatattttctggaaacaaagctatcgataacaccacagaaggctcctcttccaaatctgacgtcctcggaggtgcggtctatgctaaaacattgtttaatctcgatagcgggagctctagacgaactgtcaccttctccgggaatactgtctcttctcaatctacaacaggtcaggttgctggaggagctatctactctcctactgtaaccattgctactcctgtagtattttctaaaaactctgcaacaaacaatgctaataacactacagatactcagagaaaagacacctttggaggagctatcggagctacttctgctgtttctctatcaggaggggctcatttcttagaaaacgttgctgacctcggatctgctattgggttggtgccaggcacacaaaatacagaaacagtgaaattagagtctggctcctactactttgaaaaaaataaagctttaaaacgagctactatttacgcacctgtcgtttccattaaagcctatactgcgacatttaaccaaaacagatctctagaagaaggaagcgcgatttactttacaaaagaagcatctattgagtctttaggctctgttctcttcacaggaaacttagtaaccctaacgctaagcacaactacagaaggcacaccagccacaacctcaggagatgtaacaaaatatggtgctgctatctttggacaaatagcaagctcaaacggatctcagacggataaccttcccctgaaactcattgcttcaggaggaaatatttgtttccgaaacaatgaataccgtcctacttcttctgataccggaacctctactttctgtagtattgcgggagatgttaaattaaccatgcaagctgcaaaagggaaaacgatcagtttctttgatgcaatccggacctctactaagaaaacaggtacacaggcaactgcctacgatactctcgatattaataaatctgaggattcagaaactgtaaactctgcgtttacaggaacgattctgttctcctctgaattacatgaaaataaatcctatattccacaaaacgtagttctacacagtggatctcttgtattgaagccaaataccgagcttcatgttatttcttttgagcagaaagaaggctcttctctcgttatgacacctggatctgttctttcgaaccagactgttgctgatggagctttggtcataaataacatgaccattgatttatccagcgtagagaaaaatggtattgctgaaggaaatatctttactcctccagaattgagaatcatagacactactacaggtggaagcggtggaaccccatctacagatagtgaaagtaaccagaatagtgatgataccgaggagcaaaataataatgacgcctcgaatcaaggagaaagcgcgaatggatcgtcttctcctgcagtagctgctgcacacacatctcgtacaagaaactttgccgctgcagctacagccacacctacgacaacaccaacggctacaactacaacaagcaaccaagtaatcctaggaggagaaattaaactcatcgatcctaatgggaccttcttccagaaccctgcattaagatccgaccaacaaatctccttgttagtgctccctacagactcatcaaaaatgcaagctcagaaaatagtactgacgggtgatattgctcctcagaaaggatatacaggaacactcactctggatcctgatcaactacaaaatggaacgatctcagtgctctggaaatttgactcttatagacaatgggcttatgtacctagagacaatcatttctatgcgaactcgattctgggatctcaaatgttaatggtcacagtcaaacaaggcttgctcaacgataaaatgaatctagctcgctttgaggaagttagctataacaacctgtggatatcaggactaggaacgatgctatcgcaagtaggaacacctacttctgaagaattcacttattacagcagaggagcttctgttgccttagatgctaaaccagcccatgatgtgattgttggagctgcatttagtaagatgatcgggaaaacaaaatccttgaaaagagagaataactacactcacaaaggatccgaatattcttaccaagcatcggtatacggaggcaaaccattccactttgtaatcaataaaaaaacggaaaaatcgctaccgctattgttacaaggagtcatctcttacggatatatcaaacatgatacagtgactcactatccaacgatccgtgaacgaaacaaaggagaatgggaagacttaggatggctgacagctctccgtgtctcctctgtcttaagaactcctgcacaaggggatactaaacgtatcactgtttacggagaattggaatactccagtatccgtcagaaacaattcacagaaacagaatacgatcctcgttacttcgacaactgcacctatagaaacttagcaattcctatggggttagcattcgaaggagagctctctggtaacgatattttgatgtacaacagattctctgtagcatacatgctatcaatctatcgaaattctccaacatgcaaataccaagtgctctcttcaggagaaggcggagaaattatttgtggagtaccgacaagaaactcagctcgcggagaatacagcacgcagctgtacctgggacctttgtggactctgtatggatcctacacgatagaagcagacgcacatacactagctcatatgatgaactgcggtgctcgtatgacattctaa

SEQ ID NO: 23 - protein, 408 aa, Chlamydia trachomatis
MMKPLRFGYFFCAIYFTLLQAAFAKEPNSCPDCQNNWKEVTHTDQLPENIIHADDACYHSGYVQALIDMHFLDSCCQVIVENQTAYLFSLPTDDVTRNAIINLIKDLPFIHSVEICQASYQTCHHQGPHGKTSLPEQRSFCTKVCGKEAIWLPQNTILFSPLVADPRQATNSAGIRFNDEVLGKRVGSATFGGDFIFLRLFDISRFHGDMDIGLQGAVFSVFDLDHPEACMVNSDFFVAALCNFAVNKWSYRFRLWHLSSHLGDEFILANQLPPKKRYNRSDEAVDFFASFRYTPQIRVYGGIGYIISRDLTFPEDPLYFEGGIELRPFGLREDNLHAQPVFAMHFRFWEEHDFSIDQTYIVGMEWSKFQDVGRKVRAVLEYHQGFSHEGQFVREECDYYGFRLSYGF

SEQ ID NO: 24 - DNA, 1227 bp, Chlamydia trachomatis
atgatgaaacctctacgtttcggttatttcttttgcgcaatctattttactttgttacaggcagcgtttgctaaagaaccgaattcttgtcccgactgccagaataattggaaagaagtcacccacacggatcaactcccagaaaacatcattcatgctgatgatgcttgttatcactctggttatgtacaggctctcattgatatgcatttcttagatagctgctgccaggtcatcgttgaaaaccaaactgcttacttattttctcttcctacagatgatgttacgcgcaacgccattatcaacctaattaaagaccttccattcattcactccgtagaaatctgccaagcatcctatcaaacctgtcatcatcaaggccctcatggaaagacttctcttccagaacaacgttctttctgtacaaaggtctgtggaaaagaagctatttggttaccacagaataccatcctattctcgcctcttgtagcagatcctagacaagcaactaatagtgcaggtatccgttttaacgacgaagtcttaggaaaacgtgttggctctgctaccttcggtggagatttcatcttcttacgattatttgatatctcccgattccatggagacatggatattggtctccaaggagctgtattctctgttttcgacctggatcatccagaagcttgcatggtcaactctgacttttttgtcgccgctttgtgcaactttgcagtgaacaaatggagctaccgcttcagactatggcatctttcttctcatcttggcgacgaatttattcttgccaaccagttacctcctaaaaaacgttataatcgaagcgatgaagccgtcgatttctttgcttcttttcgttacactccacagatccgtgtttatggaggtattgggtatatcattagtcgagatttaacattccctgaagatcctctttactttgaaggaggtatcgaactacgtcctttcggattacgggaagacaaccttcatgcccaacccgtctttgctatgcattttcgcttttgggaagagcatgacttttctatagaccaaacttatatagtaggcatggagtggtccaaattccaggatgtagggagaaaagtgcgcgctgtattggaataccaccaaggtttctcccacgaaggacaatttgtccgagaagaatgcgattattatggctttcgattaagttatggcttctag

SEQ ID NO: 25 - protein, 792 aa, Chlamydia trachomatis
MLGIRKKTILQLAVLLLLTFSRSSFCSTSEGRMVVESITITTQGENTQNKRAIPKIKTKQGTLFSQADFDEDLRTLSKDFDRVEPIVEFRNGQAVISLILTAKPVIREINISGNEAIPTHKILKTLELYKNDLFDRELFFKNFDALRTLYLKRGYYDSQLSYSHNHNEKEGFIDISIEIKEGRHGRIKKLTISGITRTEASDLGDIVLTKQYSTTTSWFTGAGVYHPDMVEQDLFAITNYFQNKGYADAKVSKEVSTDAKGNITLLIVVDKGPLYTLGHVHIEGFTALSKRLLDKQLLVGPNSLYCPDKIWTGAQKIRSAYARYGYVNTNVDVSFSAHPTLPVYDVTYRVSEGSPYKIGLIKIKGNTHTKHDVILHETSLFPGDTFDRLKLEGTETRLRNTGYFKSVSVYTVRSQLDPLDSNDLYRDVFIEVKETETGNLGLFLGFSSIDHLFGGAEIAESNFDLFGARNFLKKGFKSLRGGGEYLFLKANLGDKVTDYTVKWTKPHFLNTPWILGVELDKSINKALSKDYSVDTYGGNISTTYILNDKLKYGMYYRGSQTSLSLRKKTSSSNRPGPDLDSNKGFVSAAGLNVLYDSIDNPRKPTMGIRSSLNFELSGLGGTYQFTKLTASGSIYRLLTKKGVLKVRAEAKFIKPFGTTTAQGIPVSERFFLGGETTVRGYKPFIIGPKFSPTEPQGGLSSLLLTEEFQYPLISQPCINAFVFLDSGFIGIEEYTIRLKDLCSSAGFGLRFDMMNNVPIMLGWGWPFRPTEILNNEKIDVSQRFFFALGGVF

SEQ ID NO: 26 - DNA, 2379 bp, Chlamydia trachomatis
atgcttggaatacgcaaaaaaacgattctgcaactcgctgttttactgttgctcaccttttcacgaagttctttctgttcaacttcagaaggacgtatggtcgtagagtctatcaccattacgactcaaggagagaatactcaaaataaacgagctattcctaaaataaaaacaaagcaggggacgttgttctctcaagcagattttgatgaagatctaagaacactttcgaaagattttgatcgagtagagcctatcgtagagtttcgtaatggacaagctgtgatctctctgattctgacggcaaaacctgttatcagagagatcaatatttcaggaaatgaagctatccccactcataaaattctgaaaactttagagctttataaaaatgatctttttgatcgggaattattctttaaaaattttgatgcgctaagaactctttatttgaaacgagggtactacgattctcaactctcctattctcataatcataatgagaaagagggctttatcgatatttccatcgagattaaagaaggacgtcacggtcgcataaaaaaattaacgatttcgggaattacgcgaacagaagcatcagacttaggtgacattgttttaactaaacaatactccacaacaacgagctggttcactggtgccggagtgtatcatccggacatggtagagcaagacttatttgctatcacaaattacttccaaaataaaggatatgctgatgctaaagtaagcaaagaggtctctacagatgctaaaggaaacattactttgcttatcgttgtagacaaaggacctttatacacattaggtcacgtacatatagaaggattcacagcgttatccaaaagactgctcgataaacaactattggttggacctaactccttatattgcccagataaaatttggactggagcacaaaagattcgtagcgcatacgctagatatggctacgtgaacactaacgttgatgtctccttctcagcgcaccccactctacctgtttacgatgttacctatcgagtgagtgaaggatctccctacaaaatcgggttaattaaaatcaaagggaacactcatactaagcatgatgtgattttgcatgagactagtcttttccctggagacacttttgatagattaaaactggaaggtacagagactcgtttacgcaacaccggctactttaaaagtgtaagtgtctatacggttcgttcccaattagatcctcttgattctaacgacctttatcgagatgtttttattgaagtcaaagagactgaaacaggaaatcttgggctattcttaggattcagctccattgaccatttatttggaggggcagaaattgcagaaagcaactttgatttatttggagcccgaaactttctcaaaaaaggattcaaatctttaagaggtggtggagaatacctcttcctaaaagctaatttaggagataaggtcaccgattacactgttaaatggacgaaaccacacttcttaaataccccttggattcttggagtagaattagataaatcaattaataaagctttatcaaaagactactctgtggatacctatggagggaatatcagtaccacctacattcttaacgataagttaaaatatgggatgtattaccgtggtagccaaacaagcttaagtttgcgcaaaaaaacgtccagctctaatagacctggaccagatttagatagtaataaaggatttgtttccgcagcgggactcaatgttctctatgattctattgataatcctagaaaacctactatgggaatccgcagctccttaaactttgaattatctggtttaggcggaacttaccaatttactaaactaacagctagtggttctatctatcgcttattaactaaaaaaggtgttttgaaagtccgtgcagaagctaagtttatcaaacctttcggaacaacaactgcacaaggcattcctgtcagcgaacggttcttcttaggaggtgaaaccactgttcgcggttacaaaccttttattattggaccgaaattttctcctactgaaccacaaggaggcttgtcttccctactattaacagaagaatttcaatatcctttgatttctcaaccttgcattaatgcctttgtatttctagattccggattcattgggatagaagagtacactattcgcctgaaagacctttgcagtagcgccggatttggtctacgctttgatatgatgaataatgtgccaattatgctaggctggggttggccgttccgcccaacagaaatcctcaataatgaaaaaattgatgtatctcaaagattcttttttgccttgggaggagtattctag

SEQ ID NO: 27 - protein, 254 aa, Chlamydia trachomatis
MFAFLTSKKVGILPSRWGSSRFPGKPLAKILGKTLVQRSYENALSSQSLDCVVVATDDQRIFDHVVEFGGLCVMTSTSCANGTERVEEVVSRHFPQAEIVVNIQGDEPCLSPTVIDGLVSTLENNPAADMVTSVTETTDPEAILTDHKVKCVFDKNGKALYFSRSAIPHNFKHPTPIYLHIGVYAFRKAFLSEYVKIPPSSLSLAEDLEQLRVLEIGRSIYVHVVQNATGPSVDYPEDITKVEQYLLCLSKASF

SEQ ID NO: 28 - DNA, 765 bp, Chlamydia trachomatis
gtgtttgcgtttttaaccagcaaaaaagtcggcattctcccctctagatggggaagctcccgcttccccggaaaacctctagcaaaaattctagggaaaacccttgttcaaagatcctatgaaaatgccttaagcagtcaatctctagattgcgttgttgtggcaacagatgatcaacgaatttttgaccatgtcgttgaatttggggggctctgtgtcatgactagcacatcttgcgctaacggaactgagcgagtagaagaggttgtgtctcgacattttcctcaagcagagattgttgtgaacatccaaggagacgagccctgtttatctcctaccgtcatagatgggcttgtgagcacgctagagaacaatcctgctgcagatatggtcacatctgttacagaaacaacagaccccgaagcgatattgacagatcacaaagtgaagtgtgttttcgataagaatggcaaagctctttactttagcagaagcgctattcctcacaactttaaacacccaacgcctatttatctgcatattggtgtttatgcttttagaaaagcttttctaagtgaatatgttaaaattcctccttcctcgttaagcctagccgaagatcttgaacaattacgagtattagaaataggtcgttctatctacgttcatgtcgttcagaatgcaacgggcccttctgttgattatcccgaagatataaccaaagtggagcagtatttattatgtctttcaaaagcatctttttga

SEQ ID NO: 29 - protein, 692 aa, Chlamydia trachomatis
MIRQWYGFFLCLLFSYTSCFGVEENSGRATPTVELVSESEQAVEGEVLRIGVLIAIPEGEHIYWKNPGKLGMPLRISWDLPSGCRLLEEHWPTPEIFEEDGVVYFGYKHSTMVVADIRVSKEIETRPLEIKAQVEWLSCGASCLPGSSSRVLVIPIDQGPLIPNSKETFTFSRALAAQPRPLDAAIKISYQPDGLDVFVPAGKADRATQAWFIAENTRDFAYAQEVPLEQATTYIWKLKHPEGNMPKGIGLSGILIFKDDAGKVVASYQVEENQVEQLSALSWRFLSILLMAFIGGILLNIMPCVLPLITLKVFSLIKSAADHHSSSVIGGIGFTLGAIVSFWGLAFCAFLLKVLGQNIGWGFQLQEPMFVAVLIIVFFLFALSSLGVFEMGIICLSLGKKLQEEGGASVRKNQIWGAFFNGMLTTLVTTPCTGPFLGSVFGLVMAVSFVKQLAIFTAIGLGMASPYLLFASFPKMLAILPKPGPWMSTFKQLTGFMLLATATWLIWIFGVETSATAVTILLVGLWLAAVGAWILGRWGTLVSPRNQRLLASVVFIFCILSSLVITSIGVRYFDENVPPAHSSDWQSFSPEKLADLREKGIPVFVNFTAKWCLTCQLNKPLLHANMQAFAAKGVVTLEADWTKKDPKITEELARLGRASVPSYVYYPAGNKAPLILPERLSQSALEEMVFSQ

SEQ ID NO: 30 - DNA, 2079 bp, Chlamydia trachomatis
atgattcggcaatggtatggattttttctttgcttgctgttcagctatacgtcttgttttggtgtagaagaaaatagtggaagagctacgcctacagtagaacttgttagtgaaagcgaacaagctgttgaaggagaagtgcttcgtatcggagtattgattgctattccagaaggagagcatatctactggaaaaatccagggaagcttggaatgcctttgcgcatttcttgggatttgccatcaggatgtaggttgctggaggaacattggccgactccagagattttcgaagaggatggggttgtttattttggttataaacattctacaatggtggttgcggatattcgcgtttctaaagagatagaaacgcgtccattggagataaaagcgcaagttgaatggttgtcttgcggtgcatcttgtctcccaggttcttcgtcaagggttcttgtgattcctatagatcaggggccgttaattcctaatagtaaagagacattcactttttcccgtgcgttagcggctcaacctcgacctttggatgctgccataaagatttcttatcagcctgatggcttagatgtttttgtgccagcagggaaagcggatcgggcaacccaggcatggttcattgctgaaaacacgcgagattttgcttatgctcaagaggttcctcttgagcaagcgactacgtacatatggaagttgaaacatcctgaaggaaatatgcctaagggtattgggttgtcggggattcttatattcaaggatgatgcagggaaagtagtcgcttcgtatcaagtagaagagaatcaagtcgaacagctttcggcattgagctggaggtttctctctattcttcttatggctttcattggtggaatcttattaaacatcatgccctgtgtattgcctctgattactttgaaagtatttagtttaattaaatcggcggcagatcaccattcttcctctgtgattggagggattgggtttactttaggggctattgtaagcttttggggactcgctttttgtgcgtttttgttaaaggttttagggcaaaatattggatggggattccagcttcaagaacccatgtttgttgccgttttaattattgtcttcttcttatttgctctgagttcgttaggcgtttttgagatgggaataatttgtctgagcctagggaaaaaattgcaagaagagggaggggcatcggtaaggaagaatcagatctggggagcttttttcaatgggatgttgactaccctggttacaactccttgcactgggccttttcttggctctgtatttggattagttatggcagtgtcttttgttaagcagctggcaatttttactgctataggattaggaatggcaagtccctatctattatttgcttcttttccgaagatgctagccattttacctaaacctggtccttggatgagtacgtttaaacagttgactgggtttatgttgcttgctactgcaacttggcttatctggatttttggggtagagacgagtgcaaccgctgtaactattcttcttgtaggattgtggttggctgctgtaggtgcatggattctagggagatggggaacccttgtatctccgcgtaatcagcggcttcttgcttccgttgtattcattttctgtattttaagttccttagtgattacctctataggtgtccgttattttgatgagaacgtccctcctgcacatagctctgattggcaatctttttctcccgaaaagctagctgatttacgcgaaaaagggattccagtttttgtaaatttcactgcaaagtggtgtttaacgtgtcaactcaataagcctcttcttcatgccaatatgcaagcttttgctgctaagggcgtagttactttagaagcagattggacgaaaaaagatccaaaaattacagaagaactcgctcgtttaggccgagccagtgtaccttcttatgtgtattaccctgcggggaacaaagctccgcttattcttccagaaagattatcgcaatctgctttggaagagatggttttttctcagtag

SEQ ID NO: 31 - protein, 326 aa, Chlamydia trachomatis
MFRYTLSRSLFFILALFFCSACDSRSMITHGLSGRDANEIVVLLVSKGVAAQKVPQAASSTGGSGEQLWDISVPAAQITEALAILNQAGLPRMKGTSLLDLFAKQGLVPSEMQEKIRYQEGLSEQMATTIRKMDGIVDASVQISFSPEEEDQRPLTASVYIKHRGVLDNPNSIMVSKIKRLVASAVPGLCPENVSVVSDRASYSDITINGPWGLSDEMNYVSVWGIILAKHSLTKFRLVFYFLILLLFILSCGLLWVIWKTHTLISALGGTKGFFDPAPYSQLSFTQNKPAPKETPGAAEGAEAQTASEQPSKENAEKQEENNEDA

SEQ ID NO: 32 - DNA, 981 bp, Chlamydia trachomatis
atgtttcgttatactctttctcgatccttatttttcattttggctcttttcttctgctcggcttgtgatagtcgttccatgattacacacggcttgtcaggacgtgatgctaatgaaatcgtagtgcttctagtcagtaaaggggtcgctgcacagaaagttccccaagcagcgtcctcaacaggaggatctggagaacaactctgggatatttcggttcctgcagcacaaattacagaggctctagctattctgaaccaagctgggcttccaagaatgaaaggaaccagccttcttgatctattcgctaaacaagggctggtcccttctgaaatgcaagaaaaaatccgctaccaagaaggtctttcagaacaaatggctacgaccattagaaagatggacggtatcgtcgatgcgagcgtacagatttccttttctcctgaagaagaagatcaacggccgctaacagcctctgtatatatcaaacacagaggggtattagacaaccctaacagtattatggtgtctaagattaaacgtttagttgcgagtgctgtcccaggactatgtcccgagaacgtttccgtagtcagtgaccgagcttcttatagtgacattactattaatggcccttggggactctccgatgaaatgaattatgtttctgtatgggggatcattctagctaagcattcccttactaaattccgccttgttttctatttcttaattctccttctcttcattctttcctgtgggctactctgggtcatttggaaaacacacacactgatttctgctctgggtggaacaaaaggattctttgatcctgctccttactcacagctctctttcactcagaataagccagctccaaaagaaactcctggagcagcagaaggtgcagaagcgcaaaccgcttcggaacaaccctctaaagaaaacgcagaaaaacaagaagagaataacgaggacgcttaa

SEQ ID NO: 33 - protein, 200 aa, Chlamydia trachomatis
MEKRGVIVHILVCLLTIFGTFSLPAFGAHFLAEEEQFYMDRFVFSGQYPDMETMEIHAERKKRVQFDVTGSFPKLESVVYKGSFGLLRSKIKGECPELSSVNLSCTSCRMDLDFRGEWKKNASIYIRNEQEPITIMLPKDIGVVVYTQVDMNSKVVAEGSLIKRGRGFWKKTFRNSLVGESPVTLTFHVETRNGGVIFLR

SEQ ID NO: 34 - DNA, 603 bp, Chlamydia trachomatis
atggagaagagaggcgttattgtgcatatactagtttgtttgttgacaatcttcggaacgttcagtttacccgctttcggcgcgcattttctcgcggaagaagagcagttttatatggatcggtttgttttctctgggcagtatccagatatggaaactatggaaatccatgcagaaagaaaaaaacgtgtacaatttgatgtgacgggaagcttccctaagttggagagcgtggtttataagggatcttttggattgctgcgttcgaaaataaagggagagtgtccagaactgtcttctgtaaatctttcttgtacctcctgcagaatggatttagattttcgaggggagtggaaaaagaatgcgtctatttatattcgtaatgagcaagagccaattacaattatgttgcctaaagacattggtgtagttgtctatacgcaggttgatatgaatagtaaagtagttgcagagggatcactaatcaagagaggaagaggtttttggaagaaaacttttcggaattctttggtaggagaatcccctgtgacgctaacttttcatgtagagactcgtaatggaggagttatttttctccgttag

SEQ ID NO: 35 - protein, 305 aa, Chlamydia trachomatis
MFDSDHNSIFQSDLCQRLVVHSILLTFLVILLCTSLYPSSAFIVGLLSSACAALGTYEMGAMVRIKFPFSFTRYSALGSAIFIALTCLTARCKMCFPEHIDLLPWFFLFFWTIRLVFKSRHYKLGPIGSTGLALFCMLYVSVPIRLFLHILYGFVHTDTPFVGIWWAIFLIATTKSSDIFGYFFGKAFGKKRIAPVISPNKTVVGFIAGCCGSILVSLLFYSHLPKAFADQIAVPWILIALGTVLGVSGFFGDIIESTFKRDAQIKNSSDLESIGGMLDVLDSLLLSTPIVYAILLITQNRTFLG

SEQ ID NO: 36 - DNA, 918 bp, Chlamydia trachomatis
atgttcgattcggatcataattccatttttcaaagcgatttgtgtcagcgtctggtcgtccactcgattcttcttactttccttgtcattcttctctgtacatctttatatcccagctcagcctttattgtagggcttctttcctccgcttgcgcagctctaggaacatatgagatgggggctatggttagaatcaagtttccattttctttcacacgctatagtgcattaggatccgctattttcattgctctgacctgtcttacagctcgttgtaaaatgtgttttccagagcatatagacctacttccttggttctttctcttcttttggacgattcgcttagtatttaaaagtcgccattataaacttggtcccataggctcaactgggctcgcgttgttttgtatgctttatgtatcagtccctatccgcttgttcctccacattttgtatgggtttgtgcataccgatactccatttgtaggaatttggtgggcgatttttcttatcgctacaacaaaaagctctgatatttttggttacttctttggaaaagcttttgggaaaaaacgcattgcaccagtcattagcccgaacaaaacagtagtaggcttcattgctggttgctgtggatctatcttggttagccttcttttctactcccatcttcctaaagcctttgctgatcagattgcggtgccttggatcctcattgctttaggtactgtgttgggcgttagtggattctttggagacattatcgaatctacgttcaaacgggatgcacagatcaagaacagcagtgatctggagtctatcggaggaatgctagatgtgctagactccttgcttctttcgactcctatcgtttacgctatcctccttatcactcaaaataggacatttttaggatga

SEQ ID NO: 37 - protein, 621 aa, Chlamydia trachomatis
MRRSVCYVTPSVARAGQISTWRFEYSSANFLPEGTLLKFDLGIDGRPIDWEIPSTDLSQPCNTIYLETPSEDIVAAKAVYAPGGYIPTFEFTLPCDVEAGDTFSIILGSSPNFPQEDSSGNGAQLFTQRRKPFSLYVDPSGKGSFEDPDIFTMDIRGNVLKNIRIFAPSYVIKNKRFDITVRFEDEFGNLTNFSPEETHIELSYEHLRENLNWQLFIPETGFVILPNLYFNEPGIYRIQLRNQATKEVFTSAPIKCFAETSSHLLWGLLHGESERVDSEGNIESCLRYFRDDCALNFFATSSFEIQDGLTPETIKTINQTVADFNEEDRFIALSGAQYLSEEPGEGIREVLLMKEPKSPGKHKECKLFPLSKLYKQSTSHELISIPSFTASKKFGYNFNNFHPEFERVVEIYNAWGCSERTEAEGNPFPIKGSIDSENPEGTVLSALKRNLRFGFVAGGLDDRNLYNHFFDSDQQQYSPGLTAVICNKYSRDSLLEALYQRQCYATTGQRIIVNFQITSAPMGSELSTAIKPGLVINRHISGYVAGTAKIASIEIIRNEDILHTFHPDGNNFEYEYDDLSPFAQVTLKDPQNGAPFAFYYLRVTQENGAMAWSSPIWIDLN

SEQ ID NO: 38 - DNA, 1866 bp, Chlamydia trachomatis
atgcgcagatctgtttgttacgttactccttcagttgctagggctggtcaaatttctacctggcgtttcgaatattcttcagctaatttccttcccgaaggcacattgctaaaatttgacctgggaatagacggacgccctatagactgggagattccttctacagatctttctcaaccatgtaatacaatttatttagaaacgccttccgaggatattgtggctgcaaaagctgtgtatgctcccggaggctatatccctactttcgaatttactctcccttgtgatgtggaagctggggacactttctctattattcttggctcctctcccaacttccctcaagaggactcttcaggtaatggtgctcaattatttactcaacgccgtaaacctttctctctttatgttgacccatcagggaaaggatcttttgaagatcccgatatcttcacaatggatatcagaggaaatgtattaaaaaatatccggatttttgctccttcttatgtgatcaaaaacaaacgctttgatattacagtgcgcttcgaagatgaatttgggaacttaaccaatttctccccagaagagacccatatcgagctttcgtacgaacatcttcgcgaaaacctcaattggcaattgttcatccctgaaacaggctttgtgatccttccaaacctgtatttcaatgaaccaggtatttatcgtattcaactacgcaatcaagcaacaaaagaggtctttacatcagcgcctatcaaatgttttgcagaaacctcctctcatcttttgtgggggcttctacatggagaatctgaacgtgtcgactctgaaggtaatatcgagtcttgcttgcgttattttcgtgatgactgcgcgttaaacttttttgcaacatcctctttcgaaattcaagatggcctgaccccagaaaccattaaaaccattaaccaaaccgttgctgattttaatgaagaagatcgtttcattgccttatccggagcacagtacctttctgaagagcctggcgagggaattcgtgaagtattgctgatgaaggaacccaaatccccagggaaacataaagaatgcaaactatttcctttatctaagctatataagcaatcaactagtcatgagttaatctcaatccccagcttcactgcttcaaagaaatttggatacaattttaataatttccatcctgaatttgaaagagttgttgaaatttataatgcctggggatgctctgaaagaactgaagctgaaggaaaccctttccctattaaaggttctatcgactcagaaaatccagagggaactgttctatctgctttaaagagaaacctgcgttttggattcgtagccggtggtcttgatgatagaaatctatacaatcacttttttgattccgatcaacagcaatactcccctggattaacagctgtgatctgcaataaatattctcgggattccttactcgaggcattataccaacgacaatgctatgctacaaccggccaaagaattatcgtgaatttccagattacatctgctcctatgggctccgaactctccacagccattaaaccagggctcgtgatcaatagacatatttcgggatatgtagcaggaactgccaagattgcgtcgatcgaaatcatccgcaatgaggatattctccataccttccacccagatggaaataactttgagtatgagtacgacgatctctctccttttgcacaagtcactctaaaagatcctcaaaatggagctccttttgctttttactacttacgagtcactcaagagaatggagctatggcttggagctctcctatttggatagatcttaactaa

SEQ ID NO: 39 - protein, 697 aa, Chlamydia trachomatis
MKRFFPLFIGVLLAHTLPSEGLSHQQAVQKKISYLSHFKGITGIMDVEDGVLHIHDDLRLQANKAYVENRTDCGIKIVAHGNVMVNYRGKILICDYLEYYEDTDSCLLTNGRCSLYPWFIGGSTITISPSSIIIHKGYISTSEGPQKHICLSGDYLKYSSDSVLSMGPSRLSICNTPVLLLPQISIMPMEIPKPPITFRGGSGGFLGSYLGVSYSPISKKHCSTTLFLDGFFKHGIGLGYNMRFSSQENPSNAINIKSYYAHRLAIDSSGAKDRYRLHGDFTFSKERAHLAGEFHLSDSWETVVDIFPNNFSLKNTGPTEVSLSWRDNNLFGKMTSSVKVNSFQNVKQELPQAILHHRPVRIRRSRIFLENRLEAGFLDFHFSSNIPGSNFSSWRFSSAHKVYRGLVLPIGTLTPSLSGTAIYYTRMLSPNAAHCQLSGSLSFDYRVALQKEYRHARHIVEPFCSFLKTTRPVLSSDEPHIFSIKDAFHSINLLHVGLESKVLNKHSTPSHLKLWTTYIFDEPHAKDTFPKTACWFSLPLTLQNTLSLDAEWIWKKSRWDHLNVIWEWILNDNLGLTLEFLHRSKYGFIKCAKDNYTLDVSRSLDTLLASPLSDRRNLITGKLFVRPHPHWNYNLNLRYGWHRPDSPSYLEYQMILGHKIFEHWQLFSVYEKREADKRCFFYLKLDKRKQKHRHPFG

SEQ ID NO: 40 - DNA, 2094 bp, Chlamydia trachomatis
gtgaaacgatttttcccactttttattggagtgctgctcgcgcacactttgccgtcagaaggtctttctcatcaacaagctgtccaaaaaaaaatttcttatctgagccattttaaaggcattacaggaattatggatgttgaggatggggtattacatatccatgatgatctacgtcttcaagccaataaagcctatgttgaaaatcgcacggattgtgggatcaaaatcgttgctcatggcaacgttatggtcaattatcgcgggaaaattttaatctgtgattatcttgagtactatgaagatacagattcttgtttactcaccaatggccgctgttcgttatacccatggttcattggaggatccactataacgatctcaccatcttctattatcattcataaagggtatatctcgacttctgaaggtcctcagaaacatatttgtttatccggagattatttaaaatactcttcagacagcgtattatctatgggaccctcacgtctttctatctgtaatacgcctgtgttattgcttcctcaaatctccattatgcctatggagattcctaagcctccgattacttttcgaggtgggagtggaggatttctgggatcctacttaggtgttagttattcccctatatctaaaaagcattgttctacgactttgttcttggatggtttttttaaacatggaataggtctcggctataacatgcgcttttcctctcaggaaaatccaagcaatgccataaatattaaaagctattacgcacatcgattagctattgattcatcaggagcaaaagatcgctatcgattacatggagacttcactttttccaaagaacgagcccatcttgctggtgaattccatttaagtgatagctgggaaacagttgtggatatcttcccaaataacttctctttaaaaaatacaggccctacagaagttagcctatcatggcgcgataacaatttatttgggaaaatgacttcctctgtcaaagtcaactcctttcaaaatgttaaacaagaattgcctcaagcaattcttcatcaccgaccagtacgtatcaggcgctctcgcattttcctagagaatcgcttagaagctggttttttagattttcatttcagtagtaatattccaggctctaacttctcatcatggaggttctcatccgctcacaaagtctaccgtgggcttgttcttcctataggaacgttaaccccttcgctatctggaactgctatctactatacccgcatgctctccccaaatgcagcccattgtcaattatctggatcgctatcttttgattatcgcgttgctttacaaaaagaatatcggcatgcaagacatattgtagagcctttttgctcctttttaaaaaccactcgtcctgtattatcctctgatgagcctcatattttctcgattaaagatgcttttcactctatcaaccttctacatgtaggattggagtcaaaagtcttaaacaaacattccactccttcgcatctgaaactatggacgacctatatctttgatgaacctcacgctaaggacactttccctaaaactgcttgctggttctctcttcctcttacactccaaaatactttatccttagatgcggaatggatttggaaaaaaagccgatgggatcatctcaatgtaatctgggaatggattttgaatgataatctcggtcttactttagaatttttacatagaagtaagtatggctttattaagtgcgctaaagataactacacactcgatgtaagccgatctttagacacattactagcctctcctctttccgatcgaagaaatttgattactggcaaactttttgttcgtccacatcctcattggaattataatcttaatcttcgttatggatggcatcgtccagactctccatcctatttagaataccagatgattctgggtcataaaatctttgagcactggcagctattctctgtctacgaaaaacgtgaagctgataagcgctgcttcttttatctaaaattagataaacgaaaacagaaacaccgccatccttttggataa

SEQ ID NO: 41 - protein, 1016 aa, Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 42 - DNA, 3051 bp, Chlamydia trachomatis
atgcctttttctttgagatctacatcattttgttttttagcttgtttgtgttcctattcgtatggattcgcgagctctcctcaagtgttaacacctaatgtaaccactccttttaagggggacgatgtttacttgaatggagactgcgcttttgtcaatgtctatgcaggggcagagaacggctcaattatctcagctaatggcgacaatttaacgattaccggacaaaaccatacattatcatttacagattctcaagggccagttcttcaaaattatgccttcatttcagcaggagagacacttactctgaaagatttttcgagtttgatgttctcgaaaaatgtttcttgcggagaaaagggaatgatctcagggaaaaccgtgagtatttccggagcaggcgaagtgattttttgggataactctgtggggtattctcctttgtctattgtgccagcatcgactccaactcctccagcaccagcaccagctcctgctgcttcaagctctttatctccaacagttagtgatgctcggaaagggtctattttttctgtagagactagtttggagatctcaggcgtcaaaaaaggggtcatgttcgataataatgccgggaattttggaacagtttttcgaggtaatagtaataataatgctggtagtgggggtagtgggtctgctacaacaccaagttttacagttaaaaactgtaaagggaaagtttctttcacagataacgtagcctcctgtggaggcggagtagtctacaaaggaactgtgcttttcaaagacaatgaaggaggcatattcttccgagggaacacagcatacgatgatttagggattcttgctgctactagtcgggatcagaatacggagacaggaggcggtggaggagttatttgctctccagatgattctgtaaagtttgaaggcaataaaggttctattgtttttgattacaactttgcaaaaggcagaggcggaagcatcctaacgaaagaattctctcttgtagcagatgattcggttgtctttagtaacaatacagcagaaaaaggcggtggagctatttatgctcctactatcgatataagcacgaatggaggatcgattctatttgaaagaaaccgagctgcagaaggaggcgccatctgcgtgagtgaagcaagctctggttcaactggaaatcttactttaagcgcttctgatggggatattgttttttctgggaatatgacgagtgatcgtcctggagagcgcagcgcagcaagaatcttaagtgatggaacgactgtttctttaaatgcttccggactatcgaagctgatcttttatgatcctgtagtacaaaataattcagcagcgggtgcatcgacaccatcaccatcttcttcttctatgcctggtgctgtcacgattaatcagtccggtaatggatctgtgatttttaccgccgagtcattgactccttcagaaaaacttcaagttcttaactctacttctaacttcccaggagctctgactgtgtcaggaggggagttggttgtgacggaaggagctaccttaactactgggaccattacagccacctctggacgagtgactttaggatccggagcttcgttgtctgccgttgcaggtgctgcaaataataattatacttgtacagtatctaagttggggattgatttagaatcctttttaactcctaactataagacggccatactgggtgcggatggaacagttactgttaacagcggctctactttagacctagtgatggagagtgaggcagaggtatatgataatccgctttttgtgggatcgctgacaattccttttgttactctatcttctagtagtgctagtaacggagttacaaaaaattctgtcactattaatgatgcagacgctgcgcactatgggtatcaaggctcttggtctgcagattggacgaaaccgcctctggctcctgatgctaaggggatggtacctcctaataccaataacactctgtatctgacatggagacctgcttcgaattacggtgaatatcgactggatcctcagagaaagggagaactagtacccaactctctttgggtagcgggatctgcattaagaacctttactaatggtttgaaagaacactatgtttctagagatgttggatttgtagcatctctgcatgctctcggggattatattttgaattatacgcaagatgatcgggatggctttttagctagatatgggggattccaggcgaccgcagcctcccattatgaaaatgggtcaatatttggagtggcttttggacaactctatggtcagacaaagagcagaatgtattactctaaagatgctgggaacatgacgatgttgtcctgtttcggaagaagttacgtagatattaaaggaacagaaactgttatgtattgggagacggcttatggctattctgtgcacagaatgcatacgcagtattttaatgacaaaacgcagaagttcgatcattcgaaatgtcattggcacaacaataactattatgcgtttgtgggtgccgagcataatttcttagagtactgcattcctactcgtcagttcgctagagattatgagcttacagggtttatgcgttttgaaatggccggaggatggtccagttctacacgagaaactggctccctaactagatatttcgctcgcgggtcagggcataatatgtcgcttccaataggaattgtagctcatgcagtttctcatgtgcgaagatctcctccttctaaactgacactaaatatgggatatagaccagacatttggcgtgtcactccacattgcaatatggaaattattgctaacggagtgaagacacctatacaaggatctccgctggcacggcatgccttcttcttagaagtgcatgatactttgtatattcatcattttggaagagcctatatgaactattcgctggatgctcgtcgtcgacaaacggcacattttgtatccatgggcttgaatagaatcttttaa

SEQ ID NO: 43 - protein, 553 aa, Chlamydia trachomatis
MRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 44 - DNA, 1662 bp, Chlamydia trachomatis
atgcgaataggagatcctatgaacaaactcatcagacgagcagtgacgatcttcgcggtgactagtgtggcgagtttatttgctagcggggtgttagagacctctatggcagagtctctctctacaaacgttattagcttagctgacaccaaagcgaaagacaacacttctcataaaagcaaaaaagcaagaaaaaaccacagcaaagagactcccgtagaccgtaaagaggttgctccggttcatgagtctaaagctacaggacctaaacaggattcttgctttggcagaatgtatacagtcaaagttaatgatgatcgcaatgttgaaatcacacaagctgttcctgaatatgctacggtaggatctccctatcctattgaaattactgctacaggtaaaagggattgtgttgatgttatcattactcagcaattaccatgtgaagcagagttcgtacgcagtgatccagcgacaactcctactgctgatggtaagctagtttggaaaattgaccgcttaggacaaggcgaaaagagtaaaattactgtatgggtaaaacctcttaaagaaggttgctgctttacagctgcaacagtatgcgcttgtccagagatccgttcggttacaaaatgtggacaacctgctatctgtgttaaacaagaaggcccagagaatgcttgtttgcgttgcccagtagtttacaaaattaatatagtgaaccaaggaacagcaacagctcgtaacgttgttgttgaaaatcctgttccagatggttacgctcattcttctggacagcgtgtactgacgtttactcttggagatatgcaacctggagagcacagaacaattactgtagagttttgtccgcttaaacgtggtcgtgctaccaatatagcaacggtttcttactgtggaggacataaaaatacagcaagcgtaacaactgtgatcaacgagccttgcgtacaagtaagtattgcaggagcagattggtcttatgtttgtaagcctgtagaatatgtgatctccgtttccaatcctggagatcttgtgttgcgagatgtcgtcgttgaagacactctttctcccggagtcacagttcttgaagctgcaggagctcaaatttcttgtaataaagtagtttggactgtgaaagaactgaatcctggagagtctctacagtataaagttctagtaagagcacaaactcctggacaattcacaaataatgttgttgtgaagagctgctctgactgtggtacttgtacttcttgcgcagaagcgacaacttactggaaaggagttgctgctactcatatgtgcgtagtagatacttgtgaccctgtttgtgtaggagaaaatactgtttaccgtatttgtgtcaccaacagaggttctgcagaagatacaaatgtttctttaatgcttaaattctctaaagaactgcaacctgtatccttctctggaccaactaaaggaacgattacaggcaatacagtagtattcgattcgttacctagattaggttctaaagaaactgtagagttttctgtaacattgaaagcagtatcagctggagatgctcgtggggaagcgattctttcttccgatacattgactgttccagtttctgatacagagaatacacacatctattaa

SEQ ID NO: 45 - protein, 1751 aa, Chlamydia trachomatis
MKWLSATAVFAAVLPSVSGFCFPEPKELNFSRVGTSSSTTFTETVGEAGAEYIVSGNASFTKFTNIPTTDTTTPTNSNSSSSNGETASVSEDSDSTTTTPDPKGGGAFYNAHSGVLSFMTRSGTEGSLTLSEIKITGEGGAIFSQGELLFTDLTGLTIQNNLSQLSGGAIFGESTISLSGITKATFSSNSAEVPAPVKKPTEPKAQTASETSGSSSSSGNDSVSSPSSSRAEPAAANLQSHFICATATPAAQTDTETSTPSHKPGSGGAIYAKGDLTIADSQEVLFSINKATKDGGAIFAEKDVSFENITSLKVQTNGAEEKGGAIYAKGDLSIQSSKQSLFNSNYSKQGGGALYVEGDINFQDLEEIRIKYNKAGTFETKKITLPKAQASAGNADAWASSSPQSGSGATTVSNSGDSSSGSDSDTSETVPATAKGGGLYTDKNLSITNITGIIEIANNKATDVGGGAYVKGTLTCENSHRLQFLKNSSDKQGGGIYGEDNITLSNLTGKTLFQENTAKEEGGGLFIKGTDKALTMTGLDSFCLINNTSEKHGGGAFVTKEISQTYTSDVETIPGITPVHGETVITGNKSTGGNGGGVCTKRLALSNLQSISISGNSAAENGGGAHTCPDSFPTADTAEQPAAASAATSTPESAPVVSTALSTPSSSTVSSLTLLAASSQASPATSNKETQDPNADTDLLIDYVVDTTISKNTAKKGGGIYAKKAKMSRIDQLNISENSATEIGGGICCKESLELDALVSLSVTENLVGKEGGGLHAKTVNISNLKSGFSFSNNKANSSSTGVATTASAPAAAAASLQAAAAAVPSSPATPTYSGVVGGAIYGEKVTFSQCSGTCQFSGNQAIDNNPSQSSLNVQGGAIYAKTSLSIGSSDAGTSYIFSGNSVSTGKSQTTGQIAGGAIYSPTVTLNCPATFSNNTASMATPKTSSEDGSSGNSIKDTIGGAIAGTAITLSGVSRFSGNTADLGAAIGTLANANTPSATSGSQNSITEKITLENGSFIFERNQANKRGAIYSPSVSIKGNNITFNQNTSTHDGSAIYFTKDATIESLGSVLFTGNNVTATQASSATSGQNTNTANYGAAIFGDPGTTQSSQTDAILTLLASSGNITFSNNSLQNNQGDTPASKFCSIAGYVKLSLQAAKGKTISFFDCVHTSTKKIGSTQNVYETLDINKEENSNPYTGTIVFSSELHENKSYIPQNAILHNGTLVLKEKTELHVVSFEQKEGSKLIMKPGAVLSNQNIANGALVINGLTIDLSSMGTPQAGEIFSPPELRIVATTSSASGGSGVSSSIPTNPKRISAAAPSGSAATTPTMSENKVFLTGDLTLIDPNGNFYQNPMLGSDLDVPLIKLPTNTSDVQVYDLTLSGDLFPQKGYMGTWTLDSNPQTGKLQARWTFDTYRRWVYIPRDNHFYANSILGSQNSMIVVKQGLINNMLNNARFDDIAYNNFWVSGVGTFLAQQGTPLSEEFSYYSRGTSVAIDAKPRQDFILGAAFSKMVGKTKAIKKMHNYFHKGSEYSYQASVYGGKFLYFLLNKQHGWALPFLIQGVVSYGHIKHDTTTLYPSIHERNKGDWEDLGWLADLRISMDLKEPSKDSSKRITVYGELEYSSIRQKQFTEIDYDPRHFDDCAYRNLSLPVGCAVEGAIMNCNILMYNKLALAYMPSIYRNNPVCKYRVLSSNEAGQVICGVPTRTSARAEYSTQLYLGPFWTLYGNYTIDVGMYTLSQMTSCGARMIF

SEQ ID NO: 46 - DNA, 5256 bp, Chlamydia trachomatis
atgaaatggctgtcagctactgcggtgtttgctgctgttctcccctcagtttcagggttttgcttcccagaacctaaagaattaaatttctctcgcgtaggaacttcttcctctaccacttttactgaaacagttggagaagctggggcagaatatatcgtctctggtaacgcatctttcacaaaatttaccaacattcctactaccgatacaacaactcccacgaactcaaactcctctagctctaacggagagactgcttccgtttctgaggatagtgactctacaacaacgactcctgatcctaaaggtggcggcgccttttataacgcgcactccggagttttatcctttatgacacgatcaggaacagaaggttccttaactctgtctgagataaaaataactggtgaaggcggtgctatcttctctcaaggagagctgctatttacagatctgacaggtctaaccatccaaaataacttatcccagctatccggaggagcgatttttggagaatctacaatctccctatcagggattactaaagcgactttctcctccaactctgcagaagttcctgctcctgttaagaaacctacagaacctaaagctcaaacagcaagcgaaacgtcgggttctagtagttctagcggaaatgattcggtgtcttcccccagttccagtagagctgaacccgcagcagctaatcttcaaagtcactttatttgtgctacagctactcctgctgctcaaaccgatacagaaacatcaactccctctcataagccaggatctgggggagctatctatgctaaaggcgaccttactatcgcagactctcaagaggtactattctcaataaataaagctactaaagatggaggagcgatctttgctgagaaagatgtttctttcgagaatattacatcattaaaagtacaaactaacggtgctgaagaaaagggaggagctatctatgctaaaggtgacctctcaattcaatcttctaaacagagtctttttaattctaactacagtaaacaaggtggtggggctctatatgttgaaggagatataaacttccaagatcttgaagaaattcgcattaagtacaataaagctggaacgttcgaaacaaaaaaaatcactttaccaaaagctcaagcatctgcaggaaatgcagatgcttgggcctcttcctctcctcaatctggttctggagcaactacagtctccaactcaggagactctagctctggctcagactcggatacctcagaaacagttccagccacagctaaaggcggtgggctttatactgataagaatctttcgattactaacatcacaggaattatcgaaattgcaaataacaaagcgacagatgttggaggtggtgcttacgtaaaaggaacccttacttgtgaaaactctcaccgtctacaatttttgaaaaactcttccgataaacaaggtggaggaatctacggagaagacaacatcaccctatctaatttgacagggaagactctattccaagagaatactgccaaagaagagggcggtggactcttcataaaaggtacagataaagctcttacaatgacaggactggatagtttctgtttaattaataacacatcagaaaaacatggtggtggagcctttgttaccaaagaaatctctcagacttacacctctgatgtggaaacaattccaggaatcacgcctgtacatggtgaaacagtcattactggcaataaatctacaggaggtaatggtggaggcgtgtgtacaaaacgtcttgccttatctaaccttcaaagcatttctatatccgggaattctgcagctgaaaatggtggtggagcccacacatgcccagatagcttcccaacggcggatactgcagaacagcccgcagcagcttctgccgcgacgtctactcccgagtctgccccagtggtctcaactgctctaagcacaccttcatcttctaccgtctcttcattaaccttactagcagcctcttcacaagcctctcctgcaacctctaataaggaaactcaagatcctaatgctgatacagacttattgatcgattatgtagttgatacgactatcagcaaaaacactgctaagaaaggcggtggaatctatgctaaaaaagccaagatgtcccgcatagaccaactgaatatctctgagaactccgctacagagataggtggaggtatctgctgtaaagaatctttagaactagatgccctagtctccttatctgtaacagagaaccttgttgggaaagaaggtggaggcttacatgctaaaactgtaaatatttctaatctgaaatcaggcttctctttctcgaacaacaaagcaaactcctcatccacaggagtcgcaacaacagcttcagcacctgctgcagctgctgcttccctacaagcagccgcagcagccgtaccatcatctccagcaacaccaacttattcaggtgtagtaggaggagctatctatggagaaaaggttacattctctcaatgtagcgggacttgtcagttctctgggaaccaagctatcgataacaatccctcccaatcatcgttgaacgtacaaggaggagccatctatgccaaaacctctttgtctattggatcttccgatgctggaacctcctatattttctcggggaacagtgtctccactgggaaatctcaaacaacagggcaaatagcgggaggagcgatctactcccctactgttacattgaattgtcctgcgacattctctaacaatacagcctctatggctacaccaaagacttcttctgaagatggatcctcaggaaattctattaaagataccattggaggagccattgcagggacagccattaccctatctggagtctctcgattttcagggaatacggctgatttaggagctgcaataggaactctagctaatgcaaatacacccagtgcaactagcggatctcaaaatagcattacagaaaaaattactttagaaaacggttcttttatttttgaaagaaaccaagctaataaacgtggagcgatttactctcctagcgtttccattaaagggaataatattaccttcaatcaaaatacatccactcatgatggaagtgctatctactttacaaaagatgctacgattgagtctttaggatctgttctttttacaggaaataacgttacagctacacaagctagttctgcaacatctggacaaaatacaaatactgccaactatggggcagccatctttggagatccaggaaccactcaatcgtctcaaacagatgccattttaacccttcttgcttcttctggaaacattacttttagcaacaacagtttacagaataaccaaggtgatactcccgctagcaagttttgtagtattgcaggatacgtcaaactctctctacaagccgctaaagggaagactattagctttttcgattgtgtgcacacctctaccaaaaaaataggttcaacacaaaacgtttatgaaactttagatattaataaagaagagaacagtaatccatatacaggaactattgtgttctcttctgaattacatgaaaacaaatcttacatcccacagaatgcaatccttcacaacggaactttagttcttaaagagaaaacagaactccacgtagtctcttttgagcagaaagaagggtctaaattaattatgaaacccggagctgtgttatctaaccaaaacatagctaacggagctctagttatcaatgggttaacgattgatctttccagtatggggactcctcaagcaggggaaatcttctctcctccagaattacgtatcgttgccacgacctctagtgcatccggaggaagcggggtcagcagtagtataccaacaaatcctaaaaggatttctgcagcagcgccttcaggttctgccgcaactactccaactatgagcgagaacaaagttttcctaacaggagaccttactttaatagatcctaatggaaacttttaccaaaaccctatgttaggaagcgatctagatgtaccactaattaagcttccgactaacacaagtgacgtccaagtctatgatttaactttatctggggatcttttccctcagaaagggtacatgggaacctggacattagattctaatccacaaacagggaaacttcaagccagatggacattcgatacctatcgtcgctgggtatacatacctagggataatcatttttatgcgaactctatcttaggctcccaaaactcaatgattgttgtgaagcaagggcttatcaacaacatgttgaataatgcccgcttcgatgatatcgcttacaataacttctgggtttcaggagtaggaactttcttagctcaacaaggaactcctctttccgaagaattcagttactacagccgcggaacttcagttgccatcgatgccaaacctagacaagattttatcctaggagctgcatttagtaagatggtggggaaaaccaaagccatcaaaaaaatgcataattacttccataagggctctgagtactcttaccaagcttctgtctatggaggtaaattcctgtatttcttgctcaataagcaacatggttgggcacttcctttcctaatacaaggagtcgtgtcctatggacatattaaacatgatacaacaacactttacccttctatccatgaaagaaataaaggagattgggaagatttaggatggttagcggatcttcgtatctctatggatcttaaagaaccttctaaagattcttctaaacggatcactgtctatggggaacttgagtattccagcattcgccagaaacagttcacagaaatcgattacgatccaagacacttcgatgattgtgcttacagaaatctgtcgcttcctgtgggatgcgctgtcgaaggagctatcatgaactgtaatattcttatgtataataagcttgcattagcctacatgccttctatctacagaaataatcctgtctgtaaatatcgggtattgtcttcgaatgaagctggtcaagttatctgcggagtgccaactagaacctctgctagagcagaatacagtactcaactatatcttggtcccttctggactctctacggaaactatactatcgatgtaggcatgtatacgctatcgcaaatgactagctgcggtgctcgcatgatcttctaa

SEQ ID NO: 47 - protein, 975 aa, Chlamydia trachomatis
MNRVIEIHAHYDQRQLSQSPNTNFLVHHPYLTLIPKFLLGALIVYAPYSFAEMELAISGHKQGKDRDTFTMISSCPEGTNYIINRKLILSDFSLLNKVSSGGAFRNLAGKISFLGKNSSASIHFKHININGFGAGVFSESSIEFTDLRKLVAFGSESTGGIFTAKEDISFKNNHHIAFRNNITKGNGGVIQLQGDMKGSVSFVDQRGAIIFTNNQAVTSSSMKHSGRGGAISGDFAGSRILFLNNQQITFEGNSAVHGGAIYNKNGLVEFLGNAGPLAFKENTTIANGGAIYTSNFKANQQTSPILFSQNHANKKGGAIYAQYVNLEQNQDTIRFEKNTAKEGGGAITSSQCSITAHNTIIFSDNAAGDLGGGAILLEGKKPSLTLIAHSGNIAFSGNTMLHITKKASLDRHNSILIKEAPYKIQLAANKNHSIHFFDPVMALSASSSPIQINAPEYETPFFSPKGMIVFSGANLLDDAREDVANRTSIFNQPVHLYNGTLSIENGAHLIVQSFKQTGGRISLSPGSSLALYTMNSFFHGNISSKEPLEINGLSFGVDISPSNLQAEIRAGNAPLRLSGSPSIHDPEGLFYENRDTAASPYQMEILLTSDKIVDISKFTTDSLVTNKQSGFQGAWHFSWQPNTINNTKQKILRASWLPTGEYVLESNRVGRAVPNSLWSTFLLLQTASHNLGDHLCNNRSLIPTSYFGVLIGGTGAEMSTHSSEEESFISRLGATGTSIIRLTPSLTLSGGGSHMFGDSFVADLPEHITSEGIVQNVGLTHVWGPLTVNSTLCAALDHNAMVRICSKKDHTYGKWDTFGMRGTLGASYTFLEYDQTMRVFSFANIEATNILQRAFTETGYNPRSFSKTKLLNIAIPIGIGYEFCLGNSSFALLGKGSIGYSRDIKRENPSTLAHLAMNDFAWTTNGCSVPTSAHTLANQLILRYKACSLYITAYTINREGKNLSNSLSCGGYVGF

SEQ ID NO: 48 - DNA, 2928 bp, Chlamydia trachomatis
atgaatcgagttatagaaatccatgctcactacgatcaaagacaactttctcaatctccaaatacaaacttcttagtacatcatccttatcttactcttattcccaagtttctactaggagctctaatcgtctatgctccttattcgtttgcagaaatggaattagctatttctggacataaacaaggtaaagatcgagatacctttaccatgatctcttcctgtcctgaaggcactaattacatcatcaatcgcaaactcatactcagtgatttctcgttactaaataaagtttcatcagggggagcctttcggaatctagcagggaaaatttccttcttaggaaaaaattcttctgcgtccattcattttaaacacattaatatcaatggttttggagccggagtcttttctgaatcctctattgaatttactgatttacgaaaacttgttgcttttggatctgaaagcacaggaggaatttttactgcgaaagaggacatctcttttaaaaacaaccaccacattgccttccgcaataatatcaccaaagggaatggtggcgttatccagctccaaggagatatgaaaggaagcgtatcctttgtagatcaacgtggagctatcatctttaccaataaccaagctgtaacttcttcatcaatgaaacatagtggtcgtggaggagcaattagcggtgacttcgcaggatccagaattctttttcttaataaccaacaaattactttcgaaggcaatagcgctgtgcatggaggtgctatctacaataagaatggccttgtcgagttcttaggaaatgcaggacctcttgcctttaaagagaacacaacaatagctaacgggggagctatatacacaagtaatttcaaagcgaatcaacaaacatcccccattctattctctcaaaatcatgcgaataagaaaggcggagcgatttacgcgcaatatgtgaacttagaacagaatcaagatactattcgctttgaaaaaaataccgctaaagaaggcggtggagccatcacctcttctcaatgctcaattactgctcataataccatcattttttccgataatgctgccggagatcttggaggaggagcaattcttctagaagggaaaaaaccttctctaaccttgattgctcatagtggtaatattgcatttagcggcaataccatgcttcatatcaccaaaaaagcttccctagatcgacacaattctatcttaatcaaagaagctccctataaaatccaacttgcagcgaacaaaaaccattctattcatttctttgatcctgtcatggcattgtcagcatcatcttcccctatacaaatcaatgctcctgagtatgaaactcccttcttctcacctaagggtatgatcgttttctcgggtgcgaatcttttagatgatgctagggaagatgttgcaaatagaacatcgatttttaaccaacccgttcatctatataatggcaccctatctatcgaaaatggagcccatctgattgtccaaagcttcaaacagaccggaggacgtatcagtttatctccaggatcctccttggctctatacacgatgaactcgttcttccatggcaacatatccagcaaagaacccctagaaattaatggtttaagctttggagtagatatctctccttctaatcttcaagcagagatccgtgccggcaacgctcctttacgattatccggatccccatctatccatgatcctgaaggattattctacgaaaatcgcgatactgcagcatcaccataccaaatggaaatcttgctcacctctgataaaattgtagatatctccaaatttactactgattctctagttacgaacaaacaatcaggattccaaggagcctggcattttagctggcagccaaatactataaacaatactaaacaaaaaatattaagagcttcttggctcccaacaggagaatatgtccttgaatccaatcgagtggggcgtgccgttcctaattccttatggagcacatttttacttttacagacagcctctcataacttaggcgatcatctatgtaataatcgatctcttattcctacttcatacttcggagttttaattggaggaactggagcagaaatgtctacccactcctcagaagaagaaagctttatatctcgtttaggagctacaggaacctctatcatacgcttaactccctccctgacactctctggaggaggctcacatatgttcggagattcgttcgttgcagacttaccagaacacatcacttcagaaggaattgttcagaatgtcggtttaacccatgtctggggaccccttactgtcaattctacattatgtgcagccttagatcacaacgcgatggtccgcatatgctccaaaaaagatcacacctatgggaaatgggatacattcggtatgcgaggaacattaggagcctcttatacattcctagaatatgatcaaactatgcgcgtattctcattcgccaacatcgaagccacaaatatcttgcaaagagcttttactgaaacaggctataacccaagaagtttttccaagacaaaacttctaaacatcgccatccccatagggattggttatgaattctgcttagggaatagctcttttgctctactaggtaagggatccatcggttactctcgagatattaaacgagaaaacccatccactcttgctcacctggctatgaatgattttgcttggactaccaatggctgttcagttccaacctctgcacacacattggcaaatcaattgattcttcgctataaagcatgttccttatacatcacggcatatactatcaaccgtgaagggaagaacctctccaatagcttatcctgcggaggctatgttggcttctaa

SEQ ID NO: 49 - protein, 210 aa, Chlamydia trachomatis
MYKYFIVDTSGSQPFLAYVDCRDVLEVWSLPTGPDQGVVLNFIFNSLDLPFQGIGVSVGPGGFSATRVGVAFAQGLSLAKNVPLVGYSSLEGYLSLGQEEEALLLPLGKKGGIVALNSELSLDGFLLTDTTSTPGILLSYSEALEYCLDKGCCHVISPDPTYFVELFSSRISVRKVVPCIDRIRKYVVSQFVLSQNLPLCLDYRSISSFF

SEQ ID NO: 50 - DNA, 633 bp, Chlamydia trachomatis
gtgtacaaatattttattgtagacacttccggttctcagccgtttttggcctatgtcgattgtcgagatgttttagaagtatggtctttgcctacagggccagatcaaggagttgtgttaaatttcattttcaacagcctggatttgcctttccaaggtattggagtctctgttggtcctgggggattttctgcaactagagtgggagttgcttttgctcaagggctctctctggctaaaaatgtccctttagttggctatagctctttagaaggatatctttctttgggtcaagaagaggaggctttgcttttgcctctagggaaaaagggtgggattgtagctttaaactcagagctttctcttgatggttttctgcttacagatactacttctactccgggaattttattgtcttattctgaagctctagagtattgtttagataagggatgttgtcatgtgatctctccggatccaacgtatttcgtagaactattttcttcgcgtatttcggtaaggaaggtggttccttgtatcgatcgaatccgtaagtacgttgtttcgcagtttgttctgtctcaaaatcttccgttgtgtttagattaccggagcatctcttcctttttttag

SEQ ID NO: 51 - protein, 141 aa, Chlamydia trachomatis
MIDVSVVGPVLAMALAMIGSAVGCGMAGVASHAVMSRIDEGHGKIIGLSAMPSSQSIYGLIFMLLLNDAIKDGKVSAVSGIVMGIAVGSALLLSAFMQGKCCVSAIQAYARSSAIYGKSFASIGIVESFALFAFVFALLLF

SEQ ID NO: 52 - DNA, 426 bp, Chlamydia trachomatis
atgatagatgtatcagtagtggggcctgtattggctatggctttggcaatgattggtagcgctgttggatgtggaatggctggagtcgcttctcacgcagtgatgtctcgaatcgatgaaggacacgggaagattattggtctgtctgctatgccctcatcacaatccatttacgggttgattttcatgttactgctgaatgatgcaattaaggatggaaaagtctctgcagtcagtggtatcgtaatgggtatagctgtaggatctgcgttattgctttctgcttttatgcaagggaagtgctgtgtgagtgctattcaagcctatgcgcgttcctctgcaatatatggtaaatcatttgcttcgattgggattgttgagtcttttgcgttatttgctttcgtttttgcgctattgttattctaa

SEQ ID NO: 53 - protein, 101 aa, Chlamydia trachomatis
MAKKSAVAREVKRRKLVEANFQKRAELRKLAKSLSVSEEERERAREALNKMRRDTSPSRLHNRCLLTGRPRGYLRKFAISRICFRQMASMGDIPGVVKASW

SEQ ID NO: 54 - DNA, 306 bp, Chlamydia trachomatis
atggcgaagaagtcagcagtagctagagaagttaaacgtcgaaagttagtagaagctaattttcagaagagagcagagcttcgaaaacttgcgaagagtttatctgttagcgaggaagaaagagaaagagctcgggaagctctcaataaaatgagaagagatacttctccttctcgtttacataatagatgcctattaacaggccgtcctcgtggataccttagaaagtttgctatctcaagaatttgttttagacaaatggcttctatgggagatatcccaggcgttgtgaaagcaagttggtag

SEQ ID NO: 55 - protein, 393 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 56 - DNA, 1182 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcgccacttggtgtgacgctatcagcatgcgtgttggttactacggagactttgttttcgaccgtgttttgaaaactgatgtgaataaagaatttcagatgggtgccaagcctacaactgatacaggcaatagtgcagctccatccactcttacagcaagagagaatcctgcttacggccgacatatgcaggatgctgagatgtttacaaatgccgcttgcatggcattgaatatttgggatcgttttgatgtattctgtacattaggagccaccagtggatatcttaaaggaaactctgcttctttcaatttagttggattgtttggagataatgaaaatcaaaaaacggtcaaagcggagtctgtaccaaatatgagctttgatcaatctgttgttgagttgtatacagatactacttttgcgtggagcgtcggcgctcgcgcagctttgtgggaatgtggatgtgcaactttaggagcttcattccaatatgctcaatctaaacctaaagtagaagaattaaacgttctctgcaatgcagcagagtttactattaataaacctaaagggtatgtaggtaaggagtttcctcttgatcttacagcaggaacagatgctgcgacaggaactaaggatgcctctattgattaccatgaatggcaagcaagtttagctctctcttacagactgaatatgttcactccctacattggagttaaatggtctcgagcaagctttgatgccgatacgattcgtatagcccagccaaaatcagctacagctatttttgatactaccacgcttaacccaactattgctggagctggcgatgtgaaaactggcgcagagggtcagctcggagacacaatgcaaatcgtttccttgcaattgaacaagatgaaatctagaaaatcttgcggtattgcagtaggaacaactattgtggatgcagacaaatacgcagttacagttgagactcgcttgatcgatgagagagcagctcacgtaaatgcacaattccgcttctaa

SEQ ID NO: 57 - protein, 194 aa, Chlamydia trachomatis
MLKMFWLNSLVFFSLLLSACGYTVLSPHYVEKKFSLSEGIYVCPIEGDSLGDLVSSLSYELEKRGLHTRSQGTSSGYVLKVSLFNETDENIGFAYTPQKPDEKPVKHFIVSNEGRLALSAKVQLIKNRTQEILVEKCLRKSVTFDFQPDLGTANAHQLALGQFEMHNEAIKSASRILYSQLAETIVQQVYYDLF

SEQ ID NO: 58 - DNA, 585 bp, Chlamydia trachomatis
atgctgaaaatgttttggttgaatagcctcgttttcttctcgttactactatcagcctgcggctatacagtgctctccccccactatgtagaaaagaaattctcgctttccgaaggcatctatgtctgccctatcgaaggagattcattaggagatctcgtatcctctctttcttacgaattagaaaagcgaggactccacacacgatctcaaggaacctcttctggttatgtactcaaagtctctcttttcaatgagactgatgaaaatattggattcgcatacactccccaaaaacctgatgaaaaacctgtaaaacacttcattgtctctaatgaagggcgcttagcgttatcagcaaaagtccaactaatcaaaaaccgcacacaagaaatattagtggagaaatgcctgagaaaatcggttacttttgattttcaacctgacctcggaaccgcgaatgctcatcagctagctctcggacaatttgaaatgcataatgaagcaataaaaagcgcttctcgtatattgtattcgcaattagcagagactattgtacaacaggtatactatgaccttttctga

SEQ ID NO: 59 - protein, 542 aa, Chlamydia trachomatis
MFKLVSYIILSWVLVCLAQPDVSVVASVVSCICGYSLLWAGLFALVEQLSWKKVWCIAFIWTWTVEGAHFSWMLEDLYVGTSIYFVWGILLSYLATLFASFSCLVVWCCRKQYRGALVWLPGVWVAIEAIRYYGLLSGVSFDFIGWPLTATAYGRQFGSFFGWAGQSFLVIAANICCFAVCLLKHSFSKGLWLTLCAFPYLLGGAHYEYLKKHFSDSEVLRVAIVQPGYSPHMHAGRTASAIWRGLVSLCQTIQTPVDVIVFPEVSVPFGLHRQAYTLHENQPVLESLLPNKSWGEFFTNLDWIQAIAERYQCTVIMGMERWENKGGILHLYNAAECVSREGEITSYDKRILVPGGEYIPGGKIGFSLCQTFFPEFALPFQRLPGEFSGVVNITERIKAGISICYEETFGYAIRPYKRQQADILVNLTNDGWYPRSRLPLVHFYHGMLRNQELGIPCIRACRTGVSAAVDSLGRIVGILPWESRTCPVSTGVLQVSVPLYSYHTVYARLGDAPLLLIAVCSVIGAIAYFYRKKKETPPQTFF

SEQ ID NO: 60 - DNA, 1629 bp, Chlamydia trachomatis
gtgtttaaacttgtgtcatacatcatcctttcttgggtgctggtctgtttggctcagccggatgtaagtgttgtagcttctgttgttagttgtatttgcggttacagcttactttgggctgggctttttgctttagtagagcaattatcttggaagaaagtttggtgcatcgcttttatttggacttggactgtcgaaggcgctcatttctcttggatgcttgaagatctttatgtagggacaagcatctattttgtttggggtatactgctttcttatctcgccaccctatttgctagtttttcttgtttggttgtgtggtgttgtcgcaagcaatataggggagctcttgtttggcttccaggggtttgggtggcgatagaagcaatacgctattatgggttgctttcaggagtttcttttgattttattggctggcctcttacagcgacagcctatggccggcaattcggcagcttttttggatgggctggacaaagctttctagttattgctgccaatatatgctgttttgcagtatgtttattaaaacactctttttccaaaggtttgtggttgacgttgtgcgcgttcccttatctgttaggcggagcgcattacgaatacctaaagaagcatttttccgactctgaagtgcttcgagttgccatcgtgcagcctggatatagtcctcatatgcatgcagggaggacggctagtgctatttggagaggtttggtttctttgtgccagactattcaaactcctgtagatgtgatcgttttcccagaagtaagtgttccttttggcttacatagacaagcctatactcttcatgaaaatcagcctgtattagaaagtttgcttcctaacaaatcttggggcgagtttttcacaaatttggattggatccaagcgatagctgaacgttatcaatgcaccgttatcatgggaatggaacgatgggaaaataaagggggaatactgcatttgtataatgctgctgaatgcgtatcgcgagaaggggaaataactagctatgataagcggattcttgttcctggaggtgagtacatccctggagggaaaataggtttttccttgtgtcaaacctttttcccagaatttgctcttccctttcaacgtttgccaggagagttttctggagttgtgaatataacagagcgaataaaagctgggatctctatttgttatgaggagacatttgggtatgcaattcgcccttacaaaaggcaacaagccgatattttagtaaatcttactaatgacggttggtatccgcgttcaaggctgcctctagtacatttttatcatggcatgttacgtaatcaagagttgggtataccttgtattcgcgcctgtcgcacaggagtttctgctgcagtggattctttgggtagaattgtcggcatacttccctgggaatcgagaacttgcccagtttctacaggagtactccaagtttccgtccctctttacagttatcatactgtatatgcaaggctgggtgatgctcctctgttactgattgcagtttgttcggttatcggagcgattgcctatttttataggaaaaagaaagagaccccaccacaaacatttttttga

SEQ ID NO: 61 - protein, 553 aa, Chlamydia trachomatis
MSSNKHASLCQKTPSLCRELQKAPALLLTEDIRFKALLNERIDSVAELFPCTYNSPYYKFISKSDLSAETSPLKVGVMLSGGPAPGGHNVILGLLHSIKKLHPNSQLLGFIRNGEGLLNNNTVEITDEFIEEFRNSGGFNCIGTGRTNIITEENKARCLQTANELDLDGLVIIGGDGSNTATAILAEYFAKHQAKTVLVGVPKTIDGDLQHLFLDLTFGFDTATKFYSSIISNISRDALSCKGHYHFIKLMGRSSSHITLECALQTHPNIALIGEEIAEKSISLETLIHDICETIADRAAMGKYHGVILIPEGVIEFIPEIQSLVKEIESIPEQENLYQALSLSSQQLLCQFPEDICHQLLYNRDAHGNVYVSKISVDKLLIHLVRQHLETHFRQVPFNAISHFLGYEGRSGTPTHFDNVYSYNLGYGAGVLVFNRCNGYLSTIEGLTSPIEKWRLRALPIVRMLTTKQGKDSKHYPLIKKRLVDIASPVFNKFSLYRKIWALEDSYRFVGPLQIHSPEDAHSDDFPPLILFLNHNEWQKRCSICLEIPDQDY

SEQ ID NO: 62 - DNA, 1662 bp, Chlamydia trachomatis
atgtcgtcgaataaacatgcttctctttgtcaaaagacgccttctttgtgtcgggagcttcaaaaagctcctgctcttctattaacagaagacataaggtttaaagctcttcttaatgaacgcattgactctgttgcagaactatttccatgcacttataactctccctactacaaatttatttcgaagtccgatctttccgctgagacctctccccttaaagtgggcgttatgctttctggaggcccagctcctggtgggcacaatgtcatcttaggattgctacacagtattaaaaagctccatccgaatagtcagcttttaggatttattcgcaatggagaaggacttctcaataataatactgtagaaatcacagatgaattcattgaagagtttcgtaactctggaggctttaattgcataggaacaggtcgcactaatatcataaccgaagaaaataaagcgcgctgtttacaaacagcaaatgaactcgatttagatggattagtgattattggaggcgatggttcgaatacagccacggcgattcttgctgaatattttgctaagcatcaagcaaaaacggtattagttggtgttcccaaaactattgatggagatttgcagcacctatttttagacctcacatttgggtttgatactgctactaaattttattcatccatcatcagcaacatttctagagacgcattatcgtgtaaaggccactatcattttattaaactaatgggccggtcttcttctcatatcacgctagaatgcgcactacagactcacccaaatattgctcttataggcgaagagattgcagaaaaaagcatctccttagaaacattaatccatgatatttgtgaaacaatagcagatcgagctgctatggggaaataccatggcgttattctcatccctgaaggagtcattgagtttattcctgaaatacagtctctggttaaagaaattgaatccattccagagcaggagaatctttaccaagctttatccttatcttctcagcaacttttatgccaatttccggaagatatttgccatcagctcttgtataatagagatgctcatggcaacgtctatgtatcaaaaattagtgttgataaacttctgattcatctagttcgtcaacatttagaaacacattttagacaagttcccttcaatgcaatctcccattttttaggttatgaagggcgttcaggaactcctacacattttgataatgtgtatagctataacttaggatatggtgctggggttctcgtttttaaccgctgtaatgggtatttatccacgatcgaaggtctaactagccctattgaaaaatggcgattgcgcgctttacccattgttcgaatgttgacgaccaagcaggggaaagacagtaaacattatcctctgataaaaaaaagattggtagatattgctagtcctgtttttaataagttctcactgtatcggaaaatctgggctttagaagactcctatcgctttgtagggccattacaaatacattctccggaggatgctcattctgatgattttcctcctttaattttgtttttgaatcataatgaatggcaaaaacgctgttctatttgtttagaaatccccgatcaggattattaa

SEQ ID NO: 63 - protein, 167 aa, Chlamydia trachomatis
MICCDKVLSSVQSMPVIDKCSVTKCLQTAKQAAVLALSLFAVFASGSLSILSAAVLFSGTAAVLPYLLILTTALLGFVCAVIVLLRNLSAVVQSCKKRSPEEIEGAARPSDQQESGGRLSEESASPQASPTSSTFGLESALRSIGDSVSGAFDDINKDNSRSRSHSF

SEQ ID NO: 64 - DNA, 439 bp, Chlamydia trachomatis
atgatctgctgtgacaaagtcttgtcgagcgtacaatcaatgcctgttatagataaatgctctgtaacgaaatgcttacaaacggctaagcaagcagctgttcttgcgttgtctttgtttgcggtgtttgcttcaggaagtttatccatattatcagcggcggtactgtttagtggcactgctgctgttcttccatatctgctgatattaacaacagctcttctaggatttgtttgtgctgttattgtgcttttaagaaatttatcagcagttgttcagagttgtaaaaagagatcacctgaagaaattgaaggggctgctcgtccctctgatcagcaggaatcaggaggacgtttgtccgaggagagcgcttcaccacaagcatctcctacttcgtctacttttggtcttgaatccgctttgcgctcaataggagata

SEQ ID NO: 65 - protein, 89 aa, Chlamydia trachomatis
MSKKCALTGRKPRRGYSYAIRGISKKKKGIGLKVTGRTKRRFFPNMMTKRLWSTEENRFLKLKISAAALRLVDKLGLDQVVARAKSKGF

SEQ ID NO: 66 - DNA, 270 bp, Chlamydia trachomatis
atgtcgaaaaaatgtgcgcttacaggaagaaagcctcgtcgcggttatagctatgctatccgagggatttctaaaaagaaaaaagggatcggtttgaaagttacaggaagaacaaaacgtcgattcttccctaatatgatgactaagagactatggtctactgaggaaaatcgcttcctcaaactcaaaatttctgcagcagctttacgccttgttgataaactagggttagatcaggttgttgctagagctaaaagcaagggtttttag

SEQ ID NO: 67 - protein, 157 aa, Chlamydia trachomatis
MLFGYLVGFLAADPEERMTSGGKRVVVLRLGVKSRVGSKDETVWCRCNIWNNRYDKMLPYLKKGSSVIVAGELSLESYVGRDGSPQASISVSVDTLKFNSGSSRPDARGSDEGRQRANDNVSIGFDGESLDTDSALDKEVYAGFGEDQQYASEDVPF

SEQ ID NO: 68 - DNA, 474 bp, Chlamydia trachomatis
atgttgttcggatatttggtaggatttctagctgccgatcctgaagaaagaatgacatccggaggtaaacgggttgttgttttacgtttgggtgtaaaatctcgtgtaggatctaaagatgaaacagtgtggtgcagatgcaatatctggaacaaccgttatgataagatgcttccttatttgaagaaaggttcttcagtcattgttgctggagagctttctttagaaagctatgtaggtagagacggttctccacaagcttctatttctgtaagcgtagatacattaaaatttaattccggatcttctcgtcctgatgctagaggttcagatgaaggtcgtcagagagctaatgataatgtctctattggatttgatggagaaagtttagatacagactctgcgcttgataaggaagtctatgcagggtttggagaagaccaacagtatgctagtgaggatgttcctttttag

SEQ ID NO: 69 - protein, 247 aa, Chlamydia trachomatis
MKKQEKMHPQNLLKVFIFFLAFFISYPSAEAHSPLQSSIQEKILTARPGDYAVLSRGSQKFFFLIRQSSSEATWVEMSEFASLTQQEKKLVEQSSWKNAFHQLQSSKKVYLLRISKNPLMIFVLKNAQWMPLSEKDPLPFFVKILRLPLSPAPSHLIKYKGKERTPWSPRTSLNGELITLPSSAWISVWPKDSSPLSEKNILIYFSNNERLAFPLWTSIDTPTGTVIIKTIEMGHQAASSYPALPNF

SEQ ID NO: 70 - DNA, 744 bp, Chlamydia trachomatis
atgaaaaagcaagaaaaaatgcaccctcaaaaccttcttaaagtttttatttttttcttggcatttttcatatcctatccctcggctgaagcccattctcctctccaatcatcaatccaagaaaaaattctaactgcccgccccggagactatgccgtcttaagccgaggatctcaaaaatttttctttttaattcgccaaagttcttcggaagcgacttgggtcgaaatgtctgaatttgcctccctaacacagcaagaaaaaaaattagtagaacagtcttcctggaagaatgccttccatcaactccaatcttcaaaaaaagtgtacttgttacgaatttccaaaaatcctcttatgatttttgttctcaaaaatgcgcaatggatgcctctctcagaaaaagatcctttgcctttctttgtaaaaatccttcgactccctttatctccagccccctctcacttaattaaatacaaagggaaagaacgcaccccctggtctccgcgaacatctttgaatggagaactcataacccttccttccagtgcttggatttctgtttggccaaaagattcttctcctctatcagaaaaaaatattctcatatatttttctaacaatgaacgtttagcgtttcctctatggactagtattgatactcctacagggacagtgattattaagactattgaaatggggcaccaagccgcctcctcctatccagctcttcccaatttctag

SEQ ID NO: 71 - DNA, 456 bp, Chlamydia trachomatis
aatatgagcactgtacccgttgttcaaggagctggatcttccaattcggcacaggatatttccactagaccattaacactgaaagagcgtatatcgaatcttctatcttccactgcatttaaggtgggattagtggtgataggactacttttagtgattgctactttgatattcctagtttcggcagcttcgtttgtaaatgccatctatctagtagctattcctgctattttgggatgcgtgaatatctgcgtaggaattttatccatggaaggacactgttctccggagagatggatcttatgtaagaaggtattaaagacttcagaagatatcatcgatgatgggcagataaacaactctaataaagtgtttactgatgagaggttgaatgccataggtggggtagtggaatctctatctagaagaaatagtctggtggatcagacccaatga

SEQ ID NO: 72 - protein, 151 aa, Chlamydia trachomatis
NMSTVPVVQGAGSSNSAQDISTRPLTLKERISNLLSSTAFKVGLVVIGLLLVIATLIFLVSAASFVNAIYLVAIPAILGCVNICVGILSMEGHCSPERWILCKKVLKTSEDIIDDGQINNSNKVFTDERLNAIGGVVESLSRRNSLVDQTQ

SEQ ID NO: 73 - DNA, 354 bp, Chlamydia trachomatis
gggctagtttcttttattgttaaaagaattgcttttatcgataaaagaaacttcaagagcccttttctagaaaggagtctggaagttatgaaaaaaactgctttactcgctgctttatgtagtgttgtttctttaagtagttgttgtcgtatcgttgactgttgcttcgaagatccatgcgcacctatccaatgttcaccttgtgaatctaagaagaaagacgtagacggtggttgcaactcttgtaacgggtatgtcccagcttgcaaaccttgcggaggggatacgcaccaagatgctaaacatggccctcaagctagaggaattccagttgacggcaaatgcagacaatag

SEQ ID NO: 74 - protein, 117 aa, Chlamydia trachomatis
GLVSFIVKRIAFIDKRNFKSPFLERSLEVMKKTALLAALCSVVSLSSCCRIVDCCFEDPCAPIQCSPCESKKKDVDGGCNSCNGYVPACKPCGGDTHQDAKHGPQARGIPVDGKCRQ

SEQ ID NO: 75 - DNA, 1650 bp, Chlamydia trachomatis
gctagtaagggagcccctttagtgtttaaacttgtgtcatacatcatcctttcttgggtgctggtctgtttggctcagccggatgtaagtgttgtagcttctgttgttagttgtatttgcggttacagcttactttgggctgggctttttgctttagtagagcaattatcttggaagaaagtttggtgcatcgcttttatttggacttggactgtcgaaggcgctcatttctcttggatgcttgaagatctttatgtagggacaagcatctattttgtttggggtatactgctttcttatctcgccaccctatttgctagtttttcttgtttggttgtgtggtgttgtcgcaagcaatataggggagctcttgtttggcttccaggggtttgggtggcgatagaagcaatacgctattatgggttgctttcaggagtttcttttgattttattggctggcctcttacagcgacagcctatggccggcaattcggcagcttttttggatgggctggacaaagctttctagttattgctgccaatatatgctgttttgcagtatgtttattaaaacactctttttccaaaggtttgtggttgacgttgtgcgcgttcccttatctgttaggcggagcgcattacgaatacctaaagaagcatttttccgactctgaagtgcttcgagttgccatcgtgcagcctggatatagtcctcatatgcatgcagggaggacggctagtgctatttggagaggtttggtttctttgtgccagactattcaaactcctgtagatgtgatcgttttcccagaagtaagtgttccttttggcttacatagacaagcctatactcttcatgaaaatcagcctgtattagaaagtttgcttcctaacaaatcttggggcgagtttttcacaaatttggattggatccaagcgatagctgaacgttatcaatgcaccgttatcatgggaatggaacgatgggaaaataaagggggaatactgcatttgtataatgctgctgaatgcgtatcgcgagaaggggaaataactagctatgataagcggattcttgttcctggaggtgagtacatccctggagggaaaataggtttttccttgtgtcaaacctttttcccagaatttgctcttccctttcaacgtttgccaggagagttttctggagttgtgaatataacagagcgaataaaagctgggatctctatttgttatgaggagacatttgggtatgcaattcgcccttacaaaaggcaacaagccgatattttagtaaatcttactaatgacggttggtatccgcgttcaaggctgcctctagtacatttttatcatggcatgttacgtaatcaagagttgggtataccttgtattcgcgcctgtcgcacaggagtttctgctgcagtggattctttgggtagaattgtcggcatacttccctgggaatcgagaacttgcccagtttctacaggagtactccaagtttccgtccctctttacagttatcatactgtatatgcaaggctgggtgatgctcctctgttactgattgcagtttgttcggttatcggagcgattgcctatttttataggaaaaagaaagagaccccaccacaaacatttttttga

SEQ ID NO: 76 - protein, 549 aa, Chlamydia trachomatis
ASKGAPLVFKLVSYIILSWVLVCLAQPDVSVVASVVSCICGYSLLWAGLFALVEQLSWKKVWCIAFIWTWTVEGAHFSWMLEDLYVGTSIYFVWGILLSYLATLFASFSCLVVWCCRKQYRGALVWLPGVWVAIEAIRYYGLLSGVSFDFIGWPLTATAYGRQFGSFFGWAGQSFLVIAANICCFAVCLLKHSFSKGLWLTLCAFPYLLGGAHYEYLKKHFSDSEVLRVAIVQPGYSPHMHAGRTASAIWRGLVSLCQTIQTPVDVIVFPEVSVPFGLHRQAYTLHENQPVLESLLPNKSWGEFFTNLDWIQAIAERYQCTVIMGMERWENKGGILHLYNAAECVSREGEITSYDKRILVPGGEYIPGGKIGFSLCQTFFPEFALPFQRLPGEFSGVVNITERIKAGISICYEETFGYAIRPYKRQQADILVNLTNDGWYPRSRLPLVHFYHGMLRNQELGIPCIRACRTGVSAAVDSLGRIVGILPWESRTCPVSTGVLQVSVPLYSYHTVYARLGDAPLLLIAVCSVIGAIAYFYRKKKETPPQTFF

SEQ ID NO: 77 - DNA, 645 bp, Chlamydia trachomatis
gaaaattgttataggatcaggagagaaacgtttcccatgctggggagcatttccttcacaacatataaagaaaacctcatgagaaagactatttttaaagcgtttaatttattattctcccttctttttctttcttcatgctcttatccttgcagagattgggaatgccatggttgcgactccgcaagacctcgtaaatcctcttttggattcgtacctttctactccgatgaagaaattcaacaagcttttgttgaagattttgattccaaagaagagcagctgtacaaaacgagcgcacagagtacctctttccgaaatatcactttcgctacagatagttattctattaaaggagaggataacctcacgattcttgcaagcttagttcgtcatttgcataaatctcctaaagctacgctatatatagagggccatacagatgaacgtggagctgcagcttataacctagctttaggagctcgtcgtgcgaatgctgtaaaacaatacctcatcaaacagggaatcgctgcagaccgcttattcactatttcttacggaaaagaacatcctgttcatccaggccataatgaattagcttggcaacaaaatcgtcgtactgaatttaagatccatgctcgctaa

SEQ ID NO: 78 - protein, 214 aa, Chlamydia trachomatis
ENCYRIRRETFPMLGSISFTTYKENLMRKTIFKAFNLLFSLLFLSSCSYPCRDWECHGCDSARPRKSSFGFVPFYSDEEIQQAFVEDFDSKEEQLYKTSAQSTSFRNITFATDSYSIKGEDNLTILASLVRHLHKSPKATLYIEGHTDERGAAAYNLALGARRANAVKQYLIKQGIAADRLFTISYGKEHPVHPGHNELAWQQNRRTEFKIHAR

SEQ ID NO: 79 - protein, 943 aa, Neisseria gonorrhoeae
MNKKHGFPLTLTALAIATAFPAYAAQAGAAALDAAQSQSLKEVTVRAAKVGRRSKEATGLGKIVKTSETLNKEQVLGIRDLTRYDPGVAVVEQGNGASGGYSIRGVDKNRVAVSVDGVAQIQAFTVQGSLSGYGGRGGSGAINEIEYENISTVEIDKGAGSSDHGSGALGGAVAFRTKEAADLISDGKSWGIQAKTAYGSKNRQFMKSLGAGFSKDGWEGLLIRTERQGRETRPHGDIADGVEYGIDRLDAFRQTYDIKRKTTEPFFLVEGENTLKPVAKLAGYGIYLNRQLNRWVKERIEQNQPLSAEEEAQVREAQARHENLSAQAYTGGGRILPDPMDYRSGSWLAKLGYRFGGRHYVGGVFEDTKQRYDIRDMTEKQYYGTDEAEKFRDKSGVYDGDDFRDGLYFVPNIEEWKGDKNLVKGIGLKYSRTKFIDEHHRRRRMGLLYRYENEKYSDNWADKAVLSFDKQGVATDNNTLKLNCAVYPAVDKSCRASADKPYSYDSSDRFHYREQHNVLNASFEKSLKNKWTKHHLTLGFGYDASKAVSRPEQLSHNAARISESTGFDEKNQDKYRLGKPEVVEGSVCGYIETLRSRKCVPRKINGSNIHISLNDRFSIGKYFDFSLGGRYDRKNFTTSEELVRSGRYADRSWNSGIVFKPNRHFSVSYRASSGFRTPSFQELFGIDIYHDYPKGWQRPALKSEKAANREIGLQWKGDFGFLEISSFRNRYTDMIAVADQKTKLPDSAGRLTEIDIRDYYNAQNMSLQGINILGKIDWNGVYGKLPEGLYTTLAYNRIKPKSVSNRPDLSLRSYALDAVQPSRYVLGFGYDQPEGKWGANIMLTYSKGKNPDELAYLAGDQKRYSAGRVTSSWKTADVSAYLNLKKRLTLRAAIYNIGNYRYVTWESLRQTAESTANRHGGDSNYGRYAAPGRNFSLALEMKF

SEQ ID NO: 80 - DNA, 3300 bp, Neisseria gonorrhoeae
ctcgggataacggcatcaatctttcgggaaatggttcgactaatcctcaaagtttcaaagccgacaatcttcttgtaacgggcggcttttacggcccgcaggcggcggaattgggcggcactattttcaataaggatgggaaatctcttggtataactgaagatattgaaaatgaagttgaaaatgaagctgatgttggcgaacagttagaacctgaagttaaaccccaattcggcgtggtattcggtgcgaagaaagataataaagaggtggaaaaatgaataagaaacacggttttccgctgactttgacggcgttggccattgcaaccgcttttccggcttatgctgcccaagcgggggcggcggcacttgatgcggcgcaaagtcaatcattgaaagaggttaccgtccgtgccgccaaagtgggacggcgatcgaaagaggcgacaggtttgggcaaaatcgtcaaaacgtcggaaacgttgaacaaagaacaggtactcggtatccgcgacctgacgcgctacgatccgggcgtggcggttgtcgaacagggcaacggcgcgagcggcggctactcgatacgcggcgtagataaaaaccgtgtggcggtttcggttgacggcgttgcccaaatacaggcgtttaccgtgcagggatcgttgagcggatacggcggacgcggcggcagcggcgcaatcaacgaaatcgaatatgaaaacatcagcacggtggaaatcgacaaaggcgccggttcgtccgatcacggcagcggcgcactcggcggcgcggtcgccttccgcaccaaagaggcggcagacctgatttcagacggcaaaagctgggggatacaggcaaaaaccgcttacggcagtaaaaaccgccaatttatgaagtcgctcggcgcggggttcagcaaagacggttgggaagggctgctaatccgaaccgaacgccaagggcgggaaacgcgcccgcacggcgatattgcggacggggtggaatacggcatagaccgtttggacgcgttccgccagacatacgatattaaacgcaagacaacagagccatttttcttagtagagggcgagaatacactcaagcccgtggcaaaattggcgggctacgggatatatttgaaccgccagctcaaccgctgggtaaaagaacgtattgaacaaaatcagcctttaagtgctgaagaagaggcgcaggtgcgggaggcgcaggcgcgccacgaaaacctgtccgcccaagcctacacgggcggcggcaggatattgcccgatccgatggattaccgcagcggctcttggcttgccaagctgggctaccgcttcggcggcaggcattatgtcggcggcgtgtttgaggataccaaacagcgttacgacatccgcgatatgacggaaaaacagtattacggtacggacgaggcggaaaagtttagagacaagagcggggtgtacgacggcgacgatttccgcgacggcttgtattttgtgccgaatatagaagagtggaagggcgataaaaatttggtcaagggcataggtttgaaatattcccgcaccaaatttattgacgaacatcaccgccgccgccgtatgggtttgctgtaccgttatgaaaatgagaaatactcggacaactgggcggataaggcggtgttgtcgtttgacaaacagggcgtggcaaccgacaacaacacgctgaagctgaattgcgccgtgtatcctgccgtggacaaatcctgccgcgcgtcggcggacaaaccgtattcctacgacagcagcgaccgtttccactaccgcgaacagcacaatgttttgaatgcctcgtttgagaagtcgctgaaaaacaaatggacgaaacaccatctgactttgggcttcggttacgatgcttccaaagcagtatcccgcccagaacagctttcccacaatgcggcaaggatttcggaatccacgggattcgatgaaaagaatcaagataagtaccgtttgggtaagcccgaagtcgtcgaagggtcggtctgcggctatatcgaaaccctgcgttcccgcaaatgcgtgccaagaaaaatcaacggcagcaatatccacatttctttgaacgaccgtttttcaatcggcaaatattttgatttcagcttgggcggcaggtacgaccggaaaaacttcaccacgtcggaagaactcgtccgcagcgggcggtatgccgaccgttcgtggaacagcggcatcgtgttcaaaccgaaccggcatttttccgtgtcttaccgcgcctccagcggcttcagaacgccttccttccaagaacttttcgggatagacatttatcacgattatccgaaaggctggcagcgtcccgccctgaaatcggaaaaggcagccaaccgggaaatcggtttgcagtggaagggcgatttcggctttttggaaatcagcagtttccgcaaccgttataccgatatgattgccgttgccgatcaaaaaaccaaattgccggattcagcaggacgattgacagagattgatatacgcgattattacaatgcccaaaatatgtcgcttcaaggcatcaacatcttggggaaaatcgactggaacggcgtatacggcaaactgcccgaaggcctgtacaccacattggcgtacaaccgtatcaaaccgaaatcggtatccaaccggccggacttgtccctccgcagctatgctttggatgcggtacagccgtcgcgttatgttttggggttcggatacgaccagcccgaggggaaatggggcgcaaacattatgctgacctattccaaagggaaaaaccctgacgagcttgcttatctggcaggcgatcaaaaacgatattcggcaggaagggttacgtcttcttggaaaacggcagatgtttccgcttatctgaatctgaaaaaacggctgaccttgagggcggctatctacaatatcggcaactaccgctacgttacttgggaatccttgcgccagactgcggaaagcacggcaaaccggcacggcggcgacagcaactatggaaggtatgccgcaccgggcaggaacttcagcctcgcgctcgaaatgaagttttaaaggaaatgccgtctggaagcttgatctgcaccccaaaagtcggactaaaccgccaactgattaaggtgcaggtttttttgattcaatataaacaagatttccgccgtcattcccgcgcaggcgggaatccggacattcaatgctaaggcaatttatcggaaatgactgaaactcaaaaaaccggattccca

SEQ ID NO: 81 - protein, 728 aa, Neisseria gonorrhoeae
MRKLNYYGIALLPLMLASCGGNFGVQPVVESTPTAYPVTFKSKDVPTSPPPAEPSVETTPVNRPAVGAAMRLLRRNTAFHREDGTAIPDSKQAEEKLSFKEGDVLFLYGSKGNKLQQLKSEIHKRDSDVEIRTSEKENKKYGYEFVDAGYVYTKNGKDEIEQNSGGKRFTHRFGYDGFVYYSGERPSQSLPSAGTVKYFGNWQYMTDAKRHRTGKAVASDDLGYITFYGNDIGATSYAAKDADDREKHPAEYTVDFDKKILKGELIKNQYVQKKNDPKKPLTIYNITADLNGNRFTGSAKVNTEVKTRHADKEYLFFHTDADQRLEGGFFGDNGEELAGRFISNDNGVFGVFAGKQKTNASGTNPAMPFGKHTKILDSLKISVDEATDENPRPFEVSTMPDFGHPDKLLVEGREIPLVSKEKTIDLADGRKMTVSACCDFLTYVKLGRIKTERPAVKPKAQDEEDSGINNGEESEDEEEIAEESEDEVSEDDNGEDEDEIVEEEADEAEEIEEEAEEEEPEEESPEEGNGVSDGIPPAPEALKGRDIDLFLKGIRTAEADIPKTGTAHYTGTWEARIGEPIQWDNKADKAAKAEFDVDFGNKSISGTLTEQNGVEPAFRIENGVIEGNGFHPTARTRDNGINLSGNGSTNPQSFKADNLLVTGGFYGPQAAELGGTIFNKDGKSLGITEDIENEVENEADVGEQLEPEVKPQFGVVFGAKKDNKEVEK

SEQ ID NO: 82 - protein, 907 aa, Neisseria gonorrhoeae
MQQQHLFRFNILCLSLMTALPAYAENVQAGQAQEKQLDTIQVKAKKQKTRRDNEVTGLGKLVKTADTLSKEQVLDIRDLTRYDPGIAVVEQGRGASSGYSIRGMDKNRVSLTVDGLAQIQSYTAQAALGGTRTAGSSGAINEIEYENVKAVEISKGSNSVEQGSGALAGSVAFQTKTADDVIGEGRQWGIQSKTAYSGKNRGLTQSIALAGRIGGAEALLIRTGRHAGEIRAHEAAGRGVQSFNRLAPVEDGSDYAYFVVEGECPDGYAACKDKPKKDVVGEDKRQTVSTRDYTGPNRFLADPLSYESRSWLFRPGFRFENKRHYIGGILERTQQTFDTRDMTVPAFLTKAVFDANSKQAGSLRGNGKYAGNHKYGGLFTNGENNAPVGAEYGTGVFYDETHTKSRYGLEYVYTNADKDTWADYARLSYDRQGIGLDNHFQQTHCSADGSDKYCRPSADKPFSYYKSDRVIYGESHRLLQAAFKKSFDTAKIRHNLSVNLGYDRFGSNLRHQDYYYQSANRAYSLKTPPQNNGKKTSPYWVSIGRGNVVTGQICRSGNNTYTDCTPRSINGKSYYAAVRDNVRLGRWADVGAGLRYDYRSTHSDDGSVSTGTHRTLSWNTGIVLKPADWLDLTYRTSTGFRLPSFAEMYGWRSGDKIKAVKIDPEKSFNKEAGIVFKGDFGNLEASWFDNAYRDLIVRGYEAEIKNGKEQAKGAPAYLNAQSARITGINILGKIDWNGVWDKLPEGWYSTFAYNRVRVRDIKKRADRTDIQSHLFDAIQPSRYVVGSGYDQPEGKWGVNGMLTYSKAKEITELLGSRALLNGNSRNTKATARRTRPWYIVDVSGYYTVKKHFTLRAGVYNLLNHRYVTWENVRQTAAGAVNQHKNVGVYNRYAAPGRNYTFSLEMKF

SEQ ID NO: 83 - DNA, 3035 bp, Neisseria gonorrhoeae
cgaagagttgggcggatggtttgcctatccgggcaatgaacaaacgaaaaatgcgcaagcttcatccggcaatggaaattcagcaggcagcgcgaccgtggtattcggtgcgaaacgccaaaagcttgtgcaataagcacggctgccgaacaatcgagaataaggcttcagacggcatcgttcctgccgattccgtctgaaagcgaagattagggaaacactatgcaacagcaacatttgttccgattcaatattttatgcctgtctttaatgactgcgctgcccgcttatgcagaaaatgtgcaagccggacaagcacaggaaaaacagttggacaccatacaggtaaaagccaaaaaacagaaaacccgccgcgataacgaagtaaccggtttgggcaaattggtcaaaaccgccgacacactcagcaaagaacaggtactcgacatccgcgacctgacgcgttacgaccccggcatcgccgtcgtcgaacaggggcgcggcgcaagctcgggctactcgatacgcggtatggacaaaaaccgcgtctccttgacggtggacggcttggcgcaaatacagtcctacaccgcgcaggcggcattgggcgggacgaggacggcgggcagcagcggcgcaatcaatgaaatcgagtatgaaaacgttaaggctgtcgaaatcagcaaaggctcaaactcggtcgaacaaggcagcggcgcattggcgggttcggtcgcatttcaaaccaaaaccgcagacgatgttatcggggaaggcaggcagtggggcattcagagtaaaaccgcctattccggcaaaaaccgggggcttacccaatccatcgcgctggcggggcgcatcggcggtgcggaggctttgctgatccgcaccggccggcacgcgggggaaatccgcgcccacgaagccgccggacgcggcgttcagagctttaacaggctggcgccggttgaagacggcagtgactatgcctattttgtggtcgaaggagaatgccctgatggatatgcggcttgtaaagacaaaccgaaaaaagatgttgtcggcgaagacaaacgtcaaacggtttccacccgagactacacgggccccaaccgcttccttgccgatccgctttcatacgaaagccggtcgtggctgttccgcccgggttttcgttttgagaataagcggcactacatcggcggcatactcgaacgcacgcaacaaactttcgacacgcgcgatatgacggttccggcattcctgaccaaggcggtttttgatgcaaattcaaaacaggcgggttctttgcgcggcaacggcaaatacgcgggcaaccacaaatacggcgggctgtttaccaacggcgaaaacaatgcgccggtgggcgcggaatacggtacgggcgtgttttacgacgagacgcacaccaaaagccgctacggtttggaatatgtctataccaatgccgataaagacacttgggcggattatgcccgcctctcttacgaccggcagggcatcggtttggacaaccattttcagcagacgcactgttctgccgacggttcggacaaatattgccgcccgagtgccgacaagccgttttcctattacaaatccgaccgcgtgatttacggggaaagccacaggctcttgcaggcggcattcaaaaaatccttcgataccgccaaaatccgccacaacctgagcgtgaatctcggttacgaccgcttcggctctaatctgcgccatcaggattattattatcaaagtgccaaccgcgcctattcgttgaaaacgccccctcaaaacaacggcaaaaaaaccagcccctattgggtcagcataggcaggggaaatgtcgttacggggcaaatctgccgctcgggcaacaatacttatacggactgcacgccgcgcagcatcaacggcaaaagctattacgcggcggtccgggacaatgtccgtttgggcaggtgggcggatgtcggcgcgggcttgcgctacgactaccgcagcacgcattcggacgacggcagcgtttccaccggcacgcaccgcaccctgtcctggaacaccggcatcgtcctcaaacctgccgactggctggatttgacttaccgcacttcaaccggcttccgcctgccctcgtttgcggaaatgtacggctggcggtcgggcgataaaataaaagccgtcaaaatcgatccggaaaaatcgttcaacaaagaagccggcatcgtgtttaaaggcgatttcggcaacttggaggcaagttggttcgacaatgcctaccgcgatttgattgtccggggttatgaagcggaaattaaaaacggcaaagaacaagccaaaggcgccccggcttacctcaatgcccaaagcgcgcggattaccggcatcaatattttgggcaaaatcgattggaacggcgtatgggataaattgcccgaaggttggtattctacatttgcctataatcgtgtccgtgtccgcgacatcaaaaaacgcgcagaccgcaccgatattcaatcacacctgtttgatgccatccaaccctcgcgctatgtcgtcggctcgggctatgaccaaccggaaggcaaatggggcgtgaacggtatgctgacttattccaaagccaaggaaatcacagagttgttgggcagccgggctttgctcaacggcaacagccgcaatacaaaagccaccgcgcgccgtacccgcccttggtatattgtggatgtgtccggttattacacggttaaaaaacacttcaccctccgtgcgggcgtgtacaacctcctcaaccaccgctatgttacttgggaaaatgtgcggcaaactgccgccggcgcagtcaaccaacacaaaaatgtcggcgtttacaaccgatatgccgcccccggccgcaactacacatttagcttggaaatgaagttctaaacgtccgaacgccgcaaatgccgtctgaaaggcttcagacggcgttttttacacaatccccaccgtttcccatccttcccgatacaccg

SEQ ID NO: 84 - protein, 683 aa, Neisseria gonorrhoeae
MNNPLVNQAAMVLPVFLLSACLGGGGSFDLDSVDTEAPRAAPKYQDVPSKKPEARKDQGGYGFAMRFKRRNWYRAANENEVKLKESDWEQTDDDEIKNPFKQKNIINALPGNEGELLQDSSQQGKGTSKVRDHHDFKYVWSGFFYKRIKITTKKDESHKIIEARSGPDGYIFYKGRNPSRKLPVSGEVTYKGTWDFLTDVKANQKFTDLGNASTKSGDQYSAFSGELDYIVKKEEDKKEKHKGLGLTTEITVDFEKKTLIGKLIKNNMLINNNTKPTTQYYSLEAQVTGNRFSGKAMATEKGENKQHPFVSDSSSLSGGFFGPQGEELGFRFLSDDGKVAVVGSAKTKDETASSGGTSGGASVSTSNGAAGTSSENKLTTVLDAVELTPNGKKIKDLDNFSNAAQLVVDGIMIPLLPKDSESGGSHTDKGENGKTAFIYETTYTPESDKEDAQTGMATNGVQTVSNTAGGTSGKTKTHYEVQACCSNLNYLKYGLLTRKNSSQADAKMGQVEQSMFLQGERTDEKEIPQEQNVVYSGTWYGHIATNGTSWTREASDQENGNRANFDVNFKDKRITGTLTAENRSEATFTIEAMIEGNGFKGTAKTGNGGFAPDQNSSTGTHKVHITNAAVQGGFYGPNAEELGGWFAYPGNGQTKNAQTSSGNGNSAGSATVVFGAKRQQLVK

SEQ ID NO: 85 - DNA, 2052 bp, Neisseria gonorrhoeae
atgaacaatccattggtgaatcaggctgctatggtgctgcccgtgtttttgttgagcgcttgtctgggcggaggcggcagtttcgatcttgattctgtcgataccgaagccccgcgtgccgcgccaaagtatcaagatgttccttccaaaaaaccggaagcccgaaaagaccaaggcggatacggtttcgcgatgcgcttcaagcggcggaattggtatcgggcggcaaacgaaaacgaggttaaactgaaagagagtgattgggaacaaacggatgatgatgagatcaaaaaccctttcaaacaaaaaaatattattaatgccttacctggaaatgagggggaattattgcaagattccagtcaacaaggtaagggtacatctaaggttagggaccatcacgattttaaatacgtatggtcgggttttttttataaacggattaagattacaactaaaaaagacgaatctcataaaataatcgaagccagaagcggtcctgacggttatattttttataaaggcagaaatccctcgagaaaacttcctgtttcaggggaggttacgtacaaaggtacttgggattttttaactgatgtgaaagcaaatcagaaatttacagatttaggaaatgcttctacgaaatccggagaccaatatagtgctttttccggggagttggattatatagtcaaaaaagaggaggataaaaaagaaaagcacaaaggtttgggattaacaacggaaataacggttgattttgagaaaaaaaccctgatcggaaaattaattaaaaacaacatgttaatcaataataacactaaacccaccacccaatattacagccttgaggctcaagtaacaggcaaccgcttcagcggcaaggcgatggcaaccgaaaaaggcgaaaacaaacaacatccctttgtttccgactcgtcttctctgagcggcggctttttcggcccgcagggtgaggaattgggtttccgctttttgagcgacgatggaaaagttgccgttgtcggcagcgcgaaaaccaaagacgaaaccgcaagcagtggcggcacttcgggcggtgcaagcgtttccacatcaaacggtgcggcaggcacgtcgtctgaaaacaagctgaccacggttttggatgcggttgaattgacaccaaacggcaagaaaatcaaagatctcgacaacttcagcaacgccgcccaactggttgtcgacggcattatgattccgctcctgcccaaggattccgaaagcgggggcagtcatacagataaaggtgaaaacggcaaaacagcctttatctacgaaacaacctacacgccggaaagtgataaagaagacgctcaaacaggtatggcgaccaatggcgtgcaaaccgtttcaaatacggcaggcggcacaagtggcaaaacaaaaacccattatgaagtccaagcctgctgttccaacctcaattatctgaaatacgggttgctgacgcgtaaaaacagtagtcaagctgacgctaaaatgggacaagttgaacaaagtatgttcctccaaggcgagcgcaccgatgaaaaagaaattccacaagaacaaaatgtcgtttattcaggcacttggtacgggcatattgccaccaacggcacaagttggacccgcgaagcctccgatcaggaaaatggtaatcgggcaaattttgacgtgaatttcaaagacaaaagaattaccggcacgttaaccgctgaaaacaggtcggaggcaacctttaccattgaagccatgattgagggcaacggctttaaaggtacggcgaaaaccggtaatggcggctttgcgccggatcaaaacagcagcaccggtacacataaagtgcacatcacaaatgccgcggtgcagggcggtttttacgggcctaacgccgaagagttgggcggttggtttgcctatccgggcaatggacaaacgaaaaatgcgcaaacttcatccggcaatggaaattcagcaggcagcgcgaccgtggtattcggtgcgaaacgccaacagcttgtgaaataa

SEQ ID NO: 86 - protein, 215 aa, Neisseria gonorrhoeae
MASKSSKTNLDLNNLHLLNLPARLFIALLVVAAVLGLGYAGLFKSQMESLEEYEAKETELKNTYKQKSIDAASLNNLRDELASIRSAFDIMLKQLPTDAEIPNLVQELHQAGSSNGLRLDSVMPQPPVDDGPIKKLPYSISITGNYEQISQFTRDVGSLSRIITLESLKIAQSPENGGNPDGKSSILNLSAIATTYQAKSIEELAAEAAQNAEQK

SEQ ID NO: 87 - protein, 180 aa, Neisseria gonorrhoeae
MKHYALLISFLALSACSQSSEDLNEWMAQTRREAKAEIIPFQAPTLPVAPVYSPPQLTGPNAFDFRRMETAKKGENAPDTKRIKETLEKFSLENMRYVGILKSGQKVSGFIEAEGYVYTVGVGNYLGQNYGRIESITDDSIILNELIEDSTGNWVSRKAELLLNSSDKNTEQAAQPEEQN

SEQ ID NO: 88 - protein, 720 aa, Neisseria gonorrhoeae
MNTKLTKIISGLFVATAAFQTASAGNITDIKVSSLPNKQKIVKVSFDKEIVNPTGFVTSSPARIALDFEQTGISMDQQVLEYADPLLSKISAAQNSSRARLVLNLNKPGQYNTEVRGNKVWIFINESDDTVSAPARPAVKAAPAAPAKQQAAAPFTESVVSVSAPFSPAKQQAAASAKQQAATPAKQTNIDFRKDGKNAGIIELAALGFAGQPDISQQHDHIIVTLKNHTLPTALQRSLDVADFKTPVQKVTLKRLNNDTQLIITTTGNWELVNKSAAPGYFTFQVLPKKQNLESGGVNNAPKTFTGRKISLDFQDVEIRTILQILAKESGMNIVASDSVSGKMTLSLKDVPWDQALDLVMQARNLDMRQQGNIVNMAPRRAACQRQSLLTSGKRHCRSGRAVFPKLPIEIQKCGRIPQHPALDNADTTGNRNTLVSGRGSVLIDPATNTLIVTDTRSVIEKFRKLIDELDVPAQQVMIEARIVEAADGFSRDLGVKFGATGRKKLKNETSAFGWGVNSGFGGGDKWEAKPKSTCRLPCRKQHFAGARDFSGALNLELSASESLSKTKTLANPRVLTQNRKEAKIESGYEIPFTVTTRSGGGNSTNTELKKAVLGLTVTANITPDGQIIMTVKINKDSPRQCASGNNTILCISTKSLNTQAMVENGGTLIVGGIYEENNGNTLTKVPLLATSPLSATSLKHSGKNRPPRTADFQLPPREL

SEQ ID NO: 89 - DNA, 3829 bp, Neisseria gonorrhoeae
aaacgcacaggaggaaactgaatggcttctaaatcatctaaaaccaacttggatctcaacaaccttcacctgctcaaccttcctgccaggctttttatcgccctgctggtcgttgccgccgtgctggggctcggttatgccggattgttcaaaagccagatggaatcccttgaggaatatgaagcaaaagaaaccgaactgaaaaacacctacaaacagaaaagtatcgacgcggccagcctgaacaaccttagggacgaacttgcctcaatccgctctgccttcgatatcatgttgaaacagctgccgacagatgcagaaattcccaatttggttcaagagcttcatcaggcgggttcgagcaacggtctgcgcttggacagcgttatgccccaacctcccgtagatgacggtcccatcaaaaaattaccctattccatttccattaccggaaattacgaacagatcagccaatttacccgcgatgtcggcagtctctcccgaatcattacccttgagtcgctgaaaatcgcccaatctccggaaaacggcggcaatcctgacggcaagagcagtatcctgaacctcagcgccattgccaccacctaccaagcaaaatccatagaagagcttgccgcagaagcggcacaaaatgccgagcaaaaataacttacgttagggaaaccatgaaacactatgccttactcatcagctttctggctctctccgcgtgttcccaaagttctgaagacctaaacgaatggatggcacaaacgcgacgcgaagccaaagcagaaatcatacctttccaagcacctaccctgccggttgcgccggtatacagcccgccgcagcttacagggccgaacgcattcgacttccgccgcatggaaaccgccaaaaaaggggaaaatgcccccgacaccaagcgtattaaagaaacgctggaaaaattcagtttggaaaatatgcgttatgtcggcattttgaagtccggacagaaagtctccggcttcatcgaggctgaaggttatgtctacactgtcggtgtcggcaactatttgggacaaaactacggtagaatcgaaagcattaccgacgacagcatcatcctgaacgagctgatagaagacagcacgggcaactgggtttcccgtaaagcagaactgctgttgaattcttccgacaaaaacaccgaacaagcggcacagcctgaggaacaaaattaagaagaggattactccattatgaataccaaactgacaaaaatcatttccggtctctttgtcgcaaccgccgcctttcagacggcatcggcaggaaacattacagacatcaaagtttcctccctgcccaacaaacagaaaatcgtcaaagtcagctttgacaaagagattgtcaacccgaccggcttcgtaacctcctcaccggcccgcatcgccttggactttgaacaaaccggcatttccatggatcaacaggtactcgaatatgccgatcctctgttgagcaaaatcagtgccgcacaaaacagcagccgtgcgcgtctggttctgaatttgaacaaaccgggccaatacaataccgaagtacgcgggaacaaagtttggatattcattaacgaatcggacgataccgtgtccgcccccgcccgcccagccgtaaaagccgcgcctgccgcaccggcaaaacaacaggctgccgcaccgtttaccgagtccgtagtatccgtatccgcaccgttcagcccggcaaaacaacaggcagcggcatcggcaaaacaacaggcggcgacaccggcaaaacaaaccaatatcgatttccgcaaagacggcaaaaatgccggcattatcgaattggcggcattgggctttgccgggcagcccgacatcagccaacagcacgaccacatcatcgttacgctgaaaaaccataccctgccgaccgcgctccaacgcagtttggatgtggcagacttcaaaacaccggttcaaaaggttacgctgaaacgcctcaataacgacacccagctgattatcacaacaaccggcaactgggaactcgtcaacaaatccgccgcgcccggatactttaccttccaagtcctgccgaaaaaacaaaacctcgagtcaggcggcgtgaacaatgcgcccaaaaccttcacaggccggaaaatctcccttgacttccaagatgtcgaaatccgcaccatcctgcagattttggcaaaagaatccgggatgaacattgttgccagcgactccgtcagcggcaaaatgaccctctccctcaaagacgtaccttgggatcaggctttggatttggttatgcaggcgcgcaacctcgatatgcgccagcaagggaacatcgtcaacatggccccgcgacgagctgcttgccaaagacaaagccttcttacaagcggaaaaagacattgccgatctgggcgcgctgtattcccaaaacttccaattgaaatacaaaaatgtggaagaattccgcagcatcctgctttggacaatgccgacacgaccggaaaccgcaacacgcttgtcagcggcaggggcagcgtgctgatcgatcccgccaccaacaccctgattgttaccgatacccgcagcgtcatcgaaaaattccgcaaactgattgacgaattggacgtacccgcgcaacaagtgatgattgaggcgcgtatcgtcgaagcggcagacggcttctcgcgcgatttgggcgttaagttcggcgcgacaggcaggaaaaaactgaaaaatgagacgagcgcattcggctggggcgtgaactccggcttcgggggcggcgataaatgggaggccaaaccaaaatcaacctgccggttgccgtgccgcaaacagcatttcgctggtgcgcgcgatttctccggcgcgttgaatttggaattgtccgcatccgagtcgctttcaaaaaccaaaacgcttgccaatccgcgcgtgctgacccaaaaccgcaaagaggccaaaatcgaatccggttacgaaattccttttaccgtaactacacgctcgggcggcggcaactctaccaacacggaactcaaaaaagccgtcttggggctgaccgttacggcgaacatcacgcccgacggacaaatcatcatgaccgtcaaaatcaacaaagactcgcctcgacaatgtgcttcaggcaacaacacaatcctatgtatttcgaccaaaagcctgaatacgcaggctatggttgaaaacggcggcactttgattgtcggcggtatttatgaagaaaacaacggcaatacgctgaccaaagtccccctgttggctacatccccgttatcggcaacctctttaaaacactcgggaaaaaaccgaccgccgcgaactgctgattttcaattacccccgagggaattatagatacggcgcaacagcctgcgctattgatgcgtcaaaataagggcatatgttttacagcatatgccctttctttatgctttttgccgcgaccgaaatgccgtcattcccgcgagcgaatccaacttgtccggtttcggttgtttttcgtctcgtaacttttgagccgtcattcccgcgaaatcggaaatccagtccgttcagtttcggtcatttccgataaattcctgttgcttttcatttctagattcccactttcgtggaataacggcggaagggataaatcctcgcaatccaaagcctgctcatttccacaaaaaacagcaacccgaaacaccccgtcattcccgagcaggcggaatctagaaccgcaacgccaggaatctgtcggatacggctgaaaccgaacgactggattcccg

SEQ ID NO: 90 - protein, 174 aa, Neisseria gonorrhoeae
MKKALAALIALALPAAALAEGASGFYVQADAAHAKASSSLGSAKGFSPRISAGYRINDLRFAVDYTRYKNYKAPSTDFKLYSIGASVIYDFDTQSPVKPYFGARLSLNRASAHLGGSDSFSKTSAGLGVLAGVSYAVTPNVDLDAGYRYNYVGKVNTVKNVRSGELSAGVRVKF

SEQ ID NO: 91 - DNA, 710 bp, Neisseria gonorrhoeae
cggcaaagcagccggatgccgccgcgtatcttgaggcattgaaaatattacgatgcaaaaagaaaatttcagtataatacggcaggattctttaacggattattaacaatttttctccctgaccataaaggaaccaaaatatgaaaaaagcacttgccgcactgattgccctcgcactcccggccgccgcactggcggaaggcgcatccggcttttacgtccaagccgatgccgcacacgccaaagcctcaagctctttaggttctgccaaaggcttcagcccgcgcatctccgcaggctaccgcatcaacgacctccgcttcgccgtcgattacacgcgctacaaaaactataaagccccatccaccgatttcaaactttacagcatcggcgcgtccgtcatttacgacttcgacacccaatcgcccgtcaaaccgtatttcggcgcgcgcttgagcctcaaccgcgcttccgcccacttgggcggcagcgacagcttcagcaaaacctccgccggcctcggcgtattggcgggcgtaagctatgccgttaccccgaatgtcgatttggatgccggctaccgctacaactacgtcggcaaagtcaacactgtcaaaaacgtccgttccggcgaactgtccgccggcgtgcgcgtcaaattctgatatacgcgttattccgcaaaccgccgagccttcggcggttttttg

SEQ ID NO: 92 - protein, 792 aa, Neisseria gonorrhoeae
MKLKQIASALMMLGISPLAFADFTIQDIRVEGLQRTEPSTVFNYLPVKVGDTYNDTHGSAIIKSLYATGFFDDVRVETADGLLLLTVIVCPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSDRFDRQKFAQDMEKVTDFYQNNGYFDFRILDTDIQTNEDKTRQTIKITVHEGGRFRWGKVSIEGDTNEVPKAELEKLLTMKPGKWYERQQMTAVLGEIQNRMGSAGYAYSEISVQPLPNAGTKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDIYGKAFDPRKASTSVKQYKTTTAGGGVRMGIPVTEYDRVNFGLAAEHLTVNTYNKAPKRYADFIKQYGKTDGADGSFKGLLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGRTYTAAENGNNKSVYSENAHKSTFTNELRYSAGGAVTWLSPLGPMKFIYAYPLKKKPEDEIQRFQFQLGTTF

SEQ ID NO: 93 - DNA, 2379 bp, Neisseria gonorrhoeae
atgaaactgaaacagattgcctccgcactgatgatgttgggcatatcgcctttggcatttgccgacttcaccatccaagacatccgtgtcgaaggcttgcagcgtaccgagccgagcaccgtattcaactacctgcccgtcaaagtcggcgacacctacaacgacacacacggcagtgccatcatcaaaagcctgtacgccaccggtttctttgacgacgtacgagtcgaaactgcggacgggctgcttctgctgaccgttatcgtatgccctaccatcggctcgctcaacatcaccggcgccaaaatgctgcagaacgacgccatcaagaaaaacctcgaatcgttcgggctggcgcagtcgcaatactttaatcaggcgacactcaaccaggcagtcgccggcctgaaagaagaatatctcgggcgcggcaaactcaatatccaaatcacgcccaaagtaaccaaactcgcccgcaaccgcgtcgacatcgacatcacgattgacgagggcaaatccgccaaaatcaccgacatcgaatttgaaggcaaccaagtctattccgaccgcaaactgatgcggcagatgtcgctgaccgaaggcggcatttggacatggctgacacgaagcgaccggttcgaccgccagaaattcgcccaagacatggaaaaagtaaccgacttctaccagaacaacggctacttcgatttccgtatcctcgataccgacatccaaaccaacgaagacaaaaccaggcagaccatcaaaatcaccgtccacgaaggcggacgtttccgctggggcaaagtgtcgattgaaggcgacaccaacgaagtccccaaggccgaactggaaaaactgctgaccatgaagcccggcaaatggtacgaacgccagcagatgaccgccgttttgggtgagattcagaaccgcatgggctcggcaggctacgcatacagcgaaatcagcgtacagccgctgccgaacgccggaaccaaaaccgtcgatttcgtcctgcacatcgaaccgggcagaaaaatctacgtcaacgaaatccacatcaccggcaacaacaaaacccgcgacgaagtcgtgcgccgcgaattgcgccaaatggaatccgcgccttacgacacctccaagctgcaacgctccaaagagcgcgtcgagcttttgggctacttcgacaacgtacagtttgatgccgtcccgcttgccggtacgcccgacaaagtcgatttgaacatgagcctgaccgaacgttccaccggctcgctcgacttgagcgcgggctgggttcaggataccggcttggtcatgtccgccggcgtatcgcaggacaacctgttcggtacgggcaagtcggccgccctgcgcgcctcgcgaagcaaaaccacgctcaacggctcgctgtcgtttaccgacccgtacttcacggcagacggggtcagcctgggctacgatatttacggaaaagccttcgacccgcgcaaagcatcgaccagcgtcaaacaatataaaaccaccaccgccggcggcggcgtaaggatgggtatccccgttaccgaatacgaccgcgtcaatttcgggctggcggcggaacacctgaccgtcaacacctacaacaaagcacccaaacgctatgccgactttatcaaacaatacggcaaaaccgacggcgcagacggcagcttcaaaggcctgctgtacaaaggcactgtcggctgggggcgcaacaagaccgacagcgccttatggccgacgcgcggctacctgaccggcgtaaatgccgaaatcgccctgcccggcagcaaactgcaatactactccgccacccacaaccaaacctggttcttccccttaagcaaaaccttcacgctgatgctcggcggcgaagtcggcattgcgggcggctacggcagaaccaaagaaatccccttctttgaaaacttctacggcggcggcctgggttcggtgcgcggctacgaaagcggcacgctcggcccgaaagtgtatgacgaatacggcgaaaaaatcagctacggcggcaacaaaaaagccaacgtctccgccgagctgctcttcccgatgcccggtgcgaaagacgcacgcaccgtccgcctgagcctgtttgccgacgcaggcagcgtgtgggacggcagaacctataccgccgccgaaaacggtaacaacaaatcggtttactcggaaaacgcgcataaatccacctttaccaacgaattgcgctattccgccggcggcgcggttacctggctctcgcctttgggcccgatgaaattcatctacgcctacccgctgaagaaaaaaccggaagacgaaatccaacgcttccaattccagctcggcacgacgttctaa

SEQ ID NO: 94 - DNA, 1128 bp, Neisseria gonorrhoeae
atgaatacacgaaatatgcgctatattcttttgacaggactgttgccgacggcatccgcttttggagagaccgcgctgcaatgcgccgctttgacggacaatgttacgcgtttggcgtgttacgacaggatttttgcggcacagcttccgtcttcggcagggcaggaagggcaggagtcgaaagccgtactcaatctgacggaaaccgtccgcagcagcttggataagggcgaggcggtcattgttgttgaaaaaggcggggatgcgcttcctgccgacagtgcgggcgaaaccgccgatatctatacgcctttgagcctgatgtacgacttggacaaaaacgatttgcgcgggctgttgggcgtacgcgaacacaatccgatgtaccttatgccgttttggtataacaattcgcccaactatgccccgagttcgccgacgcgcggtacgactgtacaggaaaaattcggacagcagaaacgtgcggaaaccaaattgcaggtttcgttcaaaagcaaaattgccgaaaatttgtttaaaacccgcgcggatctgtggttcggctacacccaaagatccgattggcagatttacaaccaaggcaggaaatccgcgccgttccgcaatacggattacaaacctgaaattttcctgacccagcctgtgaaggcggatttgccgttcggcggcaggctgcgtatgctcggtgcgggttttgtccaccagtccaacggacagagccgtcccgaatcgcgttcgtggaacaggatttatgccatggcaggcatggaatggggcaaattgacggtgattccgcgcgtgtgggtgcgtgcgttcgatcagagcggcgataaaaacgacaatcccgatattgccgactatatggggtatggcgacgtgaagctgcagtaccgcctgaacgacaggcagaatgtgtattccgtattgcgctacaaccccaaaacgggctacggcgcgattgaagccgcctacacgtttccgattaagggcaaactcaaaggcgtggtacgcggattccacggttacggcgagagcctgatcgactacaaccacaagcagaacggtatcggtatcgggttgatgttcaacgactgggacggcatctga

SEQ ID NO: 95 - protein, 375 aa, Neisseria gonorrhoeae
MNTRNMRYILLTGLLPTASAFGETALQCAALTDNVTRLACYDRIFAAQLPSSAGQEGQESKAVLNLTETVRSSLDKGEAVIVVEKGGDALPADSAGETADIYTPLSLMYDLDKNDLRGLLGVREHNPMYLMPFWYNNSPNYAPSSPTRGTTVQEKFGQQKRAETKLQVSFKSKIAENLFKTRADLWFGYTQRSDWQIYNQGRKSAPFRNTDYKPEIFLTQPVKADLPFGGRLRMLGAGFVHQSNGQSRPESRSWNRIYAMAGMEWGKLTVIPRVWVRAFDQSGDKNDNPDIADYMGYGDVKLQYRLNDRQNVYSVLRYNPKTGYGAIEAAYTFPIKGKLKGVVRGFHGYGESLIDYNHKQNGIGIGLMFNDWDGI

SEQ ID NO: 96 - DNA, 1000 bp, Neisseria gonorrhoeae
ttttggcttccagcgtttcgttgttttcgtacaagtcgtaagtcagcttcagattgttggcttttttaaagtcttcgaccgtactctcgtcaacataattcgaccagttgtagatgttcagagtatcggtggcagcggcttcggcattggcagcaggtgcgctgcctgcttgaggctgcacggcgtttttttcgctgccgccgcaggctgccagagacagcgcggccaaaacggctaatacggattttttcatacgggcagattcctgatgaaagaggttggaaaaaaagaaaaccccgcgccccataaacaccccggcgcaaggtttgggtattgtaaagtaaatttgtgcaaactcaaagcgatattggcctgattttcctaaaaaattaccctgtttccaaaaaagggggggaaacggccgcccgattttgccgtttttttgcgccgtcagggtgtccgacgggcggatagagaaaaaaggcttgcatataatgtaaaccccctttaaaattgcgcgtttacagaatttatttttctttcaggagattccaatatggcaaacagcgcacaagcacgcaaacgtgcccgccagtccgtcaaacaacgcgcccacaacgctagcctgcgtaccgcattccgcaccgcagtgaaaaaagtattgaaagcagtcgaagcaggcgataaagctgccgcacaagcggtttaccaagagtccgtcaaagtcatcgaccgcatcgccgacaaaggcgtgtttcataaaaacaaagcggctcgccacaaaagccgcctgtctgcaaaagtaaaagcactggcttgatttttgcaaaaccgccaaggcggttgatacgcgataagcggaaaaccctgaagcccgacggtttcggggttttctgtatttcgggggtaaagttcgaaatggcggaaagggtgcggttttttatccgaatccgctataaaatgccgtttgaaaaccaatatgccgacaatgggggcggag

SEQ ID NO: 97 - protein, 713 aa, Neisseria gonorrhoeae
MNAPFFRLSLLSLTLAAGFAHAAENNANVALDTVTVKGDRQGSKIRTNIVTLQQKDESTATDMRELLKEEPSIDFGGGNGTSQFLTLRGMGQNSVDIKVDNAYSDSQILYHQGRFIVDPALVKVVSVQKGAGSASAGIGATNGAIIAKTVDAQDLLKGLDKNWGVRLNSGFAGNNGASYGASVFGKEGNFDGLFSYNRNDEKDYEAGKGFRNDNGGKTVPYSALDKRSYLAKIGTTFGDGDHRIVLSHMKDQHRGIRTVREEFAVSEKNSRITIKRQAPSYRETTQSNTNLAYTGKDLGFVEKLDANAYVLEKKRYSADDKDNGYAGNVKGPNHTRIATRSMNFNFDSRLAEQTLLKYGINYRHQEIKPQAFLNSEFEIKDKEKATNEEKKKNRENEKIAKAYRLTNPTKTDTGAYIEAIHEIDGFTLTGGLRYDRFKVKTHDGKTVSSSSLNPSFGVIWQPREHWSFSASHNYAGRSPRLYDALQTHGKRGIISIADGTKAERARNTEIGFNYNDGTFAANGSYFRQTIKDALANPQNRHDSVAVREAVNAGYIKNHGYELGASYRTGGLTAKVGVSHSKPRFYDTHKDKLLSANPEFGAQVGRTWTASLAYRFKNPNLEIGWRGRYVQKAVGSILAAGQKDRDGKLENVVRQGFGVNDVFANWKPLGKDTLNVNLSVNNVFDKFYYPHSQRWTNTLPGVGRDVRLGVNYKF

SEQ ID NO: 98 - DNA, 2600 bp, Neisseria gonorrhoeae
aaaccggtacggcgttgccccgccttagctcaaagagaacgattccctaaggtgctgaagcaccgagtgaatcggttccgtactatttgtactgtctgcggcttcgccgccttgtcctgatttttgttagtccacatatacatttccgacaaaacctgtcaacaaaaaacaacgcttcgcaaataaaaacgataatcagctttacacaacccccccccgctaatataaacaaaaataattattattattttttcttatcctgccaaaccttaacggtttggcttaacttcccttcatacactcaaaaggacgaacaaatgaacgccccgtttttccgcctcagcctgctctcgctcacacttgccgccggctttgcccacgcggcagaaaataatgccaatgtcgcattggataccgttaccgtaaaaggcgaccgccaaggcagcaaaatccgtaccaacatcgttacgcttcaacaaaaagacgaaagcaccgcaaccgatatgcgcgaactcttaaaagaagagccctccatcgatttcggcggcggcaacggcacgtcccaattcctgacgctgcgcggtatgggtcagaactctgtcgacatcaaggtggacaacgcctattccgacagccaaatcctttaccaccaaggcagatttattgtcgatcccgctttggttaaagtcgtttccgtacagaaaggcgcgggttccgcctctgccggtatcggcgcgaccaacggcgcgatcatcgccaaaaccgtcgatgcccaagacctgctcaaaggcttggataaaaactggggcgtgcgcctcaacagcggctttgccggcaacaacggcgcaagctacggcgcaagcgtattcggaaaagagggcaacttcgacggtttgttctcttacaaccgcaacgatgaaaaagattacgaagccggcaaaggtttccgcaatgacaacggcggcaaaaccgtaccgtacagcgcgctggacaaacgcagctacctcgccaaaatcggaacaaccttcggcgacggcgaccaccgcatcgtgttgagccatatgaaagaccaacaccggggcatccgcactgtgcgtgaagagtttgccgtcagcgaaaaaaattcacggataactattaaacgccaagccccatcctaccgcgaaaccactcaatccaacaccaacttggcgtacaccggcaaagatttgggctttgtcgaaaaactggatgccaacgcctatgtgttggaaaagaaacgctattccgccgatgacaaagataacggctacgcaggcaatgtaaaaggccccaaccatacccgaatcgccactcggagtatgaacttcaacttcgacagccgccttgccgaacaaaccctgttgaaatacggcatcaactaccgccatcaggaaatcaaaccgcaagcgtttttgaactcggaatttgaaataaaagataaagaaaaagcaactaatgaagagaaaaagaagaaccgtgaaaatgaaaaaattgccaaagcctaccgcctgaccaacccgaccaaaaccgataccggcgcgtatatcgaagccattcacgagattgacggctttaccctgaccggcgggctgcgttacgaccgcttcaaggtgaaaacccacgacggcaaaaccgtttcaagcagcagcctcaacccgagtttcggcgtgatttggcagccgcgcgaacactggagcttcagcgcgagccacaactacgccggccgcagcccgcgcctgtatgacgctctgcaaacccacggcaagcgcggcatcatctcgattgccgacggcacgaaagccgaacgcgcgcgcaataccgaaatcggcttcaactacaacgacggcacgtttgccgcaaacggcagctacttccggcagaccatcaaagacgcgcttgccaatccgcaaaaccgccacgactccgtcgccgtccgcgaagccgtcaacgccggctacatcaaaaaccacggttacgaattgggcgcgtcctaccgcaccggcggcctgaccgccaaagtcggcgtaagccacagcaaaccgcgcttttacgatacgcacaaagacaagctgttgagcgcgaaccctgaatttggcgcacaagtcggccgcacttggacggcctcccttgcctaccgcttcaaaaacccgaatctggaaatcggctggcgcggtcgttatgttcaaaaagccgtgggttcgatattggcggcaggtcaaaaagaccgcgacggcaaattggaaaacgttgtacgccaaggtttcggtgtgaacgatgtcttcgccaactggaaaccgctgggcaaagacacgctcaatgttaatctttcggttaacaacgtgttcgacaagttctactatccgcacagccaacgctggaccaataccctgccgggcgtgggacgtgatgtacgcctgggcgtgaactacaagttctaaaacgcacatcccgaaaaaatgccgtctgaaagcctttcagacggcatctgtcctgataatttgatatatagtggattaacaaaaaccggtacggcgttgccccgccttagctcaaagggaacgattccctaaggtgctgaa

SEQ ID NO: 99 - protein, 236 aa, Neisseria gonorrhoeae
MTKQLKLSALFVALLASGTAVAGEASVQGYTVSGQSNEIVRNNYGECWKNAYFDKASQGRVECGDAVAVPEPEPAPVAVVEQAPQYVDETISLSAKTLFGFDKDSLRAEAQDNLKVLAQRLSRTNVQSVRVEGHTDFMGSEKYNQALSERRAYVVANNLVSNGVPASRISAVGLGESQAQMTQVCQAEVAKLGAKASKAKKREALIACIEPDRRVDVKIRSIVTRQVVPARNHHQH

SEQ ID NO: 100 - DNA, 1349 bp, Neisseria gonorrhoeae
gaattcctatccgatttgccgccatgtttctacagcggcctgtatgttggcaattcagcagttgcttctgtatctgctgtacaaatctaatgagggaataaaatgaccaaacagctgaaattaagcgcattattcgttgcattgctcgcttccggcactgctgttgcgggcgaggcgtccgttcagggttacaccgtaagcggccagtcgaacgaaatcgtacgcaacaactatggagaatgctggaaaaacgcctactttgataaagcaagccaaggtcgcgtagaatgcggcgatgcggttgccgtccccgagcccgaacccgcgcctgtcgccgttgtggagcaggctcctcaatatgttgatgaaaccatttccctgtctgccaaaaccctgttcggtttcgataaggattcattgcgcgccgaagctcaagacaacctgaaagtattggcgcaacgcctgagtcgaaccaatgtccaatctgtccgcgtcgaaggccataccgactttatgggttctgaaaaatacaatcaggctctgtccgaacgccgcgcatacgtagtggcaaacaacctggtcagcaacggcgtacctgcttctagaatttctgctgtcggcttgggcgaatctcaagcgcaaatgactcaagtttgtcaagccgaagttgccaaactgggtgcgaaagcctctaaagccaaaaaacgtgaggctctgattgcatgtatcgaacctgaccgccgcgtagatgtgaaaatccgcagcatcgtaacccgtcaggttgtgccggcacgcaatcatcaccaacactaaggctaggtaatatcttgccgatgcatgaggttagcggattttgtaccgggtactgttgcaatattcgtgaaacgtcggccggtatcgatgatgtgaaacaaaccccgcttttgcggggtttgtttttttgggtggttttctgaaacggctatcgtcagaatcggggtgcaggttcggattcggattcagattcatgtttgtgtcccattgccgcgctttatagtggattaacaaaaatcaggacaaggcgacgaagccgcagacagtacaatagtacggcaaggcgaggcaacgccgtaccggtttaaatttaatccactatatcggttgaaactctgattttaaggcggtaggatgtgggtttgcccatagcaagggaatcctttctgtatcaagccccgaaagggataattcatacaaattcacgcctttccccctcattgggaaatggatggaatcgtgcccgatgtgtgcggcactgtatgccggatatggttttatcatcatcccttttcggttgaaaccccgcggaattc

SEQ ID NO: 101 - DNA, 32 bp, Artificial Sequence
  Primer
gggaatccatatgaaaaaactcttgaaatcgg

SEQ ID NO: 102 - DNA, 29 bp, Artificial Sequence
  Primer
ggaagatctttagaagcggaattgtgcat

SEQ ID NO: 103 - DNA, 36 bp, Artificial Sequence
  Primer
ctgcagaatcgcgaatgaaaaaactcttgaaatcgg

SEQ ID NO: 104 - DNA, 35 bp, Artificial Sequence
  Primer
ctgcagaatcgcgaatgagtagcaagctagtgaac

SEQ ID NO: 105 - DNA, 29 bp, Artificial Sequence
  Primer
aggagatctttagaattggaatcctccgg

SEQ ID NO: 106 - DNA, 1194 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcaccacttggtgtgacgctatcagcatgcgcgttggttactacggagactttgttttcgaccgtgttttgaaaactgatgtgaataaagaatttcagatgggagcggcgcctactaccagcgatgtagaaggcttacaaaacgatccaacaacaaatgttgctcgtccaaatcccgcttatggcaaacacatgcaagatgctgaaatgtttacgaacgctgcttacatggcattaaatatctgggatcgttttgatgtattttgtacattgggagcaactaccggttatttaagaggaaactccgcttccttcaacttagttggattattcggaacaaaaacacaatattctaagtttaatacagcgaatcttgttcctaacactgctttggatcgagctgtggttgagctttatacagacaccacctttgcttggagcgtaggtgctcgtgcagctctctgggaatgtgggtgtgcaacgttaggagcttctttccaatatgctcaatctaaacctaaagtagaagagttaaatgttctttgtaatgcatccgaatttactattaataagccgaaaggatatgttggggtggaatttccacttgatattaccgcaggaacagaagctgcgacagggactaaggatgcctctattgactaccatgagtggcaagcaagtttagccctttcttacagattaaatatgttcactccttacattggagttaaatggtctagagtaagttttgatgccgacacgatccgtatcgctcagcctaaattggctgaagcaatcttggatgtcactactctaaacccgaccatcgctggtaaaggagctgtggtctcttccggaagcgataacgaactggctgatacaatgcaaatcgtttccttgcagttgaacaagctgaaatctagaaaatcttgcggtattgcagtaggaacgactattgtagatgcagataaatacgcagttacagttgagactcgcttgatcgatgagagagcagctcacgtaaatgcacaattccgcttctaa

SEQ ID NO: 107 - DNA, 1182 bp, Chlamydia trachomatis; identical to SEQ ID NO: 56

SEQ ID NO: 108 - DNA, 1023 bp, Chlamydia trachomatis; identical to SEQ ID NO: 1